(19) United States
(12) Patent Application Publication
(10) Pub. No.: US 2024/0209351 A1
(43) Pub. Date: Jun. 27, 2024

(54) METHODS FOR IMPROVING TRAITS IN PLANTS

(71) Applicant: PLANTARCBIO LTD., Raanana (IL)

(72) Inventors: Dror Shalitin, Raanana (IL); Noam Grimberg, Raanana (IL); Arava Shatil Cohen, Raanana (IL)

(21) Appl. No.: 18/407,953

(22) Filed: Jan. 9, 2024

Related U.S. Application Data
(63) Continuation of application No. 17/388,310, filed on Jul. 29, 2021, now Pat. No. 11,905,512.
Application No. 17/388,310 is a continuation of application No. 16/496,445, filed on Sep. 22, 2019, now Pat. No. 11,111,491.
(60) Provisional application No. 62/477,517, filed on Mar. 28, 2017.
(60) Provisional application No. 62/644,600, filed on Mar. 19, 2018.

(51) Int. Cl. C12N 15/10; C12N 15/82

(52) U.S. Cl. CPC C12N 15/1075; C12N 15/8273

(57) ABSTRACT

The present invention discloses a method for screening for and identifying a desirable plant improving trait, said method comprises steps of: (a) obtaining genetic material from a sampling of a predefined source and (b) constructing an expression library from said genetic material. The aforementioned method further comprises steps of: (c) producing plants transformed with said expression library at a transformation efficiency of at least 0.05%-30%, representing at least 10²-10¹⁰ transgenes; (d) screening for transformed plants expressing said desirable trait; and (e) identifying said transgene of said transformed plants expressing said desirable trait.

### RELATED APPLICATIONS

This application is a Continuation of U.S. patent application Ser. No. 17/388,310, filed on Jul. 29, 2021, which is a Continuation of U.S. patent application Ser. No. 16/496,445 filed on Sep. 22, 2019 (issued as U.S. Pat. No. 11,111,491), which is the U.S. National Stage of International Patent Application No. PCT/IL2018/050349, filed on Mar. 27, 2018, which claims the benefit of priority of U.S. Provisional Application Nos. 62/477,517 and 62/644,600, filed on Mar. 28, 2017 and Mar. 19, 2018, respectively. The contents of the above applications are all incorporated by reference as if fully set forth herein in their entirety.

### SEQUENCE LISTING

The official copy of the sequence listing is submitted electronically in ST.26 XML format having the file name “11184_033US-CON3_Seq_List.xml” created on Mar. 27, 2018, and having a size of 452,840 bytes, and is filed concurrently with the specification. The Sequence Listing ST.26 XML file is part of the specification and is herein incorporated by reference in its entirety.

### FIELD OF THE INVENTION

The present invention generally relates to the field of improving traits in plants. More particularly, the present invention relates to improving traits in plants by transformation of expression libraries from predefined sources into plants and screening for desirable traits.

### BACKGROUND OF THE INVENTION

The world population is estimated to be 9.2 billion in 2050. To sufficiently feed this population, the total food production will have to increase by 60%-70%. Climate models predict that warmer temperatures and increases in the frequency and duration of drought during the present century will have negative impact on agricultural productivity. For example, maize production in Africa could be at risk of significant yield losses as researchers predict that each degree-day that the crop spends above 30° C. reduces yields by 1% if the plants receive sufficient water. These predictions are similar to those reported for maize yield in the United States. It has been further shown that maize yields in Africa decreased by 1.7% for each degree-day the crop spent at temperatures of over 30° C. under drought. Wheat production in Russia decreased by almost one-third in 2010, largely due to the summer heat wave. Similarly, wheat production declined significantly in China and India in 2010, largely due to drought and sudden rise in temperature respectively, thereby causing forced maturity. These new global challenges require a more complex integrated agriculture.

In addition global warming leads to the concurrence of a number of abiotic and biotic stresses, thus affecting agricultural productivity. Occurrence of abiotic stresses can alter plant-pest interactions by enhancing host plant susceptibility to pathogenic organisms, insects, and by reducing competitive ability with weeds. On the contrary, some pests may alter plant response to abiotic stress factors.

Biotic stress factors are caused by pathogens, insects, pests, weeds, or intraspecific competition for resources. The ability of biotic stress factors to cause yield or quality loss depends on the environment and thus may vary from region to region or from one agroecology to another. For example, in Australia, barley foliar diseases are some of the major biotic stress factors causing substantial yield and quality losses. Although it is known that some plant species have resistance to various diseases, they are hard or even impossible to breed in conventional methods.

The challenge is to create crops that are resistance to biotic stress factors and are flexible and adaptable to diverse environments and populations. There are currently two major acceptable ways to adapt crops to new environments: developing new crops through conventional breeding (long-term endeavor starting with domestication) and introducing target traits into existing crops through plant breeding, which includes genetic engineering. To maintain productivity in the face of increased climatic variability, both the population and the plant cultivars will need to be continually developed to withstand “new” climate extremes and other stresses such as diseases, pathogens, insects, pests etc. In addition there is a constant need to find new herbicide tolerance or resistant genes for new chemicals and new herbicides mode of action.

Genetic engineering has the potential to address some of the most challenging biotic and abiotic constraints faced by farmers, which are not easily addressed through conventional plant breeding alone.

Advantageous outcomes of these genetic modifications include increased food production, reliability, and yields; enhanced taste and nutritional value; and decreased losses due to various biotic and abiotic stresses, such as fungal and bacterial pathogens. These objectives continue to motivate modern breeders and food scientists, who are seeking for newer genetic modification methods for identifying, selecting, and analyzing individual organisms that possess genetically enhanced features.

The option to transform plants with foreign genes and/or genes from the same specie or genus, that are hard or impossible to breed, overcomes species barriers, making it possible to exploit powerful ‘super-traits’ that are not attainable through traditional methods. However, the molecular interactions and outcomes of introduced trans-genes and endogenous genes are not predictable.

When genes coding for certain traits are transferred, typically from one plant species to another, the desired traits are not always expressed unless the environment interacts with the genes in the anticipated way triggering the desired response, which depends on the regulating sequences inserted with the gene. This means that new transgenic cultivars, developed under laboratory conditions in a controlled climate, have to be tested under field conditions, as in more traditional breeding methods, so currently there is little difference in the speed with which either method will result in the release of new cultivars.

The knowledge gained from basic plant research will underpin future crop improvements, but effective mechanisms for the rapid and effective translation of research discoveries into public good agriculture remain to be developed.

U.S. Pat. Nos. 6,030,779 and 6,368,798 disclose a process for identifying clones having a specified enzyme activity by selectively isolating target nucleic acid from genomic DNA population, by use of polynucleotide probe identifying the nucleic acid sequence encoding an enzyme having the specified enzyme activity; and transforming a host with the isolated target nucleic acid to produce a library of clones which are screened for the specified enzyme activity.

U.S. Pat. No. 6,972,183 discloses a process for screening an expression library to identify clones expressing enzymes having a desired activity. The process involves generating from genomic DNA samples of one or more microorganisms an expression library comprising a plurality of recombinant cell clones, and then introducing into capillaries in a capillary array a substrate and a subset of the clones. Interaction of the substrate and a clone expressing an enzyme having the desired activity produces an optically detectable signal, which can then be spatially detected to identify capillaries containing clones producing such a signal. The signal-producing clones can then be recovered from the identified capillaries.

EP patent application 1025262 and US patent application 20020150949 teach a process for identifying clones having a specified activity of interest, by (i) generating expression libraries derived from nucleic acid directly isolated from the environment; (ii) exposing said libraries to a particular substrate or substrates of interest; and (iii) screening said exposed libraries utilizing a fluorescence activated cell sorter to identify clones which react with the substrate or substrates.

US patent application 20100152051 relates to a method for the identification and/or characterization of clones conferring a desired biological property from an expression library. The method comprises the step of screening for the expression of at least one (poly)peptide, such as a tag expressed as a fusion protein, together with a recombinant insert of a clone of said expression library. Said (poly)peptide may be fused N-terminally or C-terminally to said insert. The method further comprises the steps of contacting a ligand specifically interacting with the (poly)peptide expressed by the insert of a clone conferring said desired biological property.

All the above methods are based upon screening a DNA library (produced from microorganisms or environmental sample) for a specific sequence or biochemical activity via interaction with a predetermined probe. In addition, the screening and selection for a clone having the predetermined sequence or activity is performed prior to transformation into plant cells and could be expressed in plant cells (tissue cultures) but not in whole plants. Thus by the up-to-date used methods, only the preselected clone is expressed in plants and the expression and effect of the selected sequence in plants is unpredictable. In addition, in the methods described above, one can screen only for known activities based on prior knowledge. Thus, these methods are limited under the scope of known enzyme activities and enzyme families and prior known function.

In view of the above, there is a long felt need for efficient methods for screening and identifying unknown sequences conferring desirable plant improving traits.

### SUMMARY OF THE INVENTION

It is therefore one object of the present invention to disclose a method for screening for and identifying a desirable plant improving trait, the method comprises steps of: (a) obtaining genetic material from a sampling of a predefined source; (b) constructing an expression library from said genetic material; wherein said method further comprises steps of: (c) producing plants transformed with said expression library at a transformation efficiency of at least 0.05%-30%, representing at least 102-1010 transgenes; (d) screening for transformed plants expressing said desirable trait; and (e) identifying said transgene of said transformed plants expressing said desirable trait.

It is a further object of the present invention to disclose the method as defined above, further comprising a step of editing a target gene in a desirable crop plant according to genetic information obtained from said transgene.

It is a further object of the present invention to disclose the method as defined in any of the above, wherein said editing of said target gene is performed using any genome editing system or method including systems using engineered nucleases selected from the group consisting of: meganucleases, zinc finger nucleases (ZFNs), transcription activator-like effector-based nucleases (TALEN), clustered regularly interspaced short palindromic repeats (CRISPR) system and any combination thereof.

It is a further object of the present invention to disclose the method as defined in any of the above, wherein said predefined source comprises plant, microbial, fungal or other organisms or parts thereof of an environmental niche.

It is a further object of the present invention to disclose the method as defined in any of the above, wherein said screening step comprises measurements of said transformed plants as compared to control plants, said measurements are selected from the group consisting of: turgor pressure measurements, plant death, leaf area, plant shoots fresh weight, leaf number, branch fresh weight, main branch length, flowers yield, pods or fruits yield, chlorosis, damage to leaves, state or performance of plants and any combination thereof.

It is a further object of the present invention to disclose the method as defined in any of the above, wherein said control plant is a plant of the same genus as said transgenic plant and lacking said transgene or a plant of the same genus as said transgenic plant, lacking said transgene and transformed with a known gene conferring said plant improving trait.

It is a further object of the present invention to disclose the method as defined in any of the above, wherein said step (a) further comprises steps of enriching said genetic material by growth on rich media or on selective media.

It is a further object of the present invention to disclose the method as defined in any of the above, wherein said step (a) further comprises steps of enhancing expression of said desirable trait by culturing said genetic material on selective media for said desirable trait.

It is a further object of the present invention to disclose the method as defined in any of the above, wherein said step (b) comprises steps of producing prokaryotic cDNA library or eukaryotic cDNA library or both.

It is a further object of the present invention to disclose the method as defined in any of the above, wherein said step (b) further comprises steps of cloning said cDNA library into at least one binary vector.

It is a further object of the present invention to disclose the method as defined in any of the above, wherein said binary vector comprises a constitutive promoter or a stress induced promoter.

It is a further object of the present invention to disclose the method as defined in any of the above, wherein said binary vector comprises bacterial selection marker and plant transformation selection marker.

It is a further object of the present invention to disclose the method as defined in any of the above, further comprises steps of transforming said cloned binary vectors into host cells.

It is a further object of the present invention to disclose the method as defined in any of the above further comprises steps of transforming said cloned binary vectors into Agrobacterium tumefaciens.

It is a further object of the present invention to disclose the method as defined in any of the above further comprises steps of introducing said transformed Agrobacterium tumefaciens into at least one of: whole plant, plant tissue and plant cell.

It is a further object of the present invention to disclose the method as defined in any of the above, comprises steps of introducing said transformed Agrobacterium tumefaciens by spraying said plants with an inoculum comprising transformed Agrobacterium.

It is a further object of the present invention to disclose the method as defined in any of the above, wherein said step (d) comprises growing said transformed plants under conditions selective for said desirable trait.

It is a further object of the present invention to disclose the method as defined in any of the above, further comprises steps of: (f) collecting T1 seeds from said transformed plants of step (d); (g) determining seed library transformation efficiency of said T1 seeds; (h) sowing said T1 seeds of step (e) under selective conditions allowing screening and selection of transformed plants expressing said desirable trait; (i) testing said selected plants expressing said desirable trait of step (g) for presence of said transgene; and (j) isolating and sequencing said transgene of said selected transformed plants positively tested for said transgene of step (h).

It is a further object of the present invention to disclose the method as defined in any of the above, further comprises steps of (k) collecting T2 seeds from said plants of (h), which are found positive for presence of said transgene; (1) growing plants of said T2 seeds under selective conditions allowing screening and selection of transformed plants expressing said desirable trait as compared to control plants transformed with known genes conferring said desirable trait; and (m) optionally, isolating and sequencing said transgene of said selected plants of step (j).

It is a further object of the present invention to disclose the method as defined in any of the above, comprises steps of (a) recloning and sequencing said isolated transgene of step (i) and /or (l); (b) transforming said recloned transgene into plants; (c) screening said transformed plants of step (b) for selection of transformed plants expressing said desirable trait; (d) isolating said transgene from said selected plants of step (c); and (e) optionally, repeating steps (a) to (d).

It is a further object of the present invention to disclose the method as defined in any of the above, wherein said environmental niche comprises ecological niche, populations, habitats, gene pools, prokaryotic culture, eukaryotic culture and any combination thereof.

It is a further object of the present invention to disclose the method as defined in any of the above, wherein said environmental niche comprises microbiome, microbiota, microbial culture, plant, yeast, algae, nematode or any other organism or combinations thereof.

It is a further object of the present invention to disclose the method as defined in any of the above, wherein said environmental niche comprises predefined biotic factors, abiotic factors and a combination thereof.

It is a further object of the present invention to disclose the method as defined in any of the above, wherein said sampling comprises soil sample, water sample, organic matter sample and any combination thereof.

It is a further object of the present invention to disclose the method as defined in any of the above, wherein said desirable trait is selected from the group consisting of resistance or tolerance to at least one biotic stress, resistance or tolerance to at least one abiotic stress, improved yield, improved biomass, improved food qualities and values, improved grain yield, herbicide or chemical resistance or tolerance and any combination thereof.

It is a further object of the present invention to disclose the method as defined in any of the above, wherein said abiotic stress is selected from the group consisting of: drought, salinity, heat, cold, fertilizer uptake, fertilizer usage efficiency and any combination thereof.

It is a further object of the present invention to disclose the method as defined in any of the above, wherein said biotic stress is selected from the group consisting of: plant diseases, pathogens, bacteria, viruses, fungi, parasites, beneficial and harmful insects, weeds, and cultivated or native plants or any combination thereof.

It is a further object of the present invention to disclose the method as defined in any of the above, wherein said step (a) comprises steps of extracting RNA from said sampling of said predefined environmental niche.

It is a further object of the present invention to disclose the method as defined in any of the above, wherein said RNA extraction is performed according to standard commercial kits or according to any other protocol for extraction of RNA from environmental sampling.

It is a further object of the present invention to disclose the method as defined in any of the above, wherein said protocol for extraction of RNA from environmental sampling comprises steps of: (a) obtaining a soil sample; (b) mixing said soil sample with an extraction buffer comprising 500 mM phosphate buffer pH 8 and 5% w/v cetyltrimethylammonium bromide (CTAB) with phenol (pH 8)/chloroform/IAA ratio of 25:24:1; (c) subjecting said mixture of step (b) to 15 min shaking at 37° C. or to a bead beater for 1 min; (d) centrifuging said mixture of step (c) at 2,500 g for about 10 minutes at room temperature to obtain an aqueous phase; (e) transferring said aqueous phase into a new tube; (f) adding to said aqueous phase within said tube of step (e) an equal amount of iso-propanol supplemented with 20 mg/ml crystal violet solution to obtain violate stained solution; (g) mixing said solution by inverting said tube of step (f) and then incubating said tube for about 30 minutes at room temperature; (h) centrifuging said tube of step (g) at 2,500 g for about 30 minutes at room temperature to obtain a violet stained layer; (i) transferring said violate stained layer into a new tube and centrifuging said tube for about 5 min at maximal speed to obtain pellet and supernatant; (j) washing said pellet with 80% v/v ice cold ethanol and centrifuging for additional 5 min to obtain pellet and supernatant; (k) removing said supernatant of step (j) and allowing said pellet to dry; and (l) suspending said dried pellet in water in a ratio of 100 μl water to 2 gr of soil of step (a).

It is a further object of the present invention to disclose a plant comprising said transgene identified by the method as defined in any of the above.

It is a further object of the present invention to disclose the plant as defined above, wherein said plant has at least one plant improving trait as compared to a plant of the same genus lacking said transgene.

It is a further object of the present invention to disclose a polynucleotide sequence obtainable by the method as defined in any of the above.

It is a further object of the present invention to disclose the polynucleotide as defined in any of the above, wherein said polynucleotide comprises a nucleotide sequence corresponding to the sequence as set forth in a polynucleotide sequence selected from the group consisting of SEQ ID NOs:1-148 and any combination thereof.

It is a further object of the present invention to disclose a polynucleotide sequence having at least 80% sequence similarity to the polynucleotide sequence as defined in any of the above.

It is a further object of the present invention to disclose a polypeptide sequence obtainable by the method as defined in any of the above.

It is a further object of the present invention to disclose the polypeptide sequence as defined in any of the above, wherein said polypeptide comprises an amino acid sequence corresponding to the sequence as set forth in a polypeptide sequence selected from the group consisting of SEQ ID NOs: 149-321 and any combination thereof.

It is a further object of the present invention to disclose a polypeptide sequence having at least 60% sequence similarity to the polypeptide sequence as defined in any of the above.

It is a further object of the present invention to disclose the use of the method as defined in any of the above for identifying genes conferring plant improving traits selected from the group consisting of resistance or tolerance to abiotic stress, resistance or tolerance to biotic stress, improved yield, improved biomass, improved food qualities and values, improved grain yield, herbicide or chemical resistance or tolerance and any combination thereof.

It is a further object of the present invention to disclose the use as defined in any of the above, wherein said abiotic stress is selected from the group consisting of: drought, salinity, heat, cold, fertilize utilization and any combination thereof.

It is a further object of the present invention to disclose the use as defined in any of the above, wherein said biotic stress is selected from the group consisting of: plant diseases, pathogens, bacteria, viruses, fungi, parasites, beneficial and harmful insects, weeds, and cultivated or native plants or any combination thereof.

It is a further object of the present invention to disclose a method for screening for and identifying a drought or salinity resistance or tolerance improving trait in plants, said method comprises steps of: (a) obtaining genetic material derived from a low moisture or a high salinity source sample; (b) constructing expression library from said genetic material; wherein said method further comprises steps of: (c) producing plants transformed with said expression library at a transformation efficiency of at least 0.5%-30% representing at least 10²-10¹⁰ transgenes; (d) screening for transformed plants resistant or tolerant to predetermined drought or salinity conditions; and (e) identifying said transgene of said drought or salinity resistant or tolerant transformed plants of step (d).

It is a further object of the present invention to disclose the method as defined in any of the above, further comprising a step of editing a target gene in a desirable crop plant according to genetic information obtained from said transgene.

It is a further object of the present invention to disclose the method as defined in any of the above, wherein said editing of said target gene is performed using any genome editing system or method including systems using engineered nucleases selected from the group consisting of: meganucleases, zinc finger nucleases (ZFNs), transcription activator-like effector-based nucleases (TALEN), clustered regularly interspaced short palindromic repeats (CRISPR) system and any combination thereof.

It is a further object of the present invention to disclose the method as defined in any of the above, wherein said predefined source comprises plant, microbial, fungal or other organisms or parts thereof of an environmental niche.

It is a further object of the present invention to disclose the method as defined in any of the above, wherein said screening step comprises measurements of said transformed plants as compared to control plants, said measurements are selected from the group consisting of: turgor pressure measurements, plant death, leaf area, plant shoots fresh weight, leaf number, branch fresh weight, main branch length, flowers yield, pods or fruits yield, chlorosis, damage to leaves, state or performance of plants and any combination thereof.

It is a further object of the present invention to disclose the method as defined in any of the above, wherein said control plant is a plant of the same genus as said transgenic plant and lacking said transgene or a plant of the same genus as said transgenic plant, lacking said transgene and transformed with a known gene conferring said plant improving trait.

It is a further object of the present invention to disclose the method as defined in any of the above, wherein said step (b) further comprises steps of cloning said expression library into at least one binary vector.

It is a further object of the present invention to disclose the method as defined in any of the above, further comprises steps of: (f) collecting T1 seeds from said transformed plants of step (c); (g) sowing said T1 seeds in soil selective for transformed plants, with water content of about 100% capacity; (h) growing plants of said T1 seeds in drought or salinity conditions and/or without irrigation until most of the plants die, to produce transformed plants surviving said drought or salinity conditions; (i) growing said drought or salinity surviving transformed plants to produce T2 seeds; (j) screening said drought or salinity surviving transformed plants of step (i) for presence of a transgene; and (k) isolating and sequencing said transgene from positively screened plants of step (j).

It is a further object of the present invention to disclose the method as defined in any of the above, further comprises steps of (l) collecting T2 seeds from each of said transgene-containing positively screened drought or salinity surviving transformed plants of step (j); (m) growing T2 plants from each of said transgene-containing T2 seeds of step (l) under predetermined drought or salinity conditions as compared to control plants of the same genus and lacking said transgene or transformed with known genes conferring drought or salinity tolerance or drought or salinity resistance; (n) performing drought tolerance or resistance screening measurements for each of said transgene-containing T2 plants as compared to said control plants, said measurements are selected from the group consisting of: turgor pressure measurements, plant death, leaf area, fresh weight, leaf number, branch fresh weight, main branch length, flowers and pods production, Chlorosis and damage to leaves, state or performance of plants and any combination thereof; (o) isolating the transgene from said screened dough or salinity resistance performing T2 plants of step (n); (p) optionally, recloning said transgene into a binary vector; (q) optionally, transforming said cloned binary vector into plants and growing said transformed plants under predetermined drought or salinity conditions; and (r) optionally, repeating steps (l) to (q).

It is a further object of the present invention to disclose the method as defined in any of the above, wherein said step of growing T2 plants comprises steps of: (a) sowing said T2 seeds in soil selective for transformed plants, with water content of about 100% capacity; and (b) irrigating said plants when water content in the soil reaches about 5-10%.

It is a further object of the present invention to disclose the method as defined in any of the above, wherein said predetermined drought or salinity conditions are selected from the group consisting of low moisture, high salinity, dry soil and heat.

It is a further object of the present invention to disclose a polynucleotide sequence obtainable by the method as defined in any of the above.

It is a further object of the present invention to disclose the polynucleotide as defined in any of the above, wherein said polynucleotide comprises a nucleotide sequence corresponding to the sequence as set forth in a polynucleotide sequence selected from the group consisting of SEQ ID NOs:1 to SEQ ID NO:148 and any combination thereof.

It is a further object of the present invention to disclose a polynucleotide sequence having at least 80% sequence similarity to the polynucleotide sequence as defined in any of the above.

It is a further object of the present invention to disclose a polypeptide sequence obtainable by the method as defined in any of the above.

It is a further object of the present invention to disclose the polypeptide sequence as defined in any of the above comprises an amino acid sequence corresponding to the sequence as set forth as set forth in polypeptide sequence selected from the group consisting of SEQ. ID Nos: 149-321 and any combination thereof.

It is a further object of the present invention to disclose a polypeptide sequence having at least 60% sequence similarity with the polypeptide sequence as defined in any of the above.

It is a further object of the present invention to disclose a method for extracting RNA from a soil sample comprising steps of: (a) obtaining a soil sample; (b) mixing said soil sample with an extraction buffer comprising 500 mM phosphate buffer pH 8 and 5% w/v cetyltrimethylammonium bromide (CTAB) with phenol (pH 8)/chloroform/IAA ratio of 25:24:1; (c) subjecting said mixture of step (b) to 15 min shaking at 37° C. or to a bead beater for 1 min; (d) centrifuging said mixture of step (c) at 2,500 g for about 10 minutes at room temperature to obtain an aqueous phase; (e) transferring said aqueous phase into a new tube; (f) adding to said aqueous phase within said tube of step (e) an equal amount of iso-propanol supplemented with 20 mg/ml crystal violet solution to obtain violate stained solution; (g) mixing said solution by inverting said tube of step (f) and then incubating said tube for about 30 minutes at room temperature; (h) centrifuging said tube of step (g) at 2,500 g for about 30 minutes at room temperature to obtain a violet stained layer; (i) transferring said violate stained layer into a new tube and centrifuging said tube for about 5 min at maximal speed to obtain pellet and supernatant; (j) washing said pellet with 80% v/v ice cold ethanol and centrifuging for additional 5 min to obtain pellet and supernatant; (k) removing said supernatant of step (j) and allowing said pellet to dry; and (l) suspending said dried pellet in water in a ratio of 100 μl water to 2 gr of soil of step (a).

It is a further object of the present invention to disclose a method for screening for and identifying a desirable plant improving trait, said method comprises steps of: (a) obtaining a sampling of a predefined source; (b) extracting RNA from said sampling according to the method of claim 60; (c) constructing an expression library from said RNA of step (b); wherein said method further comprises steps of: (d) producing plants transformed with said expression library at an efficiency of at least 0.05%-30% representing at least 10²-10¹⁰ transgenes; (e) screening for transformed plants expressing said desirable trait; and (f) identifying said transgene of said transformed plants expressing said desirable trait.

It is a further object of the present invention to disclose the method as defined in any of the above, further comprising a step of editing a target gene in a desirable crop plant according to genetic information obtained from said transgene.

It is a further object of the present invention to disclose the method as defined in any of the above, wherein said editing of said target gene is performed using any genome editing system or method including systems using engineered nucleases selected from the group consisting of: meganucleases, zinc finger nucleases (ZFNs), transcription activator-like effector-based nucleases (TALEN), clustered regularly interspaced short palindromic repeats (CRISPR) system and any combination thereof.

It is a further object of the present invention to disclose the method as defined in any of the above, wherein said predefined source comprises plant, microbial, fungal or other organisms or parts thereof of an environmental niche.

It is a further object of the present invention to disclose the method as defined in any of the above, wherein said screening step comprises measurements of said transformed plants as compared to control plants, said measurements are selected from the group consisting of: turgor pressure measurements, plant death, leaf area, plant shoots fresh weight, leaf number, branch fresh weight, main branch length, flowers yield, pods or fruits yield, chlorosis, damage to leaves, state or performance of plants and any combination thereof.

It is a further object of the present invention to disclose the method as defined in any of the above, wherein said control plant is a plant of the same genus as said transgenic plant and lacking said transgene or a plant of the same genus as said transgenic plant, lacking said transgene and transformed with a known gene conferring said plant improving trait.

It is a further object of the present invention to disclose an isolated polynucleotide having at least 80% sequence similarity to a nucleotide sequence selected from the group consisting of SEQ ID NOs:1 to SEQ ID NO:148 and any combination thereof.

It is a further object of the present invention to disclose an isolated polypeptide having at least 60% sequence similarity to an amino acid sequence selected from the group consisting of SEQ. ID Nos: 149-321 and any combination thereof.

### BRIEF DESCRIPTION OF THE DRAWINGS

In order to understand the invention and to see how it may be implemented in practice, several embodiments will now be described, by way of non-limiting example only, with reference to the accompanying drawing, in which:

FIGS. 1A-D present schematic illustrations of binary vectors used for insertion of amplified cDNA clones between the promoter(s) (35S, CBF3, Erd10 and Kin1) and the HSP terminator. FIG. 1A illustrates the pPA-35H vector, which has a constitutive CaMV 35S promoter. FIGS. 1B-D present vectors containing stress induced promoters of Arabidopsis thaliana: pPA-CH with CBF3 promoter (FIG. 1B), pPA-EH with Erd10 promoter (FIG. 1C) and pPA-KH with Kin1 promoter (FIG. 1D);

FIG. 2 presents a photographic illustration of Agrobacterium library counting for 3 different libraries on LB petri dishes;

FIGS. 3A-3C presents a photographic illustration of tobacco tissue culture transformed with a library, 7 days after transformation (FIG. 3A), 40 days after transformation (FIG. 3B) and 6-8 weeks after transformation (FIG. 3C);

FIG. 4 presents a photographic illustration demonstrating selection for phosphinothricin resistance of 10 days old Arabidopsis expressing library seedlings. The green plants are resistant to phosphinothricin while small yellow plants are absent of the transgene and therefore susceptible;

FIG. 5 presents a photographic illustration of T2 and T3 controlled experiments in the greenhouse;

FIG. 6 presents photographic results of screening for transgenic plants resistance to drought;

FIG. 7 presents a graphic illustration demonstrating loss of turgor pressure in plants expressing genes used as control relative to soil water content (dark gray), days after cessation of irrigation;

FIG. 8 presents a graphic illustration showing normalized death scale of positive control expressing transgenic plants as compared to GFP expressing plants;

FIG. 9 graphically shows results of several drought resistance genes identified by the method of the present invention; and

FIG. 10 graphically shows leaf area analysis of several transgenic plant lines expressing identified novel genes conferring drought resistance after re-cloning as compared to positive and negative controls.

### DETAILED DESCRIPTION OF THE PREFERRED EMBODIMENTS

The following description is provided, alongside all chapters of the present invention, so that to enable any person skilled in the art to make use of the invention and sets forth the best modes contemplated by the inventor of carrying out this invention. Various modifications, however, will remain apparent to those skilled in the art, since the generic principles of the present invention have been defined specifically to provide means and methods for screening and identifying a desirable plant improving trait.

It is known that some plant species have resistance to various diseases. However, such species are usually hard or impossible to breed in conventional techniques and methods.

The present invention provides a method and platform to discover and identify genes from plants that have unique and valuable features, such as disease resistance, abiotic stress resistance or tolerance, food improving qualities (e.g. improved oils, protein content, amino acids, vitamins etc.) and then to insert or express them in desired crops through gene editing, or other transformation technique.

It is therefore within the scope of the present invention to introduce target traits into existing crops through plant breeding, which includes genetic engineering and gene (genome) editing.

The present invention provides a novel method for screening and identifying a desirable plant improving trait. The method comprises steps of: (a) obtaining genetic material from a sampling of a predefined environmental niche or genetic material extracted from other sources such as plants from the same or other genus; and (b) constructing an expression library from said genetic material. According to core aspects, the present invention further comprises steps of: (c) producing plants transformed with said expression library at an efficiency of at least 0.05%-30% representing at least 10²-10¹⁰ transgenes; (d) screening for transformed plants expressing said desirable trait; and (e) identifying said transgene of said transformed plants expressing said desirable trait.

The present invention provides for the first time a method for screening for and selecting unknown sequences derived from predefined sources (e.g. ecological niches and/or plants) which confer improved traits in valuable crop plants. The current method is effective and advantageous upon common and conventional screening methods by the following aspects:
1. An expression library is prepared from genetic material or genetic pool (i.e. RNA) originating from predefined sources, such as extreme environment, plant material and other. In this way, only genes which are expressed in the preselected environmental conditions are used for the screening procedure in plants.
2. The entire expression library is transformed into plants at an efficiency of 0.05%-30% and representation of at least 10²-10¹⁰ unique transgenes.
3. In the method of the present invention, the screening of the expressed library for the desirable phenotype is performed at the target organism, which is the plant. In this way there is no preselection and new and unique genes for the desired phenotype, which are expressible in plants, are revealed.

In the conventional methods, the first step is selecting genes for a predefined trait in a source genetic material, e.g. by probing a DNA library with known sequences in prokaryotic- or eukaryotic cells, and only then the preselected gene is expressed in plants. The outcome of such a conventional method is limited and has the following drawbacks:
1. The screening is performed in a host cell/organism which is not the target organism (usually in prokaryotic or unicellular organism).
2. The screening is limited since it is performed with known sequences or probes or activity. It was shown that functional screening methods require detectable levels of enzyme activity that cannot be always achieved, for example, only about 40% of the enzymatic activities are likely to be detected in E. coli-based expression systems (Gabor et al., 2004). In addition, it is herein pointed out that despite the advanced sequencing techniques available, ˜35-60% of the total protein-coding genes display high similarities to “hypothetical proteins”, “predicted proteins” or “protein of unknown function” (Culligan, et al., 2014; Venter, et al., 2004).
3. Only the preselected clone is transformed into plants.
4. The expression and effect of a preselected clone in the target plant is unpredictable.

For the aforementioned reasons the novel method of the present invention of screening plants transformed with an expression library for a desirable phenotype is advantageous.

It is herein acknowledged that drought and salinity are considered as two abiotic stresses that have major effects on plant growth and development.

With respect to drought, it is considered the most devastating environmental stress, which decreases crop growth and productivity. Drought severely affects plant growth and development with substantial reductions in growth rate and biomass accumulation. The main consequences of drought in plants are reduced rate of cell division and expansion, leaf size, stem elongation and root proliferation, and disturbed stomatal oscillations, and water use efficiency (WUE) (Farooq et al. 2009). This phenomenon involves genetic, physiological, and environmental events and their complex interactions. The rate and amount of plant growth depend on these events, which are affected by water deficit. Cell growth is one of the most drought-sensitive physiological processes due to the reduction in turgor pressure and water availability (Taiz and Zeiger, 2006). Under water deficiencies, cell elongation of higher plants can be inhibited by interruption of water flow from the xylem to the surrounding elongating cells. Impaired mitosis, reduced cell elongation and expansion result in reduced plant height, leaf area and crop growth (Nonami, 1998).

Salinity is also considered one of the major severe abiotic factors affecting crop growth and productivity. During salt stress, all major processes such as photosynthesis, protein synthesis and energy and lipid metabolism are affected (Parida & Das, 2005). During initial exposure to salinity, plants experience water stress, which in turn reduces leaf expansion. The osmotic effects of salinity stress can be observed immediately after salt application and are believed to continue for the duration of exposure, resulting in inhibited cell expansion and cell division, as well as stomatal closure. During long-term exposure to salinity, plants experience ionic stress, which can lead to premature senescence of adult leaves, and thus a reduction in the photosynthetic area available to support continued growth. In fact, excess sodium and more importantly chloride has the potential to negatively affect plant enzymes, resulting in reduced energy production and other physiological changes. It is further acknowledged that ionic stress results in premature senescence of older leaves and in toxicity symptoms (chlorosis, necrosis) in mature leaves. Without wishing to be bound by theory, the high sodium ions affect plants by disrupting protein synthesis and interfering with enzyme activity (Carillo et al., 2011).

The present invention provides a method for efficiently screening for novel genes conferring resistance or improved tolerance to drought and/or salinity in plants and especially in valuable crops.

The method of the present invention overcomes the above drawbacks by using expressed genetic material (such as RNA or mRNA) that represent the genes that are being expressed in selected organisms, e.g. as a result of environmental conditions (such as drought or high salt), and producing a cDNA library that represents the ‘Meta-Expression’ or metatranscriptome status of a certain biological niche or other genetic source. The entire cDNA library is then transformed into plants and expressed and screened for the desirable phenotype in the plants.

A core aspect of the present invention is that an expression library is produced from various sources (including plants) and environments. The expression library is transformed into plants, which is the target organism in order to improve its traits or functions. The plant expression library is then screened for the desirable trait, such as salt or drought resistance or tolerance, improved biomass and yield production, biotic stresses (diseases and pathogens) resistance or tolerance, improved nutritional value or improved fertilizers utilization.

It is herein acknowledged that the environments (such as soils) in which plants grow are inhabited by microbial communities, e.g. one gram of soil contains about 10⁷-10⁹ microbial cells (estimates of the number of species of bacteria per gram of soil vary between 2000 and 8.3 million, https://www.ncbi.nlm.nih.gov/pmc/articles/PMC2970868/) which comprise about one gigabase of sequence information, or more. The microbial communities which inhabit environments in which plants grow (such as soils) are complex and remain poorly understood despite their economic importance. Such microbial consortia provide the ecosystem necessary for plant growth, including fixing atmospheric nitrogen, nutrient cycling, disease suppression, and sequester iron and other metals.

It is within the scope of the present invention to use functional metagenomics and metatranscriptomics approaches to explore new genes which confer improved traits to plants.

Reference is now made to metagenomics approaches, employed by the present invention according to some aspects. Metagenomics is the study of genetic material derived from environmental samples. It generally refers to as environmental genomics, eco-genomics or community genomics. While traditional microbiology and microbial genome sequencing and genomics rely upon cultivated clonal cultures, environmental gene sequencing cloned specific genes to produce a profile of diversity in a natural sample. In some aspects, metagenomics uses the study of the genomes in a microbial community to constitute the first step to studying the microbiome. Its main purpose is to infer the taxonomic profile of a microbial community. The whole-metagenome sequencing (WMS) provides data on the functional profile of a microbial community. Such work revealed that the vast majority of microbial biodiversity had been missed by cultivation-based methods. In fact it is estimated that over 99% of all microorganisms in almost every environment on earth cannot be cultivated in the laboratory.

Metagenomics is herein also refers to metatranscriptomics, which studies and correlates the transcriptomes of a group of interacting organisms or species. Metatranscriptomics involves sequencing the complete (meta)transcriptome of the microbial community. In some aspects, metatranscriptomics informs the genes that are expressed by the community as a whole. With the use of functional annotations of expressed genes, it is possible to infer the functional profile of a community under specific conditions, which are usually dependent on the status of the host. While metagenomics provides data on the composition of a microbial community under different conditions, metatrascriptomics provides data on the genes that are collectively expressed under different conditions. Metatranscriptomics involves profiling of community-wide gene expression (RNA-seq). In specific aspects, metatranscriptomics describes the genes that are expressed in a specific microbial environment. Thus, metatranscriptomics is the study of the function and activity of the complete set of transcripts (RNA-seq) from environmental samples.

It is noted that gene expression is log-like distributed, for example, top 100 genes of highest expression can cover up to 30% of all transcripts. Even a single gene can cover up to 10%. Thus, a very high sequencing depth is required to see also lower expressed genes.

By using methods such as “shotgun” or PCR directed sequencing, largely unbiased samples of the genes from the members of sampled communities can be obtained. It is herein acknowledged that metagenomics approaches provide a powerful tool for utilizing microbial ecology to improve traits in plants, for example, biological mechanisms that can be harnessed for agriculture and improved plant traits.

As used herein, the term “about” denotes ±25% of the defined amount or measure or value.

As used herein the term “similar” denotes a correspondence or resemblance range of about ±20%, particularly ±15%, more particularly about ±10% and even more particularly about ±5% .

As used herein the term “average” refers to the mean value as obtained by measuring a predetermined parameter in each plant of a certain plant population and calculating the mean value according to the number of plants in said population.

As used in this specification and the appended claims, the singular forms “a”, “an”, and “the” include plural referents unless the context clearly dictates otherwise. Thus, for example, reference to “a plant” includes one or more plants, reference to “a trait” includes one or more traits and reference to “a cell” includes mixtures of cells, tissues, and the like.

A “plant” as used herein refers to any plant at any stage of development, including a plant seed.

The term “plant” includes the whole plant or any parts or derivatives thereof, such as plant cells, plant protoplasts, plant cell tissue culture from which plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” or “tissue culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or any combination thereof.

A “plant organ” as used herein means a distinct and visibly structured and differentiated part of a plant such as a root, stem, leaf, flower, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “trait” refers to a characteristic or phenotype, particularly, to a plant improving characteristic or phenotype. A phenotypic trait may refer to the appearance or other detectable characteristic of an individual, resulting from the interaction of its genome, proteome and/or metabolome with the environment. For example, in the context of the present invention a plant improving trait or a desirable plant improving trait relates to resistance or tolerance to at least one biotic stress, resistance or tolerance to at least one abiotic stress, improved yield or biomass, improved grain yield, improved fertilizer uptake and usage efficiency and any combination thereof.

A trait may be inherited in a dominant or recessive manner, or in a partial or incomplete-dominant manner. A trait may be monogenic (i.e. determined by a single locus) or polygenic (i.e. determined by more than one locus) or may also result from the interaction of one or more genes with the environment. A dominant trait results in a complete phenotypic manifestation at heterozygous or homozygous state; conventionally, a recessive trait manifests itself only when present at homozygous state.

The term “phenotype” is understood within the scope of the present invention to refer to a distinguishable characteristic(s) of a genetically controlled trait.

As used herein, the phrase “phenotypic trait” refers to the appearance or other detectable characteristic of an individual, resulting from the interaction of its genome, proteome and/or metabolome with the environment.

It is within the scope of the current invention that “stress” may be defined as any external factor that has a negative influence on plant growth, function and/or reproduction

The term “abiotic stress” is herein generally defined as the negative impact of non-living factors on the plant in a specific environment. The non-living variable must influence the environment beyond its normal range of variation to adversely affect the plant or plant population performance or physiology in a significant way. Non limiting examples of abiotic stress factors, or stressors, or environmental factors may encompass factors such as sunlight, wind, temperature (cold, heat), salinity, over watering (flooding), drought and factors such as fertilizer uptake and fertilizer usage efficiency and any combination thereof. Abiotic stress resistance or tolerance may enhance the growth and productivity of plants and specifically crops. It has been shown that abiotic stressors are most harmful and may result in synergistic effects when they occur together, in combinations of abiotic stress factors.

The term “drought” refers hereinafter to a physical phenomenon generally caused by an extended period of below average precipitation or irrigation. For example, not enough or low moisture (at the soil or at the air), water supply shortages, dry soil, moisture regimes, high salinity, heat and any combination thereof. Dry conditions may develop for different reasons. It can have a substantial impact on the ecosystem and agriculture, e.g. reduction in yield and crop damage.

Many organisms have drought tolerance physiological and genetic adaptations.

“Biotic stress” is herein defined as stress that occurs as a result of damage done to plants by other living organisms, such as bacteria, viruses, fungi, whitefly, thrips, spidermites, nematodes, parasites, beneficial and harmful insects, weeds, and cultivated or native plants. The types of biotic stresses imposed on a plant may be depended on both geography and climate and on the host plant and its ability to resist particular stresses.

As used herein, the phrase “resistance” refers to the ability of a plant to restrict the growth and development of a specified pathogen and/or the damage caused to the plant when compared to susceptible plants under similar environmental conditions. Resistant plants may exhibit some disease symptoms or damage under pathogen or pest pressure or under abiotic stress condition.

It is further within the scope of the present invention that resistance means that a plant completely immunizes itself from a particular stress, for example to a biotrophic pathogen infection. According to specific embodiments of the invention, by transformation of an expression library to a host plant, the transformed host acquires a resistance gene which prevents the proliferation of the pathogen and/or confers resistance to a particular abiotic stress (e.g. drought).

According to some aspects, resistance is an absolute term where the plant completely immunizes itself to a particular stress. It should be noted that this does not mean that tolerance cannot be obtained in case of biotic or abiotic stress.

The term “tolerance” refers hereinafter to the characteristic of a plant that allows a plant to avoid, tolerate or recover from biotic or abiotic stressors, under conditions that would typically cause a greater amount of injury to other plants of the same species. These inheritable characteristics influence the degree of damage caused to the plant. In terms of agricultural production tolerance means that the plant can be under stress (diseased/infected/or physiologically challenged) but the extent of loss does not exceed the economic threshold level (an extent of loss which do not hamper the economic potential of the produce). According to further aspects of the present invention, tolerance is a relative term. Examples of tolerance can be found in case of plant pathogens and all abiotic stresses, especially in the case of complex traits that are governed by multiple factors.

In general, ‘resistance’ and ‘tolerance’ are the terms used to denote the ability of the plant to manage the stress, be it biotic or abiotic.

The term “transformation” used herein refers to genetic alteration or modification induced by the introduction of exogenous DNA into a cell. This includes both integration of the exogenous DNA into the host genome, and/or introduction of plasmid DNA containing the exogenous DNA into the plant cell. Such a transformation process results in the uptake, incorporation and expression of exogenous genetic material (exogenous DNA, for examples expression library prepared from ecological niche sampling). Plant transformation may refer to the introduction of exogenous genes into plant cells, tissues or organs, employing direct or indirect means developed by molecular and cellular biology.

The term “environmental niche” or “ecological niche” generally refers to the behavior of a species living under specific environmental conditions. It includes the microbes, fungi, plants or other organisms that inhabit a given environmental location (extremophiles). The ecological niche describes how an organism or population responds to the distribution of resources and competitors and how it in turn alters those same factors. The type and number of variables comprising the dimensions of an environmental niche vary from one species to another and the relative importance of particular environmental variables for a species may vary according to the geographic abiotic and biotic contexts.

According to other aspects, the term “environmental niche” or “ecological niche” describes the relational position of a species or population in an ecosystem. More specifically, it describes how a population responds to the abundance of its resources and competitors and how it affects those same factors. The abiotic or physical environment is also part of the niche because it influences how populations affect, and are affected by. resources and competition. The description of a niche may include descriptions of the organism's life history, habitat, and place in the food chain. In context of the present invention “environmental niche” or “ecological niche” can be defined according to biotic factors or abiotic factors such as high salinity, drought conditions, elevated heat. cold conditions, pH or any other extreme environmental conditions.

It is within the scope of the current invention that the genetic material is derived from a sampling of a predefined environmental niche, including from soil, water, plant biomass, microorganisms, yeast, algae, nematode, etc.

The term “microbiome” or “microbiota” as used herein refers to an ecological community of commensal, symbiotic and pathogenic microorganisms found in and on all multicellular organisms from plants to animals. A microbiota includes bacteria, archaea. protists, fungi and viruses. Microbiota has been found to be crucial for immunologic. hormonal and metabolic homeostasis of their host. The synonymous term microbiome describes either the collective genomes of the microorganisms that reside in an environmental niche or the microorganisms themselves. The microbiome and host emerged during evolution as a synergistic unit from epigenetics and genomic characteristics, sometimes collectively referred to as a holobiont.

The term “genetic material” or “genetic pool” refers hereinafter to sum of a population's genetic material at a given time. It includes all genes and combinations of genes (sum of the alleles) in the population.

The term “isolated” as used hereinafter means that material is removed from its original environment (e.g., the natural environment if it is naturally occurring). For example, a naturally-occurring polynucleotide or polypeptide which is separated from some or all of the coexisting materials in the natural system is isolated.

The nucleic acid isolated or derived from microorganisms or any organism can preferably be inserted into a vector or a plasmid. Such vectors or plasmids are preferably those containing expression regulatory sequences, including promoters, enhancers and the like suitable for expression in plants. Particularly preferred plasmids and methods for introduction and transformation into them are described in detail in the protocol set forth herein.

The term “expression library” as used hereinafter refers to a collection of vectors or viruses (such as plant viruses used as virus-vectors) or plasmids or phages containing a representative sample of cDNA or genomic fragments that are constructed in such a way that they will be transcribed and or translated by the host organism (in the context of the present invention, plants). The technique uses expression vectors to generate a library of clones, with each clone transcribing one RNA and or expressing one protein. This expression library is then screened for the property of interest and clones of interest recovered for further analysis. One and non-limiting example would be using an expression library to isolate genes that could confer resistance or tolerance to drought.

It is within the scope of the present invention that the expression library (usually derived from microbial genetic material) can be constructed in a binary vector (or transfer DNA (T-DNA) binary system or a shuttle vector) able to replicate in multiple hosts (e.g. E. coli and Agrobacterium tumefaciens) to produce genetically modified plants. These are artificial vectors that have been created from the naturally occurring Ti plasmid found in Agrobacterium tumefaciens. In some aspects, the expression libraries are transferred from Agrobacterium tumefaciens to plants.

The term “editing” or “gene editing” or “genome editing” refers hereinafter to any conventional or known genome editing system or method including systems using engineered nucleases selected from the group consisting of: meganucleases, zinc finger nucleases (ZFNs), transcription activator-like effector-based nucleases (TALEN), clustered regularly interspaced short palindromic repeats (CRISPR) system and any combination thereof. In the context of the present invention, the aforementioned gene editing techniques are used to edit a target gene in a desirable crop according to the information obtained from the transgene identified by the method of the present invention.

The term “corresponding to the sequence” refers hereinafter to sequence homology or sequence similarity. These terms relate to two or more nucleic acid or protein sequences, that are the same or have a specified percentage of amino acid residues or nucleotides that are the same, when compared and aligned for maximum correspondence, as measured using one of the available sequence comparison algorithms or by visual inspection.

According to further aspects of the invention, the term “corresponding to the nucleotide sequence” refers to variants, homologues and fragments of the indicated nucleotide sequence which possess or perform the same biological function or correlates with the same phenotypic characteristic of the indicated nucleotide sequence.

Another indication that two nucleic acid sequences are substantially similar or that a sequence is “corresponding to the nucleotide sequence” is that the two molecules hybridize to each other under stringent conditions. High stringency conditions, such as high hybridization temperature and low salt in hybridization buffers, permits only hybridization between nucleic acid sequences that are highly similar, whereas low stringency conditions, such as lower temperature and high salt, allows hybridization when the sequences are less similar.

The term “similarity” or “sequence similarity” refers hereinafter to the degree of resemblance between two sequences when they are compared. This is dependent on their identity and it shows the extent to which residues are aligned. Sequence similarity refers to an optimal matching problem (i.e. for sequence alignments). The optimal matching algorithm finds the minimal number of edit operations (inserts, deletes, and substitutions) in order to align one sequence to another sequence. Sequence similarity searches can identify “homologous” proteins or genes by detecting excess similarity, meaning, statistically significant similarity that reflects common ancestry.

It is within the scope of the current invention that similarity searching is an effective and reliable strategy or tool for identifying homologs (i.e. sequences that share a common evolutionary ancestor). Non limiting examples of similarity searching programs, include BLAST (e.g. Altschul et al. 1997); units 3.3 and 3.4), PSI-BLAST (e.g. Altschul et al., 1997), SSEARCH (e.g. Smith and Waterman, 1981); Pearson, 1991, unit 3.10), FASTA (e.g. Pearson and Lipman, 1988, unit 3.9) and the HMMER3 (e.g. Johnson et al., 2010). Such programs produce accurate statistical estimates, and can ensure that protein or nucleic acid sequences that share significant similarity also may have similar structures. Similarity searching is effective and reliable because sequences that share significant similarity can be inferred to be homologous; namely sharing a common ancestor.

Similarity is understood within the scope of the present invention to refer to a sequence similarity of at least 60%, particularly a similarity of at least 70%, preferably more than 80% and still more preferably more than 90%. The term “substantially similar” refers to a nucleic acid, which is at least 50% identical in sequence to the reference when the entire ORF (open reading frame) is compared, where the sequence similarity is preferably at least 70%, more preferably at least 80%, still more preferably at least 85%, especially more than about 90%, most preferably 95% or greater, particularly 98% or greater.

In some embodiments of the invention, such substantially similar sequences refer to polynucleotide or amino acid sequences that share at least about 60% similarity, preferably at least about 80% similarity, alternatively, about 90%, 95%, 96%, 97%, 98% or 99% similarity to the indicated polynucleotide or amino acid sequence/s.

The present invention encompasses nucleotide sequences having at least 60% similarity, preferably 70%, more preferably 80%, even more preferable 90% and especially more preferable 95% similarity to polynucleotide sequences identified by the method of the present invention or to a reference sequence.

The present invention further encompasses amino acid sequences having at least 60% similarity, preferably 70%, more preferably 80%, even more preferable 90% and especially more preferable 95% similarity to polypeptide sequences identified by the method of the present invention or to a reference sequence.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene or protein sequence.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule.

The term “identity” or “sequence identity” further refers hereinafter to the amount of characters which match exactly between two different sequences. Hereby, gaps are not counted and the measurement is relational to the shorter of the two sequences.

In other words, if two sequences, which are to be compared with each other, differ in length, sequence identity preferably relates to the percentage of the nucleotide residues of the shorter sequence, which are identical with the nucleotide residues of the longer sequence. As used herein, the percent of identity between two sequences is a function of the number of identical positions shared by the sequences, taking into account the number of gaps, and the length of each gap, which needs to be introduced for optimal alignment of the two sequences. The comparison of sequences and determination of identity percent between two sequences can be accomplished using a mathematical algorithm as known in the relevant art.

It is further within the scope that the terms “similarity” and “identity” additionally refer to local homology, identifying domains that are homologous or similar (in nucleotide and/or amino acid sequence). It is acknowledged that bioinformatics tools such as BLAST. SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

According to further embodiments, protein or polynucleotide sequences with specific location or domain sequence similarity are identified by the method of the present invention. When comparing residues with no conservation the low similarity is meaningless thus lower overall similarity sequences with high conservation in conserved region will be still considered as similar in a given range, for example of >60% (i.e. sequences showing low similarity of ˜37% to the nearest homolog but possess all the conserved substrate binding residues of a specific protein family) that can be found in hmm-based search algorithms such as HMMER3.

The term “Conserved Domain Database (CDD)” refers to a collection of sequence alignments and profiles representing protein domains. It also includes alignments of the domains to known 3-dimensional protein structures in the database (i.e. Molecular Modeling Database (MMDB).

In some embodiments of the invention, such substantially identical sequences refer to polynucleotide or amino acid sequences that share at least about 60% identity, preferably at least about 80% identity, alternatively, about 90%, 95%, 96%, 97%, 98% or 99% identity to the indicated polynucleotide or amino acid sequence/s.

Polypeptides within the scope of the present invention are at least 50% identical to the protein identified by the method of the present invention; or at least 55% identical, or at least 60% identical, or at least 65% identical, or at least 70% identical, or at least 75% identical, or at least 80% identical, or at least 85% identical or at least 90% identical or at least 95% identical to the protein identified by the method of the present invention or to a reference sequence.

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 80% sequence identity, preferably at least 85%, more preferably at least 90%, most preferably at least 95% sequence identity compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 80%, preferably at least 85%, more preferably at least 90%, and most preferably at least 95%. Preferably, optimal alignment is conducted using the homology alignment algorithm of Needleman et al. (1970. J. Mol. Biol. 48:443).

The term “homolog” as used herein, refers to a DNA or amino acid sequence having a degree of sequence similarity in terms of shared amino acid or nucleotide sequences. There may be partial similarity or complete similarity (i.e., identity). For protein sequences, amino acid similarity matrices may be used as are known in different bioinformatics programs (e.g. BLAST, FASTA, Bestfit program—Wisconsin Sequence Analysis Package, Version 8 for Unix, Genetics Computer Group, University Research Park, 575 Science Drive Madison, WI 53711, Smith Waterman). Different results may be obtained when performing a particular search with a different matrix. Degrees of similarity for nucleotide sequences are based upon identity matches with penalties made for gaps or insertions required to optimize the alignment, as is well known in the art (e.g. Altschul S. F. et al., 1990, J Mol Biol 215(3):403-10; Altschul S. F. et al., 1997, Nucleic Acids Res. 25:3389-3402). Guidance in determining which amino acid residues may be substituted, inserted, or deleted without abolishing biological or activity may be found using computer programs well known in the art, for example, DNASTAR software.

The term “polymorphism” is understood within the scope of the invention to refer to the presence in a population of two or more different forms of a gene, genetic marker, or inherited trait or a gene product obtainable, for example, through alternative splicing, DNA methylation, etc.

The present invention encompasses “High-throughput screening” or “HTS” technique, which herein refers to a method to rapidly identify genes that modulate a particular biomolecular pathway or function. It includes metatranscriptomic and metagenomic gene expression.

The present invention outlines a procedure for producing expression libraries from genetic material isolated from ecological niches, which expression libraries can be transformed into the target plant for screening for a desirable trait such as tolerance or resistance to biotic or abiotic stress and improving yield or biomass production.

According to one embodiment, the present invention provides a method for screening for and identifying a desirable plant improving trait, the method comprises steps of: (a) obtaining genetic material from a sampling of a predefined environmental niche; and (b) constructing an expression library from the genetic material. According to core embodiments, the present invention further comprises steps of: (c) producing plants transformed with the expression library at an efficiency of at least 0.05%-30%, representing at least 10²-10¹⁰ transgenes, thus creating the expressed library within the plants or seeds; (d) screening for transformed plants expressing the desirable trait; and (e) identifying the transgene of the transformed plants expressing the desirable trait.

It is further within the scope to disclose the method as defined in any of the above, wherein the step (a) further comprises steps of enriching the genetic material by growth on rich media or on selective media.

It is further within the scope to disclose the method as defined in any of the above, wherein the step (a) further comprises steps of enhancing expression of the desirable trait by culturing the genetic material on selective media for the desirable trait.

It is further within the scope to disclose the method as defined in any of the above, wherein the step (b) comprises steps of producing prokaryotic cDNA library or eukaryotic cDNA library or both.

It is further within the scope to disclose the method as defined in any of the above, wherein the step (b) further comprises steps of cloning the cDNA library into at least one binary vector.

It is further within the scope to disclose the method as defined in any of the above, wherein the binary vector comprises a constitutive promoter or a stress induced promoter.

It is further within the scope to disclose the method as defined in any of the above, wherein the binary vector comprises bacterial selection marker and plant transformation selection marker.

It is further within the scope to disclose the method as defined in any of the above, wherein the bacterial selection marker is Kanamycin resistance, or any other antibiotic resistance conferring gene, and the plant transformation selection marker is bar gene, conferring resistance to phosphinothricin containing herbicide (e.g. Basta herbicide).

Reference is now made to Glufosinate (also known as phosphinothricin and often an ammonium salt) is a naturally occurring broad-spectrum systemic herbicide produced by several species of Streptomyces soil bacteria. Glufosinate is a broad-spectrum herbicide that is used to control weeds. It is sold in formulations under brands including Basta, Rely, Finale, Challenge and Liberty. The bar gene confers resistance to the herbicide Basta (containing phosphinothricin).

It is further within the scope to disclose the method as defined in any of the above, further comprises steps of transforming the cloned binary vectors into host cells.

It is further within the scope to disclose the method as defined in any of the above, further comprises steps of transforming the cloned binary vectors into Agrobacterium tumefaciens.

It is further within the scope to disclose the method as defined in any of the above further comprises steps of introducing the transformed Agrobacterium tumefaciens into at least one of: whole plant, plant tissue and plant cell.

It is further within the scope to disclose the method as defined in any of the above, comprises steps of introducing the transformed Agrobacterium tumefaciens by spraying the plants with an inoculum comprising transformed Agrobacterium.

It is further within the scope to disclose the method as defined in any of the above, wherein the step (d) comprises growing the transformed plants under conditions selective for the desirable trait.

It is further within the scope to disclose the method as defined in any of the above, further comprises steps of:
f. collecting T1 seeds from the transformed plants of step (d);
g. determining seed library efficiency of the TI seeds by calculating ratio of phosphinothricin resistant plants to total number of plants;
h. sowing the T1 seeds of step (e) under selective conditions allowing screening and selection of transformed plants expressing the desirable trait;
i. testing the selected plants expressing the desirable trait of step (g) for presence of the transgene; and
j. isolating and sequencing the transgene of the selected transformed plants positively tested for the transgene of step (h).

It is further within the scope to disclose the method as defined in any of the above, further comprises steps of
k. collecting T2 seeds from the plants of (h), which are found positive for presence of the transgene;
l. growing plants of the T2 seeds under selective conditions allowing screening and selection of transformed plants expressing the desirable trait as compared to control plants transformed with known genes conferring the desirable trait; and
m. optionally, isolating and sequencing the transgene of the selected plants of step (j).

It is further within the scope to disclose the method as defined in any of the above, comprises steps of
a. recloning and sequencing the isolated transgene of step (i) and/or (l);
b. transforming the recloned transgene into plants;
c. screening the transformed plants of step (b) for selection of transformed plants expressing the desirable trait;
d. isolating the transgene from the selected plants of step (c); and
e. optionally, repeating steps (a) to (d).

It is further within the scope to disclose the method as defined in any of the above, wherein the environmental niche comprises samples derived from ecological niches, sources, populations, habitats, gene pools, prokaryotic culture, eukaryotic culture and any combination thereof.

It is further within the scope to disclose the method as defined in any of the above, wherein the environmental niche sampling comprises microbiome, microbiota or microbial culture, plant, yeast, algae, nematode or any other organism or combinations thereof.

It is further within the scope to disclose the method as defined in any of the above, wherein the environmental niche is defined according to biotic factors, abiotic factors and a combination thereof.

It is further within the scope to disclose the method as defined in any of the above, wherein the environmental niche sampling comprises soil sample, water sample, organic matter sample, any living organisms (such as plant, yeast, bacteria, microorganism, algae, nematode) and any combination thereof.

It is further within the scope to disclose the method as defined in any of the above, wherein the desirable trait is selected from the group consisting of resistance or tolerance to at least one biotic stress, resistance or tolerance to at least one abiotic stress, improved yield or biomass, improved grain yield, improved fertilizer uptake and improved usage efficiency and a combination thereof.

It is further within the scope to disclose the method as defined in any of the above, wherein the abiotic stress is selected from the group consisting of: drought, salinity, heat, cold, fertilizer uptake, fertilizer utilization efficiency and any combination thereof.

It is further within the scope to disclose the method as defined in any of the above, wherein the biotic stress is selected from the group consisting of: pathogens, bacteria, viruses, fungi, parasites, beneficial and harmful insects, weeds, and cultivated or native plants or any combination thereof.

It is further within the scope to disclose the method as defined in any of the above, wherein the method comprises steps of extracting RNA from the sampling of the predefined environmental niche.

It is further within the scope to disclose the method as defined in any of the above, wherein the RNA extraction is performed according to standard commercial kits or according to any other protocol for extraction of RNA from environmental sampling.

It is further within the scope to disclose the method as defined in any of the above, wherein the protocol for extraction of RNA from environmental sampling comprises steps of:
a. obtaining a soil sample;
b. mixing the soil sample with an extraction buffer comprising 500 mM phosphate buffer pH 8 and 5% w/v cetyltrimethylammonium bromide (CTAB) with phenol (pH 8)/chloroform/IAA ratio of 25:24:1;
c. subjecting the mixture of step (b) to 15 min shaking at 37° C. or to a bead beater for 1 min;
d. centrifuging the mixture of step (c) at 2,500 g for about 10 minutes at room temperature to obtain an aqueous phase;
e. transferring the aqueous phase into a new tube;
f. adding to the aqueous phase within the tube of step (e) an equal amount of iso-propanol supplemented with 20 mg/ml crystal violet solution to obtain violate stained solution;
g. mixing the solution by inverting said tube of step (f) and then incubating the tube for about 30 minutes at room temperature;
h. centrifuging the tube of step (g) at 2,500 g for about 30 minutes at room temperature to obtain a violet stained layer;
i. transferring the violate stained layer into a new tube and centrifuging the tube for about 5 min at maximal speed to obtain pellet and supernatant;
j. washing the pellet with 80% v/v ice cold ethanol and centrifuging for additional 5 min to obtain pellet and supernatant;
k. removing the supernatant of step (j) and allowing the pellet to dry; and
l. suspending the dried pellet in water in a ratio of 100 μl water to 2 gr of soil of step (a).

It is further within the scope to disclose polynucleotide sequences obtainable by the method as defined above.

It is further within the scope to disclose the polynucleotide as defined above, wherein the polynucleotide comprises a nucleotide sequence corresponding to the sequence as set forth in a polynucleotide sequence selected from the group consisting of SEQ ID NOs:1-148 and any combination thereof.

It is further within the scope to disclose a polynucleotide sequence having at least 80%, 85%, 90% or 95% sequence similarity to a polynucleotide sequence obtainable by the method as defined above.

It is further within the scope to disclose a polypeptide sequence obtainable by the method as defined above.

It is further within the scope to disclose the polypeptide sequence as defined above, wherein the polypeptide comprises an amino acid sequence corresponding to the sequence as set forth in a polypeptide sequence selected from the group consisting of SEQ ID NOs: 149-321 and any combination thereof.

It is further within the scope to disclose an amino acid sequence having at least 60%, 70%, 80% or 90% sequence similarity to an amino acid sequence obtainable by the method as defined above.

It is further within the scope to disclose the use of the method as defined above for identifying genes conferring resistance or tolerance to abiotic or biotic stress.

It is further within the scope to disclose the use of the method as defined above for identifying genes conferring improved yield and biomass, i.e. improved grain yield, in plants, for example by enhancing growth, with or without exposure to stress conditions.

It is further within the scope to disclose the use of the method as defined above for identifying genes conferring improved yield.

It is further within the scope to disclose the use as defined in any of the above, wherein the abiotic stress is selected from the group consisting of: drought, salinity, heat, cold, fertilizer utilization, fertilizer uptake and any combination thereof.

It is further within the scope to disclose the use as defined in any of the above, wherein the biotic stress is selected from the group consisting of: pathogens, bacteria, viruses, fungi, parasites, beneficial and harmful insects, weeds, and cultivated or native plants or any combination thereof.

It is further within the scope to disclose a method for screening for and identifying a drought resistance or tolerance improving trait in plants, the method comprises steps of: (a) obtaining genetic material derived from a low moisture or a high salinity environmental niche sample; and (b) constructing expression library from the genetic material. According to core embodiments, the method further comprises steps of: (c) producing plants transformed with the expression library at an efficiency of at least 0.05%-30%, representing at least 10²-10¹⁰ transgenes; (d) screening for transformed plants surviving predetermined drought conditions; and (e) identifying the transgene of the drought surviving transformed plants of step (d).

It is further within the scope to disclose the method as defined above, wherein the step (b) further comprises steps of cloning the expression library into at least one binary vector.

It is further within the scope to disclose the method as defined in any of the above, further comprises steps of:
f. collecting T1 seeds from the transformed plants of step (c);
g. sowing the T1 seeds in soil selective for transformed plants, with water content of about 100% capacity;
h. growing plants of the T1 seeds in drought condition and/or without irrigation until most of the plants die, to produce transformed plants surviving the drought conditions;
i. growing the drought surviving transformed plants to produce T2 seeds;
j. screening the drought surviving transformed plants of step (i) for presence of a transgene;
k. isolating and sequencing the transgene from positively screened plants of step (j);

It is further within the scope to disclose the method as defined in any of the above, further comprises steps of
l. collecting T2 seeds from each of the transgene-containing positively screened drought surviving transformed plants of step (j);
m. growing T2 plants from each of the transgene-containing T2 seeds of step (l) under predetermined drought conditions as compared to control plants transformed with known genes conferring drought tolerance or drought resistance;
n. performing drought tolerance or resistance screen measurements for each of the transgene-containing T2 plants as compared to the control plants selected from the group consisting of: turgor measurements, number of plants death, state of plants and any combination thereof;
o. isolating the transgene from the screened drought resistance performing T2 plants of step (n);
p. optionally, recloning the transgene into a binary vector;
q. optionally, transforming the cloned binary vector into plants and growing the transformed plants under predetermined drought conditions; and
r. optionally, repeating steps (l) to (q).

It is further within the scope to disclose the method as defined in any of the above, wherein the step of growing T2 plants comprises steps of: (a) sowing the T2 seeds in soil selective for transformed plants, with water content of about 100% capacity; and (b) irrigating the plants when water content in the soil reaches about 5-10%.

It is further within the scope to disclose the method as defined in any of the above, wherein the predetermined drought conditions are selected from the group consisting of low moisture, high salinity, dry soil and heat.

It is further within the scope to disclose polynucleotide sequences obtainable by the method as defined in any of the above.

It is further within the scope to disclose the polynucleotide as defined above, wherein the polynucleotide comprises a nucleotide sequence corresponding to the sequence as set forth in a polynucleotide sequence selected from the group consisting of SEQ ID NOs:1 to SEQ ID NO:148 and any combination thereof.

It is further within the scope to disclose polynucleotide sequences having at least 80%, 85%, 90% or 95% sequence similarity to polynucleotide sequences obtainable by the method as defined in any of the above.

It is further within the scope to disclose a polypeptide sequence obtainable by the method as defined in any of the above.

It is further within the scope to disclose the polypeptide sequence as defined above, wherein the polypeptide sequence comprises an amino acid sequence corresponding to the sequence as set forth as set forth in polypeptide sequence selected from the group consisting of SEQ. ID Nos: 149-321 and any combination thereof.

It is further within the scope to disclose polypeptide sequences having at least 60%, 70%, 80% or 90% sequence similarity to amino acid sequences obtainable by the method as defined in any of the above.

It is further within the scope of the present invention to disclose a method for extracting RNA from a soil sample comprising steps of:
m. obtaining a soil sample;
n. mixing said soil sample with an extraction buffer comprising 500 mM phosphate buffer pH 8 and 5% w/v cetyltrimethylammonium bromide (CTAB) with phenol (pH 8)/chloroform/IAA ratios of 25:24:1;
o. subjecting said mixture of step (b) to about 15 min shake at 37° C. or to a bead beater for 1 min;
p. centrifuging said mixture of step (c) at 2,500 g for about 10 minutes at room temperature to obtain an aqueous phase;
q. transferring said aqueous phase into a new tube;
r. adding to said aqueous phase within said tube of step (e) an equal amount of iso-propanol supplemented with 20 mg/ml crystal violet solution to obtain violate stained solution;
s. mixing said solution by inverting said tube of step (f) and then incubating said tube for about 30 minutes at room temperature;
t. centrifuging said tube of step (g) at 2,500 g for about 30 minutes at room temperature to obtain a violet stained layer;
u. transferring said violate stained layer of step (h) into a new tube and centrifuging said tube for about 5 min at maximal speed to obtain pellet and supernatant;
v. washing said pellet with 80% v/v ice cold ethanol and centrifuging for about additional 5 min to obtain pellet and supernatant;
w. removing said supernatant of step (j) and the pellet is left to dry; and
x. suspending said dried pellet in water in a ratio of 100 μl water to 2 gr of soil of step (a).

It is further within the scope of the present invention to disclose a method for screening for and identifying a desirable plant improving trait, said method comprises steps of:
y. obtaining a sampling of a predefined environmental niche;
z. extracting RNA from the sampling according to the method for extracting RNA from a soil sample as defined above;
aa. constructing an expression library from the RNA of step (b);

The method further comprises steps of:
bb. producing plants transformed with the expression library at an efficiency of at least 0.05%-30% representing at least 10²-10¹⁰ transgenes;
cc. screening for transformed plants expressing the desirable trait; and
dd. identifying the transgene of the transformed plants expressing the desirable trait.

It is further within the scope of the present invention to disclose an isolated polynucleotide having a nucleotide sequence corresponding to the sequence as set forth in a polynucleotide sequence selected from the group consisting of SEQ ID NOs:1 to SEQ ID NO:148 and any combination thereof.

It is further within the scope of the present invention to disclose an isolated polypeptide having an amino acid sequence corresponding to the sequence as set forth in polypeptide sequence selected from the group consisting of SEQ. ID Nos: 149-321 and any combination thereof.

In order to understand the invention and to see how it may be implemented in practice, a plurality of preferred embodiments will now be described, by way of non-limiting example only, with reference to the following examples.

### EXAMPLE 1

### A Process for Improving Traits in Plants by Transformation of Expression Libraries from Ecological Niches into Plants and Screening for Desired Traits

### 1. Sample Collection and Processing

In the first step, genetic pools of a varied environmental samples and sources such as soil, water or organic matter from different habitats have been isolated. The source is selected according to the specific desired target traits. For example, when screening for drought or salinity resistant gene, a dry land such as desert land or a high salinity land or other enforcement will be used, but not necessarily.

The microbiome found in each sample may optionally be enriched by growth on rich media or selectively grown with antibiotics. To enhance expression of potentially desired genes, the culture is grown in stress conditions or media resembling, associated with or affecting the target trait, such as salt or PEG rich media for drought or salinity resistance trait.

Sample enrichment is carried on rich growth media (e.g. YPD) for several days at 28° C.-37° C. in shaker incubator. If eukaryotic libraries are prepared, anti-bacterial antibiotics such as Penicillin-Streptomycin and Spectinomycin are added.

To induce stress resistant genes, the sample is grown under any desired environmental stress conditions. For example, to induce drought resistance genes, the sample is grown under high osmotic stress by adding PEG to the growth media (10%-30% w/v). High salt concentration media such as NaCl (5%-10% w/v) was used to induce high salinity stress. In addition, the samples are exposed to different nitrogen concentration (from 0-100 mM KNO₃ in water supplemented with 6 mM KH₂PO₄ and micro elements, see Table 1, http://www.gatfertilizers.com/properties-of-solid-and-liquid-fertilizers/ as recommended by the manufacturer), extreme temperatures (50-60° C.) and any environmental stress desired.

TABLE 1

| Element | Percentage | gr/Lt |
|---|---|---|
| Iron | 1.09 | 12.20 |
| Manganese | 0.48 | 5.47 |
| Zinc | 0.15 | 1.75 |
| Copper | 0.05 | 0.55 |
| Molybdenum | 0.02 | 0.16 |
| Boron | 0.20 | 2.00 |

### 2. RNA Extraction

Total RNA extraction has been performed according to standard commercial kits such as RNeasy PowerSoil Total RNA Kit (Qiagen) and Quick-RNA (Zymo research). In addition, a unique protocol is used for extraction of RNA from soil samples, as follows:

In a 7 ml tube, 2 g of soil is disrupted with extraction buffer (500 mM Phosphate buffer pH 8 and 5% w/v CTAB with Phenol (pH 8), chloroform, IAA (25:24:1)). The tube is subjected to 15 min shaking at 37° C. or to a bead beater for 1 min. The tube is then centrifuged at 2,500 g for 10 minutes at room temperature. The aqueous phase is transferred into a new tube and an equal amount of iso-propanol supplemented with 5 ul of crystal violate solution (20 mg/ml) is added. The tubes are mixed by inverting and left to stand for 30 minutes at room temperature, then centrifuged at 2,500 g for 30 minutes at room temperature. The violate stained layer is transferred into a new 1.5 ml tube and centrifuged for 5 min at maximal speed. The pellet is washed with 500 μl of 80% v/v ice cold ethanol and centrifuged for additional 5 min. After centrifugation, the liquid is removed, and the pellet is left to dry. The dry pellet is suspended in 100 μl water.

### 3. Construction of cDNA Libraries

3.1. Eukaryotic cDNA Libraries

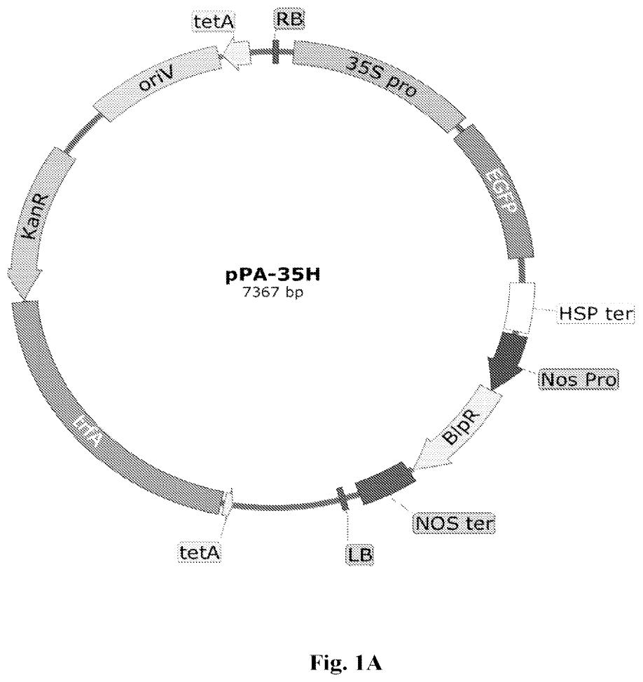

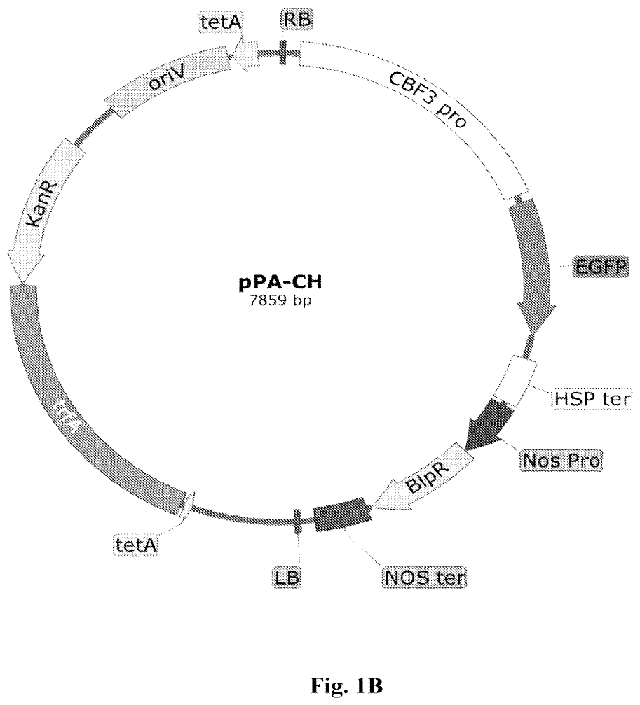

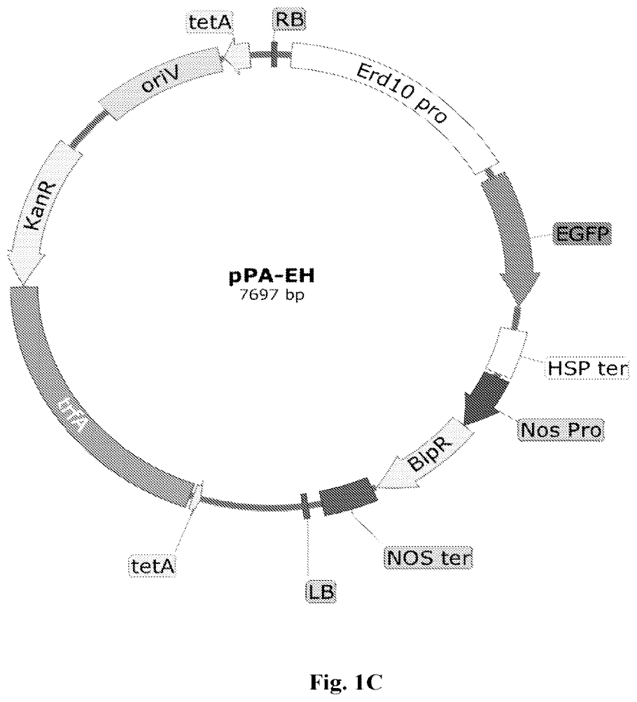

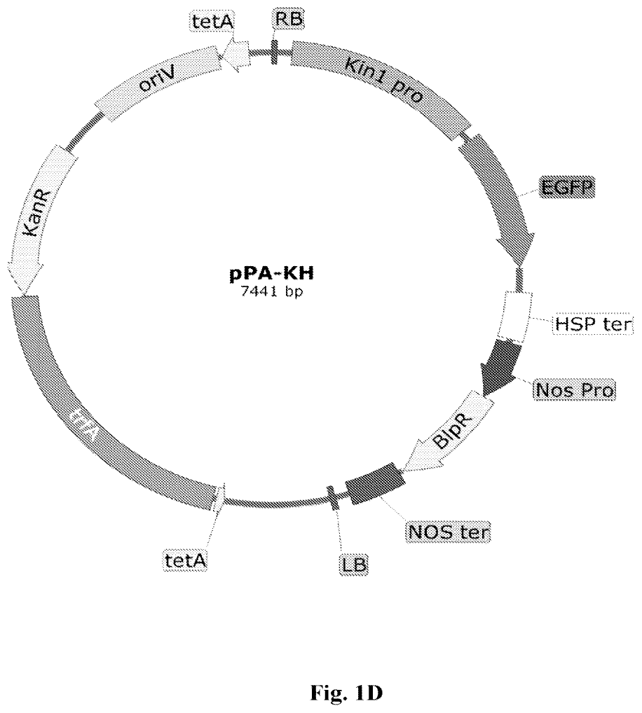

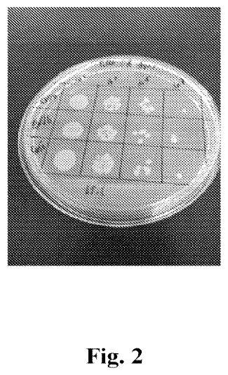

Eukaryotic cDNA libraries from total-RNA and mRNA are constructed based on template switching—reverse transcription of poly-A mRNA (SMART) or oligo-capping rapid amplification of cDNA ends (5′-RACE) methods. The reverse transcription of poly-A mRNA primers used are 5′-ATTCTAGAGCGATCGCACATGTTTTTTTTTTTTTTTTTTTTTTTTTTTTTTVN-3′ (referred to as SEQ ID NO:321) and 5′-AAGCAGTGGTATCAACGCAGAGTGGCGCGCCrGrGG-3′ (referred to as SEQ. ID NO:322). The oligo-capping rapid amplification of cDNA ends primers used are 5′-ATTCTAGAGCGATCGCACATGTTTTTTTTTTTTTTTTTTTTTTTTTTTTTTVN-3′ (referred to as SEQ. ID NO:321) and 5′-InvddT (5′ Inverted Dideoxy-T)-r (AAGCAGUGGUAUCAACGCAGAGUGGCGCGCCG)-3′ (referred to as SEQ. ID NO: 323). The amplified cDNA is inserted into binary vectors (see FIGS. 1-4) between the promoter(s) (35S, KIN1, erd10 and/or CBF3) and the HSP or NOS terminator. FIG. 1A illustrates the pPA-35H vector, which has a constitutive CaMV 35S promoter with the GFP gene cloned between the promoter and terminator as an example. FIGS. 1B-D present vectors containing stress induced promoters from Arabidopsis thaliana: pPA-CH vector with CBF3 promoter having a nucleotide sequence as set forth in SEQ. ID NO: 330 (FIG. 1B), pPA-EH with Erd10 promoter having a nucleotide sequence as set forth in SEQ. ID NO: 331 (FIG. 1C) and pPA-KH with Kin1 promoter having a nucleotide sequence as set forth in SEQ. ID NO: 332 (FIG. 1D) with the GFP gene cloned between the promoter and terminator as an example (Plant Physiol. 1997 October; 115(2): 327-334, Plant Journal (2004) 38, 982-993 incorporated herein by reference).

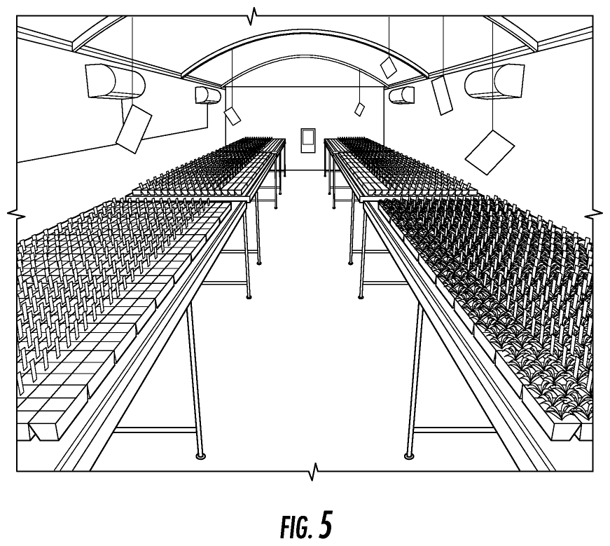

These vectors contain Kanamycin as a bacterial selection and the bar gene as a transgenic plant selection conferring resistance to the phosphinothricin herbicide. At least one of the non-limiting examples of Gibson assembly, Restriction-ligation, Restriction free or In-Fusion methods is used and then ligation products are transformed to E. coli competent cells to grow under kanamycin selection. The library size is estimated by live count of transformed bacteria sown on LB petri dishes (usually 10\hat{ }5-10\hat{ }7) (FIG. 5). Vectors of the cDNA library are purified from E. coli bacteria with standard mini-prep kits and transformed to electrocompetent Agrobacterium tumefaciens GV3101 cells. The transformed Agrobacterium are grown on LB media under kanamycin and rifampicin selection (50 μg/ml each) over night at 28° C., (250 ml per 1 m² of target plant growth area). The growth arrested on ice for at list 30 min and then centrifuged for 10 min at 8000 rpm at 4° C. The pelleted Agrobacterium are suspended in suspension buffer (5% sucrose and 0.03% L-77 Silwet, Momentive, US).

3.2. Prokaryotes cDNA Libraries

Prokaryotes cDNA libraries from total RNA are constructed based on standard 5′ and 3′ RNA modifications with ScriptSeq™ Complete Kit (epicenter). Primers used are 5′-ATTCTAGAGCGATCGCACATGTTTTTTTTTTTTTTTTTTTTTTTTTTTTTTVN-3′ (referred to as SEQ. ID NO:321) and 5′-InvddT (5′ Inverted Dideoxy-T)-r(AAGCAGUGGUAUCAACGCAGAGUGGCGCGCCG)-3′ (referred to as SEQ. ID NO:324). The amplified cDNA inserted into carrier vectors barring Kanamycin and phosphinothricin resistance and then transformed to E. coli competent cells to grow under kanamycin selection (50 μg/ml). The library size is estimated by live count of transformed hosts (usually 10\hat{ }5-10\hat{ }7). Vectors of the cDNA library are purified from host cells with standard mini-prep kit (50 μl) and transformed to electrocompetent Agrobacterium GV3103 cells (100 μl). The transformed Agrobacterium are grown on LB media under kanamycin and rifampicin selection (50 μg/ml) over night at 28° C. (100 ml per 1 m² of target plant growth area). The growth is arrested on ice for at list 30 min and then centrifuged for 5 min at 8000 rpm at 4° C. The pelleted Agrobacterium are suspended in suspension buffer (5% sucrose and 0.03% L-77 Silwet).

### 4. Growing and Transformation of Plants

### 4.1. Arabidopsis Plants

Plants are grown in controlled greenhouses as a preparation for transformation. Plants are grown in soil composed of 75% peat, 25% perlite and are being irrigated routinely with water supplemented with fertilizer (e.g. Shefer 5.3.8, ICL Israel) according to manufacturer instructions, as needed. Plants start flowering after 3-4 weeks and then they are ready for transformation. Transformed Agrobacterium with expression libraries are grown as mentioned above and suspended in suspension buffer (5% sucrose and 0.03% L-77 Silwet) and are sprayed by 2 liter sprayers (e.g. Solo, Germany) on the flowers. After 5-6 weeks of continued growth when plants become dry, seeds are collected and kept in a cool dry place for 2 weeks or until used.

### 4.2. Tobacco Plants

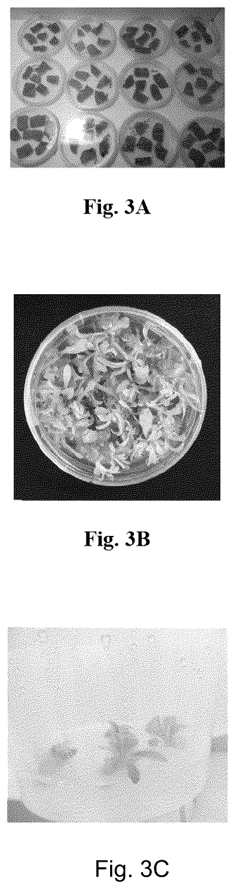

Tobacco leaves are cut into 1-2 cm² pieces and sterilized by 70% ethanol followed by 0.3% bleach treatments for 5 minutes. Leaf pieces are mixed with libraries transformed Agrobacterium (or with a any identified gene of SEQ ID 1-148 from Table 4), suspended in liquid Regeneration Medium (RM) supplemented with MS including Gamborg B5 vitamins, 3% sucrose, 2 mg/L BAP (6-Benzylaminopurine) and 0.2 mg/L NAA (Naphthalene acetic acid) (e.g. Duchefa, Netherland) for 30 minutes. Bacteria are than washed and leaf pieces are placed on RM plant-agar plates for one day in the dark. Leaf pieces are transferred to new selection RM plant-agar plates supplemented with 300 μg/ml of timentin antibiotic to kill the Agrobacterium and 1.5 μg/ml phosphoinotricin (e.g. Duchefa, Netherland) for selection of transgenic plants. FIG. 3A-B present a photographic illustration of tobacco tissue culture transformed with a library, 7 days after transformation (FIG. 3A) and 40 days after transformation (FIG. 3B). After 6-8 weeks, plantlets start to appear and are transferred to new vessels containing the same selection RM plant-agar, but BAP is excluded (see FIG. 3C). After rooting, plants are transferred to soil in the greenhouse.

### EXAMPLE 2

### A Process for Identifying Drought Resistance Traits in Plants

### A. Screening for Drought and/or Salinity Resistant Plants/Genes

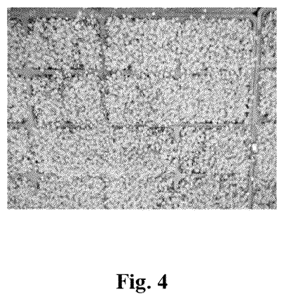

Arabidopsis T1 seeds harboring the desired expression library are being used for the screen. At the first stage, the transformation efficiency is defined for a specific seed library. 1 ml of seeds (˜50,000 seeds) is being sowed on soil irrigated with water supplemented with Basta (e.g. Bayer, Germany) according to manufacturer instructions. Seven days post sowing, the number of phosphinothricin resistant plants is counted and compared with phosphinothricin susceptible plants (FIG. 4). As demonstrated in FIG. 4, the bigger plants are resistant to phosphinothricin while small plants are absent of the transgene and therefore susceptible and will die. The seed library efficiency is represented by the ratio of the number of resistant plants to the number of total plants.

The library is then sowed according to the desired number of plants intended to be represented in the specific experiment and which represents best the library size. For example, if an expression library consists of 5×10⁴ genes, and the transformation efficiency is 1%, >5 million seeds should be sowed. In this case, in ˜20 m² of soil, 50,000 Basta resistant plants will be grown for the experiment.

Soil is irrigated once, when seeds are sown, with water supplemented with phosphinothricin and fertilizer (e.g. Shefer 5.3.8, ICL Israel) according to manufacturer instructions, and soil water content reaches 100% capacity. Plants are grown in air-conditioned controlled greenhouses, and soil is not irrigated until most of the plants die from lack of water. Surviving plants, ˜0.1% of initial phosphinothricin resistant plants, are being rescued by irrigation until they produce seeds which are being collected for T2 experiments. During their growth, the surviving plants are tested for their transgene, by gDNA extraction from one of their leaves and PCR using primers for the gene specific promoters (CaMV 35S, CBF3, Erd10 and Kin1) and terminators (NOS, HSP) (see Table 2). PCR products are being sequenced and the resulted sequence is blasted versus sequence databases such as NCBI, both for DNA comparisons (i.e. BLASTn) and for amino acid sequence comparisons (i.e. BLASTx).

Reference is now made to Table 2 presenting SEQ ID NOs of primer and promoter sequences used in the present invention:

TABLE 2
SEQ ID NOs of primer sequences

| SEQ ID NO. | Description |
|---|---|
| SEQ ID NO: 321 | Reverse primer for transcription of poly-A |
| | mRNA |
| SEQ ID NO: 322 | Forward primer for transcription of poly-A |
| | mRNA |
| SEQ ID NO: 323 | Forward primer for oligo-capping |
| | amplification of cDNA ends |
| SEQ ID NO: 324 | Forward primer for amplification of |
| | prokaryote cDNA library (e.g. derived from |
| | total RNA) |
| SEQ ID NO: 325 | Forward primer for CaMV 35S promoter |
| SEQ ID NO: 326 | Forward primer for CBF3 promoter |
| SEQ ID NO: 327 | Forward primer for Erd10 promoter |
| SEQ ID NO: 328 | Forward primer for Kin1 promoter |
| SEQ ID NO: 329 | Reverse primer for NOS/HSP terminator |
| SEQ ID NO: 330 | CBF3 promoter |
| SEQ ID NO: 331 | Erd10 promoter |
| SEQ ID NO: 332 | Kin1 promoter |

### B. Subsequent Generations (T₂, T₃) Experiments

Seeds collected from drought surviving plants are being tested again in further experiments including repeats and controls to test their resistance/tolerance to drought (see FIG. 5).

Several genes were chosen to serve as controls in the drought experiments:
1) EGFP—jellyfish green fluorescent protein, cloned under the control of the various used promoters and HSP terminator (see vector maps of FIGS. 1A-D), is being served as a negative control for drought, since it was not been shown to be associated with improving plants resistance to drought (Yang T-T, et al., 1996).
2) mtlD—mannitol-1-phosphate dehydrogenase from Escherichia coli, cloned under the control of the various used promoters and HSP terminator (see vector maps of FIGS. 1A-D), is being served as a positive control since it was shown to be associated with improving plants resistance to drought and salt (Hema R. et al., 2014).
3) HRD—The HARDY gene from Arabidopsis thaliana cloned under the control of the various used promoters and HSP terminator (see vector maps of FIGS. 1A-D), is being served as a positive control since was shown to be associated with improving plants resistance to drought and salt (Karaba A. et al., 2007).

Plants identified as expressing unique genes in the screen experiments, including all controls, are sown in trays 38×28 cm with 16 plastic inserts in each tray (e.g. Desch Plantpak, Netherland), filled with soil supplemented with fertilizer and phosphinothricin as above. In each insert several seeds are sown and after 10 days a single phosphinothricin resistant plant is being kept for further experiments. Each experiment contains 20-40 repeats of each plant, representing the expressed unique genes, which are spread in random on the greenhouse tables. Irrigation of the soil is similar to the screen experiment; it is done when the seeds are sown, except when soil is completely dry and reaches weight lower then initial weight of soil before irrigation (˜5%-10% of water content), then plants are irrigated again to check revival performance.

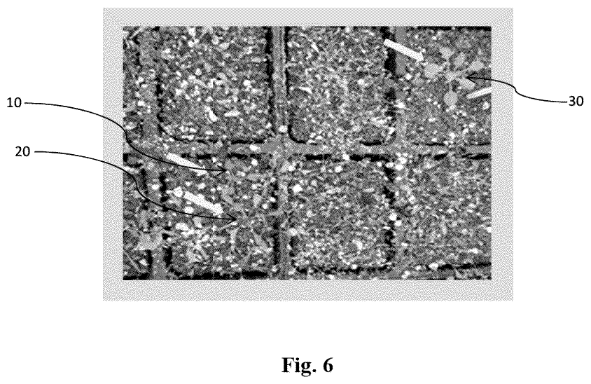

Reference is now made to FIG. 6 showing photographic results of screening for transgenic plants resistance to drought grown under the conditions as described above. This figure shows that transgenic plants carrying drought resistance genes 10, 20 and 30 survive in severe drought conditions, while other transgenic plants that do not harbor drought resistant conferring genes do not survive the stress conditions. It is noted that within the small area shown in this figure (˜15×25 cm), about 300 plants were screened while 3 survived the drought conditions.

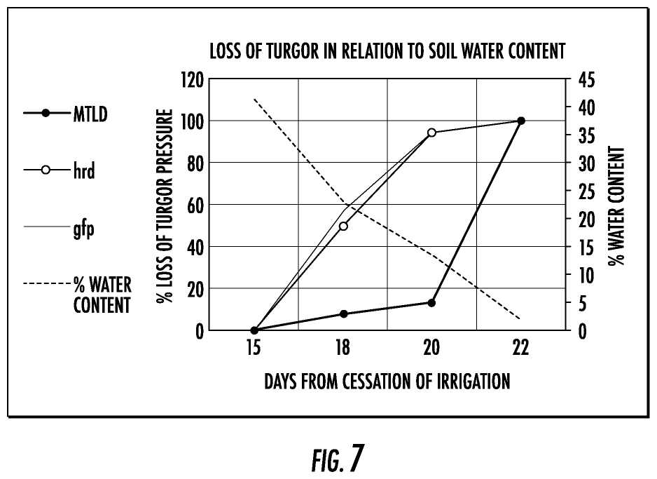

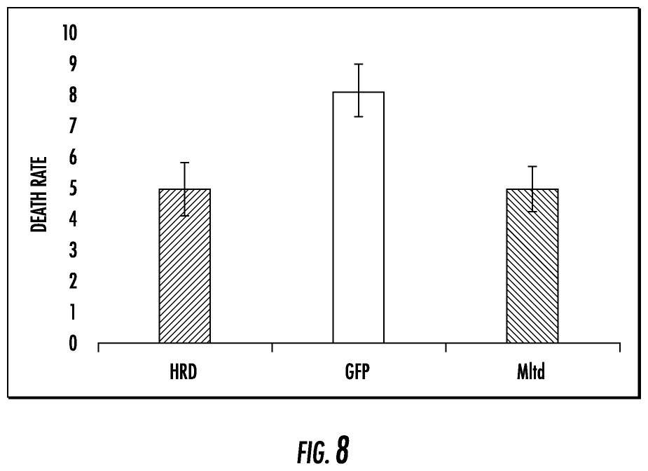

When drought conditions start to develop, various measurements are taken, as shown in Table 3:
1) turgor observation, measured by scale of 1-10, when 1 is high turgor and 10 is total loss of turgor (see FIG. 7).
2) Weight of plant and pot, by scale in grams.
3) Death of plants observation, 10=dead and 1=alive (see FIG. 8)
4) State of plants observation in a scale of 1-10, when 1 is good state and 10 is poor.

TABLE 3
Measurements taken in drought experiments

| | | |
|---|---|---|
| measurement | units | time of measurement |
| Weight of pot | Grams | Start till end of experiment |
| Turgor | Observation units 1-10, | From beginning of turgor |
| | where 1 is 0 turgor loss and | loss (~15-27 days from last |
| | 10 is 100% turgor loss | irrigation) |
| Death | Observation units 1-10 | From first death observed |
| State of plants | Observation units 1-10 | During first 2 weeks and |
| | | one day after revival |

Reference is now made to FIG. 7, showing loss of turgor pressure in plants expressing genes used as control relative to soil water content (dark gray), days after cessation of irrigation. This figure shows curves of Arabidopsis plants, expressing different genes (indicated), as a response to growth under drought conditions. Dark line indicates soil water content from 40% in day 15 after water irrigation ceased, to close to 0% at day 22 after water irrigation ceased. The negative control GFP plant's loss of turgor pressure response is similar to that of HRD expressing plants, while mtlD expressing plants turgor pressure, seem to be less effected by drought until day 20 after water irrigation ceased.

It is demonstrated in this figure that plants expressing the positive control genes mtlD and HRD showed improved resistance to drought by showing significantly reduced loss of turgor pressure effects, while transgenic plants expressing the negative control GFP gene showed elevated loss of turgor pressure effect when exposed to the same water content loss.

Reference is now made to FIG. 8 showing normalized death scale of positive control expressing transgenic plants as compared to GFP expressing plants. As can be seen plants expressing the drought resistance positive control genes HRD and mtlD showed significantly reduced death rate as compared to the negative control GFP expressing plants.

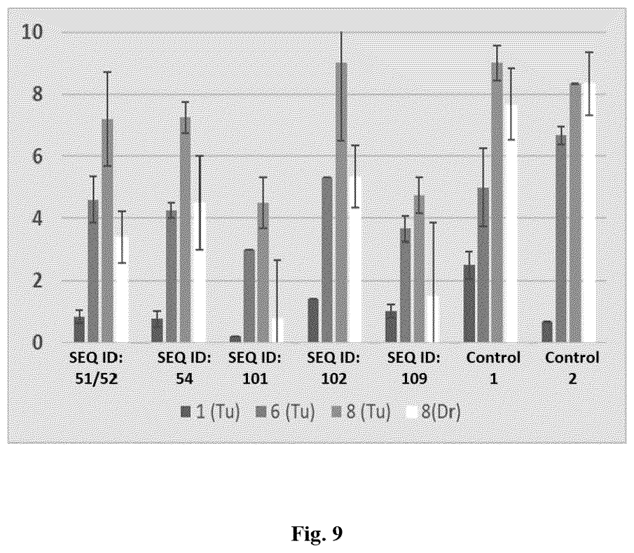

Reference is now made to FIG. 9 graphically showing phenotypic results of several drought resistance genes identified by the method of the present invention.

The graph shows average results of turgor pressure (Tu) and death rate (Dr) for several identified genes (see Table 4) under severe drought conditions. Scale for death and turgor loss is 1-10 when 10 is considered dry-brown and dead plants, or total loss of turgor, respectively. The results in the graph represent day 23 (1), day 28 (6) and day 30 (8) from sowing. Each column for each of the different expressed genes represents average of 5 repeats with 4 plants in each repeat. GFP expressing plants served as negative control and HRD as positive control. As can be seen, all tested genes identified by the method of the present invention showed significantly reduced turgor loss (by at least two fold after about 23 days from sowing) and reduced death rate (in the range of 9 to 2 fold after 30 days from sowing) as compared to plants expressing the negative control GFP gene. Moreover, plants expressing the newly discovered genes (see Table 4) demonstrated a significantly reduced death rated as compared to the positive control HRD expressing plants. These results indicate that by the method of the present invention, newly drought resistance genes are identified, which confer improved tolerance to drought in plants.

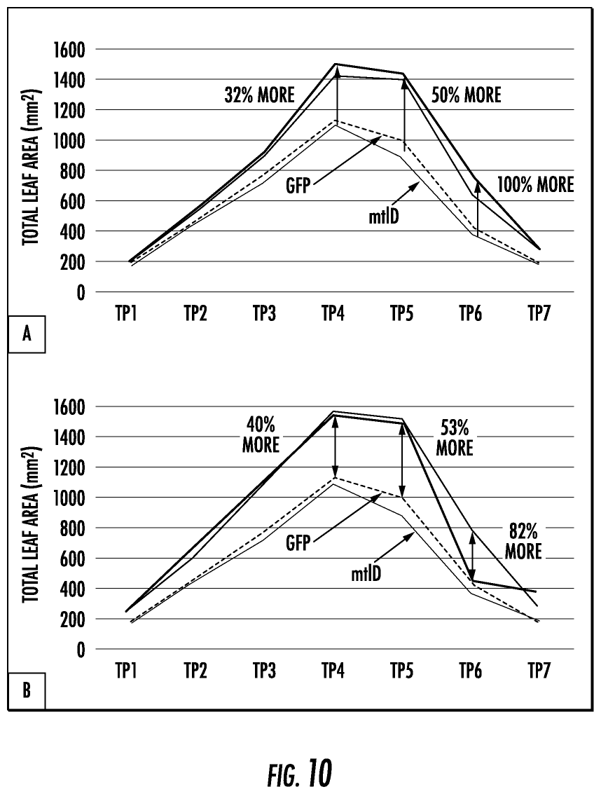

Another method used for evaluating plants performance in drought conditions is measuring their leaf area during the growth phase when drought conditions become prominent. About 10-14 days from sowing the plants, plant images were taken every 2-3 days together with a 50 mm² white surface. Image analysis was performed on pictures taken from the drought experiments and leaf area was calculated. The leaf area of several plant lines expressing novel genes identified as conferring drought resistance after re-cloning was compared to positive and negative controls (see Table 5 and FIG. 10).

The graph of FIG. 10 shows image analysis of leaf area of transformed plant lines. Two independent transformation events of the identified gene having SEQ ID NO:16 (FIG. 10A) and two independent transformation events of the identified gene having SEQ ID NO:25 (FIG. 10B) are shown in darker lines on top of each of the FIGS. 10A and 10B. These transgenic plants are compared to negative control plants expressing GFP, and positive control plants expressing mtlD, shown in lighter gray lines on the bottom of each of the FIGS. 10A and 10B. Improved performance under drought is shown as percentage from control plants at the indicated measured timepoint (TP) (arrows and percentages shown in the figure).

As can be seen in this figure, the total leaf area of plants expressing the newly identified tested genes was increased by between about 10% and about 82% (e.g. by about 45%) relative to plants expressing negative control genes.

To conclude, the present invention provides newly identified genes demonstrated to confer tolerance to drought conditions in plants.

### C. Re-Cloning and Retransformation of Selected Genes into Plants

Selected genes from section B are re-cloned into the binary vectors as described above (i.e. FIG. 1A-D) and sequenced to confirm that it has the same sequence as the original gene from T1 and T2 experiments. Plants are transformed with the re-cloned gene and seeds are collected. Experiments are repeated as in B except for each gene 3-5 individual transgenic plants with different unrelated transformation events are tested. Each individual transgenic plant/event is subjected to 5-10 times of repeats in experiments, hence for each event for every gene 20-40 plants are tested, and for every different gene 60-200 plants are tested.

### EXAMPLE 3

### Polynucleotide Sequences Identified as Improving Drought and/or Salinity Resistance in Plants

The process described above of screening of T1 transgenic seeds revealed about 1000 transgenes as candidate polynucleotide sequences for improving drought resistance in plants. Of these candidates, the screening of T2 seeds revealed about 140 best performing transgenes potentially improving drought resistance or tolerance in plants. These transgene sequences are subjected to further validation tests.

Reference is now made to Table 4, presenting examples of novel and unique polynucleotide sequences and polypeptides encoded by these sequences, found by the method of the present invention. These sequences are metatranscriptomes purified from environmentally challenged niches,

SEQ ID NO: 1 to SEQ ID NO: 148 represent polynucleotide sequences found by the method of the present invention as candidates for improving drought resistance in plants (Table 4).

SEQ ID NO:149 to SEQ ID NO:321 represent polypeptide sequences encoded by the corresponding polynucleotide sequence found by the method of the present invention as candidates for improving drought resistance in plants (see Table 4).

Note that DNA sequences SEQ ID NO:9, SEQ ID NO:11, SEQ ID NO:20, SEQ ID NO:21, SEQ ID NO:28, SEQ ID NO:36, SEQ ID NO:43, SEQ ID NO:47, SEQ ID NO:51, SEQ ID NO:54, SEQ ID NO:55, SEQ ID NO:60, SEQ ID NO:75, SEQ ID NO:78, SEQ ID NO:80, SEQ ID NO:81, SEQ ID NO:85, SEQ ID NO:87, SEQ ID NO:97, SEQ ID NO:98, SEQ ID NO:105, SEQ ID NO:106, SEQ ID NO:107, SEQ ID NO:109, SEQ ID NO:111, SEQ ID NO:112, SEQ ID NO:120, SEQ ID NO:132, SEQ ID NO:134, SEQ ID NO:135, SEQ ID NO:140, SEQ ID NO:141 encode more than one open reading frame (ORF) (referred to as SEQ. ID NO X.1p and X.2p etc.) depending on different start codons.

TABLE 4
SEQ ID Nos of polynucleotide and polypeptide sequences

| Polynucleotide SEQ ID NO. | Polynucleotide name |
|---|---|
| SEQ ID NO: 1 | A454 |
| SEQ ID NO: 2 | A456 |
| SEQ ID NO: 3 | A458.1 |
| SEQ ID NO: 4 | A458.2 |
| SEQ ID NO: 5 | A460 |
| SEQ ID NO: 6 | A462 |
| SEQ ID NO: 7 | A463 |
| SEQ ID NO: 8 | A466 |
| SEQ ID NO: 9 | A468 |
| SEQ ID NO: 10 | A470 |
| SEQ ID NO: 11 | A475 |
| SEQ ID NO: 12 | A477 |
| SEQ ID NO: 13 | A480 |
| SEQ ID NO: 14 | A481 |
| SEQ ID NO: 15 | A483 |
| SEQ ID NO: 16 | A484 |
| SEQ ID NO: 17 | A485a |
| SEQ ID NO: 18 | A485b |
| SEQ ID NO: 19 | A486 |
| SEQ ID NO: 20 | A498 |
| SEQ ID NO: 21 | A499 |
| SEQ ID NO: 22 | A501 |
| SEQ ID NO: 23 | A504.1 |
| SEQ ID NO: 24 | A504 |
| SEQ ID NO: 25 | A506 |
| SEQ ID NO: 26 | A507.1 |
| SEQ ID NO: 27 | A507.2 |
| SEQ ID NO: 28 | A510a |
| SEQ ID NO: 29 | A510b |
| SEQ ID NO: 30 | A512 |
| SEQ ID NO: 31 | A513a |
| SEQ ID NO: 32 | A513b |
| SEQ ID NO: 33 | A518 |
| SEQ ID NO: 34 | A520a |
| SEQ ID NO: 35 | AC2510 |
| SEQ ID NO: 36 | AD2607.1 |
| SEQ ID NO: 37 | AD2607.3 |
| SEQ ID NO: 38 | D860a |
| SEQ ID NO: 39 | D860b |
| SEQ ID NO: 40 | D862 |
| SEQ ID NO: 41 | D863 |
| SEQ ID NO: 42 | D881 |
| SEQ ID NO: 43 | D890 |
| SEQ ID NO: 44 | De203 |
| SEQ ID NO: 45 | De214a |
| SEQ ID NO: 46 | De215a |
| SEQ ID NO: 47 | De215b.1 |
| SEQ ID NO: 48 | De215b.4 |
| SEQ ID NO: 49 | De215c |
| SEQ ID NO: 50 | De217 |
| SEQ ID NO: 51 | De223a |
| SEQ ID NO: 52 | De223b |
| SEQ ID NO: 53 | De227 |
| SEQ ID NO: 54 | De239a |
| SEQ ID NO: 55 | De245 |
| SEQ ID NO: 56 | De250.1 |
| SEQ ID NO: 57 | De250.2 |
| SEQ ID NO: 58 | De251 |
| SEQ ID NO: 59 | De313 |
| SEQ ID NO: 60 | F1022a |
| SEQ ID NO: 61 | F1022b |
| SEQ ID NO: 62 | G1085a |
| SEQ ID NO: 63 | G1181 |
| SEQ ID NO: 64 | G1190 |
| SEQ ID NO: 65 | H1301.1 |
| SEQ ID NO: 66 | H1301.2 |
| SEQ ID NO: 67 | K1464 |
| SEQ ID NO: 68 | K1475 |
| SEQ ID NO: 69 | M603 |
| SEQ ID NO: 70 | M606.1 |
| SEQ ID NO: 71 | M606.2 |
| SEQ ID NO: 72 | M607.1 |
| SEQ ID NO: 73 | M607.2 |
| SEQ ID NO: 74 | M609a.1 |
| SEQ ID NO: 75 | M609a.2 |
| SEQ ID NO: 76 | M609b |
| SEQ ID NO: 77 | M619a |
| SEQ ID NO: 78 | M619b |
| SEQ ID NO: 79 | M622a |
| SEQ ID NO: 80 | M622b |
| SEQ ID NO: 81 | M623a |
| SEQ ID NO: 82 | M623b.1 |
| SEQ ID NO: 83 | M623b.3 |
| SEQ ID NO: 84 | M623c |
| SEQ ID NO: 85 | M624 |
| SEQ ID NO: 86 | M625a.3 |
| SEQ ID NO: 87 | M625a |
| SEQ ID NO: 88 | M625b |
| SEQ ID NO: 89 | M631 |
| SEQ ID NO: 90 | M632a |
| SEQ ID NO: 91 | M635.1 |
| SEQ ID NO: 92 | M635.2 |
| SEQ ID NO: 93 | M638 |
| SEQ ID NO: 94 | M643 |
| SEQ ID NO: 95 | M649 |
| SEQ ID NO: 96 | M650a.3 |
| SEQ ID NO: 97 | M650a |
| SEQ ID NO: 98 | M650b |
| SEQ ID NO: 99 | M657 |
| SEQ ID NO: 100 | M659a |
| SEQ ID NO: 101 | M661 |
| SEQ ID NO: 102 | M663 |
| SEQ ID NO: 103 | M664.1 |
| SEQ ID NO: 104 | M664.2 |
| SEQ ID NO: 105 | M666 |
| SEQ ID NO: 106 | M671 |
| SEQ ID NO: 107 | M673 |
| SEQ ID NO: 108 | M676.3 |
| SEQ ID NO: 109 | M676 |
| SEQ ID NO: 110 | M677a |
| SEQ ID NO: 111 | M677b.1 |
| SEQ ID NO: 112 | M677b.3 |
| SEQ ID NO: 113 | M680 |
| SEQ ID NO: 114 | M691a.1 |
| SEQ ID NO: 115 | M691a.2 |
| SEQ ID NO: 116 | M691b |
| SEQ ID NO: 117 | M693 |
| SEQ ID NO: 118 | M697 |
| SEQ ID NO: 119 | M698 |
| SEQ ID NO: 120 | M705 |
| SEQ ID NO: 121 | M706 |
| SEQ ID NO: 122 | M715a |
| SEQ ID NO: 123 | M715b |
| SEQ ID NO: 124 | M719 |
| SEQ ID NO: 125 | M724 |
| SEQ ID NO: 126 | N1503a |
| SEQ ID NO: 127 | N1527.1 |
| SEQ ID NO: 128 | N1527.2 |
| SEQ ID NO: 129 | N1529 |
| SEQ ID NO: 130 | N1530 |
| SEQ ID NO: 131 | P1611 |
| SEQ ID NO: 132 | P1620.1 |
| SEQ ID NO: 133 | P1620.3 |
| SEQ ID NO: 134 | P1623a |
| SEQ ID NO: 135 | P1623b |
| SEQ ID NO: 136 | P1625a |
| SEQ ID NO: 137 | P1625b |
| SEQ ID NO: 138 | P1731 |
| SEQ ID NO: 139 | P1744 |
| SEQ ID NO: 140 | P1747.1 |
| SEQ ID NO: 141 | P1747.3 |
| SEQ ID NO: 142 | SN8 |
| SEQ ID NO: 143 | V1906b |
| SEQ ID NO: 144 | V1906c |
| SEQ ID NO: 145 | V1907a |
| SEQ ID NO: 146 | V1907b |
| SEQ ID NO: 147 | X2005 |
| SEQ ID NO: 148 | X2026 |
| SEQ ID NO: 149 | A454p |
| SEQ ID NO: 150 | A456p |
| SEQ ID NO: 151 | A458.1p |
| SEQ ID NO: 152 | A458.2p |
| SEQ ID NO: 153 | A460p |
| SEQ ID NO: 154 | A462p |
| SEQ ID NO: 155 | A463p |
| SEQ ID NO: 156 | A466p |
| SEQ ID NO: 157 | A468.1p |
| SEQ ID NO: 158 | A468.2p |
| SEQ ID NO: 159 | A470p |
| SEQ ID NO: 160 | A475.1p |
| SEQ ID NO: 161 | A475.2p |
| SEQ ID NO: 162 | A477p |
| SEQ ID NO: 163 | A480p |
| SEQ ID NO: 164 | A481p |
| SEQ ID NO: 165 | A483p |
| SEQ ID NO: 166 | A484p |
| SEQ ID NO: 167 | A485ap |
| SEQ ID NO: 168 | A485bp |
| SEQ ID NO: 169 | A486p |
| SEQ ID NO: 170 | A498.1p |
| SEQ ID NO: 171 | A498.2p |
| SEQ ID NO: 172 | A499.1p |
| SEQ ID NO: 173 | A499.2p |
| SEQ ID NO: 174 | A501p |
| SEQ ID NO: 175 | A504.1p |
| SEQ ID NO: 176 | A504.2p |
| SEQ ID NO: 177 | A506p |
| No ORF identified | No ORF identified |
| SEQ ID NO: 178 | A507.2p |
| SEQ ID NO: 179 | A510a.1p |
| SEQ ID NO: 180 | A510a.2p |
| No ORF identified | No ORF identified |
| SEQ ID NO: 181 | A512p |
| SEQ ID NO: 182 | A513ap |
| SEQ ID NO: 183 | A513bp |
| SEQ ID NO: 184 | A518p |
| SEQ ID NO: 185 | A520ap |
| SEQ ID NO: 186 | AC2510ap |
| SEQ ID NO: 187 | AD2607.1p |
| SEQ ID NO: 188 | AD2607.2p |
| SEQ ID NO: 189 | AD2607.3p |
| SEQ ID NO: 190 | D860ap |
| SEQ ID NO: 191 | D860bp |
| SEQ ID NO: 192 | D862p |
| SEQ ID NO: 193 | D863p |
| SEQ ID NO: 194 | D881p |
| SEQ ID NO: 195 | D890.1p |
| SEQ ID NO: 196 | D890.2p |
| SEQ ID NO: 197 | De203p |
| SEQ ID NO: 198 | De214ap |
| SEQ ID NO: 199 | De215ap |
| SEQ ID NO: 200 | De215b.1p |
| SEQ ID NO: 201 | De215b.2p |
| SEQ ID NO: 202 | De215b.3p |
| SEQ ID NO: 203 | De215b.4p |
| SEQ ID NO: 204 | De215cp |
| No ORF identified | No ORF identified |
| SEQ ID NO: 205 | De223a.1p |
| SEQ ID NO: 206 | De223a.2p |
| SEQ ID NO: 207 | De223bp |
| SEQ ID NO: 208 | De227p |
| SEQ ID NO: 209 | De239a.1p |
| SEQ ID NO: 210 | De239a.2p |
| SEQ ID NO: 211 | De245.1p |
| SEQ ID NO: 212 | De245.2p |
| SEQ ID NO: 213 | De250p |
| SEQ ID NO: 214 | De250.2p |
| SEQ ID NO: 215 | De251p |
| SEQ ID NO: 216 | De313p |
| SEQ ID NO: 217 | F1022a.1p |
| SEQ ID NO: 218 | F1022a.2p |
| SEQ ID NO: 219 | F1022bp |
| SEQ ID NO: 220 | G1085ap |
| SEQ ID NO: 221 | G1181p |
| SEQ ID NO: 222 | G1190p |
| SEQ ID NO: 223 | H1301.1p |
| SEQ ID NO: 224 | H1301.2p |
| No ORF identified | No ORF identified |
| SEQ ID NO: 225 | K1475p |
| SEQ ID NO: 226 | M603p |
| SEQ ID NO: 227 | M606.1p |
| SEQ ID NO: 228 | M606.2p |
| SEQ ID NO: 229 | M607. 1p |
| SEQ ID NO: 230 | M607.2p |
| SEQ ID NO: 231 | M609a.1p |
| SEQ ID NO: 233 | M609a.3p |
| SEQ ID NO: 232 | M609a.2p |
| SEQ ID NO: 234 | M609bp |
| SEQ ID NO: 235 | M619ap |
| SEQ ID NO: 236 | M619b.1p |
| SEQ ID NO: 237 | M619b.2p |
| SEQ ID NO: 238 | M622ap |
| SEQ ID NO: 239 | M622b.1p |
| SEQ ID NO: 240 | M622b.2p |
| SEQ ID NO: 241 | M623a.1p |
| SEQ ID NO: 242 | M623a.2p |
| No ORF identified | No ORF identified |
| SEQ ID NO: 243 | M623b.3p |
| SEQ ID NO: 244 | M623cp |
| SEQ ID NO: 245 | M624. 1p |
| SEQ ID NO: 246 | M624.2p |
| SEQ ID NO: 249 | M625a.3p |
| SEQ ID NO: 247 | M625a.1p |
| SEQ ID NO: 248 | M625a.2p |
| SEQ ID NO: 250 | M625bp |
| SEQ ID NO: 251 | M631p |
| SEQ ID NO: 252 | M632ap |
| SEQ ID NO: 253 | M635.1p |
| SEQ ID NO: 254 | M635.2p |
| SEQ ID NO: 255 | M638p |
| SEQ ID NO: 256 | M643p |
| SEQ ID NO: 257 | M649p |
| SEQ ID NO: 260 | M650a.3p |
| SEQ ID NO: 258 | M650a.1p |
| SEQ ID NO: 259 | M650a.2p |
| SEQ ID NO: 261 | M650b.1p |
| SEQ ID NO: 262 | M650b.2p |
| SEQ ID NO: 263 | M657p |
| SEQ ID NO: 264 | M659ap |
| SEQ ID NO: 265 | M661p |
| SEQ ID NO: 266 | M663p |
| SEQ ID NO: 267 | M664.1p |
| SEQ ID NO: 268 | M664.2p |
| SEQ ID NO: 269 | M666.1p |
| SEQ ID NO: 270 | M666.2p |
| SEQ ID NO: 271 | M671.1p |
| SEQ ID NO: 272 | M671.2p |
| SEQ ID NO: 273 | M673.1p |
| SEQ ID NO: 274 | M673.2p |
| SEQ ID NO: 277 | M676.3p |
| SEQ ID NO: 275 | M676.1p |
| SEQ ID NO: 276 | M676.2p |
| SEQ ID NO: 278 | M677ap |
| SEQ ID NO: 279 | M677b.1p |
| SEQ ID NO: 280 | M677b.2p |
| SEQ ID NO: 281 | M677b.3p |
| SEQ ID NO: 282 | M677b.4p |
| SEQ ID NO: 283 | M680p |
| SEQ ID NO: 284 | M691a.1p |
| SEQ ID NO: 285 | M691a.2p |
| SEQ ID NO: 286 | M691bp |
| SEQ ID NO: 287 | M693p |
| SEQ ID NO: 288 | M697p |
| SEQ ID NO: 289 | M698p |
| SEQ ID NO: 290 | M705.1p |
| SEQ ID NO: 291 | M705.2p |
| SEQ ID NO: 292 | M706p |
| SEQ ID NO: 293 | M715ap |
| SEQ ID NO: 294 | M715bp |
| SEQ ID NO: 295 | M719p |
| SEQ ID NO: 296 | M724p |
| SEQ ID NO: 297 | N1503ap |
| SEQ ID NO: 298 | N1527.1p |
| SEQ ID NO: 299 | N1527.2p |
| SEQ ID NO: 300 | N1529p |
| SEQ ID NO: 301 | N1530p |
| SEQ ID NO: 302 | P1611p |
| SEQ ID NO: 303 | P1620.1p |
| SEQ ID NO: 304 | P1620.2p |
| SEQ ID NO: 305 | P1620.3p |
| SEQ ID NO: 306 | P1623a.1p |
| SEQ ID NO: 307 | P1623a.2p |
| SEQ ID NO: 308 | P1623b.1p |
| SEQ ID NO: 309 | P1623b.2p |
| SEQ ID NO: 310 | P1625ap |
| SEQ ID NO: 311 | P1625bp |
| SEQ ID NO: 312 | P1731p |
| SEQ ID NO: 313 | P1744p |
| SEQ ID NO: 314 | P1747.1p |
| SEQ ID NO: 315 | P1747.2p |
| SEQ ID NO: 316 | P1747.3p |
| SEQ ID NO: 317 | P1747.4p |
| No ORF identified | No ORF identified |
| SEQ ID NO: 318 | V1906bp |
| No ORF identified | No ORF identified |
| SEQ ID NO: 319 | V1907ap |
| No ORF identified | No ORF identified |
| SEQ ID NO: 320 | X2005p |
| SEQ ID NO: 321 | X2026p |

Reference is now made to Table 5 presenting phenotypic results of several of the identified genes in the drought tolerance experiments. Plants were grown in soil in controlled greenhouses and tested for drought tolerance under the conditions mentioned above. During their growth, measurements and images were taken (see Table 3) and image analysis was applied converting the images to leaf area per plant. Results are shown as percentage of GFP expressing plants measurements that served as a negative control during the drought phase.

TABLE 5
Results of drought experiments conducted with T2 Arabidopsis plants

| Seq ID | DR | ±SD | Seq ID | DR | ±SD | Seq ID | DR | ±SD | Seq ID | DR | ±SD | Seq ID | DR | ±SD |
|---|---|---|---|---|---|---|---|---|---|---|---|---|---|---|
| SEQ ID | 116.00 | 3.13 | SEQ ID | 137.14 | 7.05 | SEQ ID | 144.00 | 5.56 | SEQ ID | 143.23 | 7.26 | SEQ ID | 120.00 | 10.25 |
| NO: 1 | | | NO: 25 | | | NO: 55 | | | NO: 91/92 | | | NO: 122 | | |
| SEQ ID | 132.86 | 6.68 | SEQ ID | 135.00 | 7.64 | SEQ ID | 134.00 | 3.28 | SEQ ID | 133.33 | 6.84 | SEQ ID | 130.73 | 5.93 |
| NO: 2 | | | NO: 26/27 | | | NO: | | | NO: 93 | | | NO: 124 | | |
| SEQ ID | 151.43 | 10.52 | SEQ ID | 187.64 | 11.00 | 56/57 | | | SEQ ID | 102.50 | 7.08 | SEQ ID | 139.10 | 9.76 |
| NO: 17/18 | | | NO: 28 | | | SEQ ID | 151.6 | 20.7 | NO: 94 | | | NO: 125 | | |
| SEQ ID | 146.9 | 20.6 | SEQ ID | 118.57 | 1.88 | NO: 58 | | | SEQ ID | 133.41 | 7.61 | SEQ ID | 119.09 | 4.03 |
| NO: 5 | | | NO: 30 | | | SEQ ID | 134.08 | 4.45 | NO: 97 | | | NO: 127/ | | |
| SEQ ID | 118.57 | 5.26 | SEQ ID | 112.56 | 4.97 | NO: 60 | | | SEQ ID | 137.50 | 8.80 | 128 | | |
| NO: 6 | | | NO: 31 | | | SEQ ID | 99.72 | 4.71 | NO: 99 | | | SEQ ID | 135.01 | 7.89 |
| SEQ ID | 156.7 | 23.4 | SEQ ID | 167.57 | 7.20 | NO: 62 | | | SEQ ID | 160.11 | 20.12 | NO: 129 | | |
| NO: 7 | | | NO: 33 | | | SEQ ID | 277.71 | 16.80 | NO: 100 | | | SEQ ID | 196.80 | 9.06 |
| SEQ ID | 162.1 | 17.1 | SEQ ID | 118.92 | 5.31 | NO: 63 | | | SEQ ID | 182.50 | 10.00 | NO: 130 | | |
| NO: 8 | | | NO: 34 | | | SEQ ID | 136.83 | 6.62 | NO: 101 | | | SEQ ID | 113.98 | 7.65 |
| SEQ ID | 138.24 | 20.36 | SEQ ID | 115.20 | 6.60 | NO: 64 | | | SEQ ID | 136.67 | 8.72 | NO: 131 | | |
| NO: 9 | | | NO: 35 | | | SEQ ID | 107.77 | 10.82 | NO: 102 | | | SEQ ID | 110.33 | 6.64 |
| SEQ ID | 116.00 | 3.19 | SEQ ID | 109.71 | 7.79 | NO: | | | SEQ ID | 121.79 | 7.45 | NO: 132/ | | |
| NO: 10 | | | NO: 36/37 | | | 65/66 | | | NO: | | | 133 | | |
| SEQ ID | 107.14 | 2.93 | SEQ ID | 124.59 | 6.74 | SEQ ID | 131.25 | 7.04 | 103/104 | | | SEQ ID | 107.54 | 9.76 |
| NO: 11 | | | NO: 38 | | | NO: 67 | | | SEQ ID | 126.73 | 6.48 | NO: 134 | | |
| SEQ ID | 122.86 | 4.53 | SEQ ID | 154.29 | 10.83 | SEQ ID | 186.67 | 9.85 | NO: 105 | | | SEQ ID | 114.17 | 5.84 |
| NO: 12 | | | NO: 40 | | | NO: 68 | | | SEQ ID | 125.00 | 5.94 | NO: 137 | | |
| SEQ ID | 160.00 | 12.32 | SEQ ID | 117.14 | 5.71 | SEQ ID | 132.64 | 8.96 | NO: 106 | | | SEQ ID | 139.80 | 9.87 |
| NO: 13 | | | NO: 41 | | | NO: 69 | | | SEQ ID | 130.00 | 6.64 | NO: 138 | | |
| SEQ ID | 142.86 | 8.37 | SEQ ID | 118.27 | 3.56 | SEQ ID | 145.00 | 7.07 | NO: 107 | | | SEQ ID | 115.04 | 6.38 |
| NO: 14 | | | NO: 42 | | | NO: | | | SEQ ID | 175.00 | 7.38 | NO: 139 | | |
| SEQ ID | 145.71 | 7.24 | SEQ ID | 141.69 | 8.03 | 70/71 | | | NO: 109 | | | SEQ ID | 105.73 | 8.08 |
| NO: 15 | | | NO: 43 | | | SEQ ID | 134.08 | 7.08 | SEQ ID | 118.92 | 5.89 | NO: 140/ | | |
| SEQ ID | 136.13 | 8.55 | SEQ ID | 144.00 | 6.36 | NO: | | | NO: 110 | | | 141 | | |
| NO: 16 | | | NO: 44 | | | 72/73 | | | SEQ ID | 113.14 | 8.47 | SEQ ID | 141.43 | 3.65 |
| SEQ ID | 108.33 | 2.73 | SEQ ID | 142.70 | 9.33 | SEQ ID | 187.50 | 10.00 | NO: 113 | | | NO: 142 | | |
| NO: 17 | | | NO: 45 | | | NO: | | | SEQ ID | 108.04 | 5.44 | SEQ ID | 115.50 | 7.96 |
| SEQ ID | 121.67 | 5.66 | SEQ ID | 119.36 | 9.40 | 74/75/76 | | | NO: 114/ | | | NO: 143 | | |
| NO: 19 | | | NO: 46 | | | SEQ ID | 125.00 | 8.29 | 115/116 | | | SEQ ID | 112.59 | 7.32 |
| SEQ ID | 118.68 | 2.48 | SEQ ID | 110.51 | 7.81 | NO: 77 | | | SEQ ID | 167.50 | 9.13 | NO: 145 | | |
| NO: 20 | | | NO: 50 | | | SEQ ID | 123.73 | 6.78 | NO: 117 | | | SEQ ID | 121.66 | 8.81 |
| SEQ ID | 116.67 | 4.01 | SEQ ID | 158.00 | 13.73 | NO: 79 | | | SEQ ID | 131.68 | 8.77 | NO: 147 | | |
| NO: 21 | | | NO: 51 | | | SEQ ID | 159.79 | 8.45 | NO: 118 | | | SEQ ID | 121.07 | 5.86 |
| SEQ ID | 131.67 | 8.00 | SEQ ID | 119.42 | 8.70 | NO: 81 | | | SEQ ID | 121.04 | 6.30 | NO: 148 | | |
| NO: 22 | | | NO: 53 | | | SEQ ID | 180.00 | 7.07 | NO: 119 | | | GFP | 100.00 | 6.55 |
| SEQ ID | 124.29 | 6.45 | SEQ ID | 145.00 | 11.92 | NO: 85 | | | SEQ ID | 104.85 | 7.51 | | | |
| NO: 23/24 | | | NO: 54 | | | SEQ ID | 267.03 | 16.40 | NO: 120 | | | | | |
| | | | | | | NO: 87 | | | SEQ ID | 113.85 | 6.36 | | | |
| | | | | | | SEQ ID | 173.33 | 4.58 | NO:121 | | | | | |
| | | | | | | NO: 89 | | | | | | | | |
| | | | | | | SEQ ID | 135.00 | 9.29 | | | | | | |
| | | | | | | NO: 90 | | | | | | | | |

DR-performance (leaf area) under Drought shown in % of GFP expressing plants
SD-value shown ± standard deviation

As shown in Table 5, all plants expressing the tested genes identified by the method of the present invention revealed increased leaf area by about 15% to about 90% under drought conditions as compared to plants expressing the negative control gene (GFP). These results demonstrate that the method of the present invention provides novel genes conferring improved drought tolerance in plants.

Reference is now made to Table 6 presenting results of drought experiments conducted with T2 Arabidopsis plants re-cloned with the relevant Seq. IDs. Different Seq. IDs were re-cloned and re-transformed into Arabidopsis plants generating several independent events (represented by E1-3 in Table 6). Plants were grown in soil in controlled greenhouses and tested for drought tolerance under the conditions mentioned above. During their growth, images were taken and image analysis was applied, converting the images into leaf area per plant. Results are shown in Table 6 as percentage of GFP expressing plants that served as a negative control during the drought phase.

TABLE 6
Results of drought experiments conducted with
T2 Arabidopsis plants re-cloned with the relevant Seq. IDs

| Seq ID | DR RC E1 | E1 ± SD | DR RC E2 | E2 ± SD | DR RC E3 | E3 ± SD |
|---|---|---|---|---|---|---|
| SEQ ID NO: 2 | 114.05 | 12.00 | 126.33 | 6.27 | 94.42 | 14.75 |
| SEQ ID NO: 7 | 125.74 | 7.50 | 118.43 | 12.40 | 82.39 | 17.20 |
| SEQ ID NO: 8 | 126.96 | 10.73 | 110.07 | 13.09 | 132.74 | 5.34 |
| SEQ ID NO: 9 | 159.34 | 19.75 | 151.99 | 27.05 | 113.97 | 18.65 |
| SEQ ID NO: 10 | 185.23 | 19.29 | 165.97 | 30.99 | 90.04 | 9.27 |
| SEQ ID NO: 11 | 116.91 | 9.54 | 106.90 | 10.41 | 106.32 | 10.87 |
| SEQ ID NO: 12 | 178.80 | 24.09 | 107.57 | 14.72 | 157.66 | 15.22 |
| SEQ ID NO: 14 | 162.78 | 14.10 | 151.93 | 9.90 | 123.68 | 10.10 |
| SEQ ID NO: 16 | 144.23 | 8.42 | 141.32 | 7.03 | 127.03 | 8.31 |
| SEQ ID NO: 18 | 176.30 | 26.57 | 126.24 | 11.63 | 138.53 | 23.03 |
| SEQ ID NO: 22 | 113.00 | 12.14 | 109.38 | 9.14 | 105.16 | 12.38 |
| SEQ ID NO: 25 | 150.56 | 7.57 | 153.02 | 9.91 | 120.25 | 13.63 |
| SEQ ID NO: 28 | 193.32 | 28.79 | | | | |
| SEQ ID NO: 30 | 123.33 | 11.83 | 113.97 | 8.18 | 112.53 | 16.34 |
| SEQ ID NO: 33 | 141.20 | 10.90 | 127.98 | 13.30 | 112.63 | 11.50 |
| SEQ ID NO: 34 | 167.25 | 12.60 | 150.19 | 13.30 | 138.48 | 10.20 |
| SEQ ID NO: 41 | 160.43 | 11.60 | 153.92 | 14.10 | 112.83 | 10.80 |
| SEQ ID NO: 43 | 229.50 | 18.12 | 136.33 | 32.37 | 106.83 | 26.53 |
| SEQ ID NO: 51 | 178.07 | 13.10 | 170.57 | 14.60 | 146.17 | 11.20 |
| SEQ ID NO: 54 | 169.39 | 15.50 | 131.72 | 11.30 | 120.10 | 16.70 |
| SEQ ID NO: 55 | 126.72 | 16.39 | 122.48 | 18.62 | 111.94 | 17.92 |
| SEQ ID NO: 56/57 | 138.08 | 8.64 | 134.76 | 9.21 | 127.74 | 10.65 |
| SEQ ID NO: 58 | 115.36 | 11.52 | 117.79 | 13.24 | 93.16 | 11.94 |
| SEQ ID NO: 60 | 151.90 | 12.80 | 137.24 | 11.90 | 93.80 | 5.60 |
| SEQ ID NO: 61 | 140.14 | 12.10 | 116.31 | 14.70 | 114.09 | 10.30 |
| SEQ ID NO: 74/75/76 | 175.07 | 13.50 | 160.92 | 12.30 | 105.95 | 11.30 |
| SEQ ID NO: 77 | 210.21 | 18.03 | 174.80 | 18.44 | 160.93 | 29.97 |
| SEQ ID NO: 78 | 182.00 | 15.30 | 175.52 | 16.80 | 115.61 | 11.10 |
| SEQ ID NO: 85 | 132.73 | 10.80 | 119.86 | 11.50 | 114.46 | 9.90 |
| SEQ ID NO: 89 | 167.95 | 21.26 | 154.64 | 21.46 | 142.21 | 29.65 |
| SEQ ID NO: 90 | 141.50 | 24.45 | 137.53 | 17.22 | 110.29 | 32.15 |
| SEQ ID NO: 91/92 | 219.30 | 29.16 | 192.51 | 22.47 | 92.77 | 20.90 |
| SEQ ID NO: 93 | 127.73 | 16.50 | 122.99 | 11.32 | 119.54 | 17.08 |
| SEQ ID NO: 94 | 123.64 | 13.85 | 120.32 | 9.86 | 107.77 | 15.59 |
| SEQ ID NO: 95 | 129.53 | 9.05 | 108.36 | 9.42 | 98.43 | 14.09 |
| SEQ ID NO: 101 | 161.68 | 14.10 | 141.20 | 11.30 | 134.68 | 13.60 |
| SEQ ID NO: 105 | 204.51 | 27.93 | 188.14 | 5.31 | 156.19 | 17.89 |
| SEQ ID NO: 106 | 153.33 | 12.60 | 143.91 | 10.80 | 130.47 | 11.50 |
| SEQ ID NO: 109 | 141.18 | 14.20 | 134.15 | 11.60 | 124.80 | 10.30 |
| SEQ ID NO: 110 | 118.66 | 10.30 | 113.58 | 8.40 | 104.01 | 7.60 |
| SEQ ID NO: 111/112 | 228.16 | 35.62 | 202.43 | 18.73 | 132.98 | 18.32 |
| SEQ ID NO: 113 | 158.59 | 24.54 | 155.03 | 21.36 | 135.44 | 17.44 |
| SEQ ID NO: 126 | 185.07 | 13.40 | 147.37 | 16.20 | 131.05 | 10.80 |

DR-performance (leaf area) under drought shown as % of GFP expressing plants
RC E1-3-performance with re-cloned relevant Seq. ID event 1-3
SD-value shown ± standard deviation

As shown in Table 6, plants expressing the re-cloned genes identified by the method of the present invention presented enhanced leaf area as compared to plats expressing the negative control gene, in Arabidopsis plants subjected to drought conditions.

Reference is now made to Table 7 presenting results of drought experiments conducted with T2 tobacco plants. Different genes identified by the present invention were re-cloned and transformed into tobacco plants generating several independent events (represented by E1-3 in Table 7). Plants were grown in soil in controlled greenhouses and tested for drought tolerance under the conditions mentioned above. At the end of the experiment plant shoots fresh weight, leaves number, length of main branch and weight of main branch were evaluated. Results are shown in Table 7 as percentage of wild type (WT) plants that served as a negative control.

TABLE 7
Results of drought experiments conducted with T2 Tobacco plants

| Seq ID | FW | FW ± SD | LN | LN ± SD | BFW | BFW ± SD | BL | BL ± SD |
|---|---|---|---|---|---|---|---|---|
| SEQ ID NO: 1 E1 | 104.71 | 11.18 | 73.58 | 11.63 | 106.78 | 14.75 | 122.73 | 9.25 |
| SEQ ID NO: 1 E2 | 97.28 | 2.18 | 67.92 | 4.76 | 86.18 | 5.10 | 97.27 | 8.86 |
| SEQ ID NO: 1 E3 | 99.22 | 11.21 | 64.15 | 7.70 | 116.72 | 6.30 | 118.18 | 8.90 |
| SEQ ID NO: 2 E1 | 122.23 | 2.70 | 116.76 | 4.84 | 115.42 | 3.47 | 97.33 | 4.87 |
| SEQ ID NO: 2 E2 | 119.11 | 5.62 | 101.62 | 3.97 | 122.99 | 2.70 | 114.84 | 2.98 |
| SEQ ID NO: 2 E3 | 116.69 | 9.00 | 111.35 | 4.58 | 117.50 | 14.45 | 102.67 | 9.91 |
| SEQ ID NO: 15 E1 | 111.60 | 4.18 | 98.38 | 7.58 | 113.65 | 4.39 | 102.08 | 4.89 |
| SEQ ID NO: 15 E2 | 121.87 | 2.88 | 118.92 | 2.53 | 122.25 | 3.70 | 100.59 | 3.71 |
| SEQ ID NO: 15 E3 | 113.93 | 5.39 | 116.76 | 6.42 | 103.32 | 7.79 | 95.25 | 9.41 |
| SEQ ID NO: 44 E1 | 124.04 | 4.23 | 118.92 | 6.69 | 130.31 | 5.23 | 94.66 | 6.12 |
| SEQ ID NO: 44 E2 | 121.17 | 8.34 | 108.11 | 3.54 | 128.45 | 14.00 | 112.02 | 8.14 |
| SEQ ID NO: 44 E3 | 113.80 | 10.36 | 117.84 | 7.28 | 118.83 | 9.15 | 90.80 | 8.89 |
| SEQ ID NO: 55 E1 | 120.52 | 3.43 | 123.24 | 5.53 | 122.56 | 5.65 | 102.08 | 5.33 |
| SEQ ID NO: 55 E2 | 117.85 | 8.35 | 113.51 | 3.42 | 121.35 | 11.26 | 95.55 | 7.40 |
| SEQ ID NO: 55 E3 | 123.13 | 5.02 | 111.35 | 9.31 | 127.92 | 8.75 | 110.09 | 7.81 |
| SEQ ID NO: 56/57 E1 | 101.58 | 5.17 | 75.47 | 8.92 | 80.26 | 23.40 | 109.55 | 6.45 |
| SEQ ID NO: 56/57 E2 | 106.93 | 9.10 | 79.25 | 8.50 | 101.30 | 10.14 | 103.64 | 7.22 |
| SEQ ID NO: 56/57 E3 | 98.16 | 10.90 | 75.47 | 8.92 | 81.97 | 10.54 | 100.91 | 15.84 |
| SEQ ID NO: 142 E1 | 110.50 | 5.07 | 94.34 | 15.46 | 109.37 | 7.31 | 116.82 | 16.89 |
| SEQ ID NO: 142 E2 | 119.83 | 5.07 | 94.34 | 1.98 | 105.75 | 5.15 | 118.64 | 19.00 |
| SEQ ID NO: 142 E3 | 114.89 | 2.74 | 98.11 | 6.86 | 101.90 | 5.94 | 95.45 | 19.44 |
| WT | 100.00 | 2.43 | 100.00 | 1.67 | 100.00 | 9.84 | 100.00 | 11.65 |

FW-fresh weight measured in grams
LN-leaf number
BFW-branch fresh weight measured in grams
BL-main branch length measured in cm
SD-value shown +/− standard deviation as % of measured trait
E1-3-different independent events

The results presented in Table 7 show that most of the genes identified by the present invention confer improved tolerance to drought conditions in Tobacco plants, as shown by the tested parameters (e.g. fresh weight, leaf number, branch fresh weight, branch length) as compared to negative control plants.

Reference is now made to Table 8 presenting results of salinity experiments of transgenic tobacco plants as compared to control WT plants. Different tobacco lines expressing various genes identified by the method of the present invention (see Table 4), were germinated in soil. Seven days post germination; plants were irrigated with fertilized water containing 400 mM NaCl. Leaf images were taken 14 days after irrigation with salt and analyzed for leaf area for the different independent events. Results are shown in Table 8 as percentage leaf area difference from WT plants.

TABLE 8
Results of salinity experiments on tobacco plants

| Seq ID | HST | ± SD |
|---|---|---|
| SEQ ID NO: 1 | 225.12 | 12.65 |
| SEQ ID NO: 2 | 240.63 | 28.91 |
| SEQ ID NO: 15 | 505.52 | 17.57 |
| SEQ ID NO: 44 | 767.46 | 7.48 |
| SEQ ID NO: 55 | 206.71 | 26.27 |
| SEQ ID NO: 56/57 | 286.19 | 4.86 |
| SEQ ID NO: 70/71 | 1366.07 | 4.70 |
| SEQ ID NO: 142 | 318.54 | 29.75 |
| WT | 100.00 | 13.22 |

HST-high salinity tolerance shown as % difference of leaf area as compared to WT
SD-value shown +/− standard deviation between 4 independent events

The results of Table 8 clearly show that plants expressing the novel salinity tolerance genes identified by the present invention revealed significantly higher leaf area as compared to WT control plants.

Reference is now made to Table 9 presenting salinity experiments conducted on Arabidopsis plants expressing novel genes having Seq. IDs as indicated. Ten plants per event per pot were grown in soil in controlled greenhouse. After germination, all pots with plants were irrigated by submerging them with 100 mM NaCl. The results of Table 9 represent average data of 4 different events per Seq. ID and wild type plants (WT).

TABLE 9
Results of salinity experiments conducted on
Arabidopsis plants expressing novel identified genes

| | Flower | | | |
|---|---|---|---|---|
| | & Pod | | | |
| Seq ID | production | FP ± SD | Chlorosis | Chlor ± SD |
| SEQ ID NO: 1 | 2.50 | 0.50 | 4.17 | 0.00 |
| SEQ ID NO: 2 | 3.75 | 0.48 | 4.58 | 0.00 |
| SEQ ID NO: 5 | 1.00 | 0.41 | 2.33 | 1.00 |
| SEQ ID NO: 6 | 3.25 | 0.25 | 4.42 | 0.00 |
| SEQ ID NO: 7 | 3.50 | 0.50 | 4.42 | 0.25 |
| SEQ ID NO: 8 | 4.00 | 0.41 | 4.67 | 0.00 |
| SEQ ID NO: 9 | 3.25 | 0.25 | 4.33 | 0.25 |
| SEQ ID NO: 10 | 2.25 | 0.48 | 3.42 | 0.41 |
| SEQ ID NO: 11 | 1.50 | 0.50 | 3.00 | 0.50 |
| SEQ ID NO: 12 | 2.75 | 0.63 | 3.83 | 0.63 |
| SEQ ID NO: 13 | 2.75 | 0.25 | 3.58 | 0.00 |
| SEQ ID NO: 16 | 2.75 | 0.63 | 4.08 | 0.00 |
| SEQ ID NO: 18 | 1.50 | 0.29 | 2.92 | 0.25 |
| SEQ ID NO: 22 | 2.50 | 0.87 | 3.33 | 0.41 |
| SEQ ID NO: 23/24 | 3.50 | 0.29 | 4.50 | 0.00 |
| SEQ ID NO: 25 | 2.25 | 0.75 | 3.08 | 0.29 |
| SEQ ID NO: 26/27 | 1.75 | 0.48 | 3.00 | 0.25 |
| SEQ ID NO: 29 | 2.50 | 0.29 | 3.83 | 0.25 |
| SEQ ID NO: 30 | 3.25 | 0.48 | 4.25 | 0.29 |
| SEQ ID NO: 30 | 3.00 | 0.71 | 4.33 | 0.00 |
| SEQ ID NO: 33 | 2.50 | 0.87 | 3.42 | 0.25 |
| SEQ ID NO: 94 | 2.50 | 0.29 | 3.67 | 0.29 |
| SEQ ID NO: 40 | 1.25 | 0.48 | 2.33 | 1.25 |
| SEQ ID NO: 43 | 2.00 | 0.41 | 3.75 | 0.25 |
| SEQ ID NO: 44 | 3.50 | 0.29 | 4.42 | 0.25 |
| SEQ ID NO: 55 | 2.00 | 0.41 | 3.67 | 0.00 |
| SEQ ID NO: 56/57 | 2.50 | 0.29 | 3.58 | 0.25 |
| SEQ ID NO: 58 | 1.75 | 0.85 | 2.67 | 1.04 |
| SEQ ID NO: 59 | 3.00 | 0.41 | 3.75 | 0.48 |
| SEQ ID NO: 70/71 | 1.75 | 0.63 | 2.67 | 1.00 |
| SEQ ID NO: 77 | 2.00 | 0.00 | 3.50 | 0.29 |
| SEQ ID NO: 89 | 1.75 | 0.63 | 2.50 | 1.11 |
| SEQ ID NO: 90 | 3.00 | 0.71 | 3.83 | 0.25 |
| SEQ ID NO: 91/92 | 1.00 | 0.00 | 3.17 | 0.58 |
| SEQ ID NO: 93 | 2.75 | 0.25 | 4.17 | 0.25 |
| SEQ ID NO: 95 | 3.75 | 0.25 | 4.50 | 0.25 |
| SEQ ID NO: 99 | 2.00 | 0.41 | 3.00 | 0.25 |
| SEQ ID NO: 103/104 | 2.00 | 0.00 | 3.67 | 0.29 |
| SEQ ID NO: 106 | 1.00 | 0.00 | 2.25 | 0.29 |
| SEQ ID NO: 107 | 1.75 | 0.25 | 3.00 | 0.25 |
| SEQ ID NO: 110 | 1.75 | 0.25 | 2.83 | 0.25 |
| SEQ ID NO: 111/112 | 1.00 | 0.00 | 3.08 | 0.25 |
| SEQ ID NO: 113 | 1.25 | 0.75 | 1.83 | 1.44 |
| SEQ ID NO: 118 | 2.50 | 0.29 | 3.25 | 0.50 |
| SEQ ID NO: 119 | 2.25 | 1.03 | 2.75 | 1.19 |
| SEQ ID NO: 120 | 2.00 | 0.41 | 3.08 | 0.29 |
| SEQ ID NO: 124 | 1.75 | 0.25 | 3.00 | 0.00 |
| HRD | 1.25 | 0.25 | 2.83 | 0.48 |
| WT | 1.00 | 0.00 | 1.83 | 0.55 |

FP-Flowers and pods production
1-No Flowers
2-Few flowers formation with short flowering stems
3-Some flower formation almost no pods
4-Flowers and pods forming
Chlorosis-Chlorosis and damage to leaves
1-Completely dry leaves
2-Dry leaf edges
3-Yellow
4-Some Yellow
5-Green
± SD-standard deviation

As shown in Table 9, plants expressing genes identified by the method of the present invention as conferring salinity tolerance, demonstrated significantly higher flowers and pods yield and significantly reduced chlorosis and damage effects to the leaves as compared to WT control plants subjected to the same salinity stress conditions.

To conclude, the experimental results presented above clearly demonstrate that by the unique method of the present invention, highly valuable stress tolerance (e.g. drought, salinity) genes in plants can be identified. The newly identified genes confer improved tolerance or resistance to the preselected stress in plants in various important parameters such as leaf area, turgor pressure, aerial yield and quality, flowers and fruits yield etc. These results show that the present invention provides a novel screening method that identifies stress tolerance plant genes that can be expressed in desirable and important crops to enable their growth and enhance their yield under various abiotic and biotic stress conditions.

### References

Gabor, E. M., Alkema, W. B. & Janssen, D. B. (2004) Quantifying the accessibility of the metagenome by random expression cloning techniques. Environ Microbiol 6, 879-886.
Culligan, E. P., Sleator, R. D., Marchesi, J. R. & Hill, C. (2014) Metagenomics and novel gene discovery: promise and potential for novel therapeutics. Virulence 5, 399-412
Venter, J. C. et al. Environmental genome shotgun sequencing of the Sargasso Sea. Science 304, 66-74.
Farooq, M., Wahid, A., Kobayashi, N., Fujita, D. & Basra, S. M. A. (2009) Plant drought stress: effects, mechanisms and management. Agron. Sustain. Dev. 29, 185-212.
Taiz L. & Zeiger E. (2006) Plant Physiology, 4th Ed., Sinauer Associates Inc. Publishers, Massachusetts.
Nonami H. (1998) Plant water relations and control of cell elongation at low water potentials, J. Plant Res. 111, 373-382.
Parida, A. K. & Das, A. B. (2005) Salt tolerance and salinity effects on plants: A review. Ecotoxicology and Environmental Safety, 60(3), 324-349.
Carillo P, Annunziata M G, Pontecorvo G, Fuggi A, & Woodrow P. 2011. Salinity stress and salt tolerance, abiotic stress in plants—mechanisms and adaptations. In: Arun Shanker, editor Tech, DOI: 10.5772/22331.
Yang T-T, Cheng L & Kain S R (1996) Optimized codon usage and chromophore mutations provide enhanced sensitivity with the green fluorescent protein. Nucleic Acids Res 24:4592-4593
Hema R, Vemanna R S, Sreeramulu S, Reddy C P, Senthil-Kumar M, & Udayakumar M (2014) Stable Expression of mtlD Gene Imparts Multiple Stress Tolerance in Finger Millet. PLoS ONE 9(6): e99110
Karaba A, Dixit S, Greco R, Aharoni A, Trijatmiko K R, Marsch-Martinez N, Krishnan A, Nataraja K N, Udayakumar M, & Pereira A (2007) Improvement of water use efficiency in rice by expression of HARDY, an Arabidopsis drought and salt tolerance gene. Proc Natl Acad Sci USA 104:5270-15275.

What is claimed is:

1. A plant comprising a transgene encoding a polypeptide sequence having at least 95% identity to the polypeptide sequences set forth in SEQ ID NO: 165 and wherein the transgene confers to the plant one or more of the following improved/increased characteristics: improved drought resistance, increased biomass, increased salinity tolerance, increased yield, wherein said improved/increased characteristic is in comparison to a WT version of the plant or to a GFP expressing version of the plant devoid of the transgene.

2. The plant according to claim 1, wherein said one or more improved/increased characteristics includes improved drought resistance and/or increased salinity tolerance.

3. The plant according to claim 1, wherein the transgene has a polynucleotide sequence having at least 80% identity to the polynucleotide sequences set forth in SEQ ID NO: 15.

4. The plant according to claim 1, wherein said transgene is expressed under a constitutive promoter or a stress induced promoter.

5. The plant according to claim 1, wherein the plant is an agricultural crop.


SEQUENCE LISTING (333 sequences)

SEQ ID NO: 1 - DNA, 1019 bp, unidentified
  source 1..1019: mol_type = genomic DNA
ggactttctcattttcagaattattttctatactctgacaagagcaagcaataccaaacatcttccacatcgaagctttaaccattttgcccttaacatttgaacaagacgaaatggccttcttcccacactacaccactaatctgtcgcctctgctctacttgttggacgacgactatgctgtctaccgctcaacttgtccaaagtccaactaccaccacaagcaacaccacagccgccgtcagccttcgccagttcgttactttagtccgaattttgatatgcgagaggggaatgactcctactaccttgacggagagctccctggtgtcaaccagaatgatgtcgatattgaattctctgaccctcagacactggtgatcaagggtcgagtggagcggaattacaacaatctcgacggcatgaacgaggaaaaccagcaagatgaagaacaattctctgaaactctctctagcaagtcgtaccaacccactgtcgaggacgaggacgaggcgaaccattcaccacccgtggcgacaccaacctactctgagaagtctgttactgagaaaactcagaagcctgcgtacaaataccgaaattctgaacgtgctattggcgaattccaccgagccttcaatctccctacaagagtcgatcaagatgcggtcagggctacattgaggaatggaatcctctcgctggagctcccgaaggagccggcaccgaagatgaagaagattcggattgaatagaggatttcgaataaaatttttgatttgatgagtagttggtgtttattgttatgtctaattatatggggctatgtcatgattgggaaatgggacaccgcatttgtttcctttttccccatttcttcagacgccatctatattacatgtatgttgcatgaactatggtttttgctaggagcggttgcttctgctctgcattttcatgaactattttctttttattaaattaataactagcatatcaattaatgatctgtcatatgg

SEQ ID NO: 2 - DNA, 712 bp, unidentified
  source 1..712: mol_type = genomic DNA
gatcatcaatcaattaatcaatctactctactttccaaaacataactaccaaataaccagaatgcagctcctcagcaccctcaccccccttgccctcctagtcaccgtcgcttccgccaccggcaaagccgtcaataatgccgttggcaacgccgtcgtcacaaaccactgtaaagacccaatctatctctggtccgtcggctcctccgtctccccgaaacacaccatcccctccggctccaactataccgagcccttccgccacgacgacgcatctggcggcatcgcgctgaagatcacccgtaacgacaacgggctgtatgacgggagtgcgcagttagtttactcctacgctttggatggggaacaggtgtggtatgatttgtcgagtgtgtttggggatgcgtttgcaggggaggctgttgctgtgaagccggagaatgaggggtgtgggagtatttgttggcctaagggtaccacgcctggtggaagccaggttaaggtctgtgatgcggagggggatgttggattggttgtttgtgcgaaggggtgttagggggtctgagtgaaggttggtggtggtaatgagcaattgggtatgagaggggaaaggatatgttaatcgtttatgttatttacttgatcaaaatatttgtattgacgtcggttgttttgttattgttgttttaaatgcaaatgtatatgaactttc

SEQ ID NO: 3 - DNA, 580 bp, unidentified
  source 1..580: mol_type = genomic DNA
ggttcgtcaacgcgacgatccgcgggggtccaagctaggacgtggcagttgtgacacaacaagagcatgctatggcaaatgccattcgcgagctcgccactaccctgtgaactgtggccttactcctatacccgccagagtctgacttattcctgtcactggaatctggcttactgctggtgctggagtctggtcccagtattttagtatagtacaattgctagctgaagccataaggcgtggattgttggggtggcgcagggctgaagccaaatggcagcggtgttgctgctggttgagcaccgggcatagcgccagaaagtgcacccgcgaacattccctggtattgcatgaacggctgtccaggaatcgcgccaggcattggcaaggcgttgttgcgctttgcctccgctgccagcagcctctctgcttccgtaccgtgtctttcgcccttaccgtccttcttgaatgcataatcaaccgtcagaggcttgttcatcaagtactggccattcatagccgtgattgcctggtccgagctgtcaaagtcgttgtactggatgaatccatatcctttcaa

SEQ ID NO: 4 - DNA, 580 bp, unidentified
  source 1..580: mol_type = genomic DNA
ttgaaaggatatggattcatccagtacaacgactttgacagctcggaccaggcaatcacggctatgaatggccagtacttgatgaacaagcctctgacggttgattatgcattcaagaaggacggtaagggcgaaagacacggtacggaagcagagaggctgctggcagcggaggcaaagcgcaacaacgccttgccaatgcctggcgcgattcctggacagccgttcatgcaataccagggaatgttcgcgggtgcactttctggcgctatgcccggtgctcaaccagcagcaacaccgctgccatttggcttcagccctgcgccaccccaacaatccacgccttatggcttcagctagcaattgtactatactaaaatactgggaccagactccagcaccagcagtaagccagattccagtgacaggaataagtcagactctggcgggtataggagtaaggccacagttcacagggtagtggcgagctcgcgaatggcatttgccatagcatgctcttgttgtgtcacaactgccacgtcctagcttggacccccgcggatcgtcgcgttgacgaacc

SEQ ID NO: 5 - DNA, 781 bp, unidentified
  source 1..781: mol_type = genomic DNA
gaaaaaaactttagaatacagtttaatcaatcttcacagctacaaggctatatcatttgatatagcatatcaaagtggctttgatttctgtaaatttatatctaataataatagtgtttatatcagctaaatacatatttctatcctatctatatatcaccgacagaccatatttgaaactgctgttgacactattattcatatgttcggatttaattttaatacgacaaaattgttaaaaacaattctcgttgtttgttatttgcaggcaacagtgttagctgatccttatacaagagtatcttgggaagcgtatatgaatcatgtcaatggatccgacgactatcgtactcaaggggatgataccagagctacacgctttccagagactaaacctccaaaacaaggaaaagatttcctgtggtcgagtaaaccagtccccagttcagatctatttctggagttctttatgtatgagggagaaccagatgaattcagcaggacgactgaatcgtatcaatcacttccgagcaacgcgttaactgctaggcaaaatgcccttacttgtcaggacatagagtcatgttcgtatcctccacaggtgaacaactttcaagctttattcgacgacctggggccatcaacttgtaatctcataaaagacgaaactcgtgactggatattgcagcagtggcccgggttagctgtaggagccgttatatcgtttgcggtagccgttgcgggaagctcctgtgatatattatattaatcagctttggca

SEQ ID NO: 6 - DNA, 661 bp, unidentified
  source 1..661: mol_type = genomic DNA
ggtcgagctactttcaaggtcaagcaagatggtccgttacgcacacaatgctgagaacccagagaagaccgccaaggctcgtggtcagcacttgcgtacgcacttcaagaacacccgtgaagtcgctgctgctctgaccggcttgaagctttcaaaggcttacaagtacctcggcgatgtccaagagcacaaggatgtcatcccattccgtcgcttcaacggtggtgttggcagagccgctcaggctaagaaccacggtacgacccaaggtcgttggccagtcaagtcgattggcttcttgctcagacttttgaagaacgctgaggccaacgctgacgccaagtcactcgacacggaagacctcttgatcaagcacattgttgtccaacaagctccaaaaacccgtcgtcgtacttaccgtgctcacggtcgtatcaacccttaccaaggacacccatgccacattgagatcactctggctgtcccagacgagcaagtcgctcgcaacaaggacgttgaggtgaaccaaccaaagaagatccaaggcaacaagcgtcaagtcgctgctcaacgtcgcttgacctctgcataaactggctactcggttgtgtaccactctatacaaattattcagtaaaatgctatccatcttggcttcgaa

SEQ ID NO: 7 - DNA, 713 bp, unidentified
  source 1..713: mol_type = genomic DNA
agccacaaccacatcaatcctccaccactttcagctttcgacttcatcaaacaactccttctaccactactacctcaacaaccttcatcaaaatgactggacgcggcaagggcggcaagggtctcggaaagggcggcgccaagcgtcaccgcaagatcttgcgcgacaacatccagggcatcaccaagcccgccatccgccgtctggcgcgtcgtggcggtgtcaagcgtatctccgccatgatctacgaggagacccgcggtgtcctcaagaccttcctcgagggtgtcatccgcgacgccgtcacctacaccgagcacgccaagcgcaagaccgtcacctccctcgacgtcgtctacgccctcaagaggcaaggccgcaccctctacggtttcggtggttaagcagctcgctcttctctcttcgactgctttgctttcttcaaacacaataacaatcacgacaacaacaacttcatcagatatccacccacaatgcgagagttgggcttgcgggtatggcgcgaatgggcaatgggctatccgggttttttcatttttggggtttttttctcttttcctgtttcgatgctgcgaggtgagcacactgggctgcggctcatgaggctttgagtgtagaataggctcaacatcatcaaagaagcattccacgagacgtggcgctttcttcatcaaccaaatgaatattgcagc

SEQ ID NO: 8 - DNA, 1021 bp, unidentified
  source 1..1021: mol_type = genomic DNA
ggacttgcgaccacacacatctttatacctcaaaatgtcgctcgatgtcggagatgtagacgcctggatcgacacgctatcgcagtgcaagcagctatctgaatctgacgtgaagctcctctgcgacaaggccagagaaattcttatagaggagtccaacgtacagccagtcagatgccccgtcaccgtctgcggcgatattcacggtcaattccacgacttgattgagctctttagaataggcggcaactccccatccaccaattacctcttcatgggcgattacgtagacagggggtactactcggtcgaaactgtcaccctcctcgtcgccttgaagctccgctacagggaaagaatcaccatcttgcgcggtaaccacgagtcgagacagatcacccaggtctacggtttctacgacgagtgcttgagaaagtatggaaacgccaacgtctggaagttcttcaccgatctctttgactacctcccactgacggcgcttattgacaatcaaatcttctgtcttcacggtggtttgtctccttccatcgacacgctcgaccacatccgctctatcgaccgtatccaagaggtgcctcacgaaggtcctatgtgcgatctcctctggtccgatccagacgaccgctgcggctggggcatatcccctcgtggtgccggttacaccttcggtcaggacatttcagaggctttcaaccactcaaacggcttgacgctcgtagcccgtgctcaccaacttgtcatggaaggttacaactggtcccaggacaggaatgtcgtcactctcttctctgcgccaaattactgctacagatgcggtaaccaagctgcgatcatggagattgacgagaatctcaagtacactttcctccaattcgatccagcaccaagagctggcgaaccgatggtgtctcgaagagttccggactacttcttataggctcttactcactgtatttatgtttgcactgggtattgtttacttgtacaatgtgtaactacg

SEQ ID NO: 9 - DNA, 715 bp, unidentified
  source 1..715: mol_type = genomic DNA
ggagccacaaccacatcaatcctccaccactttcagctttcgacttcatcaaacaactccttctaccactactacctcaacaaccttcatcaaaatgactggacgcggcaagggcggcaagggtctcggaaagggcggcgccaagcgtcaccgcaagatcttgcgcgacaacatccagggcatcaccaagcccgccatccgccgtctggcgcgtcgtggcggtgtcaagcgtatctccgccatgatctacgaggagacccgcggtgtcctcaagaccttcctcgagggtgtcatccgcgacgccgtcacctacaccgagcacgccaagcgcaagaccgtcacctccctcgacgtcgtctacgccctcaagaggcaaggccgcaccctctacggtttcggtggttaagcagctcgctcttctctcttcgactgctttgctttcttcaaacacaataacaatcacgacaacaacaacttcatcagatatccacccacaatgcgagagttgggcttgcgggtatggcgcgaatgggcaatgggctatccgggttttttcatttttggggtttttttctcttttcctgtttcgatgctgcgaggtgagcacactgggctgcggctcatgaggctttgagtgtagaataggctcaacatcatcaaagaagcattccacgagacgtggcgctttcttcatcaaccaaatgaatattgcagc

SEQ ID NO: 10 - DNA, 503 bp, unidentified
  source 1..503: mol_type = genomic DNA
aatcacccaaatgttctctaaactcatcgccatcgcctctcttgccctcgctgccaacgctgcagtcatcgacccaagtgaccacactgtccaatacgaagctgcaccaggaaaggttgtgactgagcactacgaggttctcagccacgccgaagcatcgcgcataatcgaagccaatccacacatcagcgactatcgctacagatgcaactaccaatgcaacgatagcagcggcaactacatgagaaacctgcagcagggagttccaaaccaagcatgcatcttctctagctgctacgactgtgactggaaattccaaaactgtagctactgtcgcttgtcgactggccacaactaccgtgatatcggtggactcgagagctggtgctacaacaacggcggtactacagtgacgcacaactgtggttatactgatggcgaccaatgctaagagcggccttgtaaagtaaaacttgtactctgaatttgcctttatctttccc

SEQ ID NO: 11 - DNA, 580 bp, unidentified
  source 1..580: mol_type = genomic DNA
atcatctcaaacccaattatcttgaacacctagtttctcaagaacatcctcaaaatgcacttcaaatctctctttattgctggcgccctcttcatggtcggtgccagtgccgttgattgtgccactcctgagattcactgcgagactagtgatggcagcccctggtacgacgatgccgtccaagccactgaatactggaaagaaatccaggacgccggcaaagacagctgcggtgatgctggttgcgcacagccccatggctctggatgccacagcgacggtggtagctatggtaccgccgagatcgttctctgccaggatgactcgtcctcttcaactccccaatgtgccgactgccggtgtgtctacagctacctgaagcctcttctcgaccaatgcaagggtgccaacaacaagattggtggatatgctcatgttgacatgggaggcaactacatcaactatgaatttgttaagaaatgagcggatctcacgtgtgtgagaccatcatatagggttttgaagtctgtttcctttgtatttaacgtcgaaagacaattatgagccaggtttatactcc

SEQ ID NO: 12 - DNA, 607 bp, unidentified
  source 1..607: mol_type = genomic DNA
cgcgccgggggaaattggaccatgatagacgacctcaccactggctcagaggacagcttctccaacagctggatatcgtggttcttatctaccaaagggaacgagtacttctgtgaggtggatgaggagtacatactggacagattcaacctcactggcctcaacaacgacgtgcagaactactcgcaggcgctggagctcatcacagacagcctcgacgacgaggacctcgatgatgagcagagagacgctatcgagaacagtgccaggtatctctatggcttgatccacgccagatacatcattacctcccgcggactggcaaagatgctcttcttggtgtacccgcagcagctgccgtcaaagacgacgaactcagtgccgagcacgaagccggcaacttcagcagacgcagcggtcggggtggacaggtacctgcccaagatattcgggttcccggtgcacgagatgtccaagcacgcgaggtggcaggaggcgcagagggatctgcagatttcgaggctgcagcaaagtgcgagtgacccgtcgtacgtgtagagcgttcaaacatgtattactattggtataataatttaactttactgcc

SEQ ID NO: 13 - DNA, 1406 bp, unidentified
  source 1..1406: mol_type = genomic DNA
aagttacccggcttattagtcctagattccgagatgtcgctcactcccgaacaaaccgaaatcatcaaggccaccgtgcctgtcgttaaagaacatggcaagaccatcaccaccgttttctacaagaacatgctcgaagcgcatcctgagctgaacgccattttcaacactaccaatcaggtcaatggtcaccagcccaacgcactcgccggagccctcttcgcctacgcctccaacattgacaaccttggcgccttgggccctgccgtcgaactcatctgcaacaagcatgcttcgctgtatatccaacctgagcactacggcatcgtcggcaagttccttctcgaagcgatgggacaggttttgggtgacgccttgactccgcagatcctcgacgcctgggcagctgcctactggcagctcgccaacctctttattggtcgcgaaagtgctatctacaagcagagtgagggatggacacagtggcgcgagttccgggttgcacagaaggtccctgagtccgcggagatcacatcgttctacctcaagcctgtcgacgagaagcctttgccccgcttccgccccggacagtacatttccgtccaagtgcacgttcctcagcttgaatgcccccaagctcgccaatactccctcagcgacaagccccgcgacgattactaccgcatcagcgtgaagaaggagacgggtctcaacacagcaaagccggaggccaaggtcaacccgggttacgtctcgaatattctgcacgagaacgtcaacgagggcgacgtgatcaaggtgtcgcacccttgcggcgatttcttcttgaccgagcaggaaccgtcgcaccctgtcgtcctcatcgcagccggtgtgggtctgacgccacttacctcgatgctcaacacattggactccacccccgcggactctcagcgcaagattcacttcatccacggtgcgcgcaccacttccgtccgcgctttcaaggaccagattaagtctcgcgctgagcgactcccgaatctccaggccaccttcttcaccagctccccgtcggcagatgaaaagcaaggcgtcgactatgacgtccagggccgtatcgatgtgtccaagatggatgccagcaaggatcttttcctcgacaatgcgcagaccgagttctacatttgtggtcccacttccttcatgaatgatatcgcgaacagcttgaaagctcggggggctacctcggagcgtatccacatggaattgttcggcactggcggcgtgcctgtttagatgatggctcagttagccgtgattgggttttatttctttacgacgatatgactcaggtttctaagttagtatacataatcatgataaattcttatatagatatatcaataatacatctcctctcg

SEQ ID NO: 14 - DNA, 750 bp, unidentified
  source 1..750: mol_type = genomic DNA
gggtctcttccatttgaatttttcaacccacagcatggccttcatgaatctcccatggcccactgaatgcctgcatgccgctctcaagaacggatccttacctttctggggatttgtaatctatcgaacgacctacaccgctcagtcagatgccgcctggccgcagattatcgagcttattgcctcctatatgaaagccttactctaccacgagtataacgacaagaaaaaagatggagatgagcctacagtctacgacgaaatctgggcaaggcatcagttgacgattatggatgatagacaattcaacggagcgtctgtgtttgatatccaacttcacttcgaaaagtgggttgaggcgcagggaaagcgagatgaatctactatgtatcgcatgtgtatggtcattgatgatgaatcaatccagacgttattggaggcgccacccggggaaaataggaaactcggacgacgtatagggggccctgtacgctttgtcaaagtcgtggaggctttccccgagctagacagccttgacgaattccagggatggatgaaatgtgagatcaacgcgttatggccgctgtggaagatgatgtctgacggagatgaaatgaggatgtcatatgatgagatgaaggggaatggaaagcaggtctatggcgcaatttaatcggtttttcttcatgttatcctgatggaaaaaatggcagaacatatgtctgtacatgcagaaaataaggtgattgg

SEQ ID NO: 15 - DNA, 811 bp, unidentified
  source 1..811: mol_type = genomic DNA
gacaccacctctttttcgacaaccacaccccgtttcgcaggaagtccatttccagcagtcaaaatggcccgtcgtcccgcgagatgttaccgctactgcaagaacaagccttaccctaagtcccggttcaaccgtggtgttcccgaccccaagatccgtatcttcgacttgggtcgtaagaaggcttccgtggacgacttccccctgtgcgtccacatggtctccaacgaatacgaacagctttcctccgaagctctcgaagctgcccgtatctgtgccaacaagtacctcgtcaagatcgccggtaaggaaggtttccacctgcgtgtccgcgcccaccccttccacgtcgtccgtatcaacaagatgttgtcgtgcgctggtgccgatcgtctccagaccggtatgcgtggtgccttcggtaagcccaacggtgttgtcgcccgtgtgaacatcggccagatcctcctgtccatccgcacccgtgactccaaccgcgccgccgccgttgaggccatgcgccgctccacctacaagttccctggtcgccaaaagatcattatctccaagaactggggcttcacccccgtccgtcgtgaggagtacgtcaagctccgccaggagggcaagctcaagcaggacggtgcctacgtccagttcctgcgtggccacggtttggtcgaggagaacatgaagcgcttcccccaggcctacgagggcgttgctcagtagattgggatgaattaggtggttttatgtgctggtcgtatttatcgtttttactagggccaaatgagaacaaaaaaaggct

SEQ ID NO: 16 - DNA, 923 bp, unidentified
  source 1..923: mol_type = genomic DNA
gtctttagttggccattgaacactgacagatttgcattgcttatatttgcatctacctcacatctactcaactcctcctctccgtttgtcatgtccttctaccagtctcgtccagacactatcaagggtcctgatcctttgaccgacaattggacttatgatagtgccattgatctcttctcttggaatcccatgatgcccgatccttttacctttgacctgcccgacgatcttatgaaatttgaatctaaggatatgtctgctggcatggtcgctccttcggacattagtggttttgccattggtaaccatttgggcgaggatgctgcctcgatatctgatcccgagagtgatgaccacccatggtccccctccgctcatgctgccttcccggagctctctcccatcacatcgacagagcaagtccatcaagaaactgctcgatactcaactacccccgatgccacctcacctcaagaacaaccctcctcaccaccaacacgatctactcgccgccgatcatccgctgacggtcccgttcgcaacgctgccaaacgagcagcccacaacgtcattgaaaagcgctacagaacaaacatgaatgccaaattcgtggcactcgagaaagcaatgaatggcggtaatggcgtgcaaacatcatcaagaggcggagggtccgcgtcgcttaagaaatccgaaatcctctctaatgctattgcctacatgcatggactgcaagaggaaaatcgctatttacaaaaggagcttgctatcgttaaacagaatcttgtaccggcagggatatggcgaggggctcctagttgtaaacgggagacgagttatcgttaacttgttgatttccctgtggttgtttagattttttttacgatgttacgtgtataataatactctcccctcgggtc

SEQ ID NO: 17 - DNA, 1046 bp, unidentified
  source 1..1046: mol_type = genomic DNA
ggacaagcccatcttcaattcgagacagtcgccatgggtcgcgttatccgcaaccagaggaagggccgtggctccattttcacggctcacacccgtctgaacaaggctcccgcccagttccgtaccctcgacttcgctgagcgtcacggatacacccgtggtgttgtcaaggagatcatccacgatgccggccgtggtgctcccctcgccaaggtccagttccgccacccctacaagttcaagatggtgaccgagaccttcatcgccaacgagggcatgtacaccggtcagttcatctacgccggtaagaacgctcagctcaccgtcggcaacgttctgcccctcgcctccatgcccgagggtaccgtcatctccaacgttgaggagaagtccggtgaccgtggtgcgcttggccgtacctccggtaactacgttaccgtcattggccacaaccccgaggacggcaagacccgtgtcaagcttccctccggtgccaaaaaggtcatcaagaacaccgcccgtggtatggttggtatcgtcgccggtggtggtcgtaccgacaagcccctgctcaaggcttcccgcgccaagcacaagttcgccgtcaagcgcaactcttggcccaagactcgtggtgttgccatgaaccccgttgatcaccctcacggtggtggtaaccaccagcatatcggtaaggcctctaccatctcccgctacgccgcccagggtcaaaaggccggtctcattgctgcccggagaaccggtctgctccgtggtacccagaagaccaaggattaagcgtgatattacgtggagttttctttgtgacgggttgaaaatggacttctgctatgagacatatgtacttaggcgagtgcggataagcgtcccatgcgcccttagcgaattaaggttgtggtcaccatccttttctttttattaaatcaaaaaagggtgatggaatggggtccgaggctggcctcaagtcaaggcagaacggaaaagtcaaaaatgccccttggggttttggaaatgatacacctttg

SEQ ID NO: 18 - DNA, 780 bp, unidentified
  source 1..780: mol_type = genomic DNA
ggcgcagaggcctattactccccagtatcatcgctaataggcatgtccacgggtctaaggttcagcactttgccagctgcttccaatccacagtcgtcgtcattgatacccagccctagtgctcccatatcaacttttccatatactttaacactcacgctaactcccttgacgggatccctctcaacctcatactcattacgcgcctctcccaatctgtcatttagctctcggttcggtttcaatgtttacagttgggaaagcgagatggtagcgggatttgaactatggcgacaatcgaaaaagcccaagttggccgcgggaagcgatggcgacgatcttgaatgggcccgcaggaaggtccgtgtctgggatccctcagcttttcccctggcaccccctgaacctgaaatcccacaaccaaaccatgaagatgagtctcaagagtcagtacttaagctacgagtcgaccaatcctggaatgttcgtcttctctgggaaggtcgggtgaaggagcttttggtcagcgctggtgtcgggctcggcccgagttccttctcaccatcgtcatatgcaaatcccccgggtacagccggggctcaaggcagcggtgggggctcaccggcctcatactggaggggcgtggggggtttcggtatcatattcttcatgagggatttcttcggatctatgtacttgaatgagcactgtctagatgtatatagtttatcagattttatgagacaatagacaccatgaatctgcgttattgcgagacgg

SEQ ID NO: 19 - DNA, 896 bp, unidentified
  source 1..896: mol_type = genomic DNA
ggccctggcgtgctttctggctttcaacctcccgacctccctctaattacctcaattgaactcgatttagacgtggtgctgccacctcccgcctgccgcacaatgtttcttcgcaccgtttctcgcgctgtccctcgcagcaccgcggccatccgtgctgcaccgactgcctctgtgaacgccctgcagacccgcgctgcctcggaccatgctatccccaaccctaccctcgccaacattgagaagcgctgggaggtcatgccccctcaggagcaggccgagctctggatgcagctccgtgaccgcatgaaggttgactggcaccagatgaccctgcaggagaagaaggccgcttactacattgccttcggcgcccacggcccccgcgcccagccccccaagggtgagggcatgcgcgtgttcgccaaggtgctccagctcactgccgcctccgttgctgtcttctacgccatccacgccttcgccggcaagcagcccgccaccatgtccaaggagtggcaggaggcctccaacgaatatgccctgaaagagaagatcaaccccatccacggcatcagcaaagagggttacgaaggcaagggcttcgtccagagcccccctgccgagaagtcataggtgtaccagttgcccgaccgggaatgagttgatatctacgccggacggacggcggcacccatcgcacgatctatatgtcgatcttattacaagctactctttccatagccatgttcgacatgtctttgtgtcggaggatgggcctccgcccgtgcgcgcggccgtcgattgttccattctatcttttttggcaagcattggaaaatgcgtgtatcccgtactgtgctataatcaatgtatctcttttgtagccatagagc

SEQ ID NO: 20 - DNA, 641 bp, unidentified
  source 1..641: mol_type = genomic DNA
gtggcgcgccgggggggcatctacctcgacggcaacaacgacctggtcactatgaagggtaactacatctaccacaccagcggccgctctcctaaggttcagggtaacaccttgctgcacgctgtcaacaactactggcacgacaactccggccacgccttcgagatcggtgagggtggttacgttctggccgagggtaacgtcttccaggatgttactacccccgttgaggaccccgttgacggccagctcttcacttcccctgaccccagcaccaacgctcagtgctcgtcataccttggccgggcctgcgaaatcaacggcttcggtaactctggtaccttcaaccaggctgacactagcctgctgtctaaatttaagggtcagaacattgcttctgctgatgcttactctaaggttgcctcgagcgttgccagcaacgccggtcagggacacctgtaaaatggaaagaggaggttcagagcttaatttgctcatgtcggacgacatagccctagcggcttgctggtgaatttggcataatagcgtttctcttctcatacctactttattactccgtttggatccttattaggtaaatattagcccattgtatggttcaattcgattgactttgaggc

SEQ ID NO: 21 - DNA, 591 bp, unidentified
  source 1..591: mol_type = genomic DNA
gtggcgcgccgggattctcatcatcagataaaatcaagattaatcttactggacatcacaacgatccaacacaaagttccttcatacttcaaacaaatctctacaattgaatcaaaatgccatccaaaaccgaagcagcccgtctacaaaacgacttcggcgcagactactgggttagaaatacccaagaacgccgccactcaaccgctggccgcggactattcgccggtctccaggatgtcaagcactataacgtcgaccatggctgggcccgtcgcaagtctagcgataaccccggactccttgcttctttcttcagtcgattcaccgggggatcataccatccgccctcggaatagaattccttttcttaatgtgcgatattgggaggagtgtgatttgaattgggaataagggaaaagagtgcttggaatatttgagtctcagacttaactcgagtcaagtttcatttatgagtatactgaggtttttgtgttagtagcttggagtttgggtggtttattagtattacctattgcattaccatgtttatacatcgtgaatcatcgaatgaataccatgtcttcaatt

SEQ ID NO: 22 - DNA, 639 bp, unidentified
  source 1..639: mol_type = genomic DNA
gtggtatcaacgcagagtgacgagcccaccatccccggaggcgccgctgtcaccatccactcccgtaacgagaagaaggcccgtaaggccattggcaagctcggtctcaagcacgtccccggcatcacccgtgttactctccgccgtcctaagaacatccttttcgttgttaaccagcccgatgtctacaagtcgccttccagcaacacctggatcatcttcggtgaggccaagatcgaggacctgaactcccaggcccaggcttccgctgctcagcagcttgccgccgccgaggctgccgccggaggtgagcacgctggtcacgaccacgagcacgacatcctcggcaagggcaaggcccccgagaccgagggcaagaaggaagaagaggaggacgacggcgaggaggttgacgaggccggcctcgaggccaaggacatcgaccttgtcatggcccaggccaacgtctcccgcaagaaggccgtcaaggccctccgggagaacgacaatgatatcgtgaactcgatcatggctctcagcatatgatttggctgcctgccggcaggatgaatgagtgagctttgggcgcgaggtcacgttgatatccctgttctgggccctctcccttaagtgtatagc

SEQ ID NO: 23 - DNA, 832 bp, unidentified
  source 1..832: mol_type = genomic DNA
gctcccaacgtcaacaccccctccgccttcccctcgaccgttactcctgcccgtccgattacaacaaggagaatgttccttcagcgtacggtatctaccctcgcgaggcgcacccccgtgcggggccttgctgccgcgcgcccgttttcttcgtccgttagccgattcaacaagtacgaggttaaggaggccaagctccgttctcttgacgagatccaaactgaagaagacctcatcccccctggtgctaagcccggtaccgtccctagcgatatcgaacacgccactggtctcgagcgtctcgaactcgtcggtaaaatgcagggaattgacatcttcgacttgaggcctctggatgcttcccgcaagggaaccctcgaaaaccccattgttgtcaacggtgctggtgacgagcagtacgctggttgcactggttaccccgtcgactctcaccaggttaactggttgactgtctctcgtgagcgccccatcgagcgctgcaacgaatgcggtaacgttgtcaagctgaactatgtcggacctgaggaggaccctcacgctcacgaccacggccacggccaccaccctgcccccgaggagcccaagaccttcgccgactacgtcaagcccgagtactggtaccggtaaataccccagcagtacgacgcgagagttttcaaaaaagagaataagaaacaagcaaagggacggatcaagacgggctagtgcgggaatgtcaaacgcaacatatttaagcattgggtctactatatacgggttcattcgtccattgattcctcggtctagtgttttcttgaacgtctttagctgg

SEQ ID NO: 24 - DNA, 832 bp, unidentified
  source 1..832: mol_type = genomic DNA
ccagctaaagacgttcaagaaaacactagaccgaggaatcaatggacgaatgaacccgtatatagtagacccaatgcttaaatatgttgcgtttgacattcccgcactagcccgtcttgatccgtccctttgcttgtttcttattctcttttttgaaaactctcgcgtcgtactgctggggtatttaccggtaccagtactcgggcttgacgtagtcggcgaaggtcttgggctcctcgggggcagggtggtggccgtggccgtggtcgtgagcgtgagggtcctcctcaggtccgacatagttcagcttgacaacgttaccgcattcgttgcagcgctcgatggggcgctcacgagagacagtcaaccagttaacctggtgagagtcgacggggtaaccagtgcaaccagcgtactgctcgtcaccagcaccgttgacaacaatggggttttcgagggttcccttgcgggaagcatccagaggcctcaagtcgaagatgtcaattccctgcattttaccgacgagttcgagacgctcgagaccagtggcgtgttcgatatcgctagggacggtaccgggcttagcaccaggggggatgaggtcttcttcagtttggatctcgtcaagagaacggagcttggcctccttaacctcgtacttgttgaatcggctaacggacgaagaaaacgggcgcgcggcagcaaggccccgcacgggggtgcgcctcgcgagggtagataccgtacgctgaaggaacattctccttgttgtaatcggacgggcaggagtaacggtcgaggggaaggcggagggggtgttgacgttgggagc

SEQ ID NO: 25 - DNA, 1263 bp, unidentified
  source 1..1263: mol_type = genomic DNA
acccaaaccctgcgaagcaaaacgcgttactcgatttggtcatttcctccaaagcatcattcctctttgggcactgcctgtttcgtccattaatcaactgtgcttgaatctctaactatcttttgatataccctctttatctctctcccctttaatctttttttctctctctctcttttcctttctttttcggttactcactatcatggccgacatcactgccgtcggtgaggagaacccttctcctacccaggatgagctgcagcaggccgcggccggtaacggcgctcctgataaccgcactcccaagcgtcgcatgagtgacgatgaagaggacgaggagaagcagggtcgcgagcgcagaaagattgagatcaagttcattcaggataagtcgcgtcgccacatcaccttctccaagcggaaggcgggtatcatgaagaaggcatacgaattgtccgtcctcacaggcacccaggtgctgttgctggtcgtgtccgagaccggcctggtctatacctttaccacccctaagctccaaccattggtcaccaaggcggagggcaagaacctgattcaggcttgcctcaacgcccccgaccctaccaccagcgagaatggcgtcgatgcccccgaggtcccagcggagacccccgaggatgtcaaccacgccaacgtcaacgctgccgcagcccagcagaccaacatccctcgtcccaccggaatgcatcccggctacatgaccaacgaacaacagcagcagatggcctactaccaaaaccacctccagcagcaacagcaggccggtgggcagtaccctggcatgtctgtcggtggtcgcatgcctacgcagcaccagcctaccgcataatcttatttactcttatctacgctcccacgcacctcctctttctgatttccctgcatatggtcttgtttttagtagctgaggagtccagagttcagttgtttttgccttctttccgcatctaccctttattttcccctctttcgttattatctctctcccctgacatttgatacccgacaatcctgttgttcaatccatcgtcgcatgaaaacgggtcctataaatataatgcatccccctgtttactttcgactgcgaacgagagcatgcaaatctgaagaacagcatggtcaattgtctcagtaacctcgttaaggcgccgatgagtttggcgtttacatactctgctttggaacgtgtgatgcctttttaccgttcaatgaaagcgactc

SEQ ID NO: 26 - DNA, 1066 bp, unidentified
  source 1..1066: mol_type = genomic DNA
aggctttgatggcctcttcaaaggggaacattgttcactcgaccagagttgagtagagccaagaccattccataatctccaggggcatatcgccatgccgtcttgtcaagacgatctccttctcacacatattgatgataggtaccgttagattggactcggttgaaccgagtccaatttggaactacgactcctcccgggcctgattttgcaacgaggattccactcccgtacactacagaactatatcgacaccgtcgctgagattcaatgtattctttcattcgactggcttcttcctccgggtgagacgagctgatatttgggagaaatattggacatcccagataatctcgcgcaaagtctagtttggtctggttgatatcggaaatgatcacttgtttggctccaaacgctgtggccgtcgctgcacagaacagaccgatagtccccgacgcttgtaccaggacagtatgaccgggagtgatacctgccacccgagctccatgaattgcaacactcaacggctcgaccaaaacagtttcttcaagtgagaaattttcgggaatcggatacaccaaatcctcgggtgcgcggaatagatgggtcagagtcccatggttattggggggaggatccgcggcaaagctcatctctggaaaaatatcatatctccctgctttacattgtttacatcgtcgacgagagaaactagctcgatggcaaagcggtcgccaggaatcacttttgtcactgccggtccaattgagtgcacgatacctgatgcctcatgacccatgaccagtggttgctcgtcagagaccattcgaagtaccccgccgtgtttccagaaatgggcctatggaattggaaattagcaaaagagaccctggtgaaaggaagagggatttgtggggactcatatcgctcccatacacacccacatacgcgatgcgaactaatatatcatagggatcactgagggtagggacatcgcggtactcaagtcgagctttcccaggcccgtagagtaggcaggacaaattattctgaaattatgatcaac

SEQ ID NO: 27 - DNA, 1066 bp, unidentified
  source 1..1066: mol_type = genomic DNA
gttgatcataatttcagaataatttgtcctgcctactctacgggcctgggaaagctcgacttgagtaccgcgatgtccctaccctcagtgatccctatgatatattagttcgcatcgcgtatgtgggtgtgtatgggagcgatatgagtccccacaaatccctcttcctttcaccagggtctcttttgctaatttccaattccataggcccatttctggaaacacggcggggtacttcgaatggtctctgacgagcaaccactggtcatgggtcatgaggcatcaggtatcgtgcactcaattggaccggcagtgacaaaagtgattcctggcgaccgctttgccatcgagctagtttctctcgtcgacgatgtaaacaatgtaaagcagggagatatgatatttttccagagatgagctttgccgcggatcctccccccaataaccatgggactctgacccatctattccgcgcacccgaggatttggtgtatccgattcccgaaaatttctcacttgaagaaactgttttggtcgagccgttgagtgttgcaattcatggagctcgggtggcaggtatcactcccggtcatactgtcctggtacaagcgtcggggactatcggtctgttctgtgcagcgacggccacagcgtttggagccaaacaagtgatcatttccgatatcaaccagaccaaactagactttgcgcgagattatctgggatgtccaatatttctcccaaatatcagctcgtctcacccggaggaagaagccagtcgaatgaaagaatacattgaatctcagcgacggtgtcgatatagttctgtagtgtacgggagtggaatcctcgttgcaaaatcaggcccgggaggagtcgtagttccaaattggactcggttcaaccgagtccaatctaacggtacctatcatcaatatgtgtgagaaggagatcgtcttgacaagacggcatggcgatatgcccctggagattatggaatggtcttggctctactcaactctggtcgagtgaacaatgttcccctttgaagaggccatcaaagcct

SEQ ID NO: 28 - DNA, 564 bp, unidentified
  source 1..564: mol_type = genomic DNA
agactcaacgataattcaatacccagttgcaacactatttgttttctaaacatatcctcaagaccaattctaccttaatctccccaacacaactcaattgaaaacatacaccatgcctcgcggagccgaatacgccaacggtcctctccagagcgacaatgccatcgaagctggcgaaaataaggcccacggaacctccggtaacaccggcctcaaccgcgtcaacaaggtcgccgaattccccgaaggcgccagaggaaccggtaccgctgctaacccgctcagtggccagggtagcgccggccatcaggatggaaagggtggccatgacccgaagacccttggagagaacaagggactgggtactcaatgatcttatgattcagaagacatgagttatttgcatgagctgggctcgctgcgattctgtgggattctgtgatttgtaatatgatttgcatgggtcaggtcagacttaattaagcatgcgctattgtttccgttatgcttatgatatggatgggtccatggttggagttgataatctaatatggaattgaagtg

SEQ ID NO: 29 - DNA, 39 bp, unidentified
  source 1..39: mol_type = genomic DNA
attccatctaaagctaaatattggcctgagaccgatagc

SEQ ID NO: 30 - DNA, 577 bp, unidentified
  source 1..577: mol_type = genomic DNA
gagactatgacgacgatcacagagttcccgccattctacacgcagcagccgaatgcgagcgcgctgacgcagcagctggggctgtggcagaagcatatactgagcacgtgcaagcagcggcggcagttcaagctgagcgtgagtgatgatatctgggccaacgagaggataaagcgagctgcttctcgtgaatttatttctgtgattatctcctcgctggtgacagaagggctagcgagctatacagacgccaccaaggaggctgtgtgggtgtactggcggagtctatctgattgggcgcaggcggcgtacgcgtacgcggaaagcacagcgcagctgaacacgccgttgacgtactatgagctagtacaaggggagtacagccatctatctgagctgcatgagatgccagtagagctgctcaagcttgctgtgtcgctgctggtgaagcagaacaaagcggtgataatcaaaacgagtcaaggggaaggtgtcaaattcgtctagtatagaataacttaggttacattggaatctggtaatcaattcccttgtcattcagcttctgctgctttcc

SEQ ID NO: 31 - DNA, 606 bp, unidentified
  source 1..606: mol_type = genomic DNA
gggaacaaactctcattctaactaaatactttttactctctgctcccctatcgcattctttttaggacattcagaaggtgatcgcttgaccaaatgtctatcccaaaagcggcagctcataccgacaaggcgcctcagcctttcaaggacctctattcgcaagcagttattgctggtggcgtggtctattgctctggaattgttgccattgaccctgaaaccggtagcctgattgaaggagatgtcaaggctcatacggaacgaattttacaaagcctttctagtactctacaggccgccggtaccagtcttgatcgagctgtaaagatcaatgtttacctagcaaacatggaagacttcacatccatgaactcagtttacgaaaagtattttgtggatggagtgaaaccctgcagaacctgtgtggctgttaagtctctaccttttggcactgatgttgagatggaatgcattgcagtactgtaaatgtttagttttatgcgcaactgagaaagacggaaggatcatcctattactttttcgaatgtgctctttggatttctctgttggatacacaacaatgccacacattgggtacaaccagat

SEQ ID NO: 32 - DNA, 411 bp, unidentified
  source 1..411: mol_type = genomic DNA
ggaaccaattacatccatcaaccaaaccaacaaaatgctgagatctcaattcggtgtaatttcaaacgcagcaaagacagccgcattcctcaagcctgttcaaaccagattgtacgctagtggcgctctctcgaagggcgacatccaaactcgcatttttgatgtcctcaagtcgtttgataaggtgaaggctgataacctcactgaatcggcttctttcaccaacgacctcggcttggatagcttggacgccgttgaagtcgttatggccattgaggaggagtttgccatcgaaattccagacgctgaagctgacgcaatccaaaacgtgaaccaggctatcgaatacatcgccaaaacccctgaagcacactaaacacgctaaataattttatcaattcatttcaaacg

SEQ ID NO: 33 - DNA, 619 bp, unidentified
  source 1..619: mol_type = genomic DNA
gtggtatcaacgcagagtggcgcgccgggaacgaagaaagtttcttcgacctacctatcaaagatatgtaaggagcaacataaatcaatattcttttatctgattgaccttcactctagtgcctcgctacgatcagttacaaccttgcccgcgcgaatggaacgtatacgactttccgaacaggccgcgacgatttgcaaccaaattcgtgaaatgataccagagactgccactttgcccaatcaacctggcaaggatcaagctgaactcatgcatgaagatgaaaacgggaataagatatacggcgggaaacttttaacggagagagctgctcgactgaaagagcacatgaagattgaccaagtgagtgccagatttatctcacagtactttactaatggcattcaggactggacagagcgcttggtatattggacaaagccgacgaaactattgaaccaacggaaacaaggatatatcataccgttatctaaagacatcgttctacaacctgggggacctttagaagcaaataacggctttcgggtcacaaacgagcggattctgagttcaggagctgcccttttcattatgccccaatgatattattttgaaaccc

SEQ ID NO: 34 - DNA, 1647 bp, unidentified
  source 1..1647: mol_type = genomic DNA
accgctaccgtcatcactacaaatgctggctcgcagcttacagcaaatcagacgctcaagcaggctgagcttacaattgcgcgcctacgccagcagtccagaccgcagcgcaagcttctctaagctctcagagcaagatctcccatcactcgcctccatcttctcatcccccgacacctccctcctcaccacgctcggcgacaagccaacagccaccagcgacgatctcgagccattcaacgtcgactggatgggcaagtacaagggccactcttccataattgtgaaaccaaagacgacgcaagaagtcagcaaggtgctgcagtggtgcaacgagcgcaacgtagctgttgttccacaaggtggcaacaccggtctcgttggtggatccgtgcctttgcacgacgaggtcgtcttatctctctcctcaatgaacagcatcagacacttcgaccctctttccggttacgtttctgtcgattccggtatcgtgctcgaaaatttggataactacctcgcacaacaaggacacattgtccctctcgatctgggtgctaaaggctcctgtcagattggtggcaacgtcgcaaccaacgctggtggtctgcgcatgttgagatacggtagtttgcacggcaacgtgctcggcctcgaagtcgttctgccagatggtagagtaatcaatggtatgaagggactcaagaaggacaacactggtatcgatctcaagcagctcttcatcggctcggagggtgttctcggtgttatcactggtgtcactctcgccacacccgtcagaccatccgcaactaacgtcgctgtcttcgctttgcctgactatgagtcagtgcagactgccttctcatcagctagacgcgatctcggtgagatcttgtcggcgtttgagttcttcgatgctgcctcatacaagctcgtgcgcagccatggacacgcagctgagcgcaaaaccttcgaagatggggaagacgcaccatttttctgcttggtcgagacgtctggctcgaacaaagaccacgacgatgagaaactgggtgctttcctagagcagctcatggagtcaggtatcgtcaatgacggtgtattggcacaagacgagacgcaaattggccagctgtggtcgctgcgtgagggcattccagaagctgcaggcaaagctggtcgcgtgtacaagtacgacttgagtttaccagtcgagaagatgtactcgctggtgccagagctgcgccaaaagcttgctgagaagggtctgcttgccgctgagtcagagggtggtaatggagatgggccagtcaagacagtcttcggatttggtcaccttggcgatggcaacctgcacatcaacattgttgccgatgcttacagaaaggaggtggaggaagtcgtcgagccatacatttacgagttggtagccaagtacaatggatctatctcagcagagcatggtctcggtctgatgaaggcaccttatgtcgcatacagtcaagacgcgccatcgcttgacctcatgcgcactctcaagaagacactcgatccaaagggcattctcaacccatacaagtgcgtcaccgcggaatagattggagttatagatttacgttatatgcatgcgatcctgttacattatcc

SEQ ID NO: 35 - DNA, 669 bp, unidentified
  source 1..669: mol_type = genomic DNA
gtcatcaacttcattcagcaaaatggctctctattacggcatcgtatttggtatcttaacatttgagattattctctttttcttattcttgttgcctatcccaactcgttggcaaaaaccagtgttccgttggttagctacctcacctaccattgcacatgctcaatatatcatgaaaattgtatttgtattcatctttgtgctcttccttgattccgtcaacactctccgcgctttctacgaagtagtgaacactgaagatgagaatggtggtattccagctgccggtaactctgatttcagagctcaagttggtcaagctgcaaagaagttttatgctcaaagaaatttgtatctcactggattcaccattctgttattactcattttgaacaagatcaagaacatggctatggactatattagattggaagatcaattcattgagcttgaaggatccgtttccaaagatcccgccatcagaaaggcaagcaaagaaatcgacactactcccatcgaagaccatgttacaagactcgagcctgttgaacaagaacaggaaaacaaaaaggatatctaattcacacctgtaactaatatgtaaacatctccctcgctaaaagcgcaataaactaaaatcagcatcattgcgtatctctttcttctcac

SEQ ID NO: 36 - DNA, 873 bp, unidentified
  source 1..873: mol_type = genomic DNA
gtggcgcgccgggaggcgcctaacggtcatgaattgcctcctcgcggttatgatcccggagaaaacacttaccaagcaccacctgatgaacgtagtcaagtagatgttgcgattgaccctaaatccaaccgtcttcagctgttgaagcctttccagaagtgggacggcaaggacatcaccaatgttcctatcttgattaaggtgcaaggcaaatgcactacagatcatatttccatggccggcccttggctcaagtatcgtggtcatttggacaatatcagtaacaatttcctcattggcgccaagagtagcgaaggcaaagtcaacagcatcaagaatgcttttactggtgaatacaagggtgtccagaaacagctcgtgattacaagaaggaaggtgttcgttgggtcgtggtaggtgatgagaactatggcgaaggctcctctcgtgagcatgccgctctagaacctcgattcctcaatggagctgccatcattaccaaatcatttgctcgtatccatgaaaccaatctcaaaaagcaaggaatgcttcctttaacctttgctgatcccaaggactatgacaaggtggacgcctcagataaagttgatattcttggcttgactgatttccaagaaggaaagccattgacccttcgcttgcacaaaaaagatggatcaactgtcgatgttcctttgaaccatacattcaacggtcagcaaattgaatggttcaagcatggatctgccttgaaccttatgaaggaaaatactgccaagaacggaagcttgtaggtgcaccgttacgttatcttcacaagcatttgtatgtcaaataaactcgattagttacttgcacttttgttaagtttat

SEQ ID NO: 37 - DNA, 874 bp, unidentified
  source 1..874: mol_type = genomic DNA
gtggcgcgccgggaggcgcctaacggtcatgaattgcctcctcgcggttatgatcccggagaaaacacttaccaagcaccacctgatgaacgtagtcaagtagatgttgcgattgaccctaaatccaaccgtcttcagctgttgaagcctttccagaagtgggacggcaaggacatcaccaatgttcctatcttgattaaggtgcaaggcaaatgcactacagatcatatttccatggccggcccttggctcaagtatcgtggtcatttggacaatatcagtaacaatttcctcattggcgccaagagtagcgaaggcaaagtcaacagcatcaagaatgcttttactggtgaatacaagggtgtcccagaaacagctcgtgattacaagaaggaaggtgttcgttgggtcgtggtaggtgatgagaactatggcgaaggctcctctcgtgagcatgccgctctagaacctcgattcctcaatggagctgccatcattaccaaatcatttgctcgtatccatgaaaccaatctcaaaaagcaaggaatgcttcctttaacctttgctgatcccaaggactatgacaaggtggacgcctcagataaagttgatattcttggcttgactgatttccaagaaggaaagccattgacccttcgcttgcacaaaaaagatggatcaactgtcgatgttcctttgaaccatacattcaacggtcagcaaattgaatggttcaagcatggatctgccttgaaccttatgaaggaaaatactgccaagaacggaagcttgtaggtgcaccgttacgttatcttcacaagcatttgtatgtcaaataaactcgattagttacttgcacttttgttaagtttat

SEQ ID NO: 38 - DNA, 718 bp, unidentified
  source 1..718: mol_type = genomic DNA
ggcgcgcccttgacacaggagcacgggtttcccgtgcgcgtcatcgttccaggcgtggcgggcgcgagggccgtgaagtggttggatcacatcacagtgcagcgggaaatgagcagcaatcattatatgcatttcgactacaaggtcctaccaccagaagcggtcgatgcggaaagggcacgcaccttctggcataaagtcccgccggtgatcgacatgccagcgaattctgccatcacgtcgccacgaaatgaagacacggtggaagtggatgcagagggatttatcacggtggatgggtacgctttgccggggggagaagatgggccggtgaaaagagtcgaggtctccattgacaaggagagatgggtcgacgcggaactgtttacacatcccatggaaagcaagtggacttggaaaatctggaaggccaaagtgcaggtcgagccgggcgagcgaagatgtctctacagcagaaccactgatgaagcgggcaactcgcagccgcagcgttctcagtggaacctgagaggcgtatgttacaacggctatggagaagtgaggaatttgaaggtggtgaaaggataggcccaatcgttcattccatcatccatcaagatgtgtctgtatgtgtatgaaggcctgaagcgaccacgggaccccagggtggtcactaaacagtactcaaacggactgtttggttcgtttgacactttcg

SEQ ID NO: 39 - DNA, 160 bp, unidentified
  source 1..160: mol_type = genomic DNA
aatcaagttcgactgtcaaaatgccagccaacacgatgtctgccactctcagatccctccacgttcccgggaaaccagtcatcttcgccaatgtctgggacaccgtctccgccaaatcaatcgcacctctggattcatgcaaagctctagcaacggccag

SEQ ID NO: 40 - DNA, 1274 bp, unidentified
  source 1..1274: mol_type = genomic DNA
gccttgcccatgctactcatcaaccctcctcgcaacctcatcggccaaagtcagtctggtaccggcaagacggctgcattcaccctcaacatgctctcacgagtcgacccaaacatcatgacccctcaggctatctgtttggcaccgtcgcgagagcttgctcgacagattcaggaagtagtcgacaagattggccagttcacccagatcaagagtttcctcgctgttccgggctcttggtcgcgtaatgtcaagatcgacaagcacattcttgtcggtacgcctggtacactcgtcgacatgctttcgcgaggaggcaggatcttcgacccgaagcagattagagtctttgtgctggatgaagcggacgaaatgatcgctttgcaaggtctgggggaccagacgaagcgcatcaagaggatgctgccgcctggggtccagaacgtcctgttctccgctactttccccgacaacgtccgagactttgcaggcgacttcgcacccgaggcgaaccagatcttcctgaagaaagaggagatcactgtcgacgccatcaagcagctctacctcgagtgtgatggagaggagcagaagtacaacgccctttctgccttgtacgacatcatgtcgatcggtcagagtatcgtattctgcaagcgaaaagacacggccgaccgaattgcggcgagactgacggatgagggtcactctgtcgcttctctacacggtgacaaacagactcgagaccgtgatgacatccttgacgctttccgagatggcaaaaccaaggttctgatcaccaccaacgtcgttgctcgaggtatcgatatccagcaagtgaacatggtggtcaactatgacgttcccgatctcggtccagagggagattggaagcctgatatcgagacctatatccatcgaataggtcgaaccggtcgatttggtcgaaaaggttgttcggtcatctttgcccatgatcagaggtcgatgcaggatgttcagttcatcgccgatacgctcggcaagaaaatgagcagaatcaacgctaccaggcagactgatctcgatcagctcgaagcggctttgaaagccgccatcaagggcaatcaaccgaaagagtgaagagtggcaccgaattcgaagagacgggcgctggaagatatcctgaagcaacagggaggagctccccttatagcatgatcattgacgataaccatctagggcctgaagtacattatgatagatagcagacatcaatgcaacgtcgcgtcgcc

SEQ ID NO: 41 - DNA, 919 bp, unidentified
  source 1..919: mol_type = genomic DNA
cacctccaacctctttctagtttaccttcaaaacacatcggtgtaaggtcttgcccaacatggctaccttctcgacccgcatcaacctcgtccccacctcccgaacgctcgctagcggcgttccattcgcacccaggatcgcccttgttcatcctcccgcgtctcacggtcacggaacgagcggtcccaggagtgatgtcccacccaggtgggctggtgtccagggtggattcgcctcgaactcgagggtcaatgtactccccaccggcaacttccagcaacgattcatgtccaccacgccagcccgcaagatcgaggctcaaccccacgtccgaggtgttcccgattggtcggcatatcagtcttcgggcaagggcgagaacacccgatccctttcgtacttcatggtcggatctctcggtgtcctcgctgcttcaggtgccaagtcgaccgtcagcgacattctgagcaacatggccgcttcggctgatgttttggctttggccaagatcgaagttgagatgggtgctatccctgagggcaagaacctgatcgtcaagtggcgaggaaagcccgtcttcattcgacaccgaacggaagatgagattaacgaggcacgcgcagtcgacatcaagtctttgcgtgatccggagagcgacgaggataggacccaaaggggagagtggcttgtcatgctgggtgtctgcactcacttgggttgtgttcccattggcgaggctggtgattacggaggatggttctgcccctgtcacggatctcactacgatatctctggccgaatccgacgaggtcccgcccctctcaacttggaggttcccgagtacgctttcaacgacgacgaggagaagcttgtcattggttaggtgtagatggacatatgcagtctatggccatagcg

SEQ ID NO: 42 - DNA, 1459 bp, unidentified
  source 1..1459: mol_type = genomic DNA
ggggagcatcaccatagtgacagggactgtagctcggtcaagcgcgtattcgcacttgggcgcctggacgttactctggccgtggagtgcgggagtgtcaggacgatgtcccttcgtgacctcgcttgctacactctcacgctcaagccatctaccgagaataccctcctgaccgagctcacggctttggagggaccgagtgaggagccacgattcgcaagagtgcgggagaaggtggaaggagaggtctattcgtctgccatatacgatgcgttgacgggagccaagctggcctcggtgggtttcgcttccgaaaagcagaagaacaggaggctacagctacacaacccggatgagagtgtgccttttgacaatactagcaagctaggtttcgaatggacattcatcttcgaaggcaacaagtacaggtggacgagagagctatacggaaaagattatatctgctcactagaccggaaacccgatccaagggtggagatctgcctagctcgagacgcagattcgaaagcgcctggacgactgcagattctacactacaacatcgaacgattcccgaacgagatcaaggatttgaggggactggaaacgctactcattgctaccctcatgtgcttcgtcgacgcggccgaagatcggtccaattccggtccgacccgcacttcgcccttgcctgctaagccggttgccaatgctgcagcaggtcaaagcggcaccagcgcaagtggatcttctgatacccgagcgaaagttgcgccggtgacagtgccagtaatcactgcagaggactttgaggatgattgtgacccgaatgagatactggtaggaacggagactgatgtgggcgagcacattgcacgagctatagcgcttttggaggacccgaccatgctgttcattgtcattcgaacgcgaactgcggccgcgagctcaagagcgttagaagtctccttaggggttacaaggttccggcaccgtgagggcatgagcgagctgcatcaatacgtggtagaggaagatccggtccggaagccgaaacccattatgcctgctcagggcctcaagttgatcaacctggatgatcgaccagcggcacaatcacccaccaaaccggaatggtctgccccacctaacatcgctgtttacctatcatcgatcgagttgccagatctcacgcccaagcccaagcctgtccaggggcacacacggccgccaactcaagcacctcatgctcggcctccgccgccttctcaactaccacaaaagccgcagccgcggccacgcccgcctccatccgatggttcaggtagtagtcagactacactcgcttcaacgcgaccgccccaggacgacgggaaggattcgagaaagtctagctttggaagactctttggcaggtagtacgatacacttagcagggcatatgcaggtgtatcgacgg

SEQ ID NO: 43 - DNA, 623 bp, unidentified
  source 1..623: mol_type = genomic DNA
ggcgacgacaacaagaaaacaatacctcacctgaacatattcaataatggcgtcccaattgatgcccctggagctgatcgatcgttgcatcggatcaagaatgcgtgtgatcatgaaaggcgacaaagagttcagcggcacacttctcggattcgacgacttcgtcaatatggtgctcgaggatgtcaccgagtacgactacaccggcgcaacgaccaagcttcccaagatccttctgaacggcaacaacatctgcatgctcatcccaggtggcatgcccgagggcgagtcatgaatcacggacatatgatatcccttcttacgtctcttgaaatggcaaagcgagtctgatttaagaaccacacgtgtcatgagaaggcagactataccgctgtccagtccaagctgcttgaacaataattatcccgacggagccacgaaaacgtgacaagcggaagctcgcattcgcaaagcgccggcgcaataaaacgccttgttcagctcgccgactttgtgcatgcatgcagctcgccacaccccgcagatatcaggctgccttcttgttatcaggtatgcgtgtttatactctagcttatttcagctatgcaaaacctatatcatcc

SEQ ID NO: 44 - DNA, 489 bp, unidentified
  source 1..489: mol_type = genomic DNA
gcaaaggagttgtcgcctgacgtcaagcctgagccgacatggtcttgtggcgaggttgtcaatgtcgtcgatgagcacggcaatgtcatcaaaccgtcagacctctgggtcaagatgggtatgcagcagcaggacaatgtggacaacctattgatcgacgacctgtgtgatcagatgagggccaaggccaaatgcacagagaacggcgctcaattgaatgtcgacgacctgaaccacatgatgtcgtatgacaagtcatataagcagaaaagggtagacgacctcaaagacaagtacggctggggagcagtctttggcccgaaatgagccgcctccgcgggggcaaggtggacggacgatggtagacatgaatatgagagcaaacagacatagggtctgagtccagtagtgtgcttgtaccaccactgtaaatatttgtacgatagccctacaccacttacaattgatcatgtaactgtgtgaaccgtg

SEQ ID NO: 45 - DNA, 480 bp, unidentified
  source 1..480: mol_type = genomic DNA
gggagcctgacccgccgtgcttggttcatccaatccacctgcgccaccactggtgatggtctctacgagggtctggagtggctcgccgacactctccggaaaacgaaccgcgattaaacgcgtataatacgaaattgtgatggggaggattgtgtacgtagcagagcaagagaaataccacgggaaatctgcaaatgatggaatgatgattatggcgggagtttcttccaatgttcttctgcgaggccaaatatcccggcgatgaaaaagaattccctcaccggcatggcatggccatcctcaggagcaaggtgtttgtgtttggctcgccagggctctatttctcttcgctatgctattagcctcatttgttcttttctctctggcgccacgtcaaaattgctggtttatctccttttgattgcatgttcagtatcggtatgatctcagtataccagcacttgggttgagcattcttct

SEQ ID NO: 46 - DNA, 1139 bp, unidentified
  source 1..1139: mol_type = genomic DNA
acctcactttgtgcgaattatcctcctacagcatcagctctcttcagaaagaggctaaatctatagaccgtccggaacaggttgtcaacacgcgcgataagagaagaagggaatctactggtagacaacagatcgatcgctcttcagcaaacgcaagatggagaaccttcttcgtcagatgcaagggggaggtggtaggatgggtgcacggccaggccctggaggcgaaactatcctcgccgacaacggtgaaacagtccatatttcatctcttgctctattgaagatgctcaagcatggacgagcgggtgtgcctatggaagtcatgggtctcatgcttggcgaatttgttgatgactacactatctcctgtgtcgacgtttttgcaatgcctcaatccggtacgacagtgacggtcgaatcagtggatcacgtctttcaaaccaagatgttggatatgttaaaacagacgggccgacccgagatggtcgtcggttggtaccactcgcaccccggttttggttgttggctgtccagtgtcgatgtcaacactcagcagtctttcgaacagctacatccgcgagcagtagccgttgtcatcgaccctatccagtctgttcgtggtaaagtcgtcatcgacgctttccgatccatcaaccctcaatcacttgtcgctggacaagagtcgaggcaaacaacgagtaacattggtcatctgaacaaaccgtccattcaggctctcatacacggtctgaataggcattactacagtctggccatcgattacaggaaaacagaaggggagcagggtatgttgttgaacctgcacaagcggggatggacagagggtttgaagatgcgtgatcactcagagatgaaggagggtaatgagaaggcaatcaaggaaatgctctctcttgcctcggcctacacgaaatctgttcaggaagagacgacaatgacggccgaacagcttaaaacccgtcacgtaggaaagcttgatccaaaacgtcatttgggcgaggcggctgagaaagcgatgggtgatcaagtgacgcagagtctggccatgggtgtcctggctgagctgtagacgtagaagagggaagaaaggaaacgacatgcattgtacatatcgc

SEQ ID NO: 47 - DNA, 674 bp, unidentified
  source 1..674: mol_type = genomic DNA
ggacacaccggtgacgtcttgagcgtctcgttctcggccgacaaccgacaaatcgtttctgcttcccgagaccgaactaccaagctctggaacactctcggagagtgcaagttcaacattgttgacgatggtcactcggagtgggtctcttgcgttcgattctctcctaaccccgtcattcccgtcatcgtctctgctggttgggacaaggtcgtcaaggtctgggaattgtccaagtgcaagctcaagaccaaccaccacggtcacactggttacatcaacaccctcgccgtttcgcccgacggatcgctcgccgcatccggtggaaagtatggcatcaccatgctttgggatttgaacgatggcaaacacctctactctctagaggctggagacattgtcaactcgctcgtcttctctcctaaccgatactggctctgtgccgccactgcttcgtcaatcaagatcttagacttggagtccaagtcaatcgttgacgacctcaagccagacttctccgccgagtaccctgacaaggctcaaaagccacaatgtacttccctcgcctggtctgccgatggtcagaccctctttgccggtttctccgacaacctcgtccgagtctgggttgtcactgcttagagtcgtgaggattgtatgcatggataacgtgg

SEQ ID NO: 48 - DNA, 674 bp, unidentified
  source 1..674: mol_type = genomic DNA
ccacgttatccatgcatacaatcctcacgactctaagcagtgacaacccagactcggacgaggttgtcggagaaaccggcaaagagggtctgaccatcggcagaccaggcgagggaagtacattgtggcttttgagccttgtcagggtactcggcggagaagtctggcttgaggtcgtcaacgattgacttggactccaagtctaagatcttgattgacgaagcagtggcggcacagagccagtatcggttaggagagaagacgagcgagttgacaatgtctccagcctctagagagtagaggtgtttgccatcgttcaaatcccaaagcatggtgatgccatactttccaccggatgcggcgagcgatccgtcgggcgaaacggcgagggtgttgatgtaaccagtgtgaccgtggtggttggtcttgagcttgcacttggacaattcccagaccttgacgaccttgtcccaaccagcagagacgatgacgggaatgacggggttaggagagaatcgaacgcaagagacccactccgagtgaccatcgtcaacaatgttgaacttgcactctccgagagtgttccagagcttggtagttcggtctcgggaagcagaaacgatttgtcggttgtcggccgagaacgagacgctcaagacgtcaccggtgtgtcc

SEQ ID NO: 49 - DNA, 480 bp, unidentified; identical to SEQ ID NO: 45
  source 1..480: mol_type = genomic DNA

SEQ ID NO: 50 - DNA, 1170 bp, unidentified
  source 1..1170: mol_type = genomic DNA
aaagttgatcgaccacattgggtctgagaaaaacccaatgcttttttgtacagcagtgaggaatattggtcaatggccgaaaggctgaaccagtaacttggaagaatgaaattgtatttgtataaatacaatagtggttaaaccacataaaattctaaatagattatatataatgacaaaatctatttataagtcttgaccaaactacgtgccagcagtcgcggtaatacgtagaaggctagtgttagttatctttattgggtttaaagggtaagtagacggtaaattaaactctaaaagagtacttatttactagagttatatgagagaaggaagaattcctggagtagagataaaattttttgataccaggaggactgtcaacggcgaaggcgtccttctatgtaataactgacgttgagagacgaaggcttgggtagcaaacaggattagataccctaatagtccaagcagacaatgatgaatgtcatacattagatagattttaatgtataaacgaaagtgtaagcattccacctcaagagtactatggcaacatataaactgaaatcattagaccgtttctgaaaccagtagtgaagtatgttatttaattcgatgatccgcgaaaaaccttaccacagcttgtatagcagttatgaaaaattgttacaagcgctgcatggctgtctttagttaatgtcgtgagatttggttaactcctctaattaacgaaaaccctcactttatttatatatataaagtggttcgctattacattggttgataatagggattaagacaagtcattatggcctaaatgctgtgggctatagacgtgccacatacgcctttacaaagggatgcgatattgtgaaatggagctaacccccaaaaaaggaaatactatggatagtagtctgtaactcgactgcttgaataaggaattactagtaatcgtgaatcaccatcgtcacggtgaattatttctcagttaggtactaaccactcgtcaggcgctgaaagaagaagatgcagtaagtttgatgttttctgtgtatgattatacataaagttgttgtataactacgcagaaaagttttcgtatgcaaaactttgattggtgttaagtcgaaataaggttcgtgtaatggaaattgcacggggagc

SEQ ID NO: 51 - DNA, 1231 bp, unidentified
  source 1..1231: mol_type = genomic DNA
ggacgatatgacttcaagcagcctcagcgaattcgagacgcttctgtcacggccacgccagaatggaacctgcttgaagagatcgagtttggccgattgggcaagctcaacctttccgtcgaagagcccgaagacctcgaatcgcacggtaccctccaaggttacgacaagacgtttgaccgcatcaacactcgtaccgaaagacctctcgagatcattgatcgagcatggtacaatcaaaccacttctgacgatcccgttattgctcagctcgctcaaacgcagtctgcccaaatcttcgcgacagatgccattcttgcggttctgatgtgcaccactcgttccgtaaactcgtgggatatcattctcgagcgacgaggtaaccagcttttcctcgacaaacgagattctggtccattcgactacgtcactgttcacgaaaacgccgccgacccacctgccgactctgacgatcccaacaacgtaaactcggcttcttccctttcgctcgaggccacctacattacccgaaatttctcgtctcaagtcattgatgccaagtccaagccatattcgcctagccccaatccgttctattcggaggacgagccatcacccgtcgcttcctgcttgtacaggtaccgaaagttcgacctgtctgttggcgaggaagataccctggacctcattgtacgaaccgaagtcgacgcctatcaaggcaagaaggactctctcgtcactgtcaaggcattgaacgagtttgatcctcgagcttcaggtggtggcaaagccctagactggcgaaagtacctcgacactcaaaagggtgccattgtcgcctcggaaatgaagaacaactcggctaaactcgctcgatgggctatccagtctgtcttggccggtgccgaagtcatgaagatgggatacatctcgcgagcttcgcccagggatacaactcatcacgtcattgtcggtgtgcaaaattacaagccaaaagactttgccgctcaaatgaatgtgtccctcaacaacggttggggtatcgtccgaacgattgccgatcttgtcctcaagcagccagagggcaagtatgtcctcgtcaaggacccaaatgcaggcatcattcgtctctacagtgtgccagagaatgctttcgaggcagaggaggaggaggagcaatagtcgaaaagtctagacaggccgtgtcggacatgcatcatatacttcaagg

SEQ ID NO: 52 - DNA, 788 bp, unidentified
  source 1..788: mol_type = genomic DNA
gagcgctcaaggtccttggccccagaagccgatcaaggtgtcgcgactcagtgtcacgaagtcagatcgtcaggtagctacgacacgttcgagatcggccatcaccaacctgacagcgatacctcgggagtagctgacttgcgcacttccagtcgtatggacacctgcgacgcgcatcttctacggcgggtcaaaagctgccctcttttcagctaccgcgaagacgaggtgtccgaaactgtacaattgcctacaggcgaatggacgacgatcagagatatcactccgagtgcaccaaagatcggctttgaagtgcgcgactcgctctccgcgttcccgacagccaagcctgtcgaagcgaagcacgagtccgcctccagcatatccaatgatttaccctctcagccatcctcaaggccgctgattgagtgtccgacactggtcgccgattcacgcacaacgacggggtccaactctgtgcgcagtttcgacgcccagaccgaacgcctgagcggcttgagcgacgtgcaccacagatacatgcaggacaagccgtcacagcgttctgattcctggaccgacgtcaaatcctccgctccgtcctcccagtcgatggcagtccccaacaaagcggcttacctggctccgatcccagctggcccaaatgacagtaagacttcgagttccggtcgcgccccgtcagacgccgcgaccgaacacgagtgttcgctacaataagtcagacttgctgttggaacgtttcctacctcatgcatacctggcatgct

SEQ ID NO: 53 - DNA, 706 bp, unidentified
  source 1..706: mol_type = genomic DNA
accctccaaactccaagctcttttcaaccctttcctaccttacacaacaacttcaacaacaactatggcacccaagtccactgacaagcccgcatccaccgctggcaaggccccctctgctggaggcaaggctcctgcctccaagactgtcggtgctaagaagaccgcagcaaagaagtctgctaagtctactggcgagggcggcgagaagaagaagcgtgtcaagtccagaaaggagacctactctacctacatctacaaggtcctcaagcaggtccaccctgacactggtatctccaacaaggccatgcttatcctcaactctttcgtgaacgacattttcgagcgtattgccggtgaagcctccaagctcgctacttacaacaagaagtctaccatctcctcccgcgagatccagactgctgtccgcctgatcctccccggtgaactgtccaagcacgctatttctgagggcaccaagggtgtcaccaagtactccagctccaagtaaacttgtcttttgcttggctgagagtctttcccctttccttcttcattgtccctaccctcctgttcttcccctctccctcacattcatcatgttgtctattaggcgagctgcctgcagacttgctcgctgtcaaggctgaagcagtcgcgtagttagtgtaatggagccacaaatgtaattctagagcacatgcag

SEQ ID NO: 54 - DNA, 1203 bp, unidentified
  source 1..1203: mol_type = genomic DNA
gatgtcaagcgattcaccaaggatctgctgttcaactcggagggcaacctaaccttcaagccccacttgtggaacgacatccgtcacaccctcctccccacctttatccgacagatcggatacgttcccatcccacgagccgagttctcctcgcctgacattgaccttgtcatcgagaatctggtcctgtccggacccaacctcttccccaacgtcgtctcgctcgagagccacaactcgttcaagttctcgccttaccagcagctcaacaagggtatggacacgcatcaccacaagttcaggctgggtatgagccagatccaggccgatatccgagatgtccgattctcgttccgacgaaagactggatggcccaagctcaaggaccacggtctcgccgatgtcatccttgccggtaagggtatgtcgatcgacgtcgagctcgagtctgtcgagggacgacgagactctgttgtgcgagtcaaccacgtccacaccaccatcgacaccctcaccttctccatccgagactccaagcacgacttgctctacaagttcgtcaagtcggtggccacgggtacgatcaagaaggcaatccaggccgccgtcgacaatgccatccgtacggctgtcggtcacctcgacgaccagctcgtccaggtccgaaacaccgtcgatgacgccaagaagtctgacgagaccacccgaacgcaagccctcaaggacttgtactcgaagaaggcggacacggcacagaagaagcaggccgagtccaaggagcagcctggtactttccgaatcgtcgccaaccgagactctgttctcaaccccgacatgggcggtggcaagggcgccatgaccaacaagatgtggaagaccgaggaccttgcacactctggcaaggaatggcactctcccgctttcgacttgctcgactccaagcacccagcacgtaccggtcagacccaccccgaggccaaggagggtgctggacacggaaacagcttgagctcaaaggctcagcccggcgccaacgcggccgaccagctcaaggctactcacggtcagtctgaggctgaggccatcgctggtcagaagcgacagcaataggtggaagagagggagccgcgtattgagaagtaggaaggactagctgtatacccccttatacttttgtgtctatagtaatgaatgcgtgaaacc

SEQ ID NO: 55 - DNA, 962 bp, unidentified
  source 1..962: mol_type = genomic DNA
ggacctttcatccatcagcgtatcgcatatcagcttcctgacaaagtaagaggtaataacaagacaccacactctttcagcacgtacctcataccggaccgccatgtacacatccgccgtgacactgctctctttggtcttgctcctggcgacttccgttattgcacaagaacaagctggtcggcctggcactcagcgaggcggcgtcttctttgggtgttatgccgatcgacctacaggcaatgccaaccagcccatcactcgagtcgccaactccgacacattctttgaatgcatggagaattgtgctgcgataacgtctccttcgttgctgggatactatcaaccctcctccggtcaatgcttctgcggcaaccttttatttaaccctcaagctcaattgaacggtaacggttgtcaaggtagtgattggtcctttggccggacttcgaccaccttcaggaggttcggtgacgcttgtcgacctttcggtggtgtcggattttctgcgaatcaatacactacagtcactggtcccgtagcttgtcatgttcaatgcgcatcaaacagattcgcctatgtctggtccgatactggaagcaactcatggcaatgtgcttgcagcaacaatgtccgtgttcaggaggacttccagtacacttgtcaaggtggcggtgtatttgtgtttgaacattcagtacaagctcaggcttcttcgcttaacaggaagcggacggtggaggaacaatgggctgttccgaaagacgccctctgtccattcggaatgtcagcgtgcaaggtatcaggtgtcgataatgcttacgaggtatgcttcttttcagaccgctaggcccctggttccctggccacgaggtttgaaacacgccattgacctgtagtgcctcgatacctcagccgagctagaatcgtgcggtggttgtctgcatggtcaattgttctga

SEQ ID NO: 56 - DNA, 909 bp, unidentified
  source 1..909: mol_type = genomic DNA
gggggaggaacggtcgttagcaatgctttgctggagaatgccaagctctgcaagacccagggcaaggagagctctcttcgagtcatcgtgtgtggccgaaataggttggagaatgggtctgcacctcattgggccgaggcgtttgctacgcatggcaaattggtggaagtgaggatgccgcaaaacggcattcgcatggagggcatcaaagctatcgccgacggactggccaagtgtccgacattggaagtgcttgatttgcaggacaacacggctaccaagacaggaacacggagtattgtccgacacctctcaacttggcctaaacttcgaatactcaatctctcggactgtcttttgggttcggtcggcggtatcgctcttgcaaccgcattgtccactggctcgaacaagcacctcgaacagctcaaactgcaatatggcgagtttgacaagaggacggttgagatactgtcgacggcaattagccagcatttgccaaaattgacgacactcgaactgaatggaaaccgtttcgatgccgaagacgaatgcgttgagaccctgaagaaggcacttgagctacatgggaacgaggatgctttggacgaacttgacgatatggaggaggtggacgaggacgaagaggatgatgatgacgaggacgaggaggacgaagacgaggacaaggacactagcgccgacgatgggatcgatgcaggagctgctggagaagacgctctaccaccagtcacgaagaaggacgaggacgtacttgcggatctcctgtccaaggtccacgttcagcctagctgagtccaagcgctttccggtcggcaagtagatagactagacagcataataccttgaccctcatgatgccacccgcatgtacacatttgttctccg

SEQ ID NO: 57 - DNA, 909 bp, unidentified
  source 1..909: mol_type = genomic DNA
cggagaacaaatgtgtacatgcgggtggcatcatgagggtcaaggtattatgctgtctagtctatctacttgccgaccggaaagcgcttggactcagctaggctgaacgtggaccttggacaggagatccgcaagtacgtcctcgtccttcttcgtgactggtggtagagcgtcttctccagcagctcctgcatcgatcccatcgtcggcgctagtgtccttgtcctcgtcttcgtcctcctcgtcctcgtcatcatcatcctcttcgtcctcgtccacctcctccatatcgtcaagttcgtccaaagcatcctcgttcccatgtagctcaagtgccttcttcagggtctcaacgcattcgtcttcggcatcgaaacggtttccattcagttcgagtgtcgtcaattttggcaaatgctggctaattgccgtcgacagtatctcaaccgtcctcttgtcaaactcgccatattgcagtttgagctgttcgaggtgcttgttcgagccagtggacaatgcggttgcaagagcgataccgccgaccgaacccaaaagacagtccgagagattgagtattcgaagtttaggccaagttgagaggtgtcggacaatactccgtgttcctgtcttggtagccgtgttgtcctgcaaatcaagcacttccaatgtcggacacttggccagtccgtcggcgatagctttgatgccctccatgcgaatgccgttttgcggcatcctcacttccaccaatttgccatgcgtagcaaacgcctcggcccaatgaggtgcagacccattctccaacctatttcggccacacacgatgactcgaagagagctctccttgccctgggtcttgcagagcttggcattctccagcaaagcattgctaacgaccgttcctccccc

SEQ ID NO: 58 - DNA, 596 bp, unidentified
  source 1..596: mol_type = genomic DNA
ggatggtgaagcttagtaacagtcttgtccgtcgtcttaaatggcaacacgttcgcagtctcggcgtggtggcgctgactgcccaattgcgaggaccacaacctcagagcgccgaggacgaagattctgaagcagctggcaagaagctcaaactggctggcgaccaagctacatctgcggtcattcccaagtccgcagacaagcccgatactttccctctactcgacacactacctgctactatggctgctggcaccaggtctatgactaggcccttgcatgtcggtgatctgaggttggctgatctgcgtaaaatcatgcaggcagctggccacacggctgagttccgaggtgagggaacactactcattgacaagtccgtcgctgtcagaaaatcaggcacagggcagattgaaatcgaggcatctgctcaagcagctgcaaaccaagctactcctggccgaggtgcgagtagcttcctcgctgtcaaaagaaagatatacgagggtctcgctgttgtcacaggaagttaaatgaccgtgtaccctatattcaatttttgtataatttacgcaataccaacgatattctctcgt

SEQ ID NO: 59 - DNA, 418 bp, unidentified
  source 1..418: mol_type = genomic DNA
gaaaatgaaaattgatgtggagaagctgaataaagatatcagccttttcccgcaggtgcatccgattacggaagatatgaaaatcacgcacaaaggtgtttcgcgccttgtaatgctggacaggtattcatttaaagacactgaaaaaattacgctatctgaaggcgattttgtagtgctgacgatcaaggaagatccaaaatttcctgcaagagggctaggctacattaaagaaattgattgggaaaataaaaaggcaaaggttcaggtcgaagaagagtttcgtcatactcttgaaaagcctgaagaacgggagacgggaatcatcgttcgctctttagatgtcatcgaaaaaccgcttgaaattttttatgaacaaattgccaaaagaaatgcaacaggtcttgctgctgttgaa

SEQ ID NO: 60 - DNA, 988 bp, unidentified
  source 1..988: mol_type = genomic DNA
ggggatgcaacggtgactcaactgcgcgaaatcatggacgacccagctggctatttcttgccaaatctcaaacatggcgccgataacatgttctacgtcggtccacgcggacttgcacaagagctcgaggagctttttaccttcccaagcacaatcctcagaaagcgccaggataccagtcagcatgacgaaaggcaggcaaagaaggcgcgcacgcaagaggacgaagcggctggtgacgcgttggaggagcccgagactgggcgacgcgacagtgtgcttccgactgaacgggccgcttttggtctcgagggtgatgactcgggctttttccttggcgaccagacgatgggagacgacatgctgcctatggacgacatgggagccatggacaccggagtggaccagcgacgcatgcgaacaccatcagtcgcaccgtcggtcaccgaatcgatcgcacgtcagattcagaatgaccgaagcgctggcacacacccactggctatattcgagaaggaggcaagggacgacacgcagtcgcaatcgcaggctacgcccaacaaatcggtggcctccgagtctatcagcaagacttcttctggccaatcaaagaatactggcatggccatgggtttgttgcgaagggagattgaggcgatcgaggaggaagacaagatggtcgggtttgatcacttggcagacaaggcgtccaagcgagcagcgtctgcattcttcttcgagctgttggtgcttggtaccaaacatgcggtcaagcttgaacaagctcaggctttcggcgacatccacatacgcggcaaagacaagctgtttgcagaggttgttgcatagacaaacttgaagagccacgatcttacgcgcaacggagggagatctaatgaccatcttgatgtcgactttaatgttatttggtacttgtacatgagctgctaagagggtcttgaatgagatgatgcatcgcttcatgagg

SEQ ID NO: 61 - DNA, 614 bp, unidentified
  source 1..614: mol_type = genomic DNA
gcagaccgtctcttttaaatctcctccttgacacccgtctcctttgcacatttactacactccacatatctccataacaaccttatatctttacacaatgggtgaccacgccactaccaacgacccctccaacgccaccttcgaggagaagggcaagggcaaggacgtccaggatcaaatcgcggaggactccagcgacgaggagagtgaccaggagcctgagatggttgacgaggaagaggatgacaacaacctcgagcccatctcccaagacaacatcatctcaggtggtcgccgtacacgcggcaagatcatcgattatgccgccgaagccgagaagaacaaggatgagatggaggactctgaggatgacgaggattaccaaggcgctaatgacgacgaggatgaccagatgcgcgactaagcgcatggtcttgatgacggatctcaattaacataggactttggaggattggcgctatggtttctgaaggaggttctctcgtgcgcctttgtggttagcatctcacctatgaaatgtcatggcctgagcctggcaatggacatgactatgaataaatgaaatgaagcctgcttctgtctttgtgtaacag

SEQ ID NO: 62 - DNA, 418 bp, unidentified
  source 1..418: mol_type = genomic DNA
gaacttaagtattttaaagcagttgcactatacaacctgagccggtacttggatgcacggaaagcaatcaatgacctcattcagagctacccggacttccggcaagctgaggccctcaagtcagccattgatgacaaggtggtgcgcgatgggctgattggcgtgagtgtggcaggagcagtggtggctggcgtcgtgggcttggctgtggctcttgcacgtggcaacagaggatgatgctacaggaggcagcaggttgttggacagttcagtgcaccgtgccaatgcttcaacggtctggcacaggaggcagcaggttgtgaccctgcacaagcttgggccatgattctacagacacaccttatggcaatcaaatgtgtgtttgcatgtgcgttgaagagtgtaaatgtgctcttcc

SEQ ID NO: 63 - DNA, 94 bp, unidentified
  source 1..94: mol_type = genomic DNA
tatcccgagtagcatgggacacgtggaatcccgtgtgaatcagcgaggaccacctcgtaaggctaaatactcctgggtgaccgatagcgaaaaa

SEQ ID NO: 64 - DNA, 371 bp, unidentified
  source 1..371: mol_type = genomic DNA
cacgatttaaatacccgggggacgttttggattcgacagggatagatcgagcttaagctgcgagccggagggatcgtctccgtcatcaacgtcgcctaaagataactggcaaacaaaacaactacgctttagctgcttaatgctaaaggctcctttcttccatcgcccatgtggaggaaaaggggttcaacttaagtgggctacgcccgattccgccgtctgaggaagagggaagagacgaatcagactagctgtccggatgcctgccgacaggctaaggaacagtgaaatgttaaatatgtcggatacgctcgtagatgcttaagtggcgatatctctggacgtgggttcgattcccaccgtctccacca

SEQ ID NO: 65 - DNA, 681 bp, unidentified
  source 1..681: mol_type = genomic DNA
gccatcagcaccgcaaagctacctcatcaaccattgaaagcacgcaaataactccccaaagttaatgcccgtacgaccctacctggaagaaatggccgacatgcccgtgcccctgttcgcgtacgacgcaccgcccaccctggccgaccaccctcacgcccgcgagcaccaacacacgaccttcatgcaataccttgcgcgcaagcagccggacccaaagaactaccccaactaccctgacgtggacatccgcgacgccatcaatcactacctgatcgagctcgaatgcccggggatcaaagacgcagccgacatccactgccagtggacgagctcgcggcacctgaccgtcaccggcgacatcgcccgtcctgaggaaagccagatcgaagcgcagatcgagagcaggcccgtctacctggttctgggagagagacgcattggctctttccgtcgcaactttaccttccctgtggaggtcgagcaggaaaatatgactgccaagttggaggccggattgttgaagattgtcttgcccaagcacaagcaccatactccgaagggaacaggaaaggtcgacattgatgtcattgagtgaacgtcttttgggtctgcgattatatgcgaggagttcttagattgccggagtgggtacctgtatgggaattatgtatctgcaac

SEQ ID NO: 66 - DNA, 681 bp, unidentified
  source 1..681: mol_type = genomic DNA
gttgcagatacataattcccatacaggtacccactccggcaatctaagaactcctcgcatataatcgcagacccaaaagacgttcactcaatgacatcaatgtcgacctttcctgttcccttcggagtatggtgcttgtgcttgggcaagacaatcttcaacaatccggcctccaacttggcagtcatattttcctgctcgacctccacagggaaggtaaagttgcgacggaaagagccaatgcgtctctctcccagaaccaggtagacgggcctgctctcgatctgcgcttcgatctggctttcctcaggacgggcgatgtcgccggtgacggtcaggtgccgcgagctcgtccactggcagtggatgtcggctgcgtctttgatccccgggcattcgagctcgatcaggtagtgattgatggcgtcgcggatgtccacgtcagggtagttggggtagttctttgggtccggctgcttgcgcgcaaggtattgcatgaaggtcgtgtgttggtgctcgcgggcgtgagggtggtcggccagggtgggcggtgcgtcgtacgcgaacaggggcacgggcatgtcggccatttcttccaggtagggtcgtacgggcattaactttggggagttatttgcgtgctttcaatggttgatgaggtagctttgcggtgctgatggc

SEQ ID NO: 67 - DNA, 50 bp, unidentified
  source 1..50: mol_type = genomic DNA
gggatgatatgcatcatatagatcttgagaaatcaaggtaagtatacaaa

SEQ ID NO: 68 - DNA, 558 bp, unidentified
  source 1..558: mol_type = genomic DNA
gggaaaaaaactttagaatacagtttaatcaatcttcacagctacaaggctatatcatttgatatagcatatcaaagtggctttgatttctgtaaatttatatctaataataatagtgtttatatcagctaaatacatatttctatcctatctatatatcaccgacagaccatatttgaaactgctgttgacactattattcatatgttcggatttaattttaatacgacaaaattgttaaaaacaattctcgttgtttgttatttgcaggcaacagtgttagctgatccttatacaagagtatcttgggaagcgtatatgaatcatgtcaatggatccgacgactatcgtactcaaggggatgataccagagctacacgctttccagagactaaacctccaaaacaaggaaaagatttcctgtggtcgagtaaaccagtccccagttcagatctatttctggagttctttatgtatgagggagaaccagatgaattcagcaggacgactgaatcgtatcaatcacttccgagcaacgcgttaactgctaggcaaaaa

SEQ ID NO: 69 - DNA, 1164 bp, unidentified
  source 1..1164: mol_type = genomic DNA
ggacacatcacacaacaacaatgtctccaacaccaacatcaccacacaacaagctctcgctccccgcaagagcttcttcccacgactcgacagacggcatccgtaagcgagtatgcaaggcttgcgacaggtgtcgattgaagaagagcaaatgcgacggatcaagtccatgctctcgatgcaaagcagacaacgccatctgcgtgttcggagagcgcaaacgatcacatgataaacactatcccaaaggctatgtcgagatgctcgaacaacagcagggtcagctcgtctcaggcctcaaagaaatgtaccacagactccagaaagcctccgcctgggatggccctgtgttggacgaaagcaccggacagcctctcactcacgacatcctgtcagcattagacctcctcgaaccaaagcatgacgacagcaacgagccagaagtcttcgaagagaactgcgaaaagctgcaatcaaaattgctcgcagacggcgcgggctttgcccaccgacgaggatcgatcagttcggattctgaacacagccatcacgatcgacccaaaacatcctcacgccacgacacgcccgtgcaacccaaaccgtcgatcttcaaggagaacctgagcttcgccagcgcggcctcatcaccactcacgcaaagccccatccctcgatcgaaacccttgaacgtcatgccataccaaacgctgcaaccgtcgtcaagaccatccccactccagatgccctcagcatacaacgacccgcaactatacgcacccgaatgggcacaagcactggcagacatgagcggcgatcccaactaccgccaaagattctccatgcagcagcaacaacaaaacgacttcgacaacctgctctgggatccctcagcgcaagcgcccatggaatcgcccttcagccaaccagccttcttcaaccaggcgcaactgatcggcagcggcaacgtctttgggctgtctgacatcaacgatctgggccccaaccccgcggatggcgggatggactttgacttcagcaagttcgtgcagcagaccgaagtcatgacatgaacatgattcttgccttctgtcaatacgcgcgagaattttgcttcagagttccagtccgtgtaattcttgtgtatttattacgatacgaacacgc

SEQ ID NO: 70 - DNA, 923 bp, unidentified
  source 1..923: mol_type = genomic DNA
agagctcttcgtactccacaccaccatcttccatccgaccacactttcatcccaaatccatcaacaacccatctcaactccatctcaccacctcaccatcatcacaatgtcttccttccgcgtcgccgcccccaagatggcctccatggccgctcagtcctccgtgaaggtcgcccgcccggccttccaggctgctcagctccagaagttcacccgcgcctactccgcggtccccaagaacaccgtcttcaacaccatgaagcgcacccagatgatggcccgccaggcctcccccatcgccaagcgtgcctactcctctgagatggccaacgccctcgtccaggtctcccagaacatcggtatgggttccgccgccatcggtcttgccggtgctggtgtcggtatcggtctcgtcttcgccgccctcatccaggccgtcgcccgcaacccctccctccgtggccagcttttctcttacgccattcttggtttcgctttcgtcgaggccatcggtctcttcgacctcatggttgccatgatggccaagttcttgtaaaaatgtgcattccattacctaccgagatggagatggatgcgaaggcgattggggacggagacagtgcgttgctgcagcagcattagtaccggtgttggtcgtgtaccagtagtctgatggagacggatagatggatagaaagctggtgaatgggggctacgaagaaaacgtacctctcgatccatttgtacccatactcatgaagtatatccgtcttctttccttctatcattcgcgcgcacttccttgctggtggctttttggggttgcgctctcaccgaaaagcaacgtcactcttgtatataacttattcgaccacggccatatcttggtttggctggggaaataacaatgtctcatttgtacc

SEQ ID NO: 71 - DNA, 923 bp, unidentified
  source 1..923: mol_type = genomic DNA
ggtacaaatgagacattgttatttccccagccaaaccaagatatggccgtggtcgaataagttatatacaagagtgacgttgcttttcggtgagagcgcaaccccaaaaagccaccagcaaggaagtgcgcgcgaatgatagaaggaaagaagacggatatacttcatgagtatgggtacaaatggatcgagaggtacgttttcttcgtagcccccattcaccagctttctatccatctatccgtctccatcagactactggtacacgaccaacaccggtactaatgctgctgcagcaacgcactgtctccgtccccaatcgccttcgcatccatctccatctcggtaggtaatggaatgcacatttttacaagaacttggccatcatggcaaccatgaggtcgaagagaccgatggcctcgacgaaagcgaaaccaagaatggcgtaagagaaaagctggccacggagggaggggttgcgggcgacggcctggatgagggcggcgaagacgagaccgataccgacaccagcaccggcaagaccgatggcggcggaacccataccgatgttctgggagacctggacgagggcgttggccatctcagaggagtaggcacgcttggcgatgggggaggcctggcgggccatcatctgggtgcgcttcatggtgttgaagacggtgttcttggggaccgcggagtaggcgcgggtgaacttctggagctgagcagcctggaaggccgggcgggcgaccttcacggaggactgagcggccatggaggccatcttgggggcggcgacgcggaaggaagacattgtgatgatggtgaggtggtgagatggagttgagatgggttgttgatggatttgggatgaaagtgtggtcggatggaagatggtggtgtggagtacgaagagctct

SEQ ID NO: 72 - DNA, 1368 bp, unidentified
  source 1..1368: mol_type = genomic DNA
ggaggattctcggtcaagttcaggaccgcagaaggaaattgggactttgtggccaacaacacccccgtcttcttcctccgagacccggccaagttcccccacttcatccacacccagaagcgagatcccgccacccacttgtctggtgacgatgactcgaccatgttctgggactacctgtcgcagaaccccgagtcgatccaccaagtcatgatcctcatgggtgatcgaggtatccccaagggctggcgattcatgcatggctactacggccacaccctcaagattgtcaatgacaagggcgaatgggtctacgcccagttccacctcatctctgatcagggcacccagaacttcacgggtgacgaggctgctcagcaatccaacgattacggacagaaggatctgtacgaagccatcgagaagggagacttcccctcgtggacgatgaaggttcagatcatgaccgagaagcaagccgaggaggcatgggagcaaaagaggatcaacgtctttgatttgacccacgtctggcctcatggtgattacccacttcgaacagtcggtaaattcacccttaacgagaatgccaagaactacttcgccgaggtggaacaagtcgcattcaacccgtctcacatgattcccggtgtcgagccgtccaacgacccagtgttgcagtcgcgactgttctcttaccccgatgctcaccgacaccgaatcggagccaactatcagcaactgcccgttaaccagaatgtgtgccccttcgccttgggcaacttccagcgagacggccagatggcattctacaatcaaggtagtcgacccaactacctttcttcgattgagccaatctcattcaaggagagggcgtatgatctcaacaaggtccacggcaaattcgtcggagaagccgtcgccttcttgtctgaaatcaggccagaggacttcaatgccccaagggcactgtggcagaaagtctttagcgaggaaagcaagcagcgattcgtcgacaccgtctctggtcacatgtcgacagtccgagacaaagccatcaccgctcgaatgatgactatcttccgagaagtttcgcctgatcttggtgatcgacttgagaaggccactggtgtcaagggcgaatccaccattgccgggatgaagttcaacggaacgcacaatgggtttgacaaggccaacaagatcccggctaatgggatgaagaagggtggagaagtcatctttgacaatggtgcacctgctactgctgccaggtaaatgagcggtcaggcgtacttgatatatgttgttacgatatgtcggtctcgtagtcatgtagccaggataaatgaagcggatgtggcagtg

SEQ ID NO: 73 - DNA, 1368 bp, unidentified
  source 1..1368: mol_type = genomic DNA
cactgccacatccgcttcatttatcctggctacatgactacgagaccgacatatcgtaacaacatatatcaagtacgcctgaccgctcatttacctggcagcagtagcaggtgcaccattgtcaaagatgacttctccacccttcttcatcccattagccgggatcttgttggccttgtcaaacccattgtgcgttccgttgaacttcatcccggcaatggtggattcgcccttgacaccagtggccttctcaagtcgatcaccaagatcaggcgaaacttctcggaagatagtcatcattcgagcggtgatggctttgtctcggactgtcgacatgtgaccagagacggtgtcgacgaatcgctgcttgctttcctcgctaaagactttctgccacagtgcccttggggcattgaagtcctctggcctgatttcagacaagaaggcgacggcttctccgacgaatttgccgtggaccttgttgagatcatacgccctctccttgaatgagattggctcaatcgaagaaaggtagttgggtcgactaccttgattgtagaatgccatctggccgtctcgctggaagttgcccaaggcgaaggggcacacattctggttaacgggcagttgctgatagttggctccgattcggtgtcggtgagcatcggggtaagagaacagtcgcgactgcaacactgggtcgttggacggctcgacaccgggaatcatgtgagacgggttgaatgcgacttgttccacctcggcgaagtagttcttggcattctcgttaagggtgaatttaccgactgttcgaagtgggtaatcaccatgaggccagacgtgggtcaaatcaaagacgttgatcctcttttgctcccatgcctcctcggcttgcttctcggtcatgatctgaaccttcatcgtccacgaggggaagtctcccttctcgatggcttcgtacagatccttctgtccgtaatcgttggattgctgagcagcctcgtcacccgtgaagttctgggtgccctgatcagagatgaggtggaactgggcgtagacccattcgcccttgtcattgacaatcttgagggtgtggccgtagtagccatgcatgaatcgccagcccttggggatacctcgatcacccatgaggatcatgacttggtggatcgactcggggttctgcgacaggtagtcccagaacatggtcgagtcatcgtcaccagacaagtgggtggcgggatctcgcttctgggtgtggatgaagtgggggaacttggccgggtctcggaggaagaagacgggggtgttgttggccacaaagtcccaatttccttctgcggtcctgaacttgaccgagaatcctcc

SEQ ID NO: 74 - DNA, 1377 bp, unidentified
  source 1..1377: mol_type = genomic DNA
ggacaccattgacgcagaggtgctcgacagtttgggtgtcacccaagagaacttccagtttgcccttggcgtcagcaacccctctgcccttcgcgaggtcgcagtggtcgaggttcccaacgtcagatgggaggacattggtggtctcgaggaggtcaagagggagctcatcgagagcgtgcaataccccgtcgaccaccccgagaagttcctcaagtttggcatgtccccatcaaagggtgtgcttttctacggtccccctggtactggtaagactcttctggccaaggctgtcgccaacgagtgcgcggccaactttatttccgtcaagggtcccgagcttctctccatgtggttcggtgagtctgagagcaacattcgtgacatcttcgacaaggctcgtgctgccgcgccttgcgttgtcttcctcgacgagctggactccatcgccaagtctcgtggcggttctcagggcgatgctggcggtgcttccgaccgtgtggtcaaccagcttctcactgagatggacggtatgaccagcaagaagaacgttttcgtcatcggtgccaccaacaggcctgagcagctcgacaacgctctctgccgtcctggtcgtctcgacactctcgtctacgttcccctgcctgaccaggagggccgtgagagcattctcaaggcccagctccgcaagactcctatcgccgacgacatcgacctttcctacatggcctccaagactcacggtttctctggtgccgatcttggcttcatcacccagcgtgccgtcaagctggccatcaagcagtctattgacctggccatccagaaccaaaaggctagagaggccgagggtgacaccgccatggacgaggacatcgaggaggacgaccccgtgcccgagctgaccaaggctcactttgaggaggctatgagcatggctcgtcgctccgtcaccgacaccgagatcaggcgctacgaggctttcgcccagagcatgaagagctccggtggcggcagcgctttcttccgcttccctgagagcggtgccgatggcaacgcagccgagcagcagcaaaatggtgctggcgaggaggacctctacgactaaattggtttcacgaacctcacgacctaatcctttgctgttatcggagtaatattccagatggagagagcaatcatgcattcaggcgcgtctatggactgaaggggaagatggatagagtgttccagtagcccttttctctttttttctgggaactcttgctgtttggctggtcgcctcttatcgagtgtggttgtgctagagtaggcagttcagagtttccctgttatgttatgcctttccgggcagtatgagaataatttccttgcaaa

SEQ ID NO: 75 - DNA, 1377 bp, unidentified
  source 1..1377: mol_type = genomic DNA
tttgcaaggaaattattctcatactgcccggaaaggcataacataacagggaaactctgaactgcctactctagcacaaccacactcgataagaggcgaccagccaaacagcaagagttcccagaaaaaaagagaaaagggctactggaacactctatccatcttccccttcagtccatagacgcgcctgaatgcatgattgctctctccatctggaatattactccgataacagcaaaggattaggtcgtgaggttcgtgaaaccaatttagtcgtagaggtcctcctcgccagcaccattttgctgctgctcggctgcgttgccatcggcaccgctctcagggaagcggaagaaagcgctgccgccaccggagctcttcatgctctgggcgaaagcctcgtagcgcctgatctcggtgtcggtgacggagcgacgagccatgctcatagcctcctcaaagtgagccttggtcagctcgggcacggggtcgtcctcctcgatgtcctcgtccatggcggtgtcaccctcggcctctctagccttttggttctggatggccaggtcaatagactgcttgatggccagcttgacggcacgctgggtgatgaagccaagatcggcaccagagaaaccgtgagtcttggaggccatgtaggaaaggtcgatgtcgtcggcgataggagtcttgcggagctgggccttgagaatgctctcacggccctcctggtcaggcaggggaacgtagacgagagtgtcgagacgaccaggacggcagagagcgttgtcgagctgctcaggcctgttggtggcaccgatgacgaaaacgttcttcttgctggtcataccgtccatctcagtgagaagctggttgaccacacggtcggaagcaccgccagcatcgccctgagaaccgccacgagacttggcgatggagtccagctcgtcgaggaagacaacgcaaggcgcggcagcacgagccttgtcgaagatgtcacgaatgttgctctcagactcaccgaaccacatggagagaagctcgggacccttgacggaaataaagttggccgcgcactcgttggcgacagccttggccagaagagtcttaccagtaccagggggaccgtagaaaagcacaccctttgatggggacatgccaaacttgaggaacttctcggggtggtcgacggggtattgcacgctctcgatgagctccctcttgacctcctcgagaccaccaatgtcctcccatctgacgttgggaacctcgaccactgcgacctcgcgaagggcagaggggttgctgacgccaagggcaaactggaagttctcttgggtgacacccaaactgtcgagcacctctgcgtcaatggtgtcc

SEQ ID NO: 76 - DNA, 699 bp, unidentified
  source 1..699: mol_type = genomic DNA
gaaggtgacgacgagcagactttgcgccccacgacgatactaacgtaacgacccagcacacattaatccacaatgggtcactccgccggtctcaggaagggcactcgctatgccttctctcgcgacttcaagaagaggggcatgatccccctctccacctaccttaagcagtacaaggtcggcgacatcgtccacgtcgtctgcaacggtgccgtccagaagggcatgccccacaaggacttccacggcaagactggtgtcgtctacaacgtgaccaagtccgccgtcggcgtcatcctgtacaagcaggttggcaaccgttacatcgagaagcgcgtcaacctccgcatcgagcacgtccgcctctcccgctcgcgtgaggagttcatcgtccgcgtcaagaccaacgctgagaagaagcgcaaggccaaggaggagggcaccaccgtcttcctcaagcgccaggccgacaagccccgcgaggcccgcaccatcagcgccaaggacaacaagcccgagagcatcgctcctatcgcctacgacacccacatttaagcgtgcttgtttcgaaagggagggcgtacgggctggtatgatggcgaggctaggaggttggtatcggcggatcggattccaccggatgggaaatacctgccggatgagccagctagcttcgcaaggtgcatgaattctagcgcc

SEQ ID NO: 77 - DNA, 1664 bp, unidentified
  source 1..1664: mol_type = genomic DNA
ggggacatgggcatcggtggtcttgatacggagttctcggctatcttccgacgagcatttgccagtcgtattttcccgccgggactggtcgagaaattaggtatccagcacgtcaagggtatcttactgtttggcccgccaggaacaggaaaaaccttgatggcacggcagatcggaacgatgctcaacgccagagagcctaaggtggtcaacggtcccgaaatcctcaacaagttcgtcggtcagagtgaggagaatatcagaaagctgtttgccgatgctgagaaagagcaaaaggaaaagggggatgaaagtggcttgcacatcatcatcttcgatgagctggacgctatctgtaaacagcgaggatctacaaacagcggtaccggcgttggagactcggttgtcaatcagctgttatcgaagatggacggtgtagatcaactgaacaatgtcttgatcatcggtatgactaatcgaatggacatgatcgacgaagcgctcctccgacctggacgtctggaagtccacattgagatctcgttgcctgacgaagctggccgattccagatcctcaacattcataccaacaagatgaggacgaatggtgtcatggacagcgatgtggatctgggcgaactagcggccctgacgaagaacttctcgggtgccgagattggtggtctggtcaaatcagcgaccagtttcgctttcaaccgtcacgtcaaggttggctccgtcgccgcgtttgatgatatcgacaatatgaagatctcacgagccgacttcctccacgccctagacgaggttacacctgcgtttggtgtctccgaagaagagctgcaacaggtcgtgcagaacggtatcattcactactcgcaacacgtcaatgacacactaaacgatggaagtctgcttgtggagcaagtgcgaaaatccgaccgcaccccgcttgtctcggccctccttcacggtccatctggcgcgggcaagacggctttggcagccacgatcgccatggcatccgagttccctttcatcaagctcatctcgcctgaaacaatggttgggttttctgagccgcagaagattgctcaactcaacaaggtgttcacagacagctacaagagtccgatgagcatcatcgttgtcgacagtctcgagagattgctggactggaacccgatcggacccaggttctcgaatggtgtgcttcaggctttggttgtcctctttggcaaacgtccgcccaagggtcggcgtcttctcattctggccaccacgtcaaatcgcaacatcctcacggatatggacgtcctttcggctttcgacactgatatccccattaaccccatctcatcgatcgatgcagtggtgcacgttctagatgaggtcaagttattcccgaactcgaaggaaaagcagagagcaacacagatgcttcgcgaggcgagactgggcgaaggtggtcgaccagacttgttggtcggagtgaaaaagctgttgagtatggcagagatggcccggcaggatccggatcccacgatgaagatcgtgacgagcattctcagggaggcgagttaggatgtgtgaagcgtgatcatgatagagtgtagtccaaacaatgtactagtgcaacagaagctatgcagatgaataacgtt

SEQ ID NO: 78 - DNA, 1368 bp, unidentified; identical to SEQ ID NO: 72
  source 1..1368: mol_type = genomic DNA

SEQ ID NO: 79 - DNA, 1280 bp, unidentified
  source 1..1280: mol_type = genomic DNA
gagcacatacacacaccaccgcaatcatgcctccccgccaaccagcaacacggctctttgccctaccgcctcgcttcctctgcccttcgctgcccaccacgcaaacgcgcaccatccgctccatcgacaaacccgccccaaaacccagccgattcaatgcctcactcaatctccccgtgctgggctcctcgtccaccgccgccttcgcgcgcaaagagcactcgctccccctgcgcaccggcgcgctcgccatcaaaaagggcatgacggcactcttcgacccggtcacagcgaagcgcacgccctgcaccgtcctgcaactcgacagatgccaggtggtcagccacaagcgacgcgacatccacggctactgggcggtgcaagtgggcgcgggcgccaaagaagcgaggaacgtcacgcggccggagaggggccacttcgccgcctacaacgtgcccttgagcaggcacctggccgagttcagagtcaagaacgccgagggcctgccccccgttggctcggctattaccgccgacctgttcatcgagggccagttcatcgatgccaaagccgaccgcagaggcatgggtttcgagggtggtatgaagcgctggaacttcggcggacagcccgcgtcgcacggtaactcgctcgcgcacagattgatgggttcgtccggtggtggacagggcagcggtagcagagtcttgcccggcaagaagatgccgggtcgcatgggtggcgagcaggcgaccgttgcgaacctgagggtcatgcaggtggacaaggagaacggtatcgtggttgtgagtggcgctgtgcctggcccgaagaactgcatggtcaagctgcaggatgcgctcaagaagccttggcctgatgcgacttggccgccgtctattgagggcgcgacggaggttctgagggaggccactgagaaggcgcctgctgcgtaagggggtcggtcgaggtcaagaaatatcgttgcaatttgggagatgatgctgtccgatgcctgtcgaaaaggggttcttgtggggaggtctggagaatcatcgatgcaagcattaacatgagcgtgatctcacgagcaatcccagagaagcggttacagctgcttgctcgaaatgtacactgctcaaagcttgccggagaagttggccaaagtcatcactctcggcacaggaatatactttgtaaccatagggaaaagaggagagggtctcgagccaggatcaaaaataggaaatgtacattataattgcatatcgtcatcatcc

SEQ ID NO: 80 - DNA, 677 bp, unidentified
  source 1..677: mol_type = genomic DNA
ggaatcgacgaacgacacctcaatcgaaaccaccactcgccattgtgaatctttccacctgtcgcaatgggtatctgggacgctttcaccgatattgtcgaggctgtgacgccatggagcgtcgttgaggccgaggctcctgctgaggagccccaggaggagaacgagtccaagaccgagtccaaggacgagcccgaggaggaggaagaggatgaggaagaagaggaggatgaggatgatgaggaggagctcgtcgaccccaaggagactctcgaggaagagtgcaagaactctcctcaatgtgcccccgccaagcaccacttcgacgagtgtgttgagcgcgttcagcagcaggagagcgagggtggtgctaaggaggactgtgtcgaggagttcttccaccttgcccactgtgcgaccgcttgcgccgctcccaagctttggtctcagctcaagtaaactcacaacattgggttatcggttactacgacgacgcaatggctacatacacgtcgaaaagatgcctggagccggaacgaggcaatgctgcccactacggaaggctgttcccttgtacgaatgctcatctgccgggtatcaagtcggccagagattactctgatgtcgactctctctgtaccatacgctcttacgcctgaatagatttcttgcacttt

SEQ ID NO: 81 - DNA, 1019 bp, unidentified
  source 1..1019: mol_type = genomic DNA
gggagatactaccgtgcgcccgagatcatgttgacatggcaaaaatacgatgtcgccgtcgacatttggagcacaggatgtatcttcgccgagatgctcgagggaaagcccctgttcccgggcaaggaccacgttaatcagttctcgatcatcacagaattgctcggcacacctcctgacgatgtcatccagaccatcgcatctgagaacaccctccgattcgtccagtcgctgcccaagcgtgagaaggtcccattcactacgaaattcgccaatgccgacccgctttcgcttgacttgttggagaagatgcttgtcttcgatccacgtacccgtatctcggcatcagaagggctgtcgcacgagtaccttgcgccataccatgacccgacggatgagcccgtcgctgccgaggtgtttgactggagtttcaacgatgcggatctaccagtagacacctggaaggtcatgatgtactccgagatcctggacttccacaacttgggtgatatccagcaagaccaggccgccgaaggacccgtcactggcgacctagccccaccttccgctacgacttcggcatagacagcttgcctttaggggtttttttctcgtttttctcttctcgtctcattacgttcctagtcaacatgtgtccattagcatcccaaattattggtggtagaaaggagggaaggaattggtgcaacatgatctctcctagaaaatcgtttctcttcatctctcgtccatgatccacgctttcccaagctttatctcccccttccccttcctcacgcctcaacttctcctgtaccaacaaatcttcgctaccgctttctcgaccgtcgaacgaacatcacaaagaatcaagaaaggtagaagaggtgtgaatagaccaggaaaggcattcttggagcgaggggggaggaggaagtaatctggaacgaaagcccatcacactgttttctttgaacctacatacacggacagaggggaatgcatgtgcatggtaatgt

SEQ ID NO: 82 - DNA, 391 bp, unidentified
  source 1..391: mol_type = genomic DNA
cgctaccggtcgccggcgcgccggggttgacctcggatcaggtagggatacccgctgaacttaagcatatcaataagcggaggaaaagaaaccaaccgggattgccctagtaacggcgagtgaagcggcaagagctcaaatttgaaagctggccccctcggggtccgcattgtaatttgcagaggatgcttcgggaacggcccccatctaagtgccctggaacgggccgtcatagagggtgagaatcccgtctgggatggggtggccgcgcccgtgtgaagctccttcgacgagtcgagttgtttgggaatgcagctctaattgggtggtaaatttcatctaaagctaaatattggccggagaccgatagcgcacaagtagagtgatcgaa

SEQ ID NO: 83 - DNA, 391 bp, unidentified
  source 1..391: mol_type = genomic DNA
cgctaccggtcgccggcgcgccggggttgacctcggatcaggtagggatacccgctgaacttaagcatatcaataagcggaggaaaagaaaccaaccgggattgccctagtaacggcgagcgaagcggcaagagctcaaatttgaaagctggccccctcggggtccgcattgtaatttgcagaggatgcttcgggaacggcccccatctaagtgccctggaacgggccgtcatagagggtgagaatcccgtctgggatggggtggccgcgcccgtgtcaagctccttcgacgagtcgagttgtttgggaatgcagctcaaattgggtggtaaatttcatctaaagctaaatattggccggagaccgatagcgcacaagtagagtgatcgaa

SEQ ID NO: 84 - DNA, 124 bp, unidentified
  source 1..124: mol_type = genomic DNA
gggtctggtggcgatagcgagacggccacacccgttcccatgccaaacacggaagttaagcgtctcagcgccgaaagtagttgggggatctccccctgtgaggataggacgttgccaggcaaaa

SEQ ID NO: 85 - DNA, 717 bp, unidentified
  source 1..717: mol_type = genomic DNA
agatggagcctgaccaagaagagtctgaagaggaagaagaggaagaggatgacgagatggatgaagatgaggatgagggccagcagcaggacgccagtggcatgcagacaccctctgggctcgccacgccctcaggctatgcctctactacatctacaatgcctggtggcatggagacgcctgactttatggacttgcgcaagcagcgacagacgcgcgacgagaccgctgatcaagaggaccagggtgcaccgcgagacctctatacggtcgtgcccgagcgcagagccaccgcttctggcttcctcggttctgaccgcgcctatgacttgtccaatgcgccacagtcttccaacatgcctgtgttgggtcaagaagactcgcgcaagaagaaaggcggcagatctggtgcagacgacgtcgacctggccttggatccagctgagctcgagggcatgtctgagcaagagcttaggcagaagtacgactcgcacaggcgctcctcgtccagtcaaggcgccggcggacagcaggacaaagaagatttctcagatttcgtcgcgcaagaggtcgcaaagaagaggcagagggctcagcagcgcggcggcagtggacgcgaccgcgaaagctctcggagcaaggaaaagttcaagttttagagtgtatgtttgtattgtatgaagatcagacaaaaatgctatgggtggcgttgctgct

SEQ ID NO: 86 - DNA, 872 bp, unidentified
  source 1..872: mol_type = genomic DNA
cgtgtagcataaagctagaagtaatattcacagctaactctacagtagaacaaagttcttgtttcgttctccagatccaagtgatagcaactgcttcacaaggatggaagaaccaaattgttgaaagattcggcacctaataggcggcacccatgttgaaatcgtcagtcatgttgaaactccgtaaacccgcttttggtgccctctaatccgcaaaaccttgaaaccctatgttgaacttttggtatcatcttcgtaatcgtcataaaacagaactccccgtgccagaggcggcaggttgagaataccgccccttgaattaacacattataggaagtggaacaaaggaaaaatgagaaatgttaatgcgcacagaattactgagtgtacttctggcggtagaacttagcagcctcgaccagagaggtcagctggccgatagtctgctcagtggtacgggtcttcagaccgcagtcagggttgatccagagctgctcaggcttgaggtactggagcatctgctcgatacgctccttgatctcatccacggagggaacacgaggagagtggatatcgtagacaccaggtccaatgtgggcggggaaactctgatcaacgaagacctggaggagcttggcatcggacttgctgttctcgatggacaaaacatcggtatcaagggcagcaatagcgtggaagaagtcctggaattcactgtagcagaagtgggagtggacctgggtgctgtcggtgacaccagcagtagacagcttgaaagcattgacagcccacttaacataagcatcacgggcagcgccagtacgcagaggaagaccctcacgcagggcaggctcgtcgacttggatgacccc

SEQ ID NO: 87 - DNA, 872 bp, unidentified
  source 1..872: mol_type = genomic DNA
ggggtcatccaagtcgacgagcctgccctgcgtgagggtcttcctctgcgtactggcgctgcccgtgatgcttatgttaagtgggctgtcaatgctttcaagctgtctactgctggtgtcaccgacagcacccaggtccactcccacttctgctacagtgaattccaggacttcttccacgctattgctgcccttgataccgatgttttgtccatcgagaacagcaagtccgatgccaagctcctccaggtcttcgttgatcagagtttccccgcccacattggacctggtgtctacgatatccactctcctcgtgttccctccgtggatgagatcaaggagcgtatcgagcagatgctccagtacctcaagcctgagcagctctggatcaaccctgactgcggtctgaagacccgtaccactgagcagactatcggccagctgacctctctggtcgaggctgctaagttctaccgccagaagtacactcagtaattctgtgcgcattaacatttctcatttttcctttgttccacttcctataatgtgttaattcaaggggcggtattctcaacctgccgcctctggcacggggagttctgttttatgacgattacgaagatgataccaaaagttcaacatagggtttcaaggttttgcggattagagggcaccaaaagcgggtttacggagtttcaacatgactgacgatttcaacatgggtgccgcctattaggtgccgaatctttcaacaatttggttcttccatccttgtgaagcagttgctatcacttggatctggagaacgaaacaagaactttgttctactgtagagttagctgtgaatattacttctagctttatgctacacg

SEQ ID NO: 88 - DNA, 362 bp, unidentified
  source 1..362: mol_type = genomic DNA
gttgacctcggatcaggtagggatacccgctgaacttaagcatatcaataagcggaggaaaagaaaccaacagggattgctctagtaacggcgagtgaagcagcaatagctcaaatttgaaatctggcgtcttcggcgtccgagttgtaatttgtagaggatgcttctgggcagccaccgacctaagttccttggaacaggacgtcatagagggtgagaatcccgtatgcggtcggaaaggcaccctacacgtagctccttcgacgagtcgagttgtttgggaatgcagctctaaatgggaggtaaatttcttctaaagctaaatattggccagagaccgatagcgcacaagtagagtaacc

SEQ ID NO: 89 - DNA, 663 bp, unidentified
  source 1..663: mol_type = genomic DNA
gaccctcactctctttctccctctcttacatagcgagctggtctccatccttgttgtttgatttgatcttctttgcatttccctatcccagtgatgaagttatccaattccgctcattactcgcttttcctcctatcctccatcctcggcttctccagcgcgtcggccaactctcacctcagtgatgattctccttgtgtggcccgctcgccaacaagtgggctctattatgatctgaatgctatctcattagcaccgccggaatggaagaacgggaagaaagttgatcaggaagcgcgagatgaaagctggcatgccaaggggcatgactaccccgcgaacttcacaatcaatgtctgcgcgccggttcttgagaatgtaaccaatgttgtcggggtagatgcctctcgatgggcgaatgtcagtgctttctatgagcaagctgggaagatatactcaatgggagagcaagcctccgagcctttcttccgcggccgcaagctagtactcaactacacggacggttcgccatgtcccggtgattcgaatactgctagcggcaatagctctattcgaaccaagtccactctgatgtccttcctctgcgatcgcgcggccgaattccccgggctcgagaagcttggatccaccggatctagataa

SEQ ID NO: 90 - DNA, 1243 bp, unidentified
  source 1..1243: mol_type = genomic DNA
atgtccatccgcaatgaatggcttcaatgagaaaggcctcgacggggatgcctttggagagaagtccaatctctccgggctaaagacatttgacgctttccccaaaacaaaaacatcctacacaaccccaacccgacgaggcggccaatggaccgttctcatcctagcagtatgcacactattcagcctccacgaactccgcacctggtggcgcggcacagaagcccaccacttcagcgtggaaaaaggcgtatcccacgatctccaattaaacctcgatatggtcgttcacatgccctgtgacactctccgcataaacattcaagacgcctccggagaccgcgttttagctggcgaactcctaacccgcgaagacacaaactgggacctttggatgaagaagcgcaatttcgaatcccacggcgaacacgaataccaaacgctcaatcatgaagcggctgatcgattaagtgcgcaggatgaagacgcgcacgtacaccatgtcctgggtgaagtgcgccgtaacccgcgccgcaagttttctaagggtccacgtctacgctggggcgataacaaggattcttgtcgaatttatggaagtcttgaagggaataaagtgcaaggggatttccatattacggcacggggacatggatatatggaattggcgccgcatttggatcacgaagtcttcaatttctcccacatgattacagaactgtccttcggaccacactatccatcccttctaaaccctcttgacaagaccatcgccgaaagcgaaacccactaccagaaattccaatacttcctttccgtcgtcccgaccctctactcaaagggccacaatgcacttgacctcgtgacaacaaataaagataactccgtccgctacggccgtaacacaatcttcacaaaccaatacgcagccacaagccagagtaccgccctccctgaaatccccaccctaatcccgggaatctttttcaagtataatatcgagccgatcttgctacttgtcagcgaagagcggacgggattcttggctcttgtcattcgagtcattaataccgtttctggggtcttggttacgggtggttggatctaccagatttctgggtggattgttgagatccttgggaaaaggaaacggcagtctgagggtgttttgactgggaagcattattcggattgatttgtttctagtagtttcgtctcaatataagtttgattttccttttcc

SEQ ID NO: 91 - DNA, 1007 bp, unidentified
  source 1..1007: mol_type = genomic DNA
gaacggtgatagtagtagtaggctcgtcatctatctacaacccctctctcctcactcccctctctcgacgccatgttcacgcgtactctccgaccggccgtggcggtcgccaggactcaggctgtccagcagcaacaggccggtatggccacattgaaggaaatcgaccagcgtttgaaatccgtcaagaacattgggaagatcaccaagtcgatgaaggtcgttgcctcgaccaagttgacgcgagctgagaaggccatgcgtgaagccaagaagtacggtgccgccaacaacgttctgttcgagcagaccaaggctggtgaggaggagcccaaggagcgcaagatcctctacctcgccatgacatccgacggtggtctgtgcggtggtatccactccaacattacgcgatacatgaagaaggctgtggccaaggaacccggaatgctggctgttgtcggtgacaagcccaaggctcagctctctcgagcgatgcccaaggctttgaccatgtctttcaacggcgtcggcaaggatgtccccactttcgtcgaggccagcgctatcgccgatgagattatgaaatctgccggtgactttgacgagatccgaatcgtctctaacaagtacctttccgctatcgcctacgaacctcacaccaacgccgtcatctccgctgaggcactccgacaagccgccggtttccagcaatacgagatggaggaggatgtctccaaggacttggccgagttcgctcttgccaacgccatctacactgccctggtcgagggacacgccgccgagatctctgcaaggaggcaagctatggagaacgcttccaacaacgccaacgacatgatcaactctctccagctgcagtacaaccgtggtcgacaggctgtcattaccaccgagctgatcgatatcattaccggtgcctcggctctgtaagcgggtgtagactagatggacaaaacaacaaaaatggcatgcagcgaatgacattg

SEQ ID NO: 92 - DNA, 1007 bp, unidentified
  source 1..1007: mol_type = genomic DNA
caatgtcattcgctgcatgccatttttgttgttttgtccatctagtctacacccgcttacagagccgaggcaccggtaatgatatcgatcagctcggtggtaatgacagcctgtcgaccacggttgtactgcagctggagagagttgatcatgtcgttggcgttgttggaagcgttctccatagcttgcctccttgcagagatctcggcggcgtgtccctcgaccagggcagtgtagatggcgttggcaagagcgaactcggccaagtccttggagacatcctcctccatctcgtattgctggaaaccggcggcttgtcggagtgcctcagcggagatgacggcgttggtgtgaggttcgtaggcgatagcggaaaggtacttgttagagacgattcggatctcgtcaaagtcaccggcagatttcataatctcatcggcgatagcgctggcctcgacgaaagtggggacatccttgccgacgccgttgaaagacatggtcaaagccttgggcatcgctcgagagagctgagccttgggcttgtcaccgacaacagccagcattccgggttccttggccacagccttcttcatgtatcgcgtaatgttggagtggataccaccgcacagaccaccgtcggatgtcatggcgaggtagaggatcttgcgctccttgggctcctcctcaccagccttggtctgctcgaacagaacgttgttggcggcaccgtacttcttggcttcacgcatggccttctcagctcgcgtcaacttggtcgaggcaacgaccttcatcgacttggtgatcttcccaatgttcttgacggatttcaaacgctggtcgatttccttcaatgtggccataccggcctgttgctgctggacagcctgagtcctggcgaccgccacggccggtcggagagtacgcgtgaacatggcgtcgagagaggggagtgaggagagaggggttgtagatagatgacgagcctactactactatcaccgttc

SEQ ID NO: 93 - DNA, 1139 bp, unidentified; identical to SEQ ID NO: 46
  source 1..1139: mol_type = genomic DNA

SEQ ID NO: 94 - DNA, 526 bp, unidentified
  source 1..526: mol_type = genomic DNA
aacccaccccgccattctcaattcttcgtccgtgttcttctcgagaagctacacttcgcaaaaatgggtgccattccggaatatgatcccgaggagcccctcgagaccaagcccttcaagttcgtgactgctggttacgacgctcgtttcccccagcagaaccagaccaagcactgctggcaaaactacgtcgactactacaagtgtgtcgaggccaagggtgaagacttccgcccctgcaagcagttctaccacgctttccgctccctctgccccaaggcctggactgaccgctgggacacccagcgcgagggtggtaacttccctgctatccttaacaaatagataaccaatggctgctttgtgttggtgaattgggttatagcagattctgtattgacaaactttccaatgtactctacctggtcatgcggggatacatttcttttctgtttggatgtaattttcccactctgatgaagaaagtgtgtctataaactcgctgttttgaaactaaacgtcttcc

SEQ ID NO: 95 - DNA, 839 bp, unidentified
  source 1..839: mol_type = genomic DNA
gggccattgctcgaggagctcgatgtcgaggcgtacgccaagaagtaccgttacctgagattcatgtgccaggagacgctggaccatctcgcctttctcaaggacaaagtgaaggatgtcgaagggttctgggcatccaccttgttgaagcaccgcgatctcaggggctacatcacttcacgatccgacaaggacgcattgaagtacttgactcacattgagctcgttcaggatcccaaggatccccgtccgttcgctctcaaattctacttcaaggagaacccatacttctccgacttggtcttggagaagaagtacgatatgtccgagggttccgaacccgcacctgccgatggtagcattacggagggaatgcgcaatttcaaagaagacgagctggtcaccaaggctaccacgatcaactggaagtcggacgacaagaatctagtcgccaagcagcccagatccaaaattcccgacaatgacgacgatgaagatttcgacggggacgtcggatcgttcttcaactactttacagatgacacagatattttccagattggggccctcctgcagtcggagctactgcctgatgccatcgactactttgttggccgaggcgagcaggtggactctgaaggagaggagctagacgagctggaagaggatgatgaagacgacgatgaggatgatgagggcagtatcgacctcgaagacgaggaggagcagccgagtaaaaagaagcccaagagggcctaagaaacatttgatccgtcaacatgtacggacgaggtaatcgtgttcgaatgttaatgatcatgcatatgctagtaaattcg

SEQ ID NO: 96 - DNA, 804 bp, unidentified
  source 1..804: mol_type = genomic DNA
ggatctttcctcacccctcaacacactcacacaccattcggacgcgctatgcacaatgccttgacggtttcgaggctcaacgacaagttccaagagccgctcgttgttcttgtggagcttttgcgtgctcgtgttctgcacgaccgcaacttctcgaacagacaattctcaggtggtccttcattcgggacagacaatcagaagagaagcatgcttttgatcttccgtactctgtccattattccgctccaattcaaggccgagcactggtcaggaccattgtcaagagagctgcttgtcttcaactcattccacaagacactgtcaagatccctgagaacgctggtcgaatcaatcactatgaacgccttcctcaagaacaatgcgagaagagcacgtgacgactatcttgacattgcactttcactaccattccaaaacgataccaacactggattcggtatcttctttaagatttacttggatgcattgactacatttgcagaaggcaacatcactgaagagaacaaagacagcgagtctgtgaaagaggccaagcagtcagcaatggagatcctaggtgacgccataccgaacgtgaaggacccagaggccgagcttttgcggggtttcagattctgggatgctgtgctcgtgtgcgtccgtacactcaaagcagacagggcaatcgatctcaagctagctgagtctttcgaggcggcaaacagctaccttaatatgatgagaccaaattgatacggcgttttgtagcaatcttgagctttatgcaatctacttctgtcg

SEQ ID NO: 97 - DNA, 848 bp, unidentified
  source 1..848: mol_type = genomic DNA
ggatctttcctcacccctcaacacactcacacaccattcggacgcgctatgcacaatgccttgacggtttcgaggctcaacgacaagttccaagagccgctcgttgttcttgtggagcttttgcgtgctcgtgttctgcacgaccgcaacttctcgaacagacaattctcaggtggtccttcattcgggacagacaatcagaagagaagcatgcttttgatcttccgtactctgtccattattccgctccaattcaaggccgagcactggtcaggaccattgtcaagagagctgcttgtcttcaactcattccacaagacactgtcaagatccctgagaacgctggtcgaatcaatcactatgaacgccttcctcaagaacaatgcgagaagagcacgtgacgactatcttgacattgcactttcactaccattccaaaacgataccaacactggattcggtatcttctttaaggttggtggccttgcaggagtctgacatagcgctgacgcagacagatttacttggatgcattgactacatttgcagaaggcaacatcactgaagagaacaaagacagcgagtctgtgaaagaggccaagcagtcagcaatggagatcctaggtgacgccataccgaacgtgaaggacccagaggccgagcttttgcggggtttcagattctgggatgctgtgctcgtgtgcgtccgtacactcaaagcagacagggcaatcgatctcaagctagctgagtctttcgaggcggcaaacagctaccttaatatgatgagaccaaattgatacggcgttttgtagcaatcttgagctttatgcaatctacttctgtcg

SEQ ID NO: 98 - DNA, 628 bp, unidentified
  source 1..628: mol_type = genomic DNA
ggggcatctacctcgacggcaacaacgacctggtcactatgaagggtaactacatctaccacaccagcggccgctctcctaaggttcagggtaacaccttgctgcacgctgtcaacaactactggcacgacaactccggccacgccttcgagatcggtgagggtggttacgttctggccgagggtaacgtcttccaggatgttactacccccgttgaggaccccgttgacggccagctcctcacttcccctgaccccagcaccaacgctcagtgctcgtcataccttggccgggcctgcgaaatcaacggcttcggtaactctggtaccttcaaccaggctgacactagcctgctgtctaaatttaagggtcagaacattgcttctgctgatgcttactctaaggttgcctcgagcgttgccagcaacgccggtcagggacacctgtaaaatggaaagaggaggttcagagcttaatttgctcatgtcggacgacatagccctagcggcttgctggtgaatttggcataatagcgtttctcttctcatacctactttattactccgtttggatccttattaggtaaatattagcccattgtatggttcaattcgattgactttgaggc

SEQ ID NO: 99 - DNA, 804 bp, unidentified
  source 1..804: mol_type = genomic DNA
agttggaaatctgatcaattactctccatcttctcgttctactatctaatcctctcttccttccaaaacatatcatcatgtctgctcaacctctccgcattgtcatggcctgtgacgaggctggtgttccttacaaggatgccatcaaggccgttctcgagaagagccccctcgtcgcctccgtctctgacgtcggtgtcaacgatgcctccgataagaccgcctacccccaccccgccgtcgagggtgctcaacagatcaaggccggtaaggctgaccgtggcctcttcatctgcggtactggtctaggtgtcgctatcgccgccaacaaggttcccggtattcgtgccgttactgcccacgaccctttctccgtcgagcgttccattctgagcaacgatgctcaggtcctctgcatgggtcaacgtgtcattggcgtcgaacttgcgaagaagcttgccctcgattggctcaactaccgtttcgatcctaagagtgcctctgccgcgaaggtccaggctatctccgactacgagaccaagttcgctggctcttcttaaatgcattatcttgcatgaatgacggtcttcgtacatactttgccacatatgggttctaattgcactgcgtctgcagtctcgatatgaaaccattggattgcgatggatgtcccttttccatttgcaactttttatatactttcttttctaccaagcgcttcatgataccacgattcgattaccgagttctgctgtttgctttggtcggtagatctagatacacaatgcagtatattcgagtttc

SEQ ID NO: 100 - DNA, 782 bp, unidentified
  source 1..782: mol_type = genomic DNA
acttctccttttctgttagctttgactctactatcctgctcctcctctaaatccgtggaatccaattttttcacaataacttcgctaccataatgtccgtcactaccacttcctccgccgccgcagcctcctgcactccctcttggcagattcctgtcgacgatgttgcctgtgccggtcagatcagcggtaatatcaccaaggttttcgatacctgctgtaagggaaacagccctgtcaagtacaacgacgactgcaacatctactgtcttgcccaaggacaaaccaagcaagagttgaccgactgtttgaccgagaagagcggaaacaaccagatcttctgtggtcatggcaagcagaatgccactgctacagctgaagccaccaccaccaaggagactggcacatcgaccggcacttcaacctcttccactggcacttctaccgagaccaacgctgccgtgctcaaccaacccatctccaagaccggtcttggactcgtcgccatgctcttctgctctgccctcgttggtgttgtcgcctaagttatgactccaaaacgaacacattactgcggtatggatacggcaattatgacaaccagaggaccgcagggacggagaatggtaattgatgaacccggaaaagatacgtggtgcatggacataaatgtttgatttactcttactgtctgcttcaactttccgagaggaatattgtttcttctgtaccaatagcgatagcattaacagcatcttaattctaattttgcatatcacttc

SEQ ID NO: 101 - DNA, 693 bp, unidentified
  source 1..693: mol_type = genomic DNA
gccggggtcgatcgaggtgtcatcaccaaggacgagaaggacagcagtatcaatagactactcgtcactggttacggtctggccgaggtcatgggtacagatggtgtgaacggcatcaagacgcgaaccaatcacgtcatggagacgtgcgaggttctgggcatcgaagccgctcgacagaccatctacaacgagattcagcataccatgacatcgcacggaatgtcaatcgatcctcgacacgttatgctgctcggagacgtcatgacttacaagggcgaggtgctcggtatcactcgattcggtgtgcaaaagatgaaggactcggttctcatgttggccagtttcgagaagaccactgatcatctgttcgatgcctcgctgttttcgaaaaaggatgaaatccaaggcgtctccgagtgtatcattatgggcacacccgcgccaggttgtggcacctcacttgcatcgatcgtcacacctgcccctctcctcccacgcaaaaagcctttgctgtttgaaacagcgttcaaagctggtcaggatcgattgagctatcacgaaaacaatggcggcatggaggtggacatgtgaacccggtccctcatacatcttcttctgattgtctgtaccatacatacatcgcattgcttcttttcacatacgacacgacatgcatctgacatctacgac

SEQ ID NO: 102 - DNA, 776 bp, unidentified
  source 1..776: mol_type = genomic DNA
gcaggatcaggaggagcatattccttctctctgaccaccttctcgccttctggaaagcttgtccagatcgaacatgcattggcagcagtagcgggtggaacaacatcactgggtatcaaagctaccaacggtgttgtccttgcgactgagaagaagtcaccgtcactcctgctcgatacgtctgttctcgaaaaggtagctcctatatgtcccaacattggtttcgtctactcgggtatgggacccgatttccgagtcctggtcgccaaagctaggaagatcgcccaagcgtactataaagtgtatggcgagtacccacctacaaaggttctagtgcaggaggtggcgggcgtgatgcaaaaggctacgcaatctggtggtgtgcgaccatatggtatctccctcttgatcgctggttgggattcgcaccgaggtcagagcctgtaccaagtggatccgtcaggtagctactgggcgtggaaggcaagcgcgatcggcaagaacatggtcaacggaaagacattccttgagaagcgatacaatgacgacctgtcactcgaagatgccattcacacggcccttctcacgctgaaagaaggtttcgagggacagatgactgagaacacgatcgagatcggtgtagtgacggtaccgacggccgagcagatgcaggagaagccaggagagaggctacctcccacgttcaggaagttgacggagcaggaagtgagggactatctcgccttgtagacgatgcagacagaacatgaccatcc

SEQ ID NO: 103 - DNA, 1191 bp, unidentified
  source 1..1191: mol_type = genomic DNA
aagtctacgactcctccagccaatttactttgatccaaaatgttgaccagcgccttttctacatctgcttccaagatgctcggcaagagagcagtctcgtcttccagcgccttgaacggaaaggttgccgtcctcggtgctgctggcggtattggccagcccctctccttgctggtcaagcagaaccctgctgtctccagcctctccctttacgatgttcgcggctcccctggtgttgctgctgacattagccacatcaacacccctgctgtcaccgagggcttcctccccgacaacgatggcctcaagcaagccctcgagggtgctgaggtggtcctcattcctgctggtgttcctcgcaagcccggcatgacccgtgacgaccttttcaacaccaacgcttccatcgtcaagatgcttgctgaggcttctgccaagtactgccccaaggctatgatgctcatcattgccaaccccgtcaactccaccgtgccgatcgtcgctgagaccttcaagcgtgctggtgtctacgaccctgcccgtctcttcggtgtcaccaccctcgacgttgtccgctcttccactttcgtctctggcatcaccggtgccaagccctccgacaccgtggtccaggtcatcggtggtcactctggcgccaccatcgtgcccctgctctcccagatccctcagggcgacaagattgtcaaggctggcggccagcagtacgctgacctcgtcaagcgcatccagtttggcggtgacgaagtcgtcaaggccaaggacggcactggctccgctaccctctccatggcttacgccgctgccgtcttcaacgacgctctcctcaaggctatggacggccaaaagggtctcgttcaacccgcttacgtcgagagcccccacttcgccaaggagggtgctaagtacttcgcctccaacgtcgagctcggccccaacggtgttgagaagatcctcgacatcggcaacatgtcctctgaggagcaggagctccttaaggagtgccttccccagctcgccaagaacattgctgctggtgagaagttcgtcgctgacaactagaggatatcccacgacgttgctccctataataatgagagcaagcgagaacaagagaaataaagacatagcaaattgaatagggcttccaactgcaccaaaaagcagtgatgc

SEQ ID NO: 104 - DNA, 1191 bp, unidentified
  source 1..1191: mol_type = genomic DNA
gcatcactgctttttggtgcagttggaagccctattcaatttgctatgtctttatttctcttgttctcgcttgctctcattattatagggagcaacgtcgtgggatatcctctagttgtcagcgacgaacttctcaccagcagcaatgttcttggcgagctggggaaggcactccttaaggagctcctgctcctcagaggacatgttgccgatgtcgaggatcttctcaacaccgttggggccgagctcgacgttggaggcgaagtacttagcaccctccttggcgaagtgggggctctcgacgtaagcgggttgaacgagacccttttggccgtccatagccttgaggagagcgtcgttgaagacggcagcggcgtaagccatggagagggtagcggagccagtgccgtccttggccttgacgacttcgtcaccgccaaactggatgcgcttgacgaggtcagcgtactgctggccgccagccttgacaatcttgtcgccctgagggatctgggagagcaggggcacgatggtggcgccagagtgaccaccgatgacctggaccacggtgtcggagggcttggcaccggtgatgccagagacgaaagtggaagagcggacaacgtcgagggtggtgacaccgaagagacgggcagggtcgtagacaccagcacgcttgaaggtctcagcgacgatcggcacggtggagttgacggggttggcaatgatgagcatcatagccttggggcagtacttggcagaagcctcagcaagcatcttgacgatggaagcgttggtgttgaaaaggtcgtcacgggtcatgccgggcttgcgaggaacaccagcaggaatgaggaccacctcagcaccctcgagggcttgcttgaggccatcgttgtcggggaggaagccctcggtgacagcaggggtgttgatgtggctaatgtcagcagcaacaccaggggagccgcgaacatcgtaaagggagaggctggagacagcagggttctgcttgaccagcaaggagaggggctggccaataccgccagcagcaccgaggacggcaacctttccgttcaaggcgctggaagacgagactgctctcttgccgagcatcttggaagcagatgtagaaaaggcgctggtcaacattttggatcaaagtaaattggctggaggagtcgtagactt

SEQ ID NO: 105 - DNA, 1505 bp, unidentified
  source 1..1505: mol_type = genomic DNA
ggggcaagcgtgctgcttccggcactgcatttcgacgagcgggagcaacgacctggacgtacgtcttcgactgcaagtctgctgcagcgtcacgatcgcaatgaacctttatcgctcaactcgcactcgccgacatctgtggaccacactcccactactgcgcatttcactggtgctgaagagttgctcgcctccgacgtcggaccgaccgcgacagctgggctacccggtgatgcggagcttgagagcaagctcaagctgcttgaagaggtcaaacgtgcacgggaatcggtacatagctcgctcgagaggatcagagccggcacgcctaccccgtctatcagccagggaatgcccagcccgacaccctctggtgcccctggttacgctagaactccgtcgtctgtcggcctgtcggacgacgtgcgctcgagacgaggctcaacgaccagctccaaggttcttgacgctatcgacaagcctcgagtcgctacccaatccgaatgggacgagtacgttcgcaaccggcatgtcatctcacctccacccactcagtttgccgtattgcccacatctgctgcgatggtcgatcgtggtaccagtcgacacagccagtatgcccttgtttccgacggcgttgccaaagcgcttgacaggcgggagcgaactatttcaatgatggagccgcaagttgccgaggactggggaccaagagagacgctcgacagcactccggctcatgtctcgatgggccgtcgagccatgtcattccatgagatacctctggcatcgcctgtcgctgcctctcgacctcaggaccgctcctcctactctgccggaccacgtcaggtcatagggtcagctgctggccacacgcagcgaccgggtatcagtcaatcgagatcagcccacggccggactatgacatacgacgagctgacggagagacatcgtcagcgcttgtcggcattgcaagcgccagtcagcgccaaaatcagggagccgatggacatcgcgtccgccaaagccagctgggacaagcaaaagcgggtcgagcgggacgaaatgaagaggcgagaagccgagaagctcgctcaggctcacgcaagagagcgacgagggcccgctgtcgacaagaaggaagttctcaagtcgaccgacgaatggaggcgaagcgtccacggcggtctcgacggtttcgccgttccgcacctaccggcccacgctcgaggttccacgcagcctggtggatccggcgccaagcgatcttcactctctcaaaggcccagcaactacttcgccaactaggcataatcgaatcgcggacagtcatctgtacatagaaccgtacctgtattaccaaccctgcacttccgctcacacctgttgcctatacctcgtctaccaacgctcattccaatatcatagctacattcatttgcaaggacactatcacaccgcagtcatgactccgtatggatattcaatgcatacctcttccagag

SEQ ID NO: 106 - DNA, 1391 bp, unidentified
  source 1..1391: mol_type = genomic DNA
aacctcggccgagaggacaagattatcaagaatgggatctcctgcgcctcaccaccgccatcaatcctcacttgaaggagtcattgacttttctactggtagagggcatccattgaatccctatcaacgcgacaaggccgagagcgtttttactggcattatcaaccgcttcgaggactcgtcgaccgtagagaaaccatacaaccgtgccaagctggttcgcctgacgtatgagtatgctcgctcggaagattctcgatgcaatttcttgcaagcattcttcggatcagtaaacgttacgatggatgactctattgatttcgacgatgaagcggtagaagaggggattcgctcgagcctgaattccttcgcagatttcttggtggagaacttcttccttccactcaaggcttccgccagcaggacgcccccagccccccagcccaagttccgagcagacgtcctgctgtggggtctgtggaaagagtggcctcgctcagacgcgactgcctcatccgcgatcgacatcgttgcgtaatctctcgcaacttcgacatgaaagaagctgagcgacgtcttgacgatagcggatatgaccatgcctcggacgatgaaggacatttactgaaagatcaggagcatgggtcattcgcggaactagaagttgcgcatatacttcctcactcattgatgactacgacagcgaactccgagctgaacaagtccaaagaaacggcattgacaatacttaatatgttcgacagtggcattgtccatctaatcgacggtccagacattgatcgccctcgaaatgctcttaccttaagcattgacctccatcgacagtttggcaacttcaaggttttttttgagcctatgcctgagccccatacctaccggattgattcaaccctccgccagccatttagaaacccgattttccctgtaacccgtgcactctacctcacccctgagcgaactattgatcccccgtccggtcgacttcttgccgttcatcgcgcaatttgccacattttacatctcagtgctgctgggaattacatcgacagcatacttcgcgacatggatgacgggactgtacaagccaacggctcgactcgcctggctagcatagttcgtctgaaactggggggttggtgggatggcactgttgttggatagtcaaccacttcgaccctctccatacaccacaacggcaactcgagctgatgcatcaccgatctacctacgccattcgcgtggaggattgtcgcatatcaccactaggttcgtgcgactggatatgaaacgcggcccgtactttggggtcgtgtatccggtttcacatccagcttgtcgcatcaaggattccaatcctaacgacatgagcc

SEQ ID NO: 107 - DNA, 665 bp, unidentified
  source 1..665: mol_type = genomic DNA
ggacgaccgaaataccgtcaaaatggtcaacatcccgaagacgcgcaggacctactgcaagggcaaggaatgcaagaagcacacccagcacaaggtcacccagtacaaggctggcaaggcctccctcttcgcgcagggtaagcgtcgttacgaccgtaagcagtccggttacggtggtcagaccaagcccgtcttccacaagaaggccaagaccaccaagaaggtcgtcctcagattagaatgcacttcgtgcaagaccaaggcgcagctcgctctcaagcgctgcaagcacttcgagcttggtggtgacaagaagaccaagggtgccgctcttgtcttctagatgggtgcataacggttatggcgctagggatgatgatggagcggtctgtgcatgtagcctccttgagtacatgatcctcgagggctcggaatcaaagcttcgtttctcctacgatcgtcccactcgcaaagacatgtctcgtcatatcatggcttgcgcacaacattcttcgagggtccatcagagatgcccgaccctgccgctacgctgcgtgggatgtgactccagcacaaccgccttccagtatcatctcttcgcgtgcagaagtgaggacgattttacgacagtccatataacaaatcggaaatgccaacaagatcaa

SEQ ID NO: 108 - DNA, 1327 bp, unidentified
  source 1..1327: mol_type = genomic DNA
ggcccctcttgaactttggactttttagcatctattttctctacttctctctccctcctcctatctaccttctctatcatctccttcagctccctacaacatgaagctcacctttaaggacctgaagcaggagaagttcgtaatcgaggtcgagccctccgagactgttcgcgaagtcaagcaaaaaattgctcaagaaaaaggcgaatatgaggcggaacgaatgaaagttatctactcgggcaagatccttcaggatgacaagaccgtcgaatcatacaacatccaggagaaggatttcctagtctgtctgccttcaaagggtcctaagcccgctgcctcgtcgtctgcctcccaggcacccgccactccggcccctagagctcctgttgctactcctgctgctcctgcccccgctgctcctgcacctgctagttctacgcctgctgtccctgcgactccctcgcctgctggcgcccagaccggtccctctttcggtgacccatctgcattgaccatgggttctgcggctgagggtgccgtcactcagatggaagcaatgggatttgccagaagcgatattgaccgggccatgcgggctgcattcttcaatcctgaccgcgctgtcgattacctcttgaacggtattcccgccgatgttcaacaggaacaacagcagcggcaacaagagcaacaagcggaccgtgctgcagaacaagctcctgtgcccagcgctgaggatgctgctgctgccgccgctctgggtggcgatgagggttttaacatgttcgaggctgccgctcaggctggtgatggtcgtggtggtggtgctcggtctggaggtagcgaggcccttgcgaacctggactttctccgcagtaacccccatttccagcaactgagacagttggtccagcagcagccgcacatgctcgaacccatcctgcaacaggttgctgccggaaacccacagatttcccagatcattggccaaaactctgaacagtttctccaactgctaagtgaggagggtgatgaggaagatgcggccctgcctcctggtacacaagctatctccgttacagaggaggagcgggacgccattgagcggttgtgccgtctgggtttcccccgggattccgtcatccaggcctacttcgcctgcgacaagaacgaagaactcgcagcaaacttcctcttcgaccagccggacgatgatgaggagtaaatctgatccacgatgctgtggttcacttctttactccatgtcttatccccttccccttttgcttctttacgttctgatgaataccaagcatgcctgttg

SEQ ID NO: 109 - DNA, 1326 bp, unidentified
  source 1..1326: mol_type = genomic DNA
ggcccctcttgaactttggactttttagcatctattttctctacttctctctccctcctcctatctaccttctctatcatctccttcagctccctacaacatgaagctcacctttaaggacctgaagcaggagaagttcgtaatcgaggtcgagccctccgagactgttcgcgaagtcaagcaaaaattgctcaagaaaaaggcgaatatgaggcggaacgaatgaaagttatctactcgggcaagatccttcaggatgacaagaccgtcgaatcatacaacatccaggagaaggatttcctagtctgtctgccttcaaagggtcctaagcccgctgcctcgtcgtctgcctcccaggcacccgccactccggcccctagagctcctgttgctactcctgctgctcctgcccccgctgctcctgcacctgctagttctacgcctgctgtccctgcgactccctcgcctgctggcgcccagaccggtccctctttcggtgacccatctgcattgaccatgggttctgcggctgagggtgccgtcactcagatggaagcaatgggatttgccagaagcgatattgaccgggccatgcgggctgcattcttcaatcctgaccgcgctgtcgattacctcttgaacggtattcccgccgatgttcaacaggaacaacagcagcggcaacaagagcaacaagcggaccgtgctgcagaacaagctcctgtgcccagcgctgaggatgctgctgctgccgccgctctgggtggcgatgagggttttaacatgttcgaggctgccgctcaggctggtgatggtcgtggtggtggtgctcggtctggaggtagcgaggcccttgcgaacctggactttctccgcagtaacccccatttccagcaactgagacagttggtccagcagcagccgcacatgctcgaacccatcctgcaacaggttgctgccggaaacccacagatttcccagatcattggccaaaactctgaacagtttctccaactgctaagtgaggagggtgatgaggaagatgcggccctgcctcctggtacacaagctatctccgttacagaggaggagcgggacgccattgagcggttgtgccgtctgggtttcccccgggattccgtcatccaggcctacttcgcctgcgacaagaacgaagaactcgcagcaaacttcctcttcgaccagccggacgatgatgaggagtaaatctgatccacgatgctgtggttcacttctttactccatgtcttatccccttccccttttgcttctttacgttctgatgaataccaagcatgcctgttg

SEQ ID NO: 110 - DNA, 1162 bp, unidentified
  source 1..1162: mol_type = genomic DNA
gcgccgggggacatggagactgccgacgccaagaacagggctatgcgagccgctggcttcatcgttcccgacaccttcgaagacctgcccgaggtcctcaagaccacctacactggtctggttcaaaagggtgtcatcgttcccaaggccgagatcgacccacccaacatccccatggactaccagtgggcttccaagttgggtcttatccgaaagcccgccgccttcatctcgaccatctcggacgagcgaggtcaggagttgatgtacgccggtatgcgaatctccgacgttttcaaggaggagatcggtatcggtggtgtcatctccctcctgtggttcaagcgacgattgccacctttcgcctgcaaattcatcgagatggttctgcaattgactgccgaccacggacccgccgtttcgggtgccatgaacaccatcatcaccgctcgagcaggcaaggacctgatctcgtccctggccgctggtctcttgaccatcggtgaccgattcggtggcgctctcgatggtgccgccgccgagttctctcgaggtctcaactctggtgctaccccacgagaatttgtcgactcgatgcgaaaggccaaccgattgattcccggtatcggacacaagatcaagtcaaagaccaaccccgatctccgagtcgttctcgttgtcgattacgtcaagaagcacttcccgtctcacaagacgctcgactttgccttggccgtcgaggacgtcacgacgcaaaagtccaacacgctcatcttgaacgttgatggtgctattgccgcttccttctgtgatttgcttagcggttgcggtgctttcactgaggatgaggctgccgattacctcaagaacggtactcttaacggtcttttcgttcttggtcgatcgatcggtttcatcggtcactacctcgaccaaaggctcctcaagcagcctctctaccgacaccccgccgacgacattttcatcaacatgcaagagcgagttgtcttccagcctgggtccaactaagaggcgaccgcgactacgggtctcggccaatttctcccttgggtttcctccttcaattaaaactactgtacataccacccacatcatttatctcttctttcatgactatagacgcatgcacgggatcgctcgg

SEQ ID NO: 111 - DNA, 965 bp, unidentified
  source 1..965: mol_type = genomic DNA
ggggcatacaaggagggcaagttcaccagcgaaagcatccaaaagtcaaagctcagattccaggacatcctcgttgagctgcccctcagggttcacaactcccaccttctcaccagcttcctgcaccaggtcccgcaggcgccgccggcaaagaaccccctcgacttcccttcatcccttgcagagctttcgcgcgactccgatgtcagctccaaccccttcgcacccaaccttgacaccctggacctcagcatcgaccccttccagtactggcagcgcgccctcggccgcgagcagcagaagatcaccgcatggcaacagaagcgcaaggctgagaatgctgcacgcgccgcgagcaagcagccgccccttgacgagaatgagtggcagaagctgttcaagctgcccacggagcccagcaggctcgaggctctgcttgtcggcaggcaggtcgagcagtacgcccgccaggtcgacggattctccgccaccgtttccgccaagatgtttggcgtcaggggcaacctcctccctaacgagatcgagtagaggacgaatattacggagacgggaccggcgtttatgcatagcgaggcgttctcggctgggtggggtagagtacatgcggcatggctacaaaaaaaaggatgatgtggttccgccatcgacgagttcagggcaacgctgcatagaatcccaaaagaagaaaggattttaacgtttttgaatttggaacttcttcgcattggacgattgctttcttgacgactccgtcagttgcgcgctttttccatggcccataccctctttatctctaatgagggtgcgccaccgcagacccaccagctactcgaagaaaagtcgctattttttatttggagttattagcgagtacaaacggaggcatgtctagaggctgaggagtgtggtagtaagattatagatgtctttatgctcgatatgag

SEQ ID NO: 112 - DNA, 965 bp, unidentified
  source 1..965: mol_type = genomic DNA
ctcatatcgagcataaagacatctataatcttactaccacactcctcagcctctagacatgcctccgtttgtactcgctaataactccaaataaaaaatagcgacttttcttcgagtagctggtgggtctgcggtggcgcaccctcattagagataaagagggtatgggccatggaaaaagcgcgcaactgacggagtcgtcaagaaagcaatcgtccaatgcgaagaagttccaaattcaaaaacgttaaaatcctttcttcttttgggattctatgcagcgttgccctgaactcgtcgatggcggaaccacatcatccttttttttgtagccatgccgcatgtactctaccccacccagccgagaacgcctcgctatgcataaacgccggtcccgtctccgtaatattcgtcctctactcgatctcgttagggaggaggttgcccctgacgccaaacatcttggcggaaacggtggcggagaatccgtcgacctggcgggcgtactgctcgacctgcctgccgacaagcagagcctcgagcctgctgggctccgtgggcagcttgaacagcttctgccactcattctcgtcaaggggcggctgcttgctcgcggcgcgtgcagcattctcagccttgcgcttctgttgccatgcggtgatcttctgctgctcgcggccgagggcgcgctgccagtactggaaggggtcgatgctgaggtccagggtgtcaaggttgggtgcgaaggggttggagctgacatcggagtcgcgcgaaagctctgcaagggatgaagggaagtcgagggggttctttgccggcggcgcctgcgggacctggtgcaggaagctggtgagaaggtgggagttgtgaaccctgaggggcagctcaacgaggatgtcctggaatctgagctttgacttttggatgctttcgctggtgaacttgccctccttgtatgcccc

SEQ ID NO: 113 - DNA, 1160 bp, unidentified
  source 1..1160: mol_type = genomic DNA
gacttcggcgaaggcgatgagcaactctttcataaggcaccttccttcgactcttcgtgcgtttcgttcatctactgcattctctcttacacgttcattctcttccaccatggcatccaacgggacatccacaaatggcgttcagcatgacgctcgcaaggtcttcttcttcgacatcgacaactgtctttacccgaaatcgtatcaaatacacgacaagatggccgtgctgatcgacaactactttcaaaaccatctgtcgctgtcccaagaagatgcgaccactcttcatcagcggtactataaggactacggcctcgccatcgaggggcttgttcgccaccacaaagtcgacccacttgagtacaacgagaaggtcgacgatgcgttgcctctggatgatatcatcaaacccgatccgaaacttcgaaaattgctgcaagacatagacaccgacaaggtgaagctgtggctattcaccaacgcctacgtgaaccacgccaaaagggtgactcgcctgcttggtgtagacgatttgttcgaaggcatgactttttgcgactacgccgcggaacgcctcctctgcaagcccacgacggagatgtacaacaaggctatgcaagaggcgaacgccaccgatatcgatcagtgctactttgttgatgattcagcgctgaatgcggctgctgctatgaaatacggttggaaaactgcgcatctggtcgagcctaccgcgaagcctccgccccagcccgtctcacaacaccagatcagcaaccttgaagagctgcgcaaggtcttccctgaagtatttaagacttcatgatggcatggaaattttaacgaagacacgagtgtattttacgaaaactactcaggattcccttgccttgtaagatgcgaccatcgctactgggttgggattggagatggtgcccagcaacgcttttgcgacactatcaggtctaaggactctattgtaaaacccgggtcgatttgcatatggttaattcgaatcttccatgaacacagcatttcgtgaaccaaagagcacacacgtcgaagtgttgggatgtctttgagcagccagcttggatttcttgagaggtcggaagcaattctataggatagacagcataaatgcaataaagccactattg

SEQ ID NO: 114 - DNA, 982 bp, unidentified
  source 1..982: mol_type = genomic DNA
atacaagacttaccatcaacacaatggctcgcatctttatcactggcagcaccgacggcctcggtcttctttctgcgaagcttctctcggaacaaggccacagcgtcttcctccatgcccgcaatgccgaacgagcatcccaggccaaagcagcagtgcccaaagcccaaggtgtcatcatcggcgatctttcaaacgtctcagacgtgaagcagctcgccgccgatgccaacaaggctggaccttttgacgccgttgttcacaatgctggcctcggactcaccaccaatggccagaagactgctgagggcgtagcccagatttttgccgttaacagcatggcaccttacattctgaccgctctcatggacaagccgaagaggctcttgtacgtcagctccggactgcacttcggtggcgaccccagcctcgaggacgtcacttgggccacaagggagttccgaccatcggatgcatacaacgatacaaagatgcaaaacgtcatgctctcgaaagcagtcgccaaacgctggcctgatgtgcagagcggctctcttgacccaggctgggtgaagactaagctcggcgggtcggccgcgcctggcaccaccgacgctccagcagagatgattgctgagtacgctgccggcaaatcttgcgcaggcgatcaaacaggtgcctacttgactccgcgtggcgtggaagagccgcatgatgcgactaagctggccgagaagcaggatcgtctgatgcagatttacaaggaggtatcgggtgtttcgttcccccagtaaacacagcttcatggctttgcctcgcggagacctcacattttcaattagatctccctgccgattgcagcagaccagtactcactaggctgtgcagggggcatgttgatcaagaacgagccataacgacatgccatgtcaacggacaatgagtgggcgaagtaacacatgaaattcattatctaagcgcc

SEQ ID NO: 115 - DNA, 982 bp, unidentified
  source 1..982: mol_type = genomic DNA
ggcgcttagataatgaatttcatgtgttacttcgcccactcattgtccgttgacatggcatgtcgttatggctcgttcttgatcaacatgccccctgcacagcctagtgagtactggtctgctgcaatcggcagggagatctaattgaaaatgtgaggtctccgcgaggcaaagccatgaagctgtgtttactgggggaacgaaacacccgatacctccttgtaaatctgcatcagacgatcctgcttctcggccagcttagtcgcatcatgcggctcttccacgccacgcggagtcaagtaggcacctgtttgatcgcctgcgcaagatttgccggcagcgtactcagcaatcatctctgctggagcgtcggtggtgccaggcgcggccgacccgccgagcttagtcttcacccagcctgggtcaagagagccgctctgcacatcaggccagcgtttggcgactgctttcgagagcatgacgttttgcatctttgtatcgttgtatgcatccgatggtcggaactcccttgtggcccaagtgacgtcctcgaggctggggtcgccaccgaagtgcagtccggagctgacgtacaagagcctcttcggcttgtccatgagagcggtcagaatgtaaggtgccatgctgttaacggcaaaaatctgggctacgccctcagcagtcttctggccattggtggtgagtccgaggccagcattgtgaacaacggcgtcaaaaggtccagccttgttggcatcggcggcgagctgcttcacgtctgagacgtttgaaagatcgccgatgatgacaccttgggctttgggcactgctgctttggcctgggatgctcgttcggcattgcgggcatggaggaagacgctgtggccttgttccgagagaagcttcgcagaaagaagaccgaggccgtcggtgctgccagtgataaagatgcgagccattgtgttgatggtaagtcttgtat

SEQ ID NO: 116 - DNA, 821 bp, unidentified
  source 1..821: mol_type = genomic DNA
agcgactccgacaacaacgaccaccgggacgactcatatccttcacaatggccattggacaatcctcgcagcagcaggccgacggccagaatgtcgtcacccagggcaactctgacaaggccgccaaccccatgcgcgagctgcgcatccagaagctcgtcctcaacatctccgtcggcgagtctggtgacagacttactcgtgccgccaaggtgctcgagcagctgagcggtcagacccccgtctacagcaaggcccgctacaccgtccgtaccttcggtatccgccgtaacgagaagatctccgtccacgttaccgtccgtggcgccaaggccgaggagatcctcgagcgtggcctcaaggtcaaggagtacgagctccgcaagcgcaacttctctgccaccggtaatttcggtttcggtatctccgagcacatcgacctgggtatcaagtacgaccctgcgatcggtatctacggcatggacttctacgtcgtcatgtcccgtcccggtgagcgtgtcgcccgccgccgtcgcgcgaagacccgcgttggtgcttctcacaaggtcaacgctcccgaggtcatcaagtggtacaagaaccgcttcgagggcatcgtcaggtaaaaagcttgaaaggtggtctgggatgatgaaaaattcaacttgtggttttggcaacggcgcaaaagagcgaggctatttttccgtagcttgaggatatatccggcctatcggagctttacttttacgcttgagcaagatcgcaaaaatggaggcctcgtataccaagcgagcgtgccgcataaccattgatcgctc

SEQ ID NO: 117 - DNA, 674 bp, unidentified
  source 1..674: mol_type = genomic DNA
ggacacaccggtgacgtcttgagcgtctcgttctcggccgacaaccgacaaatcgtttctgcttcccgagaccgaactatcaagctctggaacactctcggagagtgcaagttcaacattgttgacgatggtcactcggagtgggtctcttgcgttcgattctctcctaaccccgtcattcccgtcatcgtctctgctggttgggacaaggtcgtcaaggtctgggaattgtccaagtgcaagctcaagaccaaccaccacggtcacactggttacatcaacaccctcgccgtttcgcccgacggatcgctcgccgcatccggtggaaaggatggcatcaccatgctttgggatttgaacgatggcaaacacctctactctctagaggctggagacattgtcaactcgctcgtcttctctcctaaccgatactggctctgtgccgccactgcttcgtcaatcaagatcttcgacttggagtccaagtcaatcgttgacgacctcaagccagacttctccgccgagtactctgacaaggctcaaaagccacaatgtacttccctcgcctggtctgccgatggtcagaccctctttgccggtttctccgacaacctcgtccgagtctgggttgtcactgcttagagtcgtgaggattgtatgcatggataacgtgg

SEQ ID NO: 118 - DNA, 1183 bp, unidentified
  source 1..1183: mol_type = genomic DNA
gaagtaccgttttctgtgcagttttttttaaacccagaacttgcaattgagatcacgcgtcgcaatggcaccctctacccagaagcaatggaccgttaaaaacggggagcaggactttgacggcctcgtttacggcgacgcgccagttccgactgcgggggactcggaagtcgttgtcaagctccatggtgcctcgctcaactaccgtgacctgattatccccaagggaaagtaccccttcccgctctcgttcccggtcgtccccggctctgacggtgccggtgaagtcgtcgaggtcggatccaaggtcaagcaattcaagaagggcgacaaggttgttaccctcttcaaccagctccatcagtacggtcccgttgacgctgctgcggcatcgtcgggcctcggtggtgcggttgacggaaccctgcgccagtacggtgtcttcaatgagaacggcgtcgtcagggccccgaccaacctgaacttccttgagtcgagcacactaacctgtgcgggactaacaagctggaatgcgctgtatgggctgaagccgcttcttcctggccagaccgtcctggtgcagggcactggcggtgtgagtatctttgctttgcagttcgcaaaagcagcgggcgcaactgtgatcgcaacaacctcatccgaagagaaaggcaagcgccttaaggacctcggtgccgatcacgtcattaactacaagacccaaaccaactggggcgagatcgcgcgcggtttgacgcgcgacaacatcggggttgaccacatcattgaggttggaggcgccggcaccctggagcagagcttcaagtgcatcaagttcgagggagtcattagtattattggcttcttgggcggaatgaaccccagcaccatacccaatgttctgcagaccctgagcaacatctgcactgtgcgcggtgtgtatgttggcagcaaggcgctgatgaacgacatgatcaacgccatcgaggcgaacaatatccaccctgttgtggatggaactgtgttcacccttgagaagacacgagaggcctatgagtacatgtgggcgcagaagcacttcggaaagctgaccatccagatcgcttaatcacttgatgaatataatgagggatatatgcgactaggaattatgcgctaatgaatataataaccatgcaattag

SEQ ID NO: 119 - DNA, 1563 bp, unidentified
  source 1..1563: mol_type = genomic DNA
ggggccatgctcgagcagcagtaccagatgcgaaaggagcagcaagtgcaatttacacctatggcatcgccgtccagcactccttaccacatgcatcaagatttcactgttccgggcgactttttctcccccctcacatcgcctgcgctccacgctcagaatcagccacaatcgcgacagcaattcacggctcatcaacagggctactacacgaatcccagcaccgctgcgagctcggcggctccgagtccaatcgacgcgaacggagatgtggaaatgggtggcgacggtgttgcgctgccagagtcagcgagccaaccgaagaagccttcccgaaggaagcctgcgacaccgaggactttcgccatgaacaaggtcaagcaaagtcccatacaaaaaccgcaaaaaaggaagtctgtggcgttggcacacaaggatgcagatgctgtggtgcaggacgcccaacggtctggccatatcgcgcccaaatccgcaggtctccaaatgccgcctccgtttgagagctcggaaaacgacagtgtttcgccggaagcgctgaacgacctgcctatgggccccccgcctagacctggatcggtttcgcagtcgcccgccatcgctcctcagaatcagagcgtttctggaccggccgcgactcccaaatctctcctttctatgaagggcgctcaagatatgaatgcacctgccagtactggtatttctggccaaatgggacaggcatccttagaagatctcgaacttcccgaagctgccgaaaatccaggatcgactgcgacacactcgcaagtcttgaactcgcaagagccgacacctcgcctcatgccctcccgtaaaacgccaaaactcggccctcttagcacgccttcatcgggcaagcctacttctgcttccaacagtcccgctcatgcgttgtctcccatgacagcgagtacccctgctggtctgctgaaggacaagaaggacaacaaaggcggacgtgcaaccagcaagaagcgtggtagtgtcagtaccaccaattcagcaatggtctctccggcactccgaccgaaggtcagcccgagtatcaagcctctgctacccgaaggcaccagcctcaactccccgacccatgccctcctcctcgcctccaaatccaattaccagaacctcctggaaggcaaccacctccccggcatctcctacccggactccctctcaaccggcctcaccagcaaacgcacctcgcacaaagtcgccgagcaaggccgccgcaaccgcatcaacgacgccctcaaagaaatgcaagccctcatccccgcctcgtccggcgcccgcgccgaagagctcatgaccgccgacgccggcgacgacgacagccaggaaaccaaggagaaggaccgcgacgccgctgtcaagagcaatagctccaaagccgcgaccgtcgagagtgcgaatcggtatattcgcgtgttgaaggagagcgacgcggcgcagaaggatgcgatcgcgcggccgaattccccgggctcgagaagcttggatccaccggatctagataactga

SEQ ID NO: 120 - DNA, 939 bp, unidentified
  source 1..939: mol_type = genomic DNA
gacgacacaatgagaaacatcctcctagtattggcgtctgcagcgcttgctgttgtggcacaaaagccagatctcgacgtgaaaggcacgtttggagacgcgaaccccttctccaaggtcgtcaacggccaaagcaacaagctctacctcacgctggacaaccacagccctgagtctctggtggtcaagtctatcagcgggtcatggtctgagaagacgtccgcttcatccggtcaagagaagtttcttaagaactctaccacccaagagaagctcactgtccccatccctcccaagtccgagggcgcattccagcctcctacagtcttgacctaccagttctggagcgaattcaagcctagagagttgctcttgaccgttttgggttgactatgttgatgctaccggtcactcgtacagagaaacagcctacgaaggccaagtgactgtcgttgaggccccgggatctttctttgaccccgccttgctctttgcctacgccatggtgctggctctcgtcggcggcgccggctaccttgcctacaacatctactttccacctgcccgcaagcccagaagaagcgccaacaccgcacctacagatgctcctgctgctccggctgaccctgacgaatggattcctgtccaccacaagagggccaaaaagacgtctggcggcggggccaccagtggtgaagagagcgaagccactgaaggctatgcaagcgagaagtctgccagtggagccaagaagagaggcaaaggtggcagaaaataaatactgacatgtgcctcgagctgcagacgacgctcgtcaaaagtgtagcaagttgaagaagcccagcacgaagtccccagcttgactgctgccgtttggcttaatggcacagaaagcgagtgtacgtcgtacacggcttatagtctcgaatgcaacaaagg

SEQ ID NO: 121 - DNA, 896 bp, unidentified
  source 1..896: mol_type = genomic DNA
gccaaccacgacattgatccctttcacgacttgctttcgccatgtcagataacaacgatggaaatcacgggggaggcgtaggcgcgtcttactactatggcggcatcgcgattgcgctgtgtcttgtgattgtgttgacgcttgtatcaagaatattatatcgacgacgtgtaaggaacagactcctgcgagccaacagacaagagcgcattactttgcgagaccggggagaagcgccaggcctgccaacctatcgggagtctcgcaatcagccctcattaccgcgatacacggccgaggcagactacgcacctccacccggcccgcctccttccaacagcccggacaacgaaggccaccacttccacttccatttcccctctttacatgtgcctcaggcactgcacttgcggcctaggcaggcagacgatcctgctgaccagatccccaccgtgccccctccgtcctacgagccgcccaagtatgagccgcccagtggagcgcctccagagcagcaacaagagcctgtggctagcgggagtagcgagcatcatcaccagcagtctgctttgggcgaacataccgcggcggcaacagccgctgccacaactccggcagagcacagtggcgagtcgacagagcttaggagtgcgtcgccttctcagccacaatctcaatcccaacctcaagcaccagcacaaccacaagagcaggattacggctacgacgatgccgactttatccatcctgaagagcgacgcaggatcgaggctgcgcagcgcaatgatccgcagacatgattcaaacatgtgttgtaaagtgtactactatgaactcgttgaccagtataatcgaagcgtatataacggcacaaatgcaaagctgccatcatcccg

SEQ ID NO: 122 - DNA, 697 bp, unidentified
  source 1..697: mol_type = genomic DNA
cattacggccgggaagctttcagaagctaatcgagttcttcctatccctttcaactttacacaccatgtctagaattggcgatccaacgaacaaccctgctacccagcaactgtactctgataggcccttgcatctccctggccccggcctcaagccatccaggcagctcactatcagctcggctgttgcgttccgcgaggattcgggccaaacacgcttcaacctcatcagctctgaccaccgcgaggtgttgcacattagtattcgtgcaagggacaacgttctcgtgctcaacaccaaggcccccgatggcgattggggcaaagaagagcgacatgatctcaaaccccttttcgataccccactgctgccttacatcaccgtaatggcaacgaagaacagctatatcctttctgttcctggtaaacgggagatcatcttcaataagaggaaagggttcatggagcctgctgtgaggattgagtatgactatgatgagatgtctgcgttctccgacccctgctacattacagtcccatcttcatcttaaagctttcctagttggcttggagttggcggatatggtcacattggttttttcacacggcaaacggtaaagaattacggcttctctctcctgtcatgttcagcggacgatgtatgatgtagtgttctgttcaattgatctggttgttg

SEQ ID NO: 123 - DNA, 329 bp, unidentified
  source 1..329: mol_type = genomic DNA
ggaacgaccacgagcagtttttaaaatgccacaagaaatcaaggacatcaagaacttactcgaaatcgctcgtcgtaaggacgctcgttccgcccgcatcaagaagaccaagaccgttggtgctaagggcgagccagctcaacttaccaagttcaagattcgttgctctcgctacctctacactctcgtcgtctctgacggtgagaaggcagagaagcttaagcaatcactcccaccaaccctcaacgtcgaggagattggtaaggtttcaaagaagtagattagtgatgtaatttgctgccttgattgattgtccttgttggtatttt

SEQ ID NO: 124 - DNA, 747 bp, unidentified
  source 1..747: mol_type = genomic DNA
gtacacataactcttcattcctcatcatgtctcacactttctacgatggcaccatcgtggtgcttcaaggcattcttgaaactttttctcatatccttcacaaagccgaagaaagcccaaactctagcgcttttcccgcagctcgtctgcacgaggacatgtatccattgaccgaccaaattcgcctagcaactcaattttctgagtatattctggctaaagtgaccggccgcgagccaaggaagttcgaaggcaatccattgaccttcgctgaattctatgagcgtatcgataccatgctgaagtcactcaaagaagcagataaggatgtcgtcaatgcaaatgccgacaaggaggagcttactcaagttggacctaccgcaaaaattgaattgagtaatgctatatacgcccatcgcatagccttgcccaacatttacttccatctcaacattgcttacggcattttgcggaaggagggcgtgcctcttggcaagcttgactattttgcgggctttttcccaccgagcatggctcaaggcaagtaaagaagtgatgttggttatgtttccggatggagagggtgctgatctatgagaatgagttccgagtagaccatgatggtctagatgtggacttgagctttcatttgccaaattcttgtggaaagatagcaatgacggaacaagcgatttgtatgtacatttaatgaagtctatctatagaattaatctccgatctatcgcg

SEQ ID NO: 125 - DNA, 693 bp, unidentified
  source 1..693: mol_type = genomic DNA
aagctcttcaaagctaattatcgagttctcctatctcttgcacttatacacaccatgtctagaattggcgattttgcgaacaacaaccaggctacccagcagctgttctctgatagacccatgcagctccctggccccggccttaagccgtccaggcagctcacggtcagctcagctatggcgttccgctgggactctggccaaacccgcttcaacctcatcagctctgaccgtcgtgaagtgctgcacatcagcatccgcgcaaaagacgacgtccttgtgcttaacactaaggctcctgatggcaattggggcaaggaagagcgacacgagctcaaaccccttttcgacaccccgatgctgccttatatcaccgtaacggcgactaagactagctatatcctgtccgttcctggtaatcaggagatcatcttcaataagaggaaagggttcatggagcctgctgtcaagattgagtatgactatgacgagaaccctgcgttctctgatccgtgctacgtcacagttccgcatttatcttaaggtcttattggcttggagttggcggatagtcacaccggtttttttcacacggcaaaaggcaaagtattacggcttttctctcctgtcctgtttagcggatgtacgatgtatgttgtagtagtgttctggaatttgtgttcaagttgttgg

SEQ ID NO: 126 - DNA, 1030 bp, unidentified
  source 1..1030: mol_type = genomic DNA
gagcgacttcatcaaaaatgtcagagcaacttcactacaagggttcattggccggccacggcaactgggttactgccatcgctacctctgcagagaacccagacatgatcctcactgcttcccgtgacaagtctgtcatcgtctggcaactcacccgtgacgacgctcagtacggttacccaaagagaatcctcaagggccacaaccacttcgtctctgacgtctccatctcatacgacggtcaattcgctttgtcctcctcatgggacaagaccctccgtctctgggacctcaacactggtcttaccaccagacgtttcgttggccacgaagcagacgttctctccgtctccttctccgccgacaacagacaaatcgtctctggctcccgcgaccgcaccatcaagctctggaacacccttggtgaatgcaagttcgacatcaaggatgaaggccactccgaatgggtttcatgcgttcgtttctctccaaacccaatgaacccagtcatcgtctcagctggttgggacaaggttgtcaaggtttgggaactctcaaactgcaagctcaagaccaaccactacggtcacactggctacatcaacaccgtctctgtctccccagacggatcccttgctgcctccggcggtaaggacggcatcaccatgctttgggacctcaacgagggcaagcacctctactccctcgaggctggtgacattgtcaacgcactcgtcttctcaccaaaccgttactggttgtgcgctgctactgcctcatgcatcaagatcttcgacctcgagtccaagtccatcgtcgacgagctcaagccagactttgtcgacgtcggcaagaactcccgcgagccagaagctgtctccctctcctggtccgctgatggtcaaaccctcttcgctggtttcaccgacaacgccgtccgtgtctggaccgtcgcataaaactaagctgtatctaatagacagggtattgggttttgtaacactattgcgaggaactcatgattttaccg

SEQ ID NO: 127 - DNA, 668 bp, unidentified
  source 1..668: mol_type = genomic DNA
ggggaaggtggtggtatccacggaaccaccttcaactccatcatgaagtgtgatgttgacgtccgtaaggatctctatggcaacattgtcatgtctggtggtactactatgtaccctggtattgccgaccgtatgcagaaggaaatcaccgctcttgctccttcgtcgatgaaggtcaagatcattgctcctcctgagcgtaaatactctgtgtggattggtggttccatcctggcttctctgtccaccttccagcagatgtggatctcgaagcaggagtacgacgagagcggcccttcgatcgtccaccgcaagtgcttctaagcttaagcgcatggttgatttgcttgtttgtacttcttttctggcgtatcaaaaggcaggacagtgtggcatgcggaccttttctgacctgatgacgagagggatcgcctaagaaaaaggaactttattttagttgtggaatagagacggtttatttgacgctagttctcgtccagagcatcctcgagacgatagtctgggttcgtcttaagcgatggatggtggtgattctcttcgtattgttcctgtacctgtactacatattgcctacaccatgtcctgttcatttcttctctgtttgcgttgcgttagaccttataaatttaaatgtcgtattgctcccc

SEQ ID NO: 128 - DNA, 668 bp, unidentified
  source 1..668: mol_type = genomic DNA
ggggagcaatacgacatttaaatttataaggtctaacgcaacgcaaacagagaagaaatgaacaggacatggtgtaggcaatatgtagtacaggtacaggaacaatacgaagagaatcaccaccatccatcgcttaagacgaacccagactatcgtctcgaggatgctctggacgagaactagcgtcaaataaaccgtctctattccacaactaaaataaagttcctttttcttaggcgatccctctcgtcatcaggtcagaaaaggtccgcatgccacactgtcctgccttttgatacgccagaaaagaagtacaaacaagcaaatcaaccatgcgcttaagcttagaagcacttgcggtggacgatcgaagggccgctctcgtcgtactcctgcttcgagatccacatctgctggaaggtggacagagaagccaggatggaaccaccaatccacacagagtatttacgctcaggaggagcaatgatcttgaccttcatcgacgaaggagcaagagcggtgatttccttctgcatacggtcggcaataccagggtacatagtagtaccaccagacatgacaatgttgccatagagatccttacggacgtcaacatcacacttcatgatggagttgaaggtggttccgtggataccaccaccttcccc

SEQ ID NO: 129 - DNA, 1018 bp, unidentified
  source 1..1018: mol_type = genomic DNA
gactttctcatttcagaattattttctatactctgacaagagcaagcaataccaaacatcttccacatcgaagctttaaccattttgcccttaacatttgaacaagacgaaatggccttcttcccacactacaccactaatctgtcgcctctgctctacttgttggacgacgactatgctgtctaccgctcaacttgtccaaagtccaactaccaccacaagcaacaccacagccgccgtcagccttcgccagttcgttactttagtccgaattttgatatgcgagaggggaatgactcctactaccttgacggagagctccctggtgtcaaccagaatgatgtcgatattgaattctctgaccctcagacactggtgatcaagggtcgagtggagcggaattacaacaatctcgacggcatgaacgaggaaaaccagcaagatgaagaacaattctctgaaactctctctagcaagtcgtaccaacccactgtcgaggacgaggacgaggcgaaccattcaccacccgtggcgacaccaacctactctgagaagtctgttactgagaaaactcagaagcctgcgtacaaataccgaaattctgaacgtgctattggcgaattccaccgagccttcaatctccctacaagagtcgatcaagatgcggtcagggctacattgaggaatggaatcctctcgctggagctcccgaaggagccggcaccgaagatgaagaagattcggattgaatagaggatttcgaataaaatttttgatttgatgagtagttggtgtttattgttatgtctaattatatggggctatgtcatgattgggaaatgggacaccgcatttgtttcctttttccccatttcttcagacgccatctatattgcatgtatgttgcatgaactatggtttttgctaggagcggttgcttctgctttgcattttcatgaactattttctttttattaaattaataactagcatatcaattaatgatctgtcatatggc

SEQ ID NO: 130 - DNA, 686 bp, unidentified
  source 1..686: mol_type = genomic DNA
aagctttcagaagctaatcgagttcttcctatccctttcaactttacacaccatgtctagaattggcgatccaacgaacaaccctgctacccagcaactgtactctgataggcccttgcatctccctggccccggcctcaagccatccaggcagctcactatcagctcggctgttgcgttccgcgaggattcgggccaaacacgcttcaacctcatcagctctgaccaccgcgaggtgttgcacattagtattcgtgcaagggacaacgttctcgtgctcaacaccaaggcccccgatggcgattggggcaaagaagagcgacatgatctcaaaccccttttcgataccccactgctgccttacatcaccgtaatggcaacgaagaacagctatatcctttctgttcctggtaaacgggagatcatcttcaataagaggaaagggttcatggagcctgctgtgaggattgagtatgactatgatgagatgtctgcgttctccgacccctgctacattacagtcccatcttcatcttaaagctttcctagttggcttggagttggcggatatggtcacattggttttttcacacggcaaacggtaaagaattacggcttctctctcctgtcatgttcagcggacgatgtatgatgtagtgttctgttcaattgatctggttgttgac

SEQ ID NO: 131 - DNA, 698 bp, unidentified
  source 1..698: mol_type = genomic DNA
gggcatataaccacaggtcattcgcatccgtcgcagaacttcttacatctgagctttcctgtccgttagatataggcaaaatgaaggcctactggtacgataaccaaccgggcgaccagcgcttgcctcacgactccggccgccccgtcaccgagtcctacctcgagtccatcggcgtcttctaccgccactgcccaacaattgaccttgtcgactccctggccgccgagcgcggctacaagaaccgcgacgaggtctgcgtctcgccgcagactatgggcgatgtctacgaggagaaggtgaagacgttctttagtgaacatttgcacgaggacgaggagattcggtacattcgagatggggaggggtactttgatgtgcgtgggcaggaggatgagtgggtacggattcggttgagtaaggatgatctgatcattcttccggctgggatctaccatcggtttacgacagatgataagaactacgtcaaggctatgcgtctcttccaggaggagcccaagtggacgcccttgaaccgtggccctgaggttgatgtcaaccctcaccggaagacatacctggaaaccgtccccagccctgctgtggctgcgaactaagtgagcatcgaatgctcttgttgaacaatctatttgcacatctttagcctttatacacctcaatgcatcaatggatttagg

SEQ ID NO: 132 - DNA, 884 bp, unidentified
  source 1..884: mol_type = genomic DNA
gtggctgccggctacacccccgaggccctcgagatcctctccaagaagaagggcggcaagtacctcgtcctcgagatggacgagacctacaacccccccgccgaggagacacgtactctctacggtgtccagctcacccaggcccgcaacgatgctgtcatctccccccagaagaccttcaataccatcattacccccaagaacaccgagtccctccccgagtccgccctccgcgacctcaccgtcgccaccctcgccctgaaatacacacagtccaactccgtctgctacgcgctcaacggacaggtcgtcggcctcggtgccggccagcaaagtcgtatccactgcactcgtcttgccggcgacaagaccgacaactggtggatgcgcttccacgagcgcgtgctcaacatcaagtggaagcagggcaccaagcgtgctgacaagagcaacgccatcgacctgctctgctcgggccagacgccccgcaatgacgctgagaaggtcgagtacgagcgtgtgttcgcggaggttcctgctccgttcacccaggaggagcgtgatgcttggctctcgcagttgaccaacgttgctatttcttcggatgctttcgtatgtctctcccctctgttagagcattctaagttctaagatcatgctaattggtgaaatagttccccttcatcgacaacgtcttccgagccgcccgctccggcgtcaagtacatcgctgcacccagcggttcgcagaacgacggccctgtcttcgagactgccgagaagcttggtatctcgttcgttgagcagggtactcgtctgttccaccactaacttgcttttccggtggcgtggtattatggtataaaaagaaaaagggtttggggg

SEQ ID NO: 133 - DNA, 822 bp, unidentified
  source 1..822: mol_type = genomic DNA
gtggctgccggctacacccccgaggccctcgagatcctctccaagaagaagggcggcaagtacctcgtcctcgagatggacgagacctacaacccccccgccgaggagacacgtactctctacggtgtccagctcacccaggcccgcaacgatgctgtcatctccccccagaagaccttcaataccatcattacccccaagaacaccgagtccctccccgagtccgccctccgcgacctcaccgtcgccaccctcgccctgaaatacacacagtccaactccgtctgctacgcgctcaacggacaggtcgtcggcctcggtgccggccagcaaagtcgtatccactgcactcgtcttgccggcgacaagaccgacaactggtggatgcgcttccacgagcgcgtgctcaacatcaagtggaagcagggcaccaagcgtgctgacaagagcaacgccatcgacctgctctgctcgggccagacgccccgcaatgacgctgagaaggtcgagtacgagcgtgtgttcgcggaggttcctgctccgttcacccaggaggagcgtgatgcttggctctcgcagttgaccaacgttgctatttcttcggatgctttcttccccttcatcgacaacgtcttccgagccgcccgctccggcgtcaagtacatcgctgcacccagcggttcgcagaacgacggccctgtcttcgagactgccgagaagcttggtatctcgttcgttgagcagggtactcgtctgttccaccactaacttgcttttccggtggcgtggtattatggtataaaaagaaaaagggtttggggg

SEQ ID NO: 134 - DNA, 996 bp, unidentified
  source 1..996: mol_type = genomic DNA
tgaagcagtggtatcaacgcaagcagtggtatcaacgcagaatgtgcgatcgctctagaatcggtcccaagggttgggaagcagtggtatcaacgcaagcagtggtatcaacgcaagcagtggtatcaacgcaagcagtggtatcaacgcaagcagtggtatcaacgcaagcagtggtatcaacgcagagtgcgcagcccggtgctatctctgctcctgtggcagctggtaaggacgttgagctgcagtggaccgaatggccggaaagtcatcatggccctgtcattacttacctggccaactgcaacggtgactgctctgaggtcgacaaatcctctctggagtttttcaagatcgatcagaagggtctcatcgatgacagcaatgtccctggcacatgggctaccgacaaactaatctccaacaacaacagctacaccgtcaccatccccagcgacattgctgccggtaactacgtcctccgccatgaaatcattgctctgcactccgctggcaacgaggatggtgcccagaactacccccagtgtctcaacctcaaggttactggtggtggcaacgcttctccctcaggtactcttggtaccaagctctacaacgaggacgactcgggtatccttgtcagtatctaccagcagcttgactcctacgacatccccggccctgctctgtactctggcgcttcctcgtcctccaactctggttcttcttccagcgttgcttcggccactgcttctgccacttctgccgctgcttcctctccctcgtcctctcaggcttccggtacccccgcttcccaggtcaaggctcagaccgctagctctactcctagcgcttcgtccggtgccacttccggcagtctgtccgactacttcagctctctgagcgctgaggagttcctcaacgttatcagcgagactctgtcttggttggtcactgacaagattcacgctcgtgacttgtcgaccgcataaatgg

SEQ ID NO: 135 - DNA, 823 bp, unidentified
  source 1..823: mol_type = genomic DNA
gtggtatcaacgcagagtgcgcagcccggtgctatctctgctcctgtggcagctggtaaggacgttgagctgcagtggaccgaatggccggaaagtcatcatggccctgtcattacttacctggccaactgcaacggtgactgctctgaggtcgacaaatcctctctggagtttttcaagatcgatcagaagggtctcatcgatgacagcaatgtccctggcacatgggctaccgacaaactaatctccaacaacaacagctacaccgtcaccatccccagcgacattgctgccggtaactacgtcctccgccatgaaatcattgctctgcactccgctggcaacgaggatggtgcccagaactacccccagtgtctcaacctcaaggttactggtggtggcaacgcttctccctcaggtactcttggtaccaagctctacaacgaggacgactcgggtatccttgtcagtatctaccagcagcttgactcctacgacatccccggccctgctctgtactctggcgcttcctcgtcctccaactctggttcttcttccagcgttgcttcggccactgcttctgccacttctgccgctgcttcctctccctcgtcctctcaggcttccggtacccccgcttcccaggtcaaggctcagaccgctagctctactcctagcgcttcgtccggtgccacttccggcagtctgtccgactacttcagctctctgagcgctgaggagttcctcaacgttatcagcgagactctgtcttggttggtcactgacaagattcacgctcgtgacttgtcgaccgcataaatgg

SEQ ID NO: 136 - DNA, 1000 bp, unidentified
  source 1..1000: mol_type = genomic DNA
gacggtgaagttggaatagaataaaatgttgagcatgtttaccagagtggccagaggacaggccaaggtgtttacccgcaacgcatccacagcatcatccaaaccaacgaatcaatcatccaacaaggctgccactatcgcagcttcaatttcaggtgttaccgccgcgttatacgcccaccaatacggcctcattgacagcgtcttcgctagtggcttagaagagggtttgcacgctcctcatttcccttggtcacacaatggctggttggacagctttgaccacaactccattagacgcggttaccaagtttaccgtgaggtgtgcagctcgtgtcactctttggacagaatagcgtggagaaaccttgtcgctgtgtcacacacttcagatgaagccagagcgatggctgaagagcaagagtacactgatggtccaaatgaccaaggagagtctttccaaagacctggtaaattggctgattacatgccagctccttatccaaatgaggaagcttcgagggccgccaatggtggtgctcttcctcctgatctttctctcatcgttaaagcaagacacggaggagctgattacattatggctctgctcactggttaccaggatcctcctgctggtattcaagttcaagagggcatgaacttcaacccatatttcccaggtggtggtattgccatgggtagagttttgttcgatggtctggtagaatacgacgatggcactcctgctactactacacaaatggctaaggatgtcgctactttcctcagctgggctagtgagccagaacacgacgacagaaagaagatgggcttccaagctgtcattatcctctcagctatgaccgccatctcactctacgtcaagagactcaagtggtcgcctatcaagacgaggaaactgacttacaacccaccaaagtgatctgaatgtagagaaaagtttgacccgtataaaaaatttcatcctctcctttttccg

SEQ ID NO: 137 - DNA, 544 bp, unidentified
  source 1..544: mol_type = genomic DNA
ggaggtagtccagccaaaaagagtttgataggcgcgatggaggcacaaaatctcaagactttcccaaagcaacctatcttccaaaactcaaagacccgtggtaacaagaaggtcaccaaggaccgtcgttggtacaaggacgtcggtctcggtttcaagactcctcaagaagccatcaccggtacttacatcgacaagaagtgcccatggaccggtgaggtttccatcagaggccgtatcttgtccggcaaggtggtctctaccaagatgacccgtacgatcgtcatcagaagagagtaccttcactacgtgccaaagtacaacagatacgagaagcgtcacaagaacctcccagtgcacgcatcacctgcattccgtatcgagaatggtgaccaagtcgtcgttggccaatgccgtccactttcaaagactgtgagattcaacgtcctccgtgtcatcaagaacaaggctgctgctaaggctttcgcaaagttctaaacttgttattaatgtagttggtccattcacagaattttgaaagtcc

SEQ ID NO: 138 - DNA, 938 bp, unidentified
  source 1..938: mol_type = genomic DNA
acgttcaattgacttttccattcttttgttcgttctgaagagttttcttttttctttcattgtcgcctcctttctttcgccttcccgttgtttccgatcatcgggtgttgccagagtatatatagagttggggctcccttttatctatctcaccgcaaccgtcctcctgatcctctctcctgatcctcctccttcattccttgactcctcttcacgctcctcctgaccagccaagtcttacatccctctctacaactactactcttcaaataatcctctcctctcggtgggcttgaatcccttatcttccgcctctccccacgaaccggaccggatcgtcttatcgcctccgcaccagctggcgcttactacctcatccacctctttcccgtctcgccaccgaaaccagtctacaatgcctcctcgcaagcccagatgctcctttaaggagtgcaaggaacaagcccagcgcattgtcggagactgcagcttctgcagcggtcacttctgctccaagcatcgcatgctcgaagcccactcctgctccggtctggaagactgcaagaaggagtcccacgcccgcaatgctgataagttgaacagcgagcgcacacaggttatcaagggtgtatgacgggatcacatctacactactacaacaatctcggcgcatttcgattgcatttacttgccatttttatccgacgttgagttagcgcggtgttatttacaatttcttcttttctttattttgcctacgatgtctccccctatcggtatggtggtgtctcgtttcgggagcgacatggtttacaatgattttggtttggggtggtctctcggtatttgtctattatccacttattttccggggtattatgcgcatggcgttactatatggagtttgatattctatctcgaatcgatacttttacaac

SEQ ID NO: 139 - DNA, 687 bp, unidentified
  source 1..687: mol_type = genomic DNA
aagctctcccaagctaatcgagtttttactgtcacttgcatttatacataccatgtctaggaacttcggcgatttttcgactaaccaggctactcagcagctgtactccgatagacccttgcatctccctggaaatggccttaagccggctagacagctcacgatcagttcagctgtcgcattccgctgggactctgaccaaacccgcttcaacctgatcagctctgaccgtcgcgaagtgttgcacatcagcattcgcgcaaaagacaacgttctcgtcctcaacaccaaggcgcccgatggtgactggggcagggaagagcgacacgagctcaagaaacttttcgatacccctatgctgccttacatcaccgtgacggcgacgaagatgacctataacatcactgtccctagtggtcaagaaatcatcttcaacaagaggaaaggattcatggaacctgctgtgaagattgagtatgactatgatgagcactctgcgttctccgacccatgctacattacggttccatcttcttaaaggctcgtcggcttagagttggcggatagtcacactggtttttcatacggcaaacggcaatgtattacggcttttctctcctgtcctgttcagcggtagatgtacgatgtatgttgtagtgtttcaaatttgagttcaagttgttggcc

SEQ ID NO: 140 - DNA, 601 bp, unidentified
  source 1..601: mol_type = genomic DNA
gtctgcttgcttcaacgagcgctgaatttcttgttggccggtgttgattccccggattccctgttatcgcctgctgtgttcgccttggtagcaggtgtttgaattggtcctgcaatttcgctgattgctggcctcgaactcccgatcgaacgaactctcctctcctccgtcaacgcaccttcacatatcgcaaagcacaatggtttccaagattctcttctggagtggcttcggcatcgccgtccgtctctggcaactcggtatcgaaatgcgtcccattcttgccaagcagggtctctgggcctaccccgtcttcgcaggtgtcggtggaagcttcggttactggctccagggtgtcgaggaccgtcagctgaagattcttgcgcagcgccgcgaagccatcctcgacaagcgccggagacgggacgagcgtgagggtctgagcaacattgagaaggagggtactttggctgcgaccccatgatttgttgcgttggctgttgtttattttcactgccttcggagaaagaccggcaattgcattgctgggcatgtatcataccaaaacagaggaaggttaatggtcaattgtttaatgacc

SEQ ID NO: 141 - DNA, 601 bp, unidentified
  source 1..601: mol_type = genomic DNA
ggtcattaaacaattgaccattaaccttcctctgttttggtatgatacatgcccagcaatgcaattgccggtctttctccgaaggcagtgaaaataaacaacagccaacgcaacaaatcatggggtcgcagccaaagtaccctccttctcaatgttgctcagaccctcacgctcgtcccgtctccggcgcttgtcgaggatggcttcgcggcgctgcgcaagaatcttcagctgacggtcctcgacaccctggagccagtaaccgaagcttccaccgacacctgcgaagacggggtaggcccagagaccctgcttggcaagaatgggacgcatttcgataccgagttgccagagacggacggcgatgccgaagccactccagaagagaatcttggaaaccattgtgctttgcgatatgtgaaggtgcgttgacggaggagaggagagttcgttcgatcgggagttcgaggccagcaatcagcgaaattgcaggaccaattcaaacacctgctaccaaggcgaacacagcaggcgataacagggaatccggggaatcaacaccggccaacaagaaattcagcgctcgttgaagcaagcagac

SEQ ID NO: 142 - DNA, 380 bp, unidentified
  source 1..380: mol_type = genomic DNA
gggggcgacatggcttcgacgccggtgacaatccctgaggtgcatgccgagagcgtaacgtatctcgtaaatccaacgttacgataaaatagtcgcaaacgacgacaactacgctcagggcgcgctggcagcgtaacaactgctagcttctagtccggcccggaggtgatgtgcccattcatcaccgaagggatacgagctcagactgatgggatcgcggctggctttgtcctcgcgtcagccgctaaaacttagaggaatcgcgtcgctggatcctgcccgtcggagccagaggcgctaaatcaaaagacggacctaagcatgtagagccgatgggtgagtgccggcggacgggggttcaattccccccgcctccacca

SEQ ID NO: 143 - DNA, 977 bp, unidentified
  source 1..977: mol_type = genomic DNA
gggaccagaacagcttcagctacaatgccattcatcaaggaagccaagagcaacagctacttctctcgctaccaagtcaagtaccgcagacgtcgtgaaggcaagactgacttctacgcacgtaagcgcttggtaacgcaagctaagaacaagtacaacgcaccaaagtaccgtctcgtagttagattcacgaacaaggacatcatctgtcaaatcgtgtcatcaaagcttcaaggtgacgttgttctcactcacgctcgcgcccgcgaacttccacgttacggcatcaagcacggtctcacgtcatggtcatccgcttacgcggttggtctcctcgtcgcaagaagagcgctcaccaagctcggtcttgctgacaagtacgagggtgacgttgaagctactggtgaatacaacctcaccgagccacttggcgatgatgaaccacgtcctttcaaggtcttccttgacgttggtcttaagcgtacctctactggttctagagtcttcggtgctcttaagggcgcctcagacggtggtctctacatccctcactctgagaaccgtttcccaggttacgatatcgagagcaaggaactcgacgctgaaatcttgaacaagtacatcttgggtggtcacattgctgagtacatggaggctcttgaggaggaagatgaggagagattcaaggctcaattctctacctatcttgaagacggtattggatctgaggacattgaagaaatcttctcgggcgcacacgaggctatccgtgctgacccaaccttcaagccaagtgaggctgccaagggcaccgactggaagtccgagtcaaagaagcaccgcgctgtcagactcaccaagcaacaacgcgaggacgctatccaacagcgtatcaagtactaccagcaagctggcgacctcgagtaaacggtaattgtagcggtctacatagacaatcaatgtctgttgttccttag

SEQ ID NO: 144 - DNA, 73 bp, unidentified
  source 1..73: mol_type = genomic DNA
gattacccgctgaacttaagcatatcaataagcggaggaaaagaaactaacaaggattcccctagtaacggcg

SEQ ID NO: 145 - DNA, 823 bp, unidentified
  source 1..823: mol_type = genomic DNA
gggtctcttccatttgaatttttcaacccacagcatggccttcatgaatctcccatggcccactgaatgcctacatgccgctctcaagaacggatccttacctttctggggatttgtaatctatcgaacgacctacaccgctcagtcagatgccgcctggccgcagattatcgagcttattgcctcctatatgaaagccttactctaccacgagtataacgacaagaaaaaagatggagatgagcctacagtctacgacgaaatctgggcaaggcatcagttgacgattatggatgatagacaattcaacggagcgtctgtgtttgatatccaacttcacttcgaaaagtgggttgaggcgcagggaaagcgagatgaatctactatgtatcgcatgtgtatggtcattgatgatgaatcaatccagacgttattggaggcgccacccggggaaaataggaaactcggacgacgtatagggggccctgtacgctttgtcaaagtcgtggaggctttccccgagctagacagccttgacgaattccagggatggatgaaatgtgagatcaacgcgttatggccgctgtggaagatgatgtctgacggagatgaaatgaggatgtcatatgatgagatgaaggggaatggaaagcaggtctatggcgcaatttaatcggtttttcttcatgttatcctgatggaaaaaatggcagaacatatgtctgtacatgcagaaaataaggtgattggaaaatacttgaatgctatgaagttagatagtagctgttctagcggccagataaagccgcgcatgtgaatttcg

SEQ ID NO: 146 - DNA, 181 bp, unidentified
  source 1..181: mol_type = genomic DNA
gtactaaccactcgtcaggcgctgaaagaagaagatgcagtaagtttgatgttttctgtgtatgattatacataaagttgttgtataactacgcagaaaagttttcgtatgcaaaactttgattggtgttaagtcgaaataaggttcgtgtaatggaaattgcacggggagtataaaatgt

SEQ ID NO: 147 - DNA, 734 bp, unidentified
  source 1..734: mol_type = genomic DNA
atgacagagaaactttacaccgagcaagtgaatgcgttcggaaacgaattacctcctctatcatacaaagacctggacaaactccccttacaccaaaacgtcatcaaagaaactcttcgaatccacaactcaattcatacactcatgcgtaaagtgaaaaatcccctcccagtcccaggaacaagattcgttataccaaccagtcacaccctcctcgcgtccccgggcgtaacaacccgcgacgattcacacttccgaaacgcaatgacctgggatccacaccgttgggaaacacgaagtgaggtcgaagatgatggtgagacaatcgattatggatatggggttgtttcaaaggggacgaagagtccttatttgccctttggagcgggtcgacatcgatgtattggggagaaattcgcatacttgaatcttactgttattgttgctactcttgtgaggaattttcggttttctgaacctgatgatagagagggtgttcctgaaacggattattcgtcactcttttctagacctatgcggccggcgactgctcggtgggaacgacgtggggagtactagaggtgggattattggggatttgattgctttttggaattgggatggaagagttcttgggatatattcttgttcttcgaggctttcccaggtgatttttcacagggcttggtattatcgtatttaatcaatcaattcactacacttttcgagcttgc

SEQ ID NO: 148 - DNA, 801 bp, unidentified
  source 1..801: mol_type = genomic DNA
gacatcattgcatccaatatcagatcacattccatagcctcatttactgcccaagaaggcttatggcccattggagagaggagtacctaactgcactggcagtgcgagatcagagggagaaggccaatctcagtatttacgatgcctatacccgactcgcagatagcacggccaaacttccagctacaatagatacaagtggcagcccctcaggtgataaagggccatctggtacctacgagtccgaaaagacggcattttctcaatcaaggacagccaagaagcagcagacagaagtggagccttcagttacggagcttctaaatactacacgtgcagaactagccgaagcacagcgctctcgggcagaattgcgagatcgtctagagcgagctactaacgaagcggagaaattgcggaaacagattggcaaagatggtcgacgaatacatggactggaaaatgaagttgctcaacagcaaaagcgccgcaaagatgttgaagaagagttgagaggaaaggctaagctactcaatgaattccaagacgaaattgcagctctgactctccaggtgaacatggccgagagaaaagctaagaagcttggagaggagaacgatgatcttgttaatcgttggatgaagagaatgggccaggaagctgatgcaatgaatgatgcctccaagttttcgtgactgccgaatcagaatagaatcaaatggcccagataggccttcgcattgttatgacatgatcgaattccgaggcaaattcgcctatcatggtaatgaacggataaag

SEQ ID NO: 149 - protein, 213 aa, unidentified
MAFFPHYTTNLSPLLYLLDDDYAVYRSTCPKSNYHHKQHHSRRQPSPVRYFSPNFDMREGNDSYYLDGELPGVNQNDVDIEFSDPQTLVIKGRVERNYNNLDGMNEENQQDEEQFSETLSSKSYQPTVEDEDEANHSPPVATPTYSEKSVTEKTQKPAYKYRNSERAIGEFHRAFNLPTRVDQDAVRATLRNGILSLELPKEPAPKMKKIRIE

SEQ ID NO: 150 - protein, 164 aa, unidentified
MQLLSTLTPLALLVTVASATGKAVNNAVGNAVVTNHCKDPIYLWSVGSSVSPKHTIPSGSNYTEPFRHDDASGGIALKITRNDNGLYDGSAQLVYSYALDGEQVWYDLSSVFGDAFAGEAVAVKPENEGCGSICWPKGTTPGGSQVKVCDAEGDVGLVVCAKGC

SEQ ID NO: 151 - protein, 149 aa, unidentified
LLAEAIRRGLLGWRRAEAKWQRCCCWLSTGHSARKCTREHSLVLHERLSRNRARHWQGVVALCLRCQQPLCFRTVSFALTVLLECIINRQRLVHQVLAIHSRDCLVRAVKVVVLDESISFQKKKKKKKKKTGGQVLKLPEGISPHGQKF

SEQ ID NO: 152 - protein, 119 aa, unidentified
LKGYGFIQYNDFDSSDQAITAMNGQYLMNKPLTVDYAFKKDGKGERHGTEAERLLAAEAKRNNALPMPGAIPGQPFMQYQGMFAGALSGAMPGAQPAATPLPFGFSPAPPQQSTPYGFS

SEQ ID NO: 153 - protein, 188 aa, unidentified
MFGFNFNTTKLLKTILVVCYLQATVLADPYTRVSWEAYMNHVNGSDDYRTQGDDTRATRFPETKPPKQGKDFLWSSKPVPSSDLFLEFFMYEGEPDEFSRTTESYQSLPSNALTARQNALTCQDIESCSYPPQVNNFQALFDDLGPSTCNLIKDETRDWILQQWPGLAVGAVISFAVAVAGSSCDILY

SEQ ID NO: 154 - protein, 187 aa, unidentified
MVRYAHNAENPEKTAKARGQHLRTHFKNTREVAAALTGLKLSKAYKYLGDVQEHKDVIPFRRFNGGVGRAAQAKNHGTTQGRWPVKSIGFLLRLLKNAEANADAKSLDTEDLLIKHIVVQQAPKTRRRTYRAHGRINPYQGHPCHIEITLAVPDEQVARNKDVEVNQPKKIQGNKRQVAAQRRLTSA

SEQ ID NO: 155 - protein, 103 aa, unidentified
MTGRGKGGKGLGKGGAKRHRKILRDNIQGITKPAIRRLARRGGVKRISAMIYEETRGVLKTFLEGVIRDAVTYTEHAKRKTVTSLDVVYALKRQGRTLYGFGG

SEQ ID NO: 156 - protein, 307 aa, unidentified
MSLDVGDVDAWIDTLSQCKQLSESDVKLLCDKAREILIEESNVQPVRCPVTVCGDIHGQFHDLIELFRIGGNSPSTNYLFMGDYVDRGYYSVETVTLLVALKLRYRERITILRGNHESRQITQVYGFYDECLRKYGNANVWKFFTDLFDYLPLTALIDNQIFCLHGGLSPSIDTLDHIRSIDRIQEVPHEGPMCDLLWSDPDDRCGWGISPRGAGYTFGQDISEAFNHSNGLTLVARAHQLVMEGYNWSQDRNVVTLFSAPNYCYRCGNQAAIMEIDENLKYTFLQFDPAPRAGEPMVSRRVPDYFL

SEQ ID NO: 157 - protein, 103 aa, unidentified; identical to SEQ ID NO: 155

SEQ ID NO: 158 - protein, 151 aa, unidentified
GATTTSILHHFQLSTSSNNSFYHYYLNNLHQNDWTRQGRQGSRKGRRQASPQDLARQHPGHHQARHPPSGASWRCQAYLRHDLRGDPRCPQDLPRGCHPRRRHLHRARQAQDRHLPRRRLRPQEARPHPLRFRWLSSSLFSLRLLCFLQTQ

SEQ ID NO: 159 - protein, 146 aa, unidentified
MFSKLIAIASLALAANAAVIDPSDHTVQYEAAPGKVVTEHYEVLSHAEASRIIEANPHISDYRYRCNYQCNDSSGNYMRNLQQGVPNQACIFSSCYDCDWKFQNCSYCRLSTGHNYRDIGGLESWCYNNGGTTVTHNCGYTDGDQC

SEQ ID NO: 160 - protein, 142 aa, unidentified
MHFKSLFIAGALFMVGASAVDCATPEIHCETSDGSPWYDDAVQATEYWKEIQDAGKDSCGDAGCAQPHGSGCHSDGGSYGTAEIVLCQDDSSSSTPQCADCRCVYSYLKPLLDQCKGANNKIGGYAHVDMGGNYINYEFVKK

SEQ ID NO: 161 - protein, 100 aa, unidentified
LLAPSSWSVPVPLIVPLLRFTARLVMAAPGTTMPSKPLNTGKKSRTPAKTAAVMLVAHSPMALDATATVVAMVPPRSFSARMTRPLQLPNVPTAGVSTAT

SEQ ID NO: 162 - protein, 185 aa, unidentified
RAGGNWTMIDDLTTGSEDSFSNSWISWFLSTKGNEYFCEVDEEYILDRFNLTGLNNDVQNYSQALELITDSLDDEDLDDEQRDAIENSARYLYGLIHARYIITSRGLAKMLFLVYPQQLPSKTTNSVPSTKPATSADAAVGVDRYLPKIFGFPVHEMSKHARWQEAQRDLQISRLQQSASDPSYV

SEQ ID NO: 163 - protein, 415 aa, unidentified
MSLTPEQTEIIKATVPVVKEHGKTITTVFYKNMLEAHPELNAIFNTTNQVNGHQPNALAGALFAYASNIDNLGALGPAVELICNKHASLYIQPEHYGIVGKFLLEAMGQVLGDALTPQILDAWAAAYWQLANLFIGRESAIYKQSEGWTQWREFRVAQKVPESAEITSFYLKPVDEKPLPRFRPGQYISVQVHVPQLECPQARQYSLSDKPRDDYYRISVKKETGLNTAKPEAKVNPGYVSNILHENVNEGDVIKVSHPCGDFFLTEQEPSHPVVLIAAGVGLTPLTSMLNTLDSTPADSQRKIHFIHGARTTSVRAFKDQIKSRAERLPNLQATFFTSSPSADEKQGVDYDVQGRIDVSKMDASKDLFLDNAQTEFYICGPTSFMNDIANSLKARGATSERIHMELFGTGGVPV

SEQ ID NO: 164 - protein, 212 aa, unidentified
MAFMNLPWPTECLHAALKNGSLPFWGFVIYRTTYTAQSDAAWPQIIELIASYMKALLYHEYNDKKKDGDEPTVYDEIWARHQLTIMDDRQFNGASVFDIQLHFEKWVEAQGKRDESTMYRMCMVIDDESIQTLLEAPPGENRKLGRRIGGPVRFVKVVEAFPELDSLDEFQGWMKCEINALWPLWKMMSDGDEMRMSYDEMKGNGKQVYGAI

SEQ ID NO: 165 - protein, 222 aa, unidentified
MARRPARCYRYCKNKPYPKSRFNRGVPDPKIRIFDLGRKKASVDDFPLCVHMVSNEYEQLSSEALEAARICANKYLVKIAGKEGFHLRVRAHPFHVVRINKMLSCAGADRLQTGMRGAFGKPNGVVARVNIGQILLSIRTRDSNRAAAVEAMRRSTYKFPGRQKIIISKNWGFTPVRREEYVKLRQEGKLKQDGAYVQFLRGHGLVEENMKRFPQAYEGVAQ

SEQ ID NO: 166 - protein, 252 aa, unidentified
MSFYQSRPDTIKGPDPLTDNWTYDSAIDLFSWNPMMPDPFTFDLPDDLMKFESKDMSAGMVAPSDISGFAIGNHLGEDAASISDPESDDHPWSPSAHAAFPELSPITSTEQVHQETARYSTTPDATSPQEQPSSPPTRSTRRRSSADGPVRNAAKRAAHNVIEKRYRTNMNAKFVALEKAMNGGNGVQTSSRGGGSASLKKSEILSNAIAYMHGLQEENRYLQKELAIVKQNLVPAGIWRGAPSCKRETSYR

SEQ ID NO: 167 - protein, 254 aa, unidentified
MGRVIRNQRKGRGSIFTAHTRLNKAPAQFRTLDFAERHGYTRGVVKEIIHDAGRGAPLAKVQFRHPYKFKMVTETFIANEGMYTGQFIYAGKNAQLTVGNVLPLASMPEGTVISNVEEKSGDRGALGRTSGNYVTVIGHNPEDGKTRVKLPSGAKKVIKNTARGMVGIVAGGGRTDKPLLKASRAKHKFAVKRNSWPKTRGVAMNPVDHPHGGGNHQHIGKASTISRYAAQGQKAGLIAARRTGLLRGTQKTKD

SEQ ID NO: 168 - protein, 249 aa, unidentified
GAEAYYSPVSSLIGMSTGLRFSTLPAASNPQSSSLIPSPSAPISTFPYTLTLTLTPLTGSLSTSYSLRASPNLSFSSRFGFNVYSWESEMVAGFELWRQSKKPKLAAGSDGDDLEWARRKVRVWDPSAFPLAPPEPEIPQPNHEDESQESVLKLRVDQSWNVRLLWEGRVKELLVSAGVGLGPSSFSPSSYANPPGTAGAQGSGGGSPASYWRGVGGFGIIFFMRDFFGSMYLNEHCLDVYSLSDFMRQ

SEQ ID NO: 169 - protein, 211 aa, unidentified
GPGVLSGFQPPDLPLITSIELDLDVVLPPPACRTMFLRTVSRAVPRSTAAIRAAPTASVNALQTRAASDHAIPNPTLANIEKRWEVMPPQEQAELWMQLRDRMKVDWHQMTLQEKKAAYYIAFGAHGPRAQPPKGEGMRVFAKVLQLTAASVAVFYAIHAFAGKQPATMSKEWQEASNEYALKEKINPIHGISKEGYEGKGFVQSPPAEKS

SEQ ID NO: 170 - protein, 153 aa, unidentified
VARRGGIYLDGNNDLVTMKGNYIYHTSGRSPKVQGNTLLHAVNNYWHDNSGHAFEIGEGGYVLAEGNVFQDVTTPVEDPVDGQLFTSPDPSTNAQCSSYLGRACEINGFGNSGTFNQADTSLLSKFKGQNIASADAYSKVASSVASNAGQGHL

SEQ ID NO: 171 - protein, 120 aa, unidentified
VRVVTFWPRVTSSRMLLPPLRTPLTASSSLPLTPAPTLSARHTLAGPAKSTASVTLVPSTRLTLACCLNLRVRTLLLLMLTLRLPRALPATPVRDTCKMERGGSELNLLMSDDIALAACW

SEQ ID NO: 172 - protein, 80 aa, unidentified
MPSKTEAARLQNDFGADYWVRNTQERRHSTAGRGLFAGLQDVKHYNVDHGWARRKSSDNPGLLASFFSRFTGGSYHPPSE

SEQ ID NO: 173 - protein, 137 aa, unidentified
VARRDSHHQIKSRLILLDITTIQHKVPSYFKQISTIESKCHPKPKQPVYKTTSAQTTGLEIPKNAATQPLAADYSPVSRMSSTITSTMAGPVASLAITPDSLLLSSVDSPGDHTIRPRNRIPFLNVRYWEECDLNWE

SEQ ID NO: 174 - protein, 181 aa, unidentified
VVSTQSDEPTIPGGAAVTIHSRNEKKARKAIGKLGLKHVPGITRVTLRRPKNILFVVNQPDVYKSPSSNTWIIFGEAKIEDLNSQAQASAAQQLAAAEAAAGGEHAGHDHEHDILGKGKAPETEGKKEEEEDDGEEVDEAGLEAKDIDLVMAQANVSRKKAVKALRENDNDIVNSIMALSI

SEQ ID NO: 175 - protein, 191 aa, unidentified
MFLQRTVSTLARRTPVRGLAAARPFSSSVSRFNKYEVKEAKLRSLDEIQTEEDLIPPGAKPGTVPSDIEHATGLERLELVGKMQGIDIFDLRPLDASRKGTLENPIVVNGAGDEQYAGCTGYPVDSHQVNWLTVSRERPIERCNECGNVVKLNYVGPEEDPHAHDHGHGHHPAPEEPKTFADYVKPEYWYR

SEQ ID NO: 176 - protein, 261 aa, unidentified
MNPYIVDPMLKYVAFDIPALARLDPSLCLFLILFFENSRVVLLGYLPVPVLGLDVVGEGLGLLGGRVVAVAVVVSVRVLLRSDIVQLDNVTAFVAALDGALTRDSQPVNLVRVDGVTSATSVLLVTSTVDNNGVFEGSLAGSIQRPQVEDVNSLHFTDEFETLETSGVFDIARDGTGLSTRGDEVFFSLDLVKRTELGLLNLVLVESANGRRKRARGSKAPHGGAPREGRYRTLKEHSPCCNRTGRSNGRGEGGGGVDVGS

SEQ ID NO: 177 - protein, 221 aa, unidentified
MADITAVGEENPSPTQDELQQAAAGNGAPDNRTPKRRMSDDEEDEEKQGRERRKIEIKFIQDKSRRHITFSKRKAGIMKKAYELSVLTGTQVLLLVVSETGLVYTFTTPKLQPLVTKAEGKNLIQACLNAPDPTTSENGVDAPEVPAETPEDVNHANVNAAAAQQTNIPRPTGMHPGYMTNEQQQQMAYYQNHLQQQQQAGGQYPGMSVGGRMPTQHQPTA

SEQ ID NO: 178 - protein, 176 aa, unidentified
MSFAADPPPNNHGTLTHLFRAPEDLVYPIPENFSLEETVLVEPLSVAIHGARVAGITPGHTVLVQASGTIGLFCAATATAFGAKQVIISDINQTKLDFARDYLGCPIFLPNISSSHPEEEASRMKEYIESQRRCRYSSVVYGSGILVAKSGPGGVVVPNWTRFNRVQSNGTYHQYV

SEQ ID NO: 179 - protein, 86 aa, unidentified
MPRGAEYANGPLQSDNAIEAGENKAHGTSGNTGLNRVNKVAEFPEGARGTGTAANPLSGQGSAGHQDGKGGHDPKTLGENKGLGTQ

SEQ ID NO: 180 - protein, 78 aa, unidentified
MPSKLAKIRPTEPPVTPASTASTRSPNSPKAPEEPVPLLTRSVARVAPAIRMERVAMTRRPLERTRDWVLNDLMIQKT

SEQ ID NO: 181 - protein, 166 aa, unidentified
MTTITEFPPFYTQQPNASALTQQLGLWQKHILSTCKQRRQFKLSVSDDIWANERIKRAASREFISVIISSLVTEGLASYTDATKEAVWVYWRSLSDWAQAAYAYAESTAQLNTPLTYYELVQGEYSHLSELHEMPVELLKLAVSLLVKQNKAVIIKTSQGEGVKFV

SEQ ID NO: 182 - protein, 130 aa, unidentified
MSIPKAAAHTDKAPQPFKDLYSQAVIAGGVVYCSGIVAIDPETGSLIEGDVKAHTERILQSLSSTLQAAGTSLDRAVKINVYLANMEDFTSMNSVYEKYFVDGVKPCRTCVAVKSLPFGTDVEMECIAVL

SEQ ID NO: 183 - protein, 113 aa, unidentified
MLRSQFGVISNAAKTAAFLKPVQTRLYASGALSKGDIQTRIFDVLKSFDKVKADNLTESASFTNDLGLDSLDAVEVVMAIEEEFAIEIPDAEADAIQNVNQAIEYIAKTPEAH

SEQ ID NO: 184 - protein, 200 aa, unidentified
VVSTQSGAPGTKKVSSTYLSKICKEQHKSIFFYLIDLHSSASLRSVTTLPARMERIRLSEQAATICNQIREMIPETATLPNQPGKDQAELMHEDENGNKIYGGKLLTERAARLKEHMKIDQVSARFISQYFTNGIQDWTERLVYWTKPTKLLNQRKQGYIIPLSKDIVLQPGGPLEANNGFRVTNERILSSGAALFIMPQ

SEQ ID NO: 185 - protein, 524 aa, unidentified
MLARSLQQIRRSSRLSLQLRAYASSPDRSASFSKLSEQDLPSLASIFSSPDTSLLTTLGDKPTATSDDLEPFNVDWMGKYKGHSSIIVKPKTTQEVSKVLQWCNERNVAVVPQGGNTGLVGGSVPLHDEVVLSLSSMNSIRHFDPLSGYVSVDSGIVLENLDNYLAQQGHIVPLDLGAKGSCQIGGNVATNAGGLRMLRYGSLHGNVLGLEVVLPDGRVINGMKGLKKDNTGIDLKQLFIGSEGVLGVITGVTLATPVRPSATNVAVFALPDYESVQTAFSSARRDLGEILSAFEFFDAASYKLVRSHGHAAERKTFEDGEDAPFFCLVETSGSNKDHDDEKLGAFLEQLMESGIVNDGVLAQDETQIGQLWSLREGIPEAAGKAGRVYKYDLSLPVEKMYSLVPELRQKLAEKGLLAAESEGGNGDGPVKTVFGFGHLGDGNLHINIVADAYRKEVEEVVEPYIYELVAKYNGSISAEHGLGLMKAPYVAYSQDAPSLDLMRTLKKTLDPKGILNPYKCVTAE

SEQ ID NO: 186 - protein, 185 aa, unidentified
MALYYGIVFGILTFEIILFFLFLLPIPTRWQKPVFRWLATSPTIAHAQYIMKIVFVFIFVLFLDSVNTLRAFYEVVNTEDENGGIPAAGNSDFRAQVGQAAKKFYAQRNLYLTGFTILLLLILNKIKNMAMDYIRLEDQFIELEGSVSKDPAIRKASKEIDTTPIEDHVTRLEPVEQEQENKKDI

SEQ ID NO: 187 - protein, 138 aa, unidentified
VARREAPNGHELPPRGYDPGENTYQAPPDERSQVDVAIDPKSNRLQLLKPFQKWDGKDITNVPILIKVQGKCTTDHISMAGPWLKYRGHLDNISNNFLIGAKSSEGKVNSIKNAFTGEYKGVQKQLVITRRKVFVGSW

SEQ ID NO: 188 - protein, 144 aa, unidentified
GCPETARDYKKEGVRWVVVGDENYGEGSSREHAALEPRFLNGAAIITKSFARIHETNLKKQGMLPLTFADPKDYDKVDASDKVDILGLTDFQEGKPLTLRLHKKDGSTVDVPLNHTFNGQQIEWFKHGSALNLMKENTAKNGSL

SEQ ID NO: 189 - protein, 264 aa, unidentified
VARREAPNGHELPPRGYDPGENTYQAPPDERSQVDVAIDPKSNRLQLLKPFQKWDGKDITNVPILIKVQGKCTTDHISMAGPWLKYRGHLDNISNNFLIGAKSSEGKVNSIKNAFTGEYKGVPETARDYKKEGVRWVVVGDENYGEGSSREHAALEPRFLNGAAIITKSFARIHETNLKKQGMLPLTFADPKDYDKVDASDKVDILGLTDFQEGKPLTLRLHKKDGSTVDVPLNHTFNGQQIEWFKHGSALNLMKENTAKNGSL

SEQ ID NO: 190 - protein, 195 aa, unidentified
GAPLTQEHGFPVRVIVPGVAGARAVKWLDHITVQREMSSNHYMHFDYKVLPPEAVDAERARTFWHKVPPVIDMPANSAITSPRNEDTVEVDAEGFITVDGYALPGGEDGPVKRVEVSIDKERWVDAELFTHPMESKWTWKIWKAKVQVEPGERRCLYSRTTDEAGNSQPQRSQWNLRGVCYNGYGEVRNLKVVKG

SEQ ID NO: 191 - protein, 50 aa, unidentified
MPANTMSATLRSLHVPGKPVIFANVWDTVSAKSIAPLDSCKALATASYAI

SEQ ID NO: 192 - protein, 373 aa, unidentified
ALPMLLINPPRNLIGQSQSGTGKTAAFTLNMLSRVDPNIMTPQAICLAPSRELARQIQEVVDKIGQFTQIKSFLAVPGSWSRNVKIDKHILVGTPGTLVDMLSRGGRIFDPKQIRVFVLDEADEMIALQGLGDQTKRIKRMLPPGVQNVLFSATFPDNVRDFAGDFAPEANQIFLKKEEITVDAIKQLYLECDGEEQKYNALSALYDIMSIGQSIVFCKRKDTADRIAARLTDEGHSVASLHGDKQTRDRDDILDAFRDGKTKVLITTNVVARGIDIQQVNMVVNYDVPDLGPEGDWKPDIETYIHRIGRTGRFGRKGCSVIFAHDQRSMQDVQFIADTLGKKMSRINATRQTDLDQLEAALKAAIKGNQPKE

SEQ ID NO: 193 - protein, 274 aa, unidentified
MATFSTRINLVPTSRTLASGVPFAPRIALVHPPASHGHGTSGPRSDVPPRWAGVQGGFASNSRVNVLPTGNFQQRFMSTTPARKIEAQPHVRGVPDWSAYQSSGKGENTRSLSYFMVGSLGVLAASGAKSTVSDILSNMAASADVLALAKIEVEMGAIPEGKNLIVKWRGKPVFIRHRTEDEINEARAVDIKSLRDPESDEDRTQRGEWLVMLGVCTHLGCVPIGEAGDYGGWFCPCHGSHYDISGRIRRGPAPLNLEVPEYAFNDDEEKLVIG

SEQ ID NO: 194 - protein, 472 aa, unidentified
GEHHHSDRDCSSVKRVFALGRLDVTLAVECGSVRTMSLRDLACYTLTLKPSTENTLLTELTALEGPSEEPRFARVREKVEGEVYSSAIYDALTGAKLASVGFASEKQKNRRLQLHNPDESVPFDNTSKLGFEWTFIFEGNKYRWTRELYGKDYICSLDRKPDPRVEICLARDADSKAPGRLQILHYNIERFPNEIKDLRGLETLLIATLMCFVDAAEDRSNSGPTRTSPLPAKPVANAAAGQSGTSASGSSDTRAKVAPVTVPVITAEDFEDDCDPNEILVGTETDVGEHIARAIALLEDPTMLFIVIRTRTAAASSRALEVSLGVTRFRHREGMSELHQYVVEEDPVRKPKPIMPAQGLKLINLDDRPAAQSPTKPEWSAPPNIAVYLSSIELPDLTPKPKPVQGHTRPPTQAPHARPPPPSQLPQKPQPRPRPPPSDGSGSSQTTLASTRPPQDDGKDSRKSSFGRLFGR

SEQ ID NO: 195 - protein, 82 aa, unidentified
MASQLMPLELIDRCIGSRMRVIMKGDKEFSGTLLGFDDFVNMVLEDVTEYDYTGATTKLPKILLNGNNICMLIPGGMPEGES

SEQ ID NO: 196 - protein, 116 aa, unidentified
GDDNKKTIPHLNIFNNGVPIDAPGADRSLHRIKNACDHERRQRVQRHTSRIRRLRQYGARGCHRVRLHRRNDQASQDPSERQQHLHAHPRWHARGRVMNHGHMISLLTSLEMAKRV

SEQ ID NO: 197 - protein, 108 aa, unidentified
AKELSPDVKPEPTWSCGEVVNVVDEHGNVIKPSDLWVKMGMQQQDNVDNLLIDDLCDQMRAKAKCTENGAQLNVDDLNHMMSYDKSYKQKRVDDLKDKYGWGAVFGPK

SEQ ID NO: 198 - protein, 38 aa, unidentified
GSLTRRAWFIQSTCATTGDGLYEGLEWLADTLRKTNRD

SEQ ID NO: 199 - protein, 311 aa, unidentified
MENLLRQMQGGGGRMGARPGPGGETILADNGETVHISSLALLKMLKHGRAGVPMEVMGLMLGEFVDDYTISCVDVFAMPQSGTTVTVESVDHVFQTKMLDMLKQTGRPEMVVGWYHSHPGFGCWLSSVDVNTQQSFEQLHPRAVAVVIDPIQSVRGKVVIDAFRSINPQSLVAGQESRQTTSNIGHLNKPSIQALIHGLNRHYYSLAIDYRKTEGEQGMLLNLHKRGWTEGLKMRDHSEMKEGNEKAIKEMLSLASAYTKSVQEETTMTAEQLKTRHVGKLDPKRHLGEAAEKAMGDQVTQSLAMGVLAEL

SEQ ID NO: 200 - protein, 213 aa, unidentified
GHTGDVLSVSFSADNRQIVSASRDRTTKLWNTLGECKFNIVDDGHSEWVSCVRFSPNPVIPVIVSAGWDKVVKVWELSKCKLKTNHHGHTGYINTLAVSPDGSLAASGGKYGITMLWDLNDGKHLYSLEAGDIVNSLVFSPNRYWLCAATASSIKILDLESKSIVDDLKPDFSAEYPDKAQKPQCTSLAWSADGQTLFAGFSDNLVRVWVVTA

SEQ ID NO: 201 - protein, 76 aa, unidentified
MVTRSGSLAFDSLLTPSFPSSSLLVGTRSSRSGNCPSASSRPTTTVTLVTSTPSPFRPTDRSPHPVESMASPCFGI

SEQ ID NO: 202 - protein, 99 aa, unidentified
MLWDLNDGKHLYSLEAGDIVNSLVFSPNRYWLCAATASSIKILDLESKSIVDDLKPDFSAEYPDKAQKPQCTSLAWSADGQTLFAGFSDNLVRVWVVTA

SEQ ID NO: 203 - protein, 124 aa, unidentified
VFAIVQIPKHGDAILSTGCGERSVGRNGEGVDVTSVTVVVGLELALGQFPDLDDLVPTSRDDDGNDGVRRESNARDPLRVTIVNNVELALSESVPELGSSVSGSRNDLSVVGRERDAQDVTGVS

SEQ ID NO: 204 - protein, 38 aa, unidentified; identical to SEQ ID NO: 198

SEQ ID NO: 205 - protein, 393 aa, unidentified
GRYDFKQPQRIRDASVTATPEWNLLEEIEFGRLGKLNLSVEEPEDLESHGTLQGYDKTFDRINTRTERPLEIIDRAWYNQTTSDDPVIAQLAQTQSAQIFATDAILAVLMCTTRSVNSWDIILERRGNQLFLDKRDSGPFDYVTVHENAADPPADSDDPNNVNSASSLSLEATYITRNFSSQVIDAKSKPYSPSPNPFYSEDEPSPVASCLYRYRKFDLSVGEEDTLDLIVRTEVDAYQGKKDSLVTVKALNEFDPRASGGGKALDWRKYLDTQKGAIVASEMKNNSAKLARWAIQSVLAGAEVMKMGYISRASPRDTTHHVIVGVQNYKPKDFAAQMNVSLNNGWGIVRTIADLVLKQPEGKYVLVKDPNAGIIRLYSVPENAFEAEEEEEQ

SEQ ID NO: 206 - protein, 106 aa, unidentified
MTSSSLSEFETLLSRPRQNGTCLKRSSLADWASSTFPSKSPKTSNRTVPSKVTTRRLTASTLVPKDLSRSLIEHGTIKPLLTIPLLLSSLKRSLPKSSRQMPFLRF

SEQ ID NO: 207 - protein, 245 aa, unidentified
ERSRSLAPEADQGVATQCHEVRSSGSYDTFEIGHHQPDSDTSGVADLRTSSRMDTCDAHLLRRVKSCPLFSYREDEVSETVQLPTGEWTTIRDITPSAPKIGFEVRDSLSAFPTAKPVEAKHESASSISNDLPSQPSSRPLIECPTLVADSRTTTGSNSVRSFDAQTERLSGLSDVHHRYMQDKPSQRSDSWTDVKSSAPSSQSMAVPNKAAYLAPIPAGPNDSKTSSSGRAPSDAATEHECSLQ

SEQ ID NO: 208 - protein, 148 aa, unidentified
MAPKSTDKPASTAGKAPSAGGKAPASKTVGAKKTAAKKSAKSTGEGGEKKKRVKSRKETYSTYIYKVLKQVHPDTGISNKAMLILNSFVNDIFERIAGEASKLATYNKKSTISSREIQTAVRLILPGELSKHAISEGTKGVTKYSSSK

SEQ ID NO: 209 - protein, 369 aa, unidentified
DVKRFTKDLLFNSEGNLTFKPHLWNDIRHTLLPTFIRQIGYVPIPRAEFSSPDIDLVIENLVLSGPNLFPNVVSLESHNSFKFSPYQQLNKGMDTHHHKFRLGMSQIQADIRDVRFSFRRKTGWPKLKDHGLADVILAGKGMSIDVELESVEGRRDSVVRVNHVHTTIDTLTFSIRDSKHDLLYKFVKSVATGTIKKAIQAAVDNAIRTAVGHLDDQLVQVRNTVDDAKKSDETTRTQALKDLYSKKADTAQKKQAESKEQPGTFRIVANRDSVLNPDMGGGKGAMTNKMWKTEDLAHSGKEWHSPAFDLLDSKHPARTGQTHPEAKEGAGHGNSLSSKAQPGANAADQLKATHGQSEAEAIAGQKRQQ

SEQ ID NO: 210 - protein, 173 aa, unidentified
MSDSRSDERLDGPSSRTTVSPMSSLPVRVCRSTSSSSLSRDDETLLCESTTSTPPSTPSPSPSETPSTTCSTSSSSRWPRVRSRRQSRPPSTMPSVRLSVTSTTSSSRSETPSMTPRSLTRPPERKPSRTCTRRRRTRHRRSRPSPRSSLVLSESSPTETLFSTPTWAVARAP

SEQ ID NO: 211 - protein, 248 aa, unidentified
MYTSAVTLLSLVLLLATSVIAQEQAGRPGTQRGGVFFGCYADRPTGNANQPITRVANSDTFFECMENCAAITSPSLLGYYQPSSGQCFCGNLLFNPQAQLNGNGCQGSDWSFGRTSTTFRRFGDACRPFGGVGFSANQYTTVTGPVACHVQCASNRFAYVWSDTGSNSWQCACSNNVRVQEDFQYTCQGGGVFVFEHSVQAQASSLNRKRTVEEQWAVPKDALCPFGMSACKVSGVDNAYEVCFFSDR

SEQ ID NO: 212 - protein, 116 aa, unidentified
MFNAHQTDSPMSGPILEATHGNVLAATMSVFRRTSSTLVKVAVYLCLNIQYKLRLLRLTGSGRWRNNGLFRKTPSVHSECQRARYQVSIMLTRYASFQTARPLVPWPRGLKHAIDL

SEQ ID NO: 213 - protein, 271 aa, unidentified
GGGTVVSNALLENAKLCKTQGKESSLRVIVCGRNRLENGSAPHWAEAFATHGKLVEVRMPQNGIRMEGIKAIADGLAKCPTLEVLDLQDNTATKTGTRSIVRHLSTWPKLRILNLSDCLLGSVGGIALATALSTGSNKHLEQLKLQYGEFDKRTVEILSTAISQHLPKLTTLELNGNRFDAEDECVETLKKALELHGNEDALDELDDMEEVDEDEEDDDDEDEEDEDEDKDTSADDGIDAGAAGEDALPPVTKKDEDVLADLLSKVHVQPS

SEQ ID NO: 214 - protein, 222 aa, unidentified
LDRRSASTSSSFFVTGGRASSPAAPASIPSSALVSLSSSSSSSSSSSSSSSSSSTSSISSSSSKASSFPCSSSAFFRVSTHSSSASKRFPFSSSVVNFGKCWLIAVDSISTVLLSNSPYCSLSCSRCLFEPVDNAVARAIPPTEPKRQSERLSIRSLGQVERCRTILRVPVLVAVLSCKSSTSNVGHLASPSAIALMPSMRMPFCGILTSTNLPCVANASAQ

SEQ ID NO: 215 - protein, 176 aa, unidentified
MVKLSNSLVRRLKWQHVRSLGVVALTAQLRGPQPQSAEDEDSEAAGKKLKLAGDQATSAVIPKSADKPDTFPLLDTLPATMAAGTRSMTRPLHVGDLRLADLRKIMQAAGHTAEFRGEGTLLIDKSVAVRKSGTGQIEIEASAQAAANQATPGRGASSFLAVKRKIYEGLAVVTGS

SEQ ID NO: 216 - protein, 139 aa, unidentified
KMKIDVEKLNKDISLFPQVHPITEDMKITHKGVSRLVMLDRYSFKDTEKITLSEGDFVVLTIKEDPKFPARGLGYIKEIDWENKKAKVQVEEEFRHTLEKPEERETGIIVRSLDVIEKPLEIFYEQIAKRNATGLAAVE

SEQ ID NO: 217 - protein, 282 aa, unidentified
GDATVTQLREIMDDPAGYFLPNLKHGADNMFYVGPRGLAQELEELFTFPSTILRKRQDTSQHDERQAKKARTQEDEAAGDALEEPETGRRDSVLPTERAAFGLEGDDSGFFLGDQTMGDDMLPMDDMGAMDTGVDQRRMRTPSVAPSVTESIARQIQNDRSAGTHPLAIFEKEARDDTQSQSQATPNKSVASESISKTSSGQSKNTGMAMGLLRREIEAIEEEDKMVGFDHLADKASKRAASAFFFELLVLGTKHAVKLEQAQAFGDIHIRGKDKLFAEVVA

SEQ ID NO: 218 - protein, 261 aa, unidentified
MAPITCSTSVHADLHKSSRSFLPSQAQSSESARIPVSMTKGRQRRRARKRTKRLVTRWRSPRLGDATVCFRLNGPLLVSRVMTRAFSLATRRWETTCCLWTTWEPWTPEWTSDACEHHQSHRRSPNRSHVRFRMTEALAHTHWLYSRRRQGTTRSRNRRLRPTNRWPPSLSARLLLANQRILAWPWVCCEGRLRRSRRKTRWSGLITWQTRRPSEQRLHSSSSCWCLVPNMRSSLNKLRLSATSTYAAKTSCLQRLLHRQT

SEQ ID NO: 219 - protein, 108 aa, unidentified
MGDHATTNDPSNATFEEKGKGKDVQDQIAEDSSDEESDQEPEMVDEEEDDNNLEPISQDNIISGGRRTRGKIIDYAAEAEKNKDEMEDSEDDEDYQGANDDEDDQMRD

SEQ ID NO: 220 - protein, 78 aa, unidentified
ELKYFKAVALYNLSRYLDARKAINDLIQSYPDFRQAEALKSAIDDKVVRDGLIGVSVAGAVVAGVVGLAVALARGNRG

SEQ ID NO: 221 - protein, 27 aa, unidentified
MGHVESRVNQRGPPRKAKYSWVTDSEK

SEQ ID NO: 222 - protein, 27 aa, unidentified
MLKAPFFHRPCGGKGVQLKWATPDSAV

SEQ ID NO: 223 - protein, 177 aa, unidentified
MPVRPYLEEMADMPVPLFAYDAPPTLADHPHAREHQHTTFMQYLARKQPDPKNYPNYPDVDIRDAINHYLIELECPGIKDAADIHCQWTSSRHLTVTGDIARPEESQIEAQIESRPVYLVLGERRIGSFRRNFTFPVEVEQENMTAKLEAGLLKIVLPKHKHHTPKGTGKVDIDVIE

SEQ ID NO: 224 - protein, 163 aa, unidentified
MVLVLGQDNLQQSGLQLGSHIFLLDLHREGKVATERANASLSQNQVDGPALDLRFDLAFLRTGDVAGDGQVPRARPLAVDVGCVFDPRAFELDQVVIDGVADVHVRVVGVVLWVRLLARKVLHEGRVLVLAGVRVVGQGGRCVVREQGHGHVGHFFQVGSYGH

SEQ ID NO: 225 - protein, 118 aa, unidentified
MFGFNFNTTKLLKTILVVCYLQATVLADPYTRVSWEAYMNHVNGSDDYRTQGDDTRATRFPETKPPKQGKDFLWSSKPVPSSDLFLEFFMYEGEPDEFSRTTESYQSLPSNALTARQK

SEQ ID NO: 226 - protein, 349 aa, unidentified
MSPTPTSPHNKLSLPARASSHDSTDGIRKRVCKACDRCRLKKSKCDGSSPCSRCKADNAICVFGERKRSHDKHYPKGYVEMLEQQQGQLVSGLKEMYHRLQKASAWDGPVLDESTGQPLTHDILSALDLLEPKHDDSNEPEVFEENCEKLQSKLLADGAGFAHRRGSISSDSEHSHHDRPKTSSRHDTPVQPKPSIFKENLSFASAASSPLTQSPIPRSKPLNVMPYQTLQPSSRPSPLQMPSAYNDPQLYAPEWAQALADMSGDPNYRQRFSMQQQQQNDFDNLLWDPSAQAPMESPFSQPAFFNQAQLIGSGNVFGLSDINDLGPNPADGGMDFDFSKFVQQTEVMT

SEQ ID NO: 227 - protein, 149 aa, unidentified
MSSFRVAAPKMASMAAQSSVKVARPAFQAAQLQKFTRAYSAVPKNTVFNTMKRTQMMARQASPIAKRAYSSEMANALVQVSQNIGMGSAAIGLAGAGVGIGLVFAALIQAVARNPSLRGQLFSYAILGFAFVEAIGLFDLMVAMMAKFL

SEQ ID NO: 228 - protein, 165 aa, unidentified
MECTFLQELGHHGNHEVEETDGLDESETKNGVREKLATEGGVAGDGLDEGGEDETDTDTSTGKTDGGGTHTDVLGDLDEGVGHLRGVGTLGDGGGLAGHHLGALHGVEDGVLGDRGVGAGELLELSSLEGRAGDLHGGLSGHGGHLGGGDAEGRHCDDGEVVRWS

SEQ ID NO: 229 - protein, 425 aa, unidentified
GGFSVKFRTAEGNWDFVANNTPVFFLRDPAKFPHFIHTQKRDPATHLSGDDDSTMFWDYLSQNPESIHQVMILMGDRGIPKGWRFMHGYYGHTLKIVNDKGEWVYAQFHLISDQGTQNFTGDEAAQQSNDYGQKDLYEAIEKGDFPSWTMKVQIMTEKQAEEAWEQKRINVFDLTHVWPHGDYPLRTVGKFTLNENAKNYFAEVEQVAFNPSHMIPGVEPSNDPVLQSRLFSYPDAHRHRIGANYQQLPVNQNVCPFALGNFQRDGQMAFYNQGSRPNYLSSIEPISFKERAYDLNKVHGKFVGEAVAFLSEIRPEDFNAPRALWQKVFSEESKQRFVDTVSGHMSTVRDKAITARMMTIFREVSPDLGDRLEKATGVKGESTIAGMKFNGTHNGFDKANKIPANGMKKGGEVIFDNGAPATAAR

SEQ ID NO: 230 - protein, 239 aa, unidentified
VSVSIGVREQSRLQHWVVGRLDTGNHVRRVECDLFHLGEVVLGILVKGEFTDCSKWVITMRPDVGQIKDVDPLLLPCLLGLLLGHDLNLHRPRGEVSLLDGFVQILLSVIVGLLSSLVTREVLGALIRDEVELGVDPFALVIDNLEGVAVVAMHESPALGDTSITHEDHDLVDRLGVLRQVVPEHGRVIVTRQVGGGISLLGVDEVGELGRVSEEEDGGVVGHKVPISFCGPELDRESS

SEQ ID NO: 231 - protein, 368 aa, unidentified
DTIDAEVLDSLGVTQENFQFALGVSNPSALREVAVVEVPNVRWEDIGGLEEVKRELIESVQYPVDHPEKFLKFGMSPSKGVLFYGPPGTGKTLLAKAVANECAANFISVKGPELLSMWFGESESNIRDIFDKARAAAPCVVFLDELDSIAKSRGGSQGDAGGASDRVVNQLLTEMDGMTSKKNVFVIGATNRPEQLDNALCRPGRLDTLVYVPLPDQEGRESILKAQLRKTPIADDIDLSYMASKTHGFSGADLGFITQRAVKLAIKQSIDLAIQNQKAREAEGDTAMDEDIEEDDPVPELTKAHFEEAMSMARRSVTDTEIRRYEAFAQSMKSSGGGSAFFRFPESGADGNAAEQQQNGAGEEDLYD

SEQ ID NO: 232 - protein, 317 aa, unidentified
MHDCSLHLEYYSDNSKGLGREVRETNLVVEVLLASTILLLLGCVAIGTALREAEESAAATGALHALGESLVAPDLGVGDGATSHAHSLLKVSLGQLGHGVVLLDVLVHGGVTLGLSSLLVLDGQVNRLLDGQLDGTLGDEAKIGTRETVSLGGHVGKVDVVGDRSLAELGLENALTALLVRQGNVDESVETTRTAESVVELLRPVGGTDDENVLLAGHTVHLSEKLVDHTVGSTASIALRTATRLGDGVQLVEEDNARRGSTSLVEDVTNVALRLTEPHGEKLGTLDGNKVGRALVGDSLGQKSLTSTRGTVEKHTL

SEQ ID NO: 233 - protein, 166 aa, unidentified
VSPKRTSSLPLASATPLPFARSQWSRFPTSDGRTLVVSRRSRGSSSRACNTPSTTPRSSSSLACPHQRVCFSTVPLVLVRLFWPRLSPTSARPTLFPSRVPSFSPCGSVSLRATFVTSSTRLVLPRLALSSSTSWTPSPSLVAVLRAMLAVLPTVWSTSFSLRWTV

SEQ ID NO: 234 - protein, 160 aa, unidentified
MGHSAGLRKGTRYAFSRDFKKRGMIPLSTYLKQYKVGDIVHVVCNGAVQKGMPHKDFHGKTGVVYNVTKSAVGVILYKQVGNRYIEKRVNLRIEHVRLSRSREEFIVRVKTNAEKKRKAKEEGTTVFLKRQADKPREARTISAKDNKPESIAPIAYDTHI

SEQ ID NO: 235 - protein, 526 aa, unidentified
DMGIGGLDTEFSAIFRRAFASRIFPPGLVEKLGIQHVKGILLFGPPGTGKTLMARQIGTMLNAREPKVVNGPEILNKFVGQSEENIRKLFADAEKEQKEKGDESGLHIIIFDELDAICKQRGSTNSGTGVGDSVVNQLLSKMDGVDQLNNVLIIGMTNRMDMIDEALLRPGRLEVHIEISLPDEAGRFQILNIHTNKMRTNGVMDSDVDLGELAALTKNFSGAEIGGLVKSATSFAFNRHVKVGSVAAFDDIDNMKISRADFLHALDEVTPAFGVSEEELQQVVQNGIIHYSQHVNDTLNDGSLLVEQVRKSDRTPLVSALLHGPSGAGKTALAATIAMASEFPFIKLISPETMVGFSEPQKIAQLNKVFTDSYKSPMSIIVVDSLERLLDWNPIGPRFSNGVLQALVVLFGKRPPKGRRLLILATTSNRNILTDMDVLSAFDTDIPINPISSIDAVVHVLDEVKLFPNSKEKQRATQMLREARLGEGGRPDLLVGVKKLLSMAEMARQDPDPTMKIVTSILREAS

SEQ ID NO: 236 - protein, 425 aa, unidentified; identical to SEQ ID NO: 229

SEQ ID NO: 237 - protein, 122 aa, unidentified
MLTDTESEPTISNCPLTRMCAPSPWATSSETARWHSTIKVVDPTTFLRLSQSHSRRGRMISTRSTANSSEKPSPSCLKSGQRTSMPQGHCGRKSLARKASSDSSTPSLVTCRQSETKPSPLE

SEQ ID NO: 238 - protein, 309 aa, unidentified
MPPRQPATRLFALPPRFLCPSLPTTQTRTIRSIDKPAPKPSRFNASLNLPVLGSSSTAAFARKEHSLPLRTGALAIKKGMTALFDPVTAKRTPCTVLQLDRCQVVSHKRRDIHGYWAVQVGAGAKEARNVTRPERGHFAAYNVPLSRHLAEFRVKNAEGLPPVGSAITADLFIEGQFIDAKADRRGMGFEGGMKRWNFGGQPASHGNSLAHRLMGSSGGGQGSGSRVLPGKKMPGRMGGEQATVANLRVMQVDKENGIVVVSGAVPGPKNCMVKLQDALKKPWPDATWPPSIEGATEVLREATEKAPAA

SEQ ID NO: 239 - protein, 130 aa, unidentified
MGIWDAFTDIVEAVTPWSVVEAEAPAEEPQEENESKTESKDEPEEEEEDEEEEEDEDDEEELVDPKETLEEECKNSPQCAPAKHHFDECVERVQQQESEGGAKEDCVEEFFHLAHCATACAAPKLWSQLK

SEQ ID NO: 240 - protein, 136 aa, unidentified
MRKKRRMRMMRRSSSTPRRLSRKSARTLLNVPPPSTTSTSVLSAFSSRRARVVLRRTVSRSSSTLPTVRPLAPLPSFGLSSSKLTTLGYRLLRRRNGYIHVEKMPGAGTRQCCPLRKAVPLYECSSAGYQVGQRLL

SEQ ID NO: 241 - protein, 194 aa, unidentified
GRYYRAPEIMLTWQKYDVAVDIWSTGCIFAEMLEGKPLFPGKDHVNQFSIITELLGTPPDDVIQTIASENTLRFVQSLPKREKVPFTTKFANADPLSLDLLEKMLVFDPRTRISASEGLSHEYLAPYHDPTDEPVAAEVFDWSFNDADLPVDTWKVMMYSEILDFHNLGDIQQDQAAEGPVTGDLAPPSATTSA

SEQ ID NO: 242 - protein, 134 aa, unidentified
MSPSTFGAQDVSSPRCSRESPCSRARTTLISSRSSQNCSAHLLTMSSRPSHLRTPSDSSSRCPSVRRSHSLRNSPMPTRFRLTCWRRCLSSIHVPVSRHQKGCRTSTLRHTMTRRMSPSLPRCLTGVSTMRIYQ

SEQ ID NO: 243 - protein, 111 aa, unidentified
GYPLNLSISISGGKETNRDCPSNGERSGKSSNLKAGPLGVRIVICRGCFGNGPHLSALERAVIEGENPVWDGVAAPVSSSFDESSCLGMQLKLGGKFHLKLNIGRRPIAHK

SEQ ID NO: 244 - protein, 41 aa, unidentified
GLVAIARRPHPFPCQTRKLSVSAPKVVGGSPPVRIGRCQAK

SEQ ID NO: 245 - protein, 218 aa, unidentified
MEPDQEESEEEEEEEDDEMDEDEDEGQQQDASGMQTPSGLATPSGYASTTSTMPGGMETPDFMDLRKQRQTRDETADQEDQGAPRDLYTVVPERRATASGFLGSDRAYDLSNAPQSSNMPVLGQEDSRKKKGGRSGADDVDLALDPAELEGMSEQELRQKYDSHRRSSSSQGAGGQQDKEDFSDFVAQEVAKKRQRAQQRGGSGRDRESSRSKEKFKF

SEQ ID NO: 246 - protein, 210 aa, unidentified
MTRWMKMRMRASSRTPVACRHPLGSPRPQAMPLLHLQCLVAWRRLTLWTCASSDRRATRPLIKRTRVHRETSIRSCPSAEPPLLASSVLTAPMTCPMRHSLPTCLCWVKKTRARRKAADLVQTTSTWPWIQLSSRACLSKSLGRSTTRTGAPRPVKAPADSRTKKISQISSRKRSQRRGRGLSSAAAVDATAKALGARKSSSFRVYVCIV

SEQ ID NO: 247 - protein, 164 aa, unidentified
GVIQVDEPALREGLPLRTGAARDAYVKWAVNAFKLSTAGVTDSTQVHSHFCYSEFQDFFHAIAALDTDVLSIENSKSDAKLLQVFVDQSFPAHIGPGVYDIHSPRVPSVDEIKERIEQMLQYLKPEQLWINPDCGLKTRTTEQTIGQLTSLVEAAKFYRQKYTQ

SEQ ID NO: 248 - protein, 125 aa, unidentified
VRVFLCVLALPVMLMLSGLSMLSSCLLLVSPTAPRSTPTSATVNSRTSSTLLLPLIPMFCPSRTASPMPSSSRSSLIRVSPPTLDLVSTISTLLVFPPWMRSRSVSSRCSSTSSLSSSGSTLTAV

SEQ ID NO: 249 - protein, 171 aa, unidentified
MLMRTELLSVLLAVELSSLDQRGQLADSLLSGTGLQTAVRVDPELLRLEVLEHLLDTLLDLIHGGNTRRVDIVDTRSNVGGETLINEDLEELGIGLAVLDGQNIGIKGSNSVEEVLEFTVAEVGVDLGAVGDTSSRQLESIDSPLNISITGSASTQRKTLTQGRLVDLDDP

SEQ ID NO: 250 - protein, 66 aa, unidentified
LKSGVFGVRVVICRGCFWAATDLSSLEQDVIEGENPVCGRKGTLHVAPSTSRVVWECSSKWEVNFF

SEQ ID NO: 251 - protein, 189 aa, unidentified
MKLSNSAHYSLFLLSSILGFSSASANSHLSDDSPCVARSPTSGLYYDLNAISLAPPEWKNGKKVDQEARDESWHAKGHDYPANFTINVCAPVLENVTNVVGVDASRWANVSAFYEQAGKIYSMGEQASEPFFRGRKLVLNYTDGSPCPGDSNTASGNSSIRTKSTLMSFLCDRAAEFPGLEKLGSTGSR

SEQ ID NO: 252 - protein, 393 aa, unidentified
MNGFNEKGLDGDAFGEKSNLSGLKTFDAFPKTKTSYTTPTRRGGQWTVLILAVCTLFSLHELRTWWRGTEAHHFSVEKGVSHDLQLNLDMVVHMPCDTLRINIQDASGDRVLAGELLTREDTNWDLWMKKRNFESHGEHEYQTLNHEAADRLSAQDEDAHVHHVLGEVRRNPRRKFSKGPRLRWGDNKDSCRIYGSLEGNKVQGDFHITARGHGYMELAPHLDHEVFNFSHMITELSFGPHYPSLLNPLDKTIAESETHYQKFQYFLSVVPTLYSKGHNALDLVTTNKDNSVRYGRNTIFTNQYAATSQSTALPEIPTLIPGIFFKYNIEPILLLVSEERTGFLALVIRVINTVSGVLVTGGWIYQISGWIVEILGKRKRQSEGVLTGKHYSD

SEQ ID NO: 253 - protein, 292 aa, unidentified
MFTRTLRPAVAVARTQAVQQQQAGMATLKEIDQRLKSVKNIGKITKSMKVVASTKLTRAEKAMREAKKYGAANNVLFEQTKAGEEEPKERKILYLAMTSDGGLCGGIHSNITRYMKKAVAKEPGMLAVVGDKPKAQLSRAMPKALTMSFNGVGKDVPTFVEASAIADEIMKSAGDFDEIRIVSNKYLSAIAYEPHTNAVISAEALRQAAGFQQYEMEEDVSKDLAEFALANAIYTALVEGHAAEISARRQAMENASNNANDMINSLQLQYNRGRQAVITTELIDIITGASAL

SEQ ID NO: 254 - protein, 317 aa, unidentified
MSFAACHFCCFVHLVYTRLQSRGTGNDIDQLGGNDSLSTTVVLQLERVDHVVGVVGSVLHSLPPCRDLGGVSLDQGSVDGVGKSELGQVLGDILLHLVLLETGGLSECLSGDDGVGVRFVGDSGKVLVRDDSDLVKVTGRFHNLIGDSAGLDESGDILADAVERHGQSLGHRSRELSLGLVTDNSQHSGFLGHSLLHVSRNVGVDTTAQTTVGCHGEVEDLALLGLLLTSLGLLEQNVVGGTVLLGFTHGLLSSRQLGRGNDLHRLGDLPNVLDGFQTLVDFLQCGHTGLLLLDSLSPGDRHGRSESTREHGVERGE

SEQ ID NO: 255 - protein, 311 aa, unidentified; identical to SEQ ID NO: 199

SEQ ID NO: 256 - protein, 93 aa, unidentified
MGAIPEYDPEEPLETKPFKFVTAGYDARFPQQNQTKHCWQNYVDYYKCVEAKGEDFRPCKQFYHAFRSLCPKAWTDRWDTQREGGNFPAILNK

SEQ ID NO: 257 - protein, 252 aa, unidentified
GPLLEELDVEAYAKKYRYLRFMCQETLDHLAFLKDKVKDVEGFWASTLLKHRDLRGYITSRSDKDALKYLTHIELVQDPKDPRPFALKFYFKENPYFSDLVLEKKYDMSEGSEPAPADGSITEGMRNFKEDELVTKATTINWKSDDKNLVAKQPRSKIPDNDDDEDFDGDVGSFFNYFTDDTDIFQIGALLQSELLPDAIDYFVGRGEQVDSEGEELDELEEDDEDDDEDDEGSIDLEDEEEQPSKKKPKRA

SEQ ID NO: 258 - protein, 165 aa, unidentified
GSFLTPQHTHTPFGRAMHNALTVSRLNDKFQEPLVVLVELLRARVLHDRNFSNRQFSGGPSFGTDNQKRSMLLIFRTLSIIPLQFKAEHWSGPLSRELLVFNSFHKTLSRSLRTLVESITMNAFLKNNARRARDDYLDIALSLPFQNDTNTGFGIFFKVGGLAGV

SEQ ID NO: 259 - protein, 94 aa, unidentified
QIYLDALTTFAEGNITEENKDSESVKEAKQSAMEILGDAIPNVKDPEAELLRGFRFWDAVLVCVRTLKADRAIDLKLAESFEAANSYLNMMRPN

SEQ ID NO: 260 - protein, 251 aa, unidentified
GSFLTPQHTHTPFGRAMHNALTVSRLNDKFQEPLVVLVELLRARVLHDRNFSNRQFSGGPSFGTDNQKRSMLLIFRTLSIIPLQFKAEHWSGPLSRELLVFNSFHKTLSRSLRTLVESITMNAFLKNNARRARDDYLDIALSLPFQNDTNTGFGIFFKIYLDALTTFAEGNITEENKDSESVKEAKQSAMEILGDAIPNVKDPEAELLRGFRFWDAVLVCVRTLKADRAIDLKLAESFEAANSYLNMMRPN

SEQ ID NO: 261 - protein, 148 aa, unidentified
GIYLDGNNDLVTMKGNYIYHTSGRSPKVQGNTLLHAVNNYWHDNSGHAFEIGEGGYVLAEGNVFQDVTTPVEDPVDGQLLTSPDPSTNAQCSSYLGRACEINGFGNSGTFNQADTSLLSKFKGQNIASADAYSKVASSVASNAGQGHL

SEQ ID NO: 262 - protein, 120 aa, unidentified; identical to SEQ ID NO: 171

SEQ ID NO: 263 - protein, 160 aa, unidentified
MSAQPLRIVMACDEAGVPYKDAIKAVLEKSPLVASVSDVGVNDASDKTAYPHPAVEGAQQIKAGKADRGLFICGTGLGVAIAANKVPGIRAVTAHDPFSVERSILSNDAQVLCMGQRVIGVELAKKLALDWLNYRFDPKSASAAKVQAISDYETKFAGSS

SEQ ID NO: 264 - protein, 150 aa, unidentified
MSVTTTSSAAAASCTPSWQIPVDDVACAGQISGNITKVFDTCCKGNSPVKYNDDCNIYCLAQGQTKQELTDCLTEKSGNNQIFCGHGKQNATATAEATTTKETGTSTGTSTSSTGTSTETNAAVLNQPISKTGLGLVAMLFCSALVGVVA

SEQ ID NO: 265 - protein, 197 aa, unidentified
AGVDRGVITKDEKDSSINRLLVTGYGLAEVMGTDGVNGIKTRTNHVMETCEVLGIEAARQTIYNEIQHTMTSHGMSIDPRHVMLLGDVMTYKGEVLGITRFGVQKMKDSVLMLASFEKTTDHLFDASLFSKKDEIQGVSECIIMGTPAPGCGTSLASIVTPAPLLPRKKPLLFETAFKAGQDRLSYHENNGGMEVDM

SEQ ID NO: 266 - protein, 249 aa, unidentified
AGSGGAYSFSLTTFSPSGKLVQIEHALAAVAGGTTSLGIKATNGVVLATEKKSPSLLLDTSVLEKVAPICPNIGFVYSGMGPDFRVLVAKARKIAQAYYKVYGEYPPTKVLVQEVAGVMQKATQSGGVRPYGISLLIAGWDSHRGQSLYQVDPSGSYWAWKASAIGKNMVNGKTFLEKRYNDDLSLEDAIHTALLTLKEGFEGQMTENTIEIGVVTVPTAEQMQEKPGERLPPTFRKLTEQEVRDYLAL

SEQ ID NO: 267 - protein, 346 aa, unidentified
MLTSAFSTSASKMLGKRAVSSSSALNGKVAVLGAAGGIGQPLSLLVKQNPAVSSLSLYDVRGSPGVAADISHINTPAVTEGFLPDNDGLKQALEGAEVVLIPAGVPRKPGMTRDDLFNTNASIVKMLAEASAKYCPKAMMLIIANPVNSTVPIVAETFKRAGVYDPARLFGVTTLDVVRSSTFVSGITGAKPSDTVVQVIGGHSGATIVPLLSQIPQGDKIVKAGGQQYADLVKRIQFGGDEVVKAKDGTGSATLSMAYAAAVFNDALLKAMDGQKGLVQPAYVESPHFAKEGAKYFASNVELGPNGVEKILDIGNMSSEEQELLKECLPQLAKNIAAGEKFVADN

SEQ ID NO: 268 - protein, 373 aa, unidentified
MSLFLLFSLALIIIGSNVVGYPLVVSDELLTSSNVLGELGKALLKELLLLRGHVADVEDLLNTVGAELDVGGEVLSTLLGEVGALDVSGLNETLLAVHSLEESVVEDGSGVSHGEGSGASAVLGLDDFVTAKLDALDEVSVLLAASLDNLVALRDLGEQGHDGGARVTTDDLDHGVGGLGTGDARDESGRADNVEGGDTEETGRVVDTSTLEGLSDDRHGGVDGVGNDEHHSLGAVLGRSLSKHLDDGSVGVEKVVTGHAGLARNTSRNEDHLSTLEGLLEAIVVGEEALGDSRGVDVANVSSNTRGAANIVKGEAGDSRVLLDQQGEGLANTASSTEDGNLSVQGAGRRDCSLAEHLGSRCRKGAGQHFGSK

SEQ ID NO: 269 - protein, 435 aa, unidentified
GASVLLPALHFDEREQRPGRTSSTASLLQRHDRNEPLSLNSHSPTSVDHTPTTAHFTGAEELLASDVGPTATAGLPGDAELESKLKLLEEVKRARESVHSSLERIRAGTPTPSISQGMPSPTPSGAPGYARTPSSVGLSDDVRSRRGSTTSSKVLDAIDKPRVATQSEWDEYVRNRHVISPPPTQFAVLPTSAAMVDRGTSRHSQYALVSDGVAKALDRRERTISMMEPQVAEDWGPRETLDSTPAHVSMGRRAMSFHEIPLASPVAASRPQDRSSYSAGPRQVIGSAAGHTQRPGISQSRSAHGRTMTYDELTERHRQRLSALQAPVSAKIREPMDIASAKASWDKQKRVERDEMKRREAEKLAQAHARERRGPAVDKKEVLKSTDEWRRSVHGGLDGFAVPHLPAHARGSTQPGGSGAKRSSLSQRPSNYFAN

SEQ ID NO: 270 - protein, 225 aa, unidentified
GQACCFRHCISTSGSNDLDVRLRLQVCCSVTIAMNLYRSTRTRRHLWTTLPLLRISLVLKSCSPPTSDRPRQLGYPVMRSLRASSSCLKRSNVHGNRYIARSRGSEPARLPRLSARECPARHPLVPLVTLELRRLSACRTTCARDEAQRPAPRFLTLSTSLESLPNPNGTSTFATGMSSHLHPLSLPYCPHLLRWSIVVPVDTASMPLFPTALPKRLTGGSELFQ

SEQ ID NO: 271 - protein, 168 aa, unidentified
MGSPAPHHRHQSSLEGVIDFSTGRGHPLNPYQRDKAESVFTGIINRFEDSSTVEKPYNRAKLVRLTYEYARSEDSRCNFLQAFFGSVNVTMDDSIDFDDEAVEEGIRSSLNSFADFLVENFFLPLKASASRTPPAPQPKFRADVLLWGLWKEWPRSDATASSAIDIVA

SEQ ID NO: 272 - protein, 253 aa, unidentified
QDAPSPPAQVPSRRPAVGSVERVASLRRDCLIRDRHRCVISRNFDMKEAERRLDDSGYDHASDDEGHLLKDQEHGSFAELEVAHILPHSLMTTTANSELNKSKETALTILNMFDSGIVHLIDGPDIDRPRNALTLSIDLHRQFGNFKVFFEPMPEPHTYRIDSTLRQPFRNPIFPVTRALYLTPERTIDPPSGRLLAVHRAICHILHLSAAGNYIDSILRDMDDGTVQANGSTRLASIVRLKLGGWWDGTVVG

SEQ ID NO: 273 - protein, 106 aa, unidentified
MVNIPKTRRTYCKGKECKKHTQHKVTQYKAGKASLFAQGKRRYDRKQSGYGGQTKPVFHKKAKTTKKVVLRLECTSCKTKAQLALKRCKHFELGGDKKTKGAALVF

SEQ ID NO: 274 - protein, 84 aa, unidentified
MILEGSESKLRFSYDRPTRKDMSRHIMACAQHSSRVHQRCPTLPLRCVGCDSSTTAFQYHLFACRSEDDFTTVHITNRKCQQDQ

SEQ ID NO: 275 - protein, 41 aa, unidentified
MKLTFKDLKQEKFVIEVEPSETVREVKQKLLKKKANMRRNE

SEQ ID NO: 276 - protein, 369 aa, unidentified
GPEAGEVRNRGRALRDCSRSQAKIAQEKGEYEAERMKVIYSGKILQDDKTVESYNIQEKDFLVCLPSKGPKPAASSSASQAPATPAPRAPVATPAAPAPAAPAPASSTPAVPATPSPAGAQTGPSFGDPSALTMGSAAEGAVTQMEAMGFARSDIDRAMRAAFFNPDRAVDYLLNGIPADVQQEQQQRQQEQQADRAAEQAPVPSAEDAAAAAALGGDEGFNMFEAAAQAGDGRGGGARSGGSEALANLDFLRSNPHFQQLRQLVQQQPHMLEPILQQVAAGNPQISQIIGQNSEQFLQLLSEEGDEEDAALPPGTQAISVTEEERDAIERLCRLGFPRDSVIQAYFACDKNEELAANFLFDQPDDDEE

SEQ ID NO: 277 - protein, 375 aa, unidentified
MKLTFKDLKQEKFVIEVEPSETVREVKQKIAQEKGEYEAERMKVIYSGKILQDDKTVESYNIQEKDFLVCLPSKGPKPAASSSASQAPATPAPRAPVATPAAPAPAAPAPASSTPAVPATPSPAGAQTGPSFGDPSALTMGSAAEGAVTQMEAMGFARSDIDRAMRAAFFNPDRAVDYLLNGIPADVQQEQQQRQQEQQADRAAEQAPVPSAEDAAAAAALGGDEGFNMFEAAAQAGDGRGGGARSGGSEALANLDFLRSNPHFQQLRQLVQQQPHMLEPILQQVAAGNPQISQIIGQNSEQFLQLLSEEGDEEDAALPPGTQAISVTEEERDAIERLCRLGFPRDSVIQAYFACDKNEELAANFLFDQPDDDEE

SEQ ID NO: 278 - protein, 342 aa, unidentified
APGDMETADAKNRAMRAAGFIVPDTFEDLPEVLKTTYTGLVQKGVIVPKAEIDPPNIPMDYQWASKLGLIRKPAAFISTISDERGQELMYAGMRISDVFKEEIGIGGVISLLWFKRRLPPFACKFIEMVLQLTADHGPAVSGAMNTIITARAGKDLISSLAAGLLTIGDRFGGALDGAAAEFSRGLNSGATPREFVDSMRKANRLIPGIGHKIKSKTNPDLRVVLVVDYVKKHFPSHKTLDFALAVEDVTTQKSNTLILNVDGAIAASFCDLLSGCGAFTEDEAADYLKNGTLNGLFVLGRSIGFIGHYLDQRLLKQPLYRHPADDIFINMQERVVFQPGSN

SEQ ID NO: 279 - protein, 182 aa, unidentified
GAYKEGKFTSESIQKSKLRFQDILVELPLRVHNSHLLTSFLHQVPQAPPAKNPLDFPSSLAELSRDSDVSSNPFAPNLDTLDLSIDPFQYWQRALGREQQKITAWQQKRKAENAARAASKQPPLDENEWQKLFKLPTEPSRLEALLVGRQVEQYARQVDGFSATVSAKMFGVRGNLLPNEIE

SEQ ID NO: 280 - protein, 171 aa, unidentified
MSAPTPSHPTLTPWTSASTPSSTGSAPSAASSRRSPHGNRSARLRMLHAPRASSRPLTRMSGRSCSSCPRSPAGSRLCLSAGRSSSTPARSTDSPPPFPPRCLASGATSSLTRSSRGRILRRRDRRLCIARRSRLGGVEYMRHGYKKKDDVVPPSTSSGQRCIESQKKKGF

SEQ ID NO: 281 - protein, 217 aa, unidentified
MRRSSKFKNVKILSSFGILCSVALNSSMAEPHHPFFCSHAACTLPHPAENASLCINAGPVSVIFVLYSISLGRRLPLTPNILAETVAENPSTWRAYCSTCLPTSRASSLLGSVGSLNSFCHSFSSRGGCLLAARAAFSALRFCCHAVIFCCSRPRARCQYWKGSMLRSRVSRLGAKGLELTSESRESSARDEGKSRGFFAGGACGTWCRKLVRRWEL

SEQ ID NO: 282 - protein, 150 aa, unidentified
MYSTPPSRERLAMHKRRSRLRNIRPLLDLVREEVAPDAKHLGGNGGGESVDLAGVLLDLPADKQSLEPAGLRGQLEQLLPLILVKGRLLARGACSILSLALLLPCGDLLLLAAEGALPVLEGVDAEVQGVKVGCEGVGADIGVARKLCKG

SEQ ID NO: 283 - protein, 269 aa, unidentified
MSNSFIRHLPSTLRAFRSSTAFSLTRSFSSTMASNGTSTNGVQHDARKVFFFDIDNCLYPKSYQIHDKMAVLIDNYFQNHLSLSQEDATTLHQRYYKDYGLAIEGLVRHHKVDPLEYNEKVDDALPLDDIIKPDPKLRKLLQDIDTDKVKLWLFTNAYVNHAKRVTRLLGVDDLFEGMTFCDYAAERLLCKPTTEMYNKAMQEANATDIDQCYFVDDSALNAAAAMKYGWKTAHLVEPTAKPPPQPVSQHQISNLEELRKVFPEVFKTS

SEQ ID NO: 284 - protein, 256 aa, unidentified
MARIFITGSTDGLGLLSAKLLSEQGHSVFLHARNAERASQAKAAVPKAQGVIIGDLSNVSDVKQLAADANKAGPFDAVVHNAGLGLTTNGQKTAEGVAQIFAVNSMAPYILTALMDKPKRLLYVSSGLHFGGDPSLEDVTWATREFRPSDAYNDTKMQNVMLSKAVAKRWPDVQSGSLDPGWVKTKLGGSAAPGTTDAPAEMIAEYAAGKSCAGDQTGAYLTPRGVEEPHDATKLAEKQDRLMQIYKEVSGVSFPQ

SEQ ID NO: 285 - protein, 201 aa, unidentified
MKLCLLGERNTRYLLVNLHQTILLLGQLSRIMRLFHATRSQVGTCLIACARFAGSVLSNHLCWSVGGARRGRPAELSLHPAWVKRAALHIRPAFGDCFREHDVLHLCIVVCIRWSELPCGPSDVLEAGVATEVQSGADVQEPLRLVHESGQNVRCHAVNGKNLGYALSSLLAIGGESEASIVNNGVKRSSLVGIGGELLHV

SEQ ID NO: 286 - protein, 192 aa, unidentified
MAIGQSSQQQADGQNVVTQGNSDKAANPMRELRIQKLVLNISVGESGDRLTRAAKVLEQLSGQTPVYSKARYTVRTFGIRRNEKISVHVTVRGAKAEEILERGLKVKEYELRKRNFSATGNFGFGISEHIDLGIKYDPAIGIYGMDFYVVMSRPGERVARRRRAKTRVGASHKVNAPEVIKWYKNRFEGIVR

SEQ ID NO: 287 - protein, 213 aa, unidentified
GHTGDVLSVSFSADNRQIVSASRDRTIKLWNTLGECKFNIVDDGHSEWVSCVRFSPNPVIPVIVSAGWDKVVKVWELSKCKLKTNHHGHTGYINTLAVSPDGSLAASGGKDGITMLWDLNDGKHLYSLEAGDIVNSLVFSPNRYWLCAATASSIKIFDLESKSIVDDLKPDFSAEYSDKAQKPQCTSLAWSADGQTLFAGFSDNLVRVWVVTA

SEQ ID NO: 288 - protein, 347 aa, unidentified
MAPSTQKQWTVKNGEQDFDGLVYGDAPVPTAGDSEVVVKLHGASLNYRDLIIPKGKYPFPLSFPVVPGSDGAGEVVEVGSKVKQFKKGDKVVTLFNQLHQYGPVDAAAASSGLGGAVDGTLRQYGVFNENGVVRAPTNLNFLESSTLTCAGLTSWNALYGLKPLLPGQTVLVQGTGGVSIFALQFAKAAGATVIATTSSEEKGKRLKDLGADHVINYKTQTNWGEIARGLTRDNIGVDHIIEVGGAGTLEQSFKCIKFEGVISIIGFLGGMNPSTIPNVLQTLSNICTVRGVYVGSKALMNDMINAIEANNIHPVVDGTVFTLEKTREAYEYMWAQKHFGKLTIQIA

SEQ ID NO: 289 - protein, 518 aa, unidentified
MLEQQYQMRKEQQVQFTPMASPSSTPYHMHQDFTVPGDFFSPLTSPALHAQNQPQSRQQFTAHQQGYYTNPSTAASSAAPSPIDANGDVEMGGDGVALPESASQPKKPSRRKPATPRTFAMNKVKQSPIQKPQKRKSVALAHKDADAVVQDAQRSGHIAPKSAGLQMPPPFESSENDSVSPEALNDLPMGPPPRPGSVSQSPAIAPQNQSVSGPAATPKSLLSMKGAQDMNAPASTGISGQMGQASLEDLELPEAAENPGSTATHSQVLNSQEPTPRLMPSRKTPKLGPLSTPSSGKPTSASNSPAHALSPMTASTPAGLLKDKKDNKGGRATSKKRGSVSTTNSAMVSPALRPKVSPSIKPLLPEGTSLNSPTHALLLASKSNYQNLLEGNHLPGISYPDSLSTGLTSKRTSHKVAEQGRRNRINDALKEMQALIPASSGARAEELMTADAGDDDSQETKEKDRDAAVKSNSSKAATVESANRYIRVLKESDAAQKDAIARPNSPGSRSLDPPDLDN

SEQ ID NO: 290 - protein, 126 aa, unidentified
MRNILLVLASAALAVVAQKPDLDVKGTFGDANPFSKVVNGQSNKLYLTLDNHSPESLVVKSISGSWSEKTSASSGQEKFLKNSTTQEKLTVPIPPKSEGAFQPPTVLTYQFWSEFKPRELLLTVLG

SEQ ID NO: 291 - protein, 91 aa, unidentified
MVLALVGGAGYLAYNIYFPPARKPRRSANTAPTDAPAAPADPDEWIPVHHKRAKKTSGGGATSGEESEATEGYASEKSASGAKKRGKGGRK

SEQ ID NO: 292 - protein, 252 aa, unidentified
MSDNNDGNHGGGVGASYYYGGIAIALCLVIVLTLVSRILYRRRVRNRLLRANRQERITLRDRGEAPGLPTYRESRNQPSLPRYTAEADYAPPPGPPPSNSPDNEGHHFHFHFPSLHVPQALHLRPRQADDPADQIPTVPPPSYEPPKYEPPSGAPPEQQQEPVASGSSEHHHQQSALGEHTAAATAAATTPAEHSGESTELRSASPSQPQSQSQPQAPAQPQEQDYGYDDADFIHPEERRRIEAAQRNDPQT

SEQ ID NO: 293 - protein, 161 aa, unidentified
MSRIGDPTNNPATQQLYSDRPLHLPGPGLKPSRQLTISSAVAFREDSGQTRFNLISSDHREVLHISIRARDNVLVLNTKAPDGDWGKEERHDLKPLFDTPLLPYITVMATKNSYILSVPGKREIIFNKRKGFMEPAVRIEYDYDEMSAFSDPCYITVPSSS

SEQ ID NO: 294 - protein, 84 aa, unidentified
MPQEIKDIKNLLEIARRKDARSARIKKTKTVGAKGEPAQLTKFKIRCSRYLYTLVVSDGEKAEKLKQSLPPTLNVEEIGKVSKK

SEQ ID NO: 295 - protein, 173 aa, unidentified
MSHTFYDGTIVVLQGILETFSHILHKAEESPNSSAFPAARLHEDMYPLTDQIRLATQFSEYILAKVTGREPRKFEGNPLTFAEFYERIDTMLKSLKEADKDVVNANADKEELTQVGPTAKIELSNAIYAHRIALPNIYFHLNIAYGILRKEGVPLGKLDYFAGFFPPSMAQGK

SEQ ID NO: 296 - protein, 162 aa, unidentified
MSRIGDFANNNQATQQLFSDRPMQLPGPGLKPSRQLTVSSAMAFRWDSGQTRFNLISSDRREVLHISIRAKDDVLVLNTKAPDGNWGKEERHELKPLFDTPMLPYITVTATKTSYILSVPGNQEIIFNKRKGFMEPAVKIEYDYDENPAFSDPCYVTVPHLS

SEQ ID NO: 297 - protein, 313 aa, unidentified
MSEQLHYKGSLAGHGNWVTAIATSAENPDMILTASRDKSVIVWQLTRDDAQYGYPKRILKGHNHFVSDVSISYDGQFALSSSWDKTLRLWDLNTGLTTRRFVGHEADVLSVSFSADNRQIVSGSRDRTIKLWNTLGECKFDIKDEGHSEWVSCVRFSPNPMNPVIVSAGWDKVVKVWELSNCKLKTNHYGHTGYINTVSVSPDGSLAASGGKDGITMLWDLNEGKHLYSLEAGDIVNALVFSPNRYWLCAATASCIKIFDLESKSIVDELKPDFVDVGKNSREPEAVSLSWSADGQTLFAGFTDNAVRVWTVA

SEQ ID NO: 298 - protein, 107 aa, unidentified
GEGGGIHGTTFNSIMKCDVDVRKDLYGNIVMSGGTTMYPGIADRMQKEITALAPSSMKVKIIAPPERKYSVWIGGSILASLSTFQQMWISKQEYDESGPSIVHRKCF

SEQ ID NO: 299 - protein, 111 aa, unidentified
MRLSLEALAVDDRRAALVVLLLRDPHLLEGGQRSQDGTTNPHRVFTLRRSNDLDLHRRRSKSGDFLLHTVGNTRVHSSTTRHDNVAIEILTDVNITLHDGVEGGSVDTTTF

SEQ ID NO: 300 - protein, 213 aa, unidentified; identical to SEQ ID NO: 149

SEQ ID NO: 301 - protein, 161 aa, unidentified; identical to SEQ ID NO: 293

SEQ ID NO: 302 - protein, 178 aa, unidentified
MKAYWYDNQPGDQRLPHDSGRPVTESYLESIGVFYRHCPTIDLVDSLAAERGYKNRDEVCVSPQTMGDVYEEKVKTFFSEHLHEDEEIRYIRDGEGYFDVRGQEDEWVRIRLSKDDLIILPAGIYHRFTTDDKNYVKAMRLFQEEPKWTPLNRGPEVDVNPHRKTYLETVPSPAVAAN

SEQ ID NO: 303 - protein, 216 aa, unidentified
VAAGYTPEALEILSKKKGGKYLVLEMDETYNPPAEETRTLYGVQLTQARNDAVISPQKTFNTIITPKNTESLPESALRDLTVATLALKYTQSNSVCYALNGQVVGLGAGQQSRIHCTRLAGDKTDNWWMRFHERVLNIKWKQGTKRADKSNAIDLLCSGQTPRNDAEKVEYERVFAEVPAPFTQEERDAWLSQLTNVAISSDAFVCLSPLLEHSKF

SEQ ID NO: 304 - protein, 51 aa, unidentified
FPFIDNVFRAARSGVKYIAAPSGSQNDGPVFETAEKLGISFVEQGTRLFHH

SEQ ID NO: 305 - protein, 255 aa, unidentified
VAAGYTPEALEILSKKKGGKYLVLEMDETYNPPAEETRTLYGVQLTQARNDAVISPQKTFNTIITPKNTESLPESALRDLTVATLALKYTQSNSVCYALNGQVVGLGAGQQSRIHCTRLAGDKTDNWWMRFHERVLNIKWKQGTKRADKSNAIDLLCSGQTPRNDAEKVEYERVFAEVPAPFTQEERDAWLSQLTNVAISSDAFFPFIDNVFRAARSGVKYIAAPSGSQNDGPVFETAEKLGISFVEQGTRLFHH

SEQ ID NO: 306 - protein, 316 aa, unidentified
MCDRSRIGPKGWEAVVSTQAVVSTQAVVSTQAVVSTQAVVSTQAVVSTQSAQPGAISAPVAAGKDVELQWTEWPESHHGPVITYLANCNGDCSEVDKSSLEFFKIDQKGLIDDSNVPGTWATDKLISNNNSYTVTIPSDIAAGNYVLRHEIIALHSAGNEDGAQNYPQCLNLKVTGGGNASPSGTLGTKLYNEDDSGILVSIYQQLDSYDIPGPALYSGASSSSNSGSSSSVASATASATSAAASSPSSSQASGTPASQVKAQTASSTPSASSGATSGSLSDYFSSLSAEEFLNVISETLSWLVTDKIHARDLSTA

SEQ ID NO: 307 - protein, 138 aa, unidentified
MKSLLCTPLATRMVPRTTPSVSTSRLLVVATLLPQVLLVPSSTTRTTRVSLSVSTSSLTPTTSPALLCTLALPRPPTLVLLPALLRPLLLPLLPLLPLPRPLRLPVPPLPRSRLRPLALLLALRPVPLPAVCPTTSAL

SEQ ID NO: 308 - protein, 272 aa, unidentified
VVSTQSAQPGAISAPVAAGKDVELQWTEWPESHHGPVITYLANCNGDCSEVDKSSLEFFKIDQKGLIDDSNVPGTWATDKLISNNNSYTVTIPSDIAAGNYVLRHEIIALHSAGNEDGAQNYPQCLNLKVTGGGNASPSGTLGTKLYNEDDSGILVSIYQQLDSYDIPGPALYSGASSSSNSGSSSSVASATASATSAAASSPSSSQASGTPASQVKAQTASSTPSASSGATSGSLSDYFSSLSAEEFLNVISETLSWLVTDKIHARDLSTA

SEQ ID NO: 309 - protein, 138 aa, unidentified; identical to SEQ ID NO: 307

SEQ ID NO: 310 - protein, 305 aa, unidentified
MLSMFTRVARGQAKVFTRNASTASSKPTNQSSNKAATIAASISGVTAALYAHQYGLIDSVFASGLEEGLHAPHFPWSHNGWLDSFDHNSIRRGYQVYREVCSSCHSLDRIAWRNLVAVSHTSDEARAMAEEQEYTDGPNDQGESFQRPGKLADYMPAPYPNEEASRAANGGALPPDLSLIVKARHGGADYIMALLTGYQDPPAGIQVQEGMNFNPYFPGGGIAMGRVLFDGLVEYDDGTPATTTQMAKDVATFLSWASEPEHDDRKKMGFQAVIILSAMTAISLYVKRLKWSPIKTRKLTYNPPK

SEQ ID NO: 311 - protein, 165 aa, unidentified
GGSPAKKSLIGAMEAQNLKTFPKQPIFQNSKTRGNKKVTKDRRWYKDVGLGFKTPQEAITGTYIDKKCPWTGEVSIRGRILSGKVVSTKMTRTIVIRREYLHYVPKYNRYEKRHKNLPVHASPAFRIENGDQVVVGQCRPLSKTVRFNVLRVIKNKAAAKAFAKF

SEQ ID NO: 312 - protein, 72 aa, unidentified
MPPRKPRCSFKECKEQAQRIVGDCSFCSGHFCSKHRMLEAHSCSGLEDCKKESHARNADKLNSERTQVIKGV

SEQ ID NO: 313 - protein, 161 aa, unidentified
MSRNFGDFSTNQATQQLYSDRPLHLPGNGLKPARQLTISSAVAFRWDSDQTRFNLISSDRREVLHISIRAKDNVLVLNTKAPDGDWGREERHELKKLFDTPMLPYITVTATKMTYNITVPSGQEIIFNKRKGFMEPAVKIEYDYDEHSAFSDPCYITVPSS

SEQ ID NO: 314 - protein, 119 aa, unidentified
LLASNSRSNELSSPPSTHLHISQSTMVSKILFWSGFGIAVRLWQLGIEMRPILAKQGLWAYPVFAGVGGSFGYWLQGVEDRQLKILAQRREAILDKRRRRDEREGLSNIEKEGTLAATP

SEQ ID NO: 315 - protein, 91 aa, unidentified
LNWSCNFADCWPRTPDRTNSPLLRQRTFTYRKAQWFPRFSSGVASASPSVSGNSVSKCVPFLPSRVSGPTPSSQVSVEASVTGSRVSRTVS

SEQ ID NO: 316 - protein, 182 aa, unidentified
MIHAQQCNCRSFSEGSENKQQPTQQIMGSQPKYPPSQCCSDPHARPVSGACRGWLRGAAQESSADGPRHPGASNRSFHRHLRRRGRPRDPAWQEWDAFRYRVARDGRRCRSHSRRESWKPLCFAICEGALTEERRVRSIGSSRPAISEIAGPIQTPATKANTAGDNRESGESTPANKKFSAR

SEQ ID NO: 317 - protein, 127 aa, unidentified
MPSNAIAGLSPKAVKINNSQRNKSWGRSQSTLLLNVAQTLTLVPSPALVEDGFAALRKNLQLTVLDTLEPVTEASTDTCEDGVGPETLLGKNGTHFDTELPETDGDAEATPEENLGNHCALRYVKVR

SEQ ID NO: 318 - protein, 300 aa, unidentified
MPFIKEAKSNSYFSRYQVKYRRRREGKTDFYARKRLVTQAKNKYNAPKYRLVVRFTNKDIICQIVSSKLQGDVVLTHARARELPRYGIKHGLTSWSSAYAVGLLVARRALTKLGLADKYEGDVEATGEYNLTEPLGDDEPRPFKVFLDVGLKRTSTGSRVFGALKGASDGGLYIPHSENRFPGYDIESKELDAEILNKYILGGHIAEYMEALEEEDEERFKAQFSTYLEDGIGSEDIEEIFSGAHEAIRADPTFKPSEAAKGTDWKSESKKHRAVRLTKQQREDAIQQRIKYYQQAGDLE

SEQ ID NO: 319 - protein, 212 aa, unidentified; identical to SEQ ID NO: 164

SEQ ID NO: 320 - protein, 192 aa, unidentified
MTEKLYTEQVNAFGNELPPLSYKDLDKLPLHQNVIKETLRIHNSIHTLMRKVKNPLPVPGTRFVIPTSHTLLASPGVTTRDDSHFRNAMTWDPHRWETRSEVEDDGETIDYGYGVVSKGTKSPYLPFGAGRHRCIGEKFAYLNLTVIVATLVRNFRFSEPDDREGVPETDYSSLFSRPMRPATARWERRGEY

SEQ ID NO: 321 - protein, 210 aa, unidentified
MAHWREEYLTALAVRDQREKANLSIYDAYTRLADSTAKLPATIDTSGSPSGDKGPSGTYESEKTAFSQSRTAKKQQTEVEPSVTELLNTTRAELAEAQRSRAELRDRLERATNEAEKLRKQIGKDGRRIHGLENEVAQQQKRRKDVEEELRGKAKLLNEFQDEIAALTLQVNMAERKAKKLGEENDDLVNRWMKRMGQEADAMNDASKFS

SEQ ID NO: 322 - DNA, 53 bp, synthetic construct
  misc_feature 1..53: note = poly-A mRNA primer
  misc_feature 52..53
  misc_feature 52: note = a,c or g
  misc_feature 53: note = n is a, c, g, or t
  source 1..53: mol_type = other DNA
attctagagcgatcgcacatgttttttttttttttttttttttttttttttvn

SEQ ID NO: 323 - DNA, 34 bp, synthetic construct
  misc_feature 1..34: note = poly-A mRNA primer
  misc_feature 32..33: note = Ribonucleotide Guanosine
  source 1..34: mol_type = other DNA
  misc_RNA 34: note = Locked Nucleic Acid (LNA)
aagcagtggtatcaacgcagagtggcgcgccggg

SEQ ID NO: 324 - DNA, 34 bp, synthetic construct; identical to SEQ ID NO: 323
  misc_feature 1..32: note = oligo-capping rapid amplification of cDNA ends primers
  misc_feature 1: note = 5' Inverted Dideoxy-T
  misc_feature 2..32: note = Ribonucleotide
  source 1..34: mol_type = other DNA

SEQ ID NO: 325 - DNA, 33 bp, synthetic construct
  misc_feature 1..33: note = primer for amplification of prokaryote cDNA library
  misc_feature 1: note = 5' Inverted Dideoxy-T
  misc_feature 2..33: note = Ribonucleotide
  source 1..33: mol_type = other DNA
taagcagtggtatcaacgcagagtggcgcgccg

SEQ ID NO: 326 - DNA, 20 bp, synthetic construct
  misc_feature 1..20: note = primer for CaMV 35S promoter
  source 1..20: mol_type = other DNA
ccactgacgtaagggatgac

SEQ ID NO: 327 - DNA, 20 bp, synthetic construct
  misc_feature 1..20: note = primer for CBF3 promoter
  source 1..20: mol_type = other DNA
cagcatgctctcactccaac

SEQ ID NO: 328 - DNA, 20 bp, synthetic construct
  misc_feature 1..20: note = primer for Erd10 promoter
  source 1..20: mol_type = other DNA
cgtgagaatgacacaaccac

SEQ ID NO: 329 - DNA, 18 bp, synthetic construct
  misc_feature 1..18: note = primer for Kin1 promoter
  source 1..18: mol_type = other DNA
ctcgtggcaccacactcc

SEQ ID NO: 330 - DNA, 19 bp, synthetic construct
  misc_feature 1..19: note = primer for NOS/HSP terminator
  source 1..19: mol_type = other DNA
ggaaattcgcctcgagatc

SEQ ID NO: 331 - DNA, 1391 bp, Arabidopsis thaliana
  source 1..1391: mol_type = genomic DNA
ataccggagctcagagaatcatatgactaaggacgtggtggttgaaggaaatgagagaatacatgaagaagagaaacttctttgagtgagaaggaagtgcgctggctggagagaaaagagagaaaagagtttcgagtgagagagagggcgttgagattgtgatcaacttaatgtaatatgttcttttattacattttctttttgtcatatactcaaaccttttactattttgtctcataaatctaacacaccccaccatttgttaatgcatgatggtagaaaatattaaatataattaactacttttatgtgatcaaaattaggtttcagactcgtttcgcgatccgatctacaattacaactgcatgcttctaattgatctaaattctaaattttttatacatattaaaaaaacaactttttgttaaattctcaatcatcatttttgtgattaacaattttttataactctaaaccaataatatttgattatttattttatatgtataatgatgattgagaattttaattagcagtctatttagggttttcctaaagttacaatatgttgttacccttctagttaaattttccaaaataccatatttcataacttttcaaactgtttattaattcaaccgtaaaaagcactaaaatgttacatttgatcattcacccaaattaaattcaaaagtttttccgccaaaactacttggtgacttacgtgcttatatacggacgactattattatgttctatacttttttatactttgttgcacaaatatctactctcccaattcatattctagaaggatgtgctataagaatgggagaaattacacaagaagagcatctttaaatatcctctcacaatctttatgtctaatacacgggtgaacaattaacgacaatttctttattcaggaatataataatgaataacggttaccctacacctagtacactaaatccttaacagccacacattcatacgcaaagagtttataaaactcataaaggtataataataacgagtgaataagtcaaaaaaagtcttctctggacacatggcagatcttaatgagtgaatccttaaactactcattttacaattgcttcgctgtgtatagtttacgtggcattaccagagacacaaactccgtcttcgccttttcttttgcctctaaaatatcttccgccattataaaacagcatgctctcactccaacttttatttatctacaaacattaaatccacctgaactagaacagaaagagagagaaactattatttcagcaaaccataccaacaaaaaagacagagatcttttagttaccttatccagtttcttgaaacagagtactcttctgatcaggcgcgccgccata

SEQ ID NO: 332 - DNA, 1215 bp, Arabidopsis thaliana
  source 1..1215: mol_type = genomic DNA
ataccggagctcgactaggtggacaaaataatttgttaattaaataaaaattagttcaatatagaaatgaaaacgattgctttgtttggtatgtgtcggtacagtgaccatcctaatgcctaatataaaagattcgatcggtatgttacaagttgcgtgtatatgaaaacgtcacattttattaagtggcacgtcgttgtgatgaatctttcaaccgaacacgattcataatctataagcaaaatccgaaaatggtgccttctcaatgccccctatctgttcaatctttttttttttttttttttttgtatatctgttcaatcttatttaaatgtaatgacaaattaaatgaagtttacgttagtaatataagctgacaaacaacaccacattacatacataaaattaaattcttttaagtatttcaataacgtttctttatcttaaaaattaaatttacttcgagagcttcctacttcgtcaaaaataaaattttactttgctgcatgtttttactttctttttgtaacgtcttaaaaaggtgattaacgtcaacttaattcaccgaaagtctctgcaattgatattttctgccgacgtggcataagaagtccgattggcccacatgaccgacatccacgcttaaaccaatcaaaaccggccattcagttccatctgtgggctcctgaaacgttctcttgacacgtgtttaccatatattggcttaatccatccatagtctttctatttactgacaggtagtatttttcctatcaattatttattttcacgtggcatgatggctatggctagttgaacctgtgaataacttggtcatatctactctctatttatttagatgattcattttcttgaaggacttgcaattttatccccttacttttatttctttgagagataacctaaaattctcaaaatgagttggaaacatccctttgaagttcctctacaggctttctatgtgcataagaatctgcttaacattggaaataatatatgcattcttctccaattctcctagttggatacatatatgaagtctataaattacacatatttcccacaaaaattattgtaagagtttatatttcaacatatagtatgcaaacttaaatcgtgagaatgacacaaccactaattcaaaccactacattatatattctaatccattcaaattcatggcgcgccgccata

SEQ ID NO: 333 - DNA, 960 bp, Arabidopsis thaliana
  source 1..960: mol_type = genomic DNA
ataccggagctcggtaacttgaattcaaccatgaactgtttggattggcaaacataaactcaaataaaatatctaggtataattgtggttcatacaagaattacttcatactgttgggccaaaggacgtatccttccccgcacctccaaaccatgggcttactactgatccgacatcaaaaccgtgttagttgcaaccaacgaatgataagtcaataagattcaacttgtcaacaaatatacagcttatatgacatgtctggctccaaactgaattttagtagaaagttactaattcataaaattaatttatatacaatttttcaatttttattttataaattaaagaaaaaaacatgaaaaatacgggaggttcggcaaacacaacatttaacttgccaaacgtatcatctaactttcccaccttatacaaggaaccattttttcaataataaagtttttttttttttgtcttcgcaaataagagcacgaaatgtttgccaaacgcatatgcaacaaacccacgttacataattctgtttacagccatagagcaagctatattgttaaagacctaaaaaaaactttactataacatatagaggcttcgagatatttcgaaagactcaacttatatataaataaactcaaaaagaaaacacggaggcgagaggatcatactctcacacagaaagagtcacattattatatcctctaaaaaaccaaactaaaacgacacgtgaagtcttgatcagccgataaatagctaccgacataaggcaaaactgatcgtaccatcaaatgtaatccacgtggttttagattactcgtggcaccacactccctttagcctataaatataaaccattaagcccacatctcttctcatcatcactaaccaaaacacacttcaaaaacgattttacaagaaataaatatctgaaaaaggcgcgccgccata